(19) United States
(12) Patent Application Publication
(10) Pub. No.: US 2024/0317868 A1
(43) Pub. Date: Sep. 26, 2024

(54) ANTI-IL1RAP ANTIBODIES

(71) Applicant: Boehringer Ingelheim International GmbH, Ingelheim am Rhein (DE)

(72) Inventors: Tammy J. BIGWARFE, Newtown, CT (US); Karim Christian EL KASMI, Biberach an der Riss (DE); Ye GU, Winchester, MA (US); Stefan HOERER, Biberach an der Riss (DE); Daniel PETER, Ummendorf (DE); Melanie GRONER, Ummendorf (DE); Felix SCHIELE, Bellenberg (DE); Anita BLOCHING, Laupheim (DE)

(21) Appl. No.: 18/275,616

(22) PCT Filed: Feb. 4, 2022
(86) PCT No.: PCT/US2022/015163

§ 371 Date: Aug. 3, 2023

Related U.S. Application Data
(60) Provisional application No. 63/146,051, filed on Feb. 5, 2021.

(51) Int. Cl. C07K 16/28; A61P 37/06

(52) U.S. Cl. CPC C07K 16/2866; A61P 37/06; C07K 2317/24; C07K 2317/565; C07K 2317/76; C07K 2317/92

(57) ABSTRACT

The present invention relates to anti-IL1RAP binding compounds, in particular new anti-IL1RAP antibodies and therapeutic and diagnostic methods and compositions for using the same.

### SEQUENCE LISTING

The instant application contains a Sequence Listing which has been submitted electronically in ASCII format and is hereby incorporated by reference in its entirety. Said ASCII copy, created on Feb. 3, 2022, is named 09-0717-WO-1_SL.txt and is 194,305 bytes in size.

### TECHNICAL FIELD OF THE INVENTION

This invention generally relates to anti-IL1RAP antibodies for diagnostic and therapeutic use. The antibodies can be used in pharmaceutical compositions and kits comprising such compounds. The antibodies are useful in methods for the treatment of various diseases or disorders, for example immunological, inflammatory, autoimmune, fibrotic and respiratory diseases in humans.

### BACKGROUND OF THE INVENTION

The IL-1 family of cytokines is composed of 11 different ligands, namely, IL-1α (also termed IL-1F1), IL-1β (IL-1F2), IL-1 receptor antagonist (IL-1Ra or IL-1F3), IL-18 (IL-1F4), IL-1F5 to IL-1F10, and IL-1F11 (or IL-33). IL-1α and IL-1β are known to induce pro-inflammatory activities on binding to type I IL-1 receptor (IL-1RI) and recruitment of the common co-receptor IL-1 receptor accessory protein (IL-1RAcP), whereas IL-1Ra acts as a competitive inhibitor of IL-1 binding to IL-1RI, thus exerting anti-inflammatory activity. Numerous studies reported that IL-18 is a pro-inflammatory cytokine that is an inducer of IFN-γ, whereas IL-33 was described as an immunoregulatory cytokine involved in particular in the control of Th2 responses.

The IL1-receptor accessory protein, IL1RAP, is the co-receptor for the primary receptors (IL1R1, IL33R and IL36R) required for signaling of the cognate IL1 family cytokines IL1 (α, β), IL33 and IL36 (α, β, γ).

IL1 family cytokines IL1 α and IL1 β, IL33, and IL36 α, IL36 β, and IL36γ bind to their respective receptors IL1R1, IL33R, and IL36R. The cognate receptors then bind to the IL1-receptor accessory protein, IL1RAP (also designated IL-1RAP), which serves as the co-receptor that results in downstream activation mediated by IL1, IL33 and IL36, respectively.

IL1RAP is expressed in tissues where IL1-, IL33-, or IL36-receptors are present, such as lymph nodes (thymus, tonsil), bone marrow, brain, lung, skin, gut, liver and placenta. Myeloid leukemia stem cells in CML reportedly also express IL1RAP (Jaras, et al. (2010). Isolation and killing of candidate chronic myeloid leukemia stem cells by antibody targeting of IL-1 receptor accessory protein. Proc Natl Acad Sci USA 107, 16280-16285).

The importance of IL1RAP in the signaling of the IL1 family of alarmins has been confirmed by genetic deletion of the co-receptor IL1RAP that led to a complete loss of signaling for IL1 (IL-1 i.p. injection (Cullinan, et al. (1998). IL-1 receptor accessory protein is an essential component of the IL-1 receptor. J Immunol 161, 5614-5620)); and IL33 (mast cells (Palmer, et al. (2008). The IL-1 receptor accessory protein (AcP) is required for IL-33 signaling and soluble AcP enhances the ability of soluble ST2 to inhibit IL-33. Cytokine 42, 358-364.)) as well as by transfection studies/pharmacological intervention for IL36 (Towne, et al. (2011). Interleukin-36 (IL-36) ligands require processing for full agonist (IL-36alpha, IL-36beta, and IL-36gamma) or antagonist (IL-36Ra) activity. J Biol Chem 286, 42594-42602). Individual cytokine inhibitors have been well reported however, when tested in vivo in clinical trials, these inhibitors failed implying a single neutralizing antibody against one or two IL1 cytokines may not be sufficient to a desired clinical efficacy.

In published literature, only a handful of IL1RAP antibodies have been described. Of note, most of these antibodies delivered only some level of bioactivity against some of the ligands, and it is considered challenging to identify an antibody with the requisite potency against all of the potential ligands IL1-α, IL1-β, IL-33 and IL-36α, β, γ suggesting that the IL1RAP binding interface may differ between the different co-receptors. As such, the binding epitope appears to be is crucial for the effect since not all antibodies have the ability to block signaling even if they do have the ability to bind IL1RAP and mediate efficient ADCC (Agerstam, et al. (2015) PNAS 112 (34): 10786-91)

The diverse nature of inflammatory disease may explain the less than optimal outcomes or even failures in clinical trials when researchers have only attempt to neutralize a single disease-driving cytokine at a time, particularly when in normal cytokines synergize and or provide feedback on other cytokine members in the pathway. The IL-1 family may represent such a convergence in many inflammatory and disease states. Diseases where more than one IL-1 family member has been attributed a prominent role, such as IL-1β and IL-33 in asthma (Lappalainen, et al. (2005) Interleukin-1β causes pulmonary inflammation, emphysema, and airway remodeling in the adult murine lung. Am. J. Respir. Cell Mol. Biol. 32, 311-318; Prefontaine, D. et al. (2009) Increased expression of IL-33 in severe asthma: evidence of expression by airway smooth muscle cells. J. Immunol. 183, 5094-5103), and IL-1α and IL-36 in psoriasis (Towne, J. E. & Sims, J. E. (2012) IL-36 in psoriasis. Curr. Opin. Pharmacol. 12, 486-490 (2012); and Tortola, L. et al. (2012) Psoriasiform dermatitis is driven by IL-36-mediated DC-keratinocyte crosstalk. J. Clin. Invest. 122, 3965-3976). Thus, optimal outcomes might be expected by blockade of multiple IL-1 associated pathways.

Recent publications disclosed the findings in vitro that their IL1RAP antibodies specifically inhibits the signaling of all the six IL-1 family member (IL-1, IL-33, and IL-36) signaling. Recently, Højen et al published their finding of a mAb against IL1RAP that specifically inhibits IL-1, IL-33 and IL-36 signaling in vitro, and also significantly attenuated heterogeneous cytokine-driven inflammation and disease severity (Højen et al (2019) IL-1R3 blockage broadly attenuates the function of six members of the IL-1 family, revealing their contribution to models of disease. Nature Immunol. 20:1138-1149).

As IL1RAP is the co-receptor in three signaling pathways that involve six cytokines of the IL-1 family (IL-1α, IL-1β, IL-33, IL36α, IL-36β and IL-36γ and many disease are driven by these cytokines, a single antagonistic agent, such an antibody that could inhibit all pathways, would be of considerable therapeutic benefit, and particularly for useful in the treatment of inflammatory diseases.

Therefore, what is necessary is an IL1RAP antibody having a broader simultaneous inhibition across multiple or all IL1 cytokines and a more potent bioactivity.

### SUMMARY OF THE INVENTION

The present invention addresses the above need by providing biotherapeutics, in particular antibodies, which bind to IL1RAP. In one aspect, the antibodies of the present invention block IL1RAP-mediated cytokine signaling via the IL-33 and IL-36 signaling pathway family of key inflammatory cytokines. In one aspect the antibodies of the present invention are useful, for example for the treatment of epithelial-mediated inflammation/fibrosis in diseases such as psoriasis, asthma, autoimmune disease, acute infection scleroderma, COPD, and chronic kidney disease.

In one aspect, the present invention provides an anti-IL1RAP antibody having one or more of the properties below.

In one aspect, an anti-IL1RAPR antibody of the present invention has high molecular/cellular binding potency. In one aspect, an anti-IL1RAP antibody of the present invention binds to human IL1RAP at a K_{D}<1.0 nM. In a further aspect, an anti-IL1RAP antibody of the present invention, in particular a humanized anti-IL1RAP antibody, binds to human and cynomolgus monkey IL1RAP at a K_{D}<200 pM.

In embodiment two, the present invention provides an anti-IL1RAP antibody or antigen-binding fragment thereof according to embodiment one, wherein the said antibody or antigen-binding fragment is a monoclonal antibody or antigen-binding fragment thereof.

In embodiment three, the present invention provides an anti-IL1RAP antibody or antigen-binding fragment thereof according to embodiment one or two, wherein the said antibody or antigen-binding fragment is a humanized antibody or antigen-binding fragment thereof.

In embodiment four, the present invention provides an anti-IL1RAP antibody or antigen-binding fragment thereof according to embodiment three, which blocks IL-1, IL-33 and IL-36 signaling.

In embodiment five, the present invention provides an anti-IL1RAP antibody or antigen-binding fragment thereof according to any one of embodiment one to four, which does not bind to human IL-1R1, IL-33R, and IL-36R.

In embodiment six, the present invention provides an anti-IL1RAP antibody or antigen-binding fragment thereof according to embodiment one, wherein the antibody or antigen-binding fragment thereof comprises:
a) a light chain variable region comprising the amino acid sequence of SEQ ID NO: 3, 6, 117, 118, 119, 121, 122, 123, 124, 125, 126, 127, 128, 129, 130, 131, 132, 133, 134, or 135 (L-CDR1); the amino acid sequence of SEQ ID NO: 4, 7, 136, 137, 138, 139, 140, or 141 (L-CDR2); the amino acid sequence of SEQ ID NO: 5 (L-CDR3); and
b) a heavy chain variable region comprising the amino acid sequence of SEQ ID NO: 8, 11, 12, 14, 142, 143, 144, 145, 146, or 147 (H-CDR1); the amino acid sequence of SEQ ID NO: 9, 13, 15, 148, 149, 150, 151, 152, 153, 154, 155, 156, 157, 158, 159, 160, 161, 162, 163, 164, 165, 166, or 167 (H-CDR2); the amino acid sequence of SEQ ID NO: 10, or 16 (H-CDR3).

In embodiment seven, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
f) a heavy chain variable region comprising the amino acid sequence of SEQ ID NO: 112 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

In embodiment eight, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 112 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO: 7 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

In embodiment nine, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 112 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO:139 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

In embodiment ten, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO:9 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO:9 (H-CDR2); the amino acid sequence of SEQ ID NO: 10 (H-CDR3), or.
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO:13 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO:15 (H-CDR2); the amino acid sequence of SEQ ID NO: 16 (H-CDR3).

In embodiment eleven, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment twelve, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 153 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 13 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 15 (H-CDR2); the amino acid sequence of SEQ ID NO: 16 (H-CDR3).

In embodiment thirteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment fourteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment fifteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment sixteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 151 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 151 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 163 (H-CDR2); the amino acid sequence of SEQ ID NO: 16 (H-CDR3).

In embodiment seventeen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO:9 (H-CDR2); the amino acid sequence of SEQ ID NO: 10 (H-CDR3), or
c) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO:9 (H-CDR2); the amino acid sequence of SEQ ID NO: 10 (H-CDR3), or
e) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO:13 (H-CDR2); the amino acid sequence of SEQ ID NO: 10 (H-CDR3), or
g) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO:15 (H-CDR2); the amino acid sequence of SEQ ID NO: 16 (H-CDR3).

In embodiment eighteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO: 7 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment nineteen, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and d) a heavy chain variable region comprising the amino acid sequence of SEQ ID NO: 144 (H-CDR1); the amino acid sequence of SEQ ID NO: 153 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 13 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO: 7 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 15 (H-CDR2); the amino acid sequence of SEQ ID NO: 16 (H-CDR3).

In embodiment twenty, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO: 7 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment twenty one, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO:139 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment twenty two, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO:139 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

In embodiment twenty three, the present invention provides an anti-IL1RAP antibody or antigen-binding fragment thereof according to embodiment six, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 151 (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 151 (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO:139 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 163 (H-CDR2); the amino acid sequence of SEQ ID NO: 16 (H-CDR3).

In embodiment twenty-four, the present invention provides an anti-IL1RAP antibody or antigen-binding fragment thereof according to embodiments seventeen-twenty three, respectively, wherein the antibody or antigen-binding fragment thereof comprises a light chain variable region comprising the amino acid sequence of SEQ ID NO:17; and a heavy chain variable region comprising the amino acid sequence of SEQ ID NO: 67; or
a light chain variable region comprising the amino acid sequence of SEQ ID NO: 36; and a heavy chain variable region comprising the amino acid sequence of SEQ ID NO: 86; or
a light chain variable region comprising the amino acid sequence of SEQ ID NO: 40; and a heavy chain variable region comprising the amino acid sequence of SEQ ID NO: 90; or
a light chain variable region comprising the amino acid sequence of SEQ ID NO: 47; and a heavy chain variable region comprising the amino acid sequence of SEQ ID NO: 97; or
a light chain variable region comprising the amino acid sequence of SEQ ID NO: 50; and a heavy chain variable region comprising the amino acid sequence of SEQ ID NO: 100; or
a light chain variable region comprising the amino acid sequence of SEQ ID NO: 51; and a heavy chain variable region comprising the amino acid sequence of SEQ ID NO: 101; or
a light chain variable region comprising the amino acid sequence of SEQ ID NO: 52; and a heavy chain variable region comprising the amino acid sequence of SEQ ID NO: 102.

In embodiment twenty-five, the present invention provides an anti-IL1RAP antibody, wherein the antibody comprises a light chain comprising the amino acid sequence of any one of SEQ ID NO: 170, 171, 172, 173, 174, 175, or 176; and a heavy chain comprising the amino acid sequence of any one of SEQ ID NO: 177, 178, 179, 180, 181, 182, or 183.

In embodiment twenty six, the present invention provides an anti-IL1RAP antibody according to embodiment twenty five, wherein the antibody comprises a light chain comprising the amino acid sequence of SEQ ID NO: 170; and a heavy chain comprising the amino acid sequence of SEQ ID NO: 177.

In embodiment twenty seven, the present invention provides an anti-IL1RAP antibody according to embodiment twenty five, wherein the antibody comprises a light chain comprising the amino acid sequence of SEQ ID NO: 171; and a heavy chain comprising the amino acid sequence of SEQ ID NO: 178.

In embodiment twenty eight, the present invention provides an anti-IL1RAP antibody according to embodiment twenty five, wherein the antibody comprises a light chain comprising the amino acid sequence of SEQ ID NO: 172; and a heavy chain comprising the amino acid sequence of SEQ ID NO: 179.

In embodiment twenty nine, the present invention provides an anti-IL1RAP antibody according to embodiment twenty five, wherein the antibody comprises a light chain comprising the amino acid sequence of SEQ ID NO: 173; and a heavy chain comprising the amino acid sequence of SEQ ID NO: 180.

In embodiment thirty, the present invention provides an anti-IL1RAP antibody according to embodiment twenty five, wherein the antibody comprises a light chain comprising the amino acid sequence of SEQ ID NO: 174; and a heavy chain comprising the amino acid sequence of SEQ ID NO: 181.

In embodiment thirty one, the present invention provides an anti-IL1RAP antibody according to embodiment twenty five, wherein the antibody comprises a light chain comprising the amino acid sequence of SEQ ID NO: 175; and a heavy chain comprising the amino acid sequence of SEQ ID NO: 182.

In embodiment thirty two, the present invention provides an anti-IL1RAP antibody according to embodiment twenty five, wherein the antibody comprises a light chain comprising the amino acid sequence of SEQ ID NO: 176; and a heavy chain comprising the amino acid sequence of SEQ ID NO: 183.

In one embodiment, an antibody or antigen-binding fragment thereof according to any one of embodiments one to thirty two is a monoclonal antibody. In one embodiment, an antibody or antigen-binding fragment thereof according to any one of embodiments one to twenty-five is a humanized antibody. In one embodiment, an antibody or antigen-binding fragment thereof according to any one of embodiments one to twenty-five is a monoclonal humanized antibody.

In embodiment thirty three, the present invention provides an anti-IL1RAP antibody or antigen-binding fragment thereof, wherein the antibody or antigen-binding fragment thereof comprises:
a light chain variable region comprising the amino acid sequence of SEQ ID NO: 3, 117, 118, 119, 120, 121, 122, 123, 124, 125, or 134 (L-CDR1); the amino acid sequence of SEQ ID NO: 4, 136, 137, 138, or 140 (L-CDR2); the amino acid sequence of SEQ ID NO: 5 (L-CDR3); and a heavy chain variable region comprising the amino acid sequence of SEQ ID NO: 8, 142, 143, or 146 (H-CDR1); the amino acid sequence of SEQ ID NO: 9, 148, 149, 150, 151, 152, 153, 154, 155, 156, 157, 158, 159, 160, or 165 (H-CDR2); the amino acid sequence of SEQ ID NO: 10 (H-CDR3), or
a light chain variable region comprising the amino acid sequence of SEQ ID NO: 3, 117, 118, 119, 120, 121, 122, 123, 124, 125, or 134 (L-CDR1); the amino acid sequence of SEQ ID NO: 4, 136, 137, 138, or 140 (L-CDR2); the amino acid sequence of SEQ ID NO: 5 (L-CDR3); and a heavy chain variable region comprising the amino acid sequence of SEQ ID NO: 11, 144, 145, or 147 (H-CDR1); the amino acid sequence of SEQ ID NO: 9, 148, 149, 150, 151, 152, 153, 154, 155, 156, 157, 158, 159, 160, or 165 (H-CDR2); the amino acid sequence of SEQ ID NO: 10 (H-CDR3), or
a light chain variable region comprising the amino acid sequence of SEQ ID NO: 3, 117, 118, 119, 120, 121, 122, 123, 124, 125, or 134 (L-CDR1); the amino acid sequence of SEQ ID NO: 4, 136, 137, 138, or 140 (L-CDR2); the amino acid sequence of SEQ ID NO: 5 (L-CDR3); and a heavy chain variable region comprising the amino acid sequence of SEQ ID NO: 12, 142, 143, or 146 (H-CDR1); the amino acid sequence of SEQ ID NO: 13, 161 (H-CDR2); the amino acid sequence of SEQ ID NO: 10 (H-CDR3), or
a light chain variable region comprising the amino acid sequence of SEQ ID NO: 6, 127, 128, 129, 130, 131, 132, 133, 134, or 135 (L-CDR1); the amino acid sequence of SEQ ID NO: 7, 139, 141 (L-CDR2); the amino acid sequence of SEQ ID NO: 5 (L-CDR3); and a heavy chain variable region comprising the amino acid sequence of SEQ ID NO: 14 (H-CDR1); the amino acid sequence of SEQ ID NO: 15, 162, 163, 164, or 167 (H-CDR2); the amino acid sequence of SEQ ID NO: 16 (H-CDR3).

In embodiment thirty four, the present invention provides an anti-IL1RAP antibody or antigen-binding fragment thereof, wherein the antibody or antigen-binding fragment thereof comprises a light chain variable region comprising the amino acid sequence of SEQ ID NO: 17; and a heavy chain variable region comprising the amino acid sequence of SEQ ID NO: 67; or
a light chain variable region comprising the amino acid sequence of SEQ ID NO: 18; and a heavy chain variable region comprising the amino acid sequence of SEQ ID NO: 68; or
a light chain variable region comprising the amino acid sequence of SEQ ID NO: 19; and a heavy chain variable region comprising the amino acid sequence of SEQ ID NO: 69; or
a light chain variable region comprising the amino acid sequence of SEQ ID NO: 20; and a heavy chain variable region comprising the amino acid sequence of SEQ ID NO: 70; or
a light chain variable region comprising the amino acid sequence of SEQ ID NO: 21; and a heavy chain variable region comprising the amino acid sequence of SEQ ID NO: 71; or
a light chain variable region comprising the amino acid sequence of SEQ ID NO: 22; and a heavy chain variable region comprising the amino acid sequence of SEQ ID NO: 72; or
a light chain variable region comprising the amino acid sequence of SEQ ID NO: 23; and a heavy chain variable region comprising the amino acid sequence of SEQ ID NO: 73; or
a light chain variable region comprising the amino acid sequence of SEQ ID NO: 24; and a heavy chain variable region comprising the amino acid sequence of SEQ ID NO: 74; or
a light chain variable region comprising the amino acid sequence of SEQ ID NO: 25; and a heavy chain variable region comprising the amino acid sequence of SEQ ID NO: 75; or
a light chain variable region comprising the amino acid sequence of SEQ ID NO: 26; and a heavy chain variable region comprising the amino acid sequence of SEQ ID NO: 76; or
a light chain variable region comprising the amino acid sequence of SEQ ID NO: 27; and a heavy chain variable region comprising the amino acid sequence of SEQ ID NO: 77; or
a light chain variable region comprising the amino acid sequence of SEQ ID NO: 28; and a heavy chain variable region comprising the amino acid sequence of SEQ ID NO: 78; or
a light chain variable region comprising the amino acid sequence of SEQ ID NO: 29; and a heavy chain variable region comprising the amino acid sequence of SEQ ID NO: 79; or
a light chain variable region comprising the amino acid sequence of SEQ ID NO: 30; and a heavy chain variable region comprising the amino acid sequence of SEQ ID NO: 80; or
a light chain variable region comprising the amino acid sequence of SEQ ID NO: 31; and a heavy chain variable region comprising the amino acid sequence of SEQ ID NO: 81; or
a light chain variable region comprising the amino acid sequence of SEQ ID NO: 32; and a heavy chain variable region comprising the amino acid sequence of SEQ ID NO: 82; or
a light chain variable region comprising the amino acid sequence of SEQ ID NO: 33; and a heavy chain variable region comprising the amino acid sequence of SEQ ID NO: 83; or
a light chain variable region comprising the amino acid sequence of SEQ ID NO: 34; and a heavy chain variable region comprising the amino acid sequence of SEQ ID NO: 84; or
a light chain variable region comprising the amino acid sequence of SEQ ID NO: 35; and a heavy chain variable region comprising the amino acid sequence of SEQ ID NO: 85; or
a light chain variable region comprising the amino acid sequence of SEQ ID NO: 36; and a heavy chain variable region comprising the amino acid sequence of SEQ ID NO: 86; or
a light chain variable region comprising the amino acid sequence of SEQ ID NO: 37; and a heavy chain variable region comprising the amino acid sequence of SEQ ID NO: 87; or
a light chain variable region comprising the amino acid sequence of SEQ ID NO: 38; and a heavy chain variable region comprising the amino acid sequence of SEQ ID NO: 88; or
a light chain variable region comprising the amino acid sequence of SEQ ID NO: 39; and a heavy chain variable region comprising the amino acid sequence of SEQ ID NO: 89; or
a light chain variable region comprising the amino acid sequence of SEQ ID NO: 40; and a heavy chain variable region comprising the amino acid sequence of SEQ ID NO: 90; or
a light chain variable region comprising the amino acid sequence of SEQ ID NO: 41; and a heavy chain variable region comprising the amino acid sequence of SEQ ID NO: 91; or
a light chain variable region comprising the amino acid sequence of SEQ ID NO: 42; and a heavy chain variable region comprising the amino acid sequence of SEQ ID NO: 92; or
a light chain variable region comprising the amino acid sequence of SEQ ID NO: 43; and a heavy chain variable region comprising the amino acid sequence of SEQ ID NO: 93; or
a light chain variable region comprising the amino acid sequence of SEQ ID NO: 44; and a heavy chain variable region comprising the amino acid sequence of SEQ ID NO: 94; or
a light chain variable region comprising the amino acid sequence of SEQ ID NO: 45; and a heavy chain variable region comprising the amino acid sequence of SEQ ID NO: 95; or
a light chain variable region comprising the amino acid sequence of SEQ ID NO: 46; and a heavy chain variable region comprising the amino acid sequence of SEQ ID NO: 96; or
a light chain variable region comprising the amino acid sequence of SEQ ID NO: 47; and a heavy chain variable region comprising the amino acid sequence of SEQ ID NO: 97; or
a light chain variable region comprising the amino acid sequence of SEQ ID NO: 48; and a heavy chain variable region comprising the amino acid sequence of SEQ ID NO: 98; or
a light chain variable region comprising the amino acid sequence of SEQ ID NO: 49; and a heavy chain variable region comprising the amino acid sequence of SEQ ID NO: 99; or
a light chain variable region comprising the amino acid sequence of SEQ ID NO: 50; and a heavy chain variable region comprising the amino acid sequence of SEQ ID NO: 100; or
a light chain variable region comprising the amino acid sequence of SEQ ID NO: 51; and a heavy chain variable region comprising the amino acid sequence of SEQ ID NO: 101; or
a light chain variable region comprising the amino acid sequence of SEQ ID NO: 52; and a heavy chain variable region comprising the amino acid sequence of SEQ ID NO: 102; or
a light chain variable region comprising the amino acid sequence of SEQ ID NO: 53; and a heavy chain variable region comprising the amino acid sequence of SEQ ID NO: 103; or
a light chain variable region comprising the amino acid sequence of SEQ ID NO: 54; and a heavy chain variable region comprising the amino acid sequence of SEQ ID NO: 104; or
a light chain variable region comprising the amino acid sequence of SEQ ID NO: 55; and a heavy chain variable region comprising the amino acid sequence of SEQ ID NO: 105; or
a light chain variable region comprising the amino acid sequence of SEQ ID NO: 56; and a heavy chain variable region comprising the amino acid sequence of SEQ ID NO: 106; or
a light chain variable region comprising the amino acid sequence of SEQ ID NO: 57; and a heavy chain variable region comprising the amino acid sequence of SEQ ID NO: 107; or
a light chain variable region comprising the amino acid sequence of SEQ ID NO: 58; and a heavy chain variable region comprising the amino acid sequence of SEQ ID NO: 108; or
a light chain variable region comprising the amino acid sequence of SEQ ID NO: 59; and a heavy chain variable region comprising the amino acid sequence of SEQ ID NO: 109; or
a light chain variable region comprising the amino acid sequence of SEQ ID NO: 60; and a heavy chain variable region comprising the amino acid sequence of SEQ ID NO: 110; or
a light chain variable region comprising the amino acid sequence of SEQ ID NO: 61; and a heavy chain variable region comprising the amino acid sequence of SEQ ID NO: 111; or
a light chain variable region comprising the amino acid sequence of SEQ ID NO: 62; and a heavy chain variable region comprising the amino acid sequence of SEQ ID NO: 112; or
a light chain variable region comprising the amino acid sequence of SEQ ID NO: 63; and a heavy chain variable region comprising the amino acid sequence of SEQ ID NO: 113; or
a light chain variable region comprising the amino acid sequence of SEQ ID NO: 64; and a heavy chain variable region comprising the amino acid sequence of SEQ ID NO: 114; or
a light chain variable region comprising the amino acid sequence of SEQ ID NO: 65; and a heavy chain variable region comprising the amino acid sequence of SEQ ID NO: 115; or
a light chain variable region comprising the amino acid sequence of SEQ ID NO: 66; and a heavy chain variable region comprising the amino acid sequence of SEQ ID NO: 116.

In a further embodiment thirty five, the present invention provides a pharmaceutical composition comprising an antibody or antigen-binding fragment according to any one of the previous embodiments and a pharmaceutically acceptable carrier.

In a further embodiment thirty six the present invention provides an antibody or antigen-binding fragment or pharmaceutical composition according to any one of the previous embodiments, for use as a medicine.

In a further embodiment thirty seven the present invention provides an antibody or antigen-binding fragment according to any one of the embodiments 1-34 or pharmaceutical composition, wherein the use is the treatment of an inflammatory disease, of an autoimmune disease, of a respiratory disease, of a metabolic disorder, of an epithelial mediated inflammatory disorder, of fibrosis or of cancer.

In a further embodiment thirty eight the present invention provides an anti-IL1RAP antibody or antigen-binding fragment according to any one of embodiments 1 to 34 for use in treating a disease, wherein the disease is selected from an inflammatory disease, an autoimmune disease, a respiratory disease, a metabolic disorder, an epithelial mediated inflammatory disorder, fibrosis and cancer.

In a further embodiment thirty nine the present invention provides use of the anti-IL1RAP antibody or antigen-binding fragment according to any one of embodiments 1 to 34 in manufacture of a medicament for treating a disease, wherein the disease is selected from an inflammatory disease, an autoimmune disease, a respiratory disease, a metabolic disorder, an epithelial mediated inflammatory disorder, fibrosis and cancer.

In a further embodiment forty the present invention provides an antibody or antigen-binding fragment according to any one of the embodiments 1-34, the anti-IL1RAP antibody or antigen-binding fragment according to embodiment 38, or the use of the anti-IL1RAP antibody or antigen-binding fragment according to embodiment 39, wherein the use is for the treatment of psoriasis, psoriatic arthritis, multiple sclerosis, rheumatoid arthritis, COPD, chronic asthma or ankylosing spondylitis.

In another embodiment forty one, the present invention provides a method of treating a disease comprising administering the antibody or antigen-binding fragment according to any one of the embodiments 1-34 or pharmaceutical composition, to a patient in need thereof, wherein the disease is selected from an inflammatory disease, an autoimmune disease, a respiratory disease, a metabolic disorder, an epithelial mediated inflammatory disorder, fibrosis and cancer.

In another embodiment forty two, the present invention provides a method according to embodiment 41 wherein the disease is selected from psoriasis, psoriatic arthritis, multiple sclerosis, rheumatoid arthritis, COPD, chronic asthma and ankylosing spondylitis.

Further embodiments of the invention encompass:
An isolated polynucleotide comprising a sequence encoding an anti-IL1RAP antibody or antigen-binding fragment according to the invention, preferably a DNA or RNA sequence;
an isolated polynucleotide according to the invention, encoding a sequence as defined by one or more of SEQ ID NOs: 1 to 167, or 170-183;
a vector comprising a polynucleotide according to the invention, preferably an expression vector, more preferred a vector comprising the polynucleotide according to the invention in functional association with an expression control sequence;
a host cell comprising a polynucleotide according to the invention and/or a vector according to the invention;
a method for the production of an anti-IL1RAP antibody or antigen-binding fragment according to the invention, preferably a recombinant production method comprising the use of a polynucleotide according to the invention, and/or of a vector according to the invention and/or of a host cell according to the invention;
such a method preferably comprises the steps (a) cultivating the host cell under conditions allowing the expression of the anti-IL1RAP antibody or antigen-binding fragment and (b) recovering the anti-IL1RAP antibody or antigen-binding fragment;
a diagnostic kit or diagnostic method comprising an anti-IL1RAP antibody or antigen-binding fragment according to the invention, or the use thereof;
a Diagnostic kit or diagnostic method according the invention, for the diagnosis of an inflammatory disease, an autoimmune disease, a respiratory disease, a metabolic disorder, an epithelial mediated inflammatory disorder, fibrosis, cancer, psoriasis, psoriatic arthritis, multiple sclerosis, rheumatoid arthritis, COPD, chronic asthma, or ankylosing spondylitis.

### BRIEF DESCRIPTION OF THE FIGURES

FIG. 1: Comparison of chimeric and engrafted Fabs containing anti-IL1RAP Parent clone GO11 VH/VL CDRs

FIG. 2A/D: Optimized VK clone variants and VH clone variants were selected and screened for binding to huIL1RAP by ELISA as compared to the chimeric parent anti-IL1RAP GO11 Fab. (A) Comparison of VK clones 405-03, 405-07, 405-04, 405-05, 405-10, demonstrating selected clone 405-10 (SEQ ID NO: 18) had similar binding to chimeric parent clone GO11; (B) Comparison of VK clones 405-11, 406-02, 405-12, 406-01, 406-03, demonstrating selected VK clone 405-12 (SEQ ID NO:12) had similar binding to chimeric parent clone GO11; (C) Comparison of VH clones 406-04, 406-15, 406-08, 406-10, 406-18, demonstrating selected VH clone 405-18 (SEQ ID NO:68) had similar binding to the chimeric parent clone GO11; and (D) Comparison of VH clones 406-20, 406-21, 406-26, demonstrating selected VH clone 406-20 (SEQ ID NO:69) had similar binding to chimeric parent clone GO11.

FIG. 3A/B: Comparison of Germline optimized anti-IL1RAP VL CDRs (A)/VH (B) CDRS

FIG. 4A/D: A. Inhibition of IL-12p40 secretion by an anti-IL1RAP Ab in cytokine stimulated MDMs (depicted are mean values±SD of technical triplicates from one representative of two experiments with MDMs obtained from different donors). B. Suppression of of IL-8 (IC50=3.95 nM), TNF (IC50=6.45 nM), and IL-6 (IC50=9.41 nM) protein production in IL-36α, β, γ cytokine stimulated human monocyte derived dendritic cells (MoDCs) by anti-IL1Rap Ab. C. Suppression of myeloid cell derived mediator Mip-1b in IL-36γ stimulated whole blood by anti-IL1RAP Ab (IC50=4.37), and IL-36γ+IL-33 stimulated whole blood (IC50-3.43). D. Inhibition of IFNγ production in IL-33/IL-12 stimulated whole blood cells by anti-IL1RAP Ab (IC50=4.84 nM).

FIG. 5A/D: Structural features of the IL-1RAcP:anti-IL1RAP Ab #A2 and the IL-1RAcP-IL-1RI-IL1β complexes in two different views related by a 90° rotation. A. The IL-1 RAcP is shown in pale blue with semi-transparent surface. The #A2 Fab is shown as ribbons. The heavy and light chains are colored in dark and light grey, respectively. B. Location of the Epitope: The Fab is shown as semitransparent ribbon for clarity. The epitope of the Fab on IL-1RAcP is shown in grey. C. Structure of the human IL-1RAcP-IL-1RI-IL1β ternary complex (pdb 4dep) for comparison in the same orientation as (a). IL-1RI is colored in red and IL-1β in green. D. The IL-1RAcP-side of the IL-1RAcP-IL-1RI interface is shown in orange. IL-1RI and IL1β are shown as semitransparent ribbons for clarity.

FIG. 6A/B: Comparison of the HDX and the X-ray epitope of IL1RAP. A. THE HDX epitope is shown in yellow. It comprises amino acid residues 226-262 and 269-273. B. The X-ray epitope as shown in FIG. 5B where the snit-IL1RAP Fab is removed for clarity.

FIG. 7A/C: A. anti-IL-RAP free drug level in monkey serum after IV administration (drug concentration (nM) vs. Timepoint in hours). B. anti-IL1RAP total drug level in monkey serum after IV administration (drug concentration (nM) vs. Timepoint in hours). C. Soluble IL1RAP target level in cynomolgus money after IV administration (Conc (nM) vs Time in hours).

### DESCRIPTION OF THE INVENTION

This invention relates to anti-IL1RAP antibodies. In one aspect, the antibodies of the present invention are for diagnostic and therapeutic use, for example in humans.

The present invention provides antibodies that bind to IL1RAP, in particular human IL1RAP. The present invention also relates to humanized antibodies that bind IL1RAP. In specific embodiments, the sequence of these humanized antibodies has been identified based on the sequences of certain lead mouse antibodies.

Without wishing to be bound by this theory it is believed that anti-IL1RAP antibodies or antigen-binding fragments thereof bind to human IL1RAP and thus interfere with the binding of IL1RAP agonists, and in doing so block at least partially the signaling cascade from the IL1RAP to inflammatory mediators.

In one aspect, the present invention provides an anti-IL1RAP antibody having one or more of the properties below.

In one aspect, an anti-IL1RAP antibody of the present invention has high molecular/cellular binding potency. In one aspect, an anti-IL1RAP antibody of the present invention binds to human IL1RAP at a K_{D}<0.1 nM. In a further aspect, an anti-IL1RAP antibody of the present invention, in particular a humanized anti-IL1RAP antibody, binds to human and cynomolgus IL1RAP at a K_{D}<200 pM.

In another aspect, an anti-IL1RAP antibody of the present invention has high cell-based functional blocking potency.

In one aspect, an anti-IL1RAP antibody of the present invention is a humanized antibody. In one aspect, an anti-IL1RAP antibody of the present invention is a monoclonal antibody. In one aspect, an anti-IL1RAP antibody of the present invention is a full length antibody. In one aspect, an anti-IL1RAP antibody of the present invention is a humanized monoclonal antibody, for example a full length humanized monoclonal antibody.

An antibody or antigen-binding fragment thereof of the present invention recognizes specific “IL1RAP antigen epitope” or “IL1RAP epitope” or “IL-1RAP epitope”. As used herein these terms refer to a molecule (e.g., a peptide) or a fragment of a molecule capable of immunoreactivity with an anti-IL1RAP antibody.

The epitopes are most commonly proteins, short oligopeptides, oligopeptide mimics (i.e., organic compounds that mimic antibody binding properties of the IL1RAP antigen), or combinations thereof. The minimum size of a peptide or polypeptide epitope for an antibody is thought to be about four to five amino acids. Peptide or polypeptide epitopes contain for example at least seven amino acids or for example at least nine amino acids or for example between about 15 to about 20 amino acids. Since an antibody can recognize an antigenic peptide or polypeptide in its tertiary form, the amino acids comprising an epitope need not be contiguous, and in some cases, may not even be on the same peptide chain. Epitopes may be determined by various techniques known in the art, such as X-ray crystallography, Hydrogen/Deuterium Exchange Mass Spectrometry (HXMS), site-directed mutagenesis, alanine scanning mutagenesis, and peptide screening methods.

The generalized structure of antibodies or immunoglobulin is well known to those of skill in the art. These molecules are heterotetrameric glycoproteins, typically of about 150,000 daltons, composed of two identical light (L) chains and two identical heavy (H) chains and are typically referred to as full length antibodies. Each light chain is covalently linked to a heavy chain by one disulfide bond to form a heterodimer, and the heterotrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}) and a carboxy-terminal constant domain (C_{L}). The V_{L} domain associates non-covalently with the V_{H} domain, whereas the C_{L} domain is commonly covalently linked to the C_{H1} domain via a disulfide bond. Particular amino acid residues are believed to form an interface between the light and heavy chain variable domains (Chothia et al., 1985, J. Mol. Biol. 186:651-663). Variable domains are also referred herein as variable regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5^{th} Ed. Public Health Service, National Institutes of Health, Bethesda, Md., whereas HVLs (also referred herein as CDRs) are structurally defined according to the three-dimensional structure of the variable domain, as described by Chothia and Lesk, 1987, J. Mol. Biol. 196:901-917. These two methods result in slightly different identifications of a CDR. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. The exact residue numbers that encompass a particular CDR will vary depending on the sequence and size of the CDR. While the Kabat and Chothia schemes treated separately the different families of immunoglobulin domains, Lefranc and colleagues proposed a unified numbering scheme, referred to as IMGT numbering scheme, for immunoglobulin variable domain genomic sequences, including Ab light and heavy variable domains, as well as T-cell receptor variable domains (Lefranc M P, et al. IMGT unique numbering for immunoglobulin and T cell receptor variable domains and Ig superfamily V-like domains and Ig superfamily V-like domains. Dev Comp Immunol (2003) 27:55-77).

As mentioned above, there are differences and drawbacks amongst the Kabat, Chothia and IMGT numbering schemes wherein the CDR length variable is only taken into account the most common loop lengths and therefore there are some inconsistencies depending on the numbering scheme used. In some cases some of the CDRs identified by the four methods included herein are almost identical (e.g. L3, H3), while in other CDRs (e.g. L2, H1, and H2) there are substantial differences between the methods. Thus, for the sake of completeness, the CDRs of the present embodiments are indicated according to each of the numbering conventions to best identify the critical Ag binding residues (see Tables 1-4 below). Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

### Sequences of the CDR According to Four Different Nomenclatures

CDRs 1-3 of the variable light chain and the variable heavy chain regions for antibodies A #1-A #7 are enumerated by SEQ ID NO and presented according to the Kabat nomenclature in Table 1 below.

TABLE 1
CDRS according to Kabat Nomenclature

| | KABAT SEQ | SEQ ID NO: | Amino Acid # |
|---|---|---|---|
| | A#1 | | |
| HCDR1 | TYWMN | 8 | 31-35 |
| HCDR2 | QIFPASDSTYYNEMFKD | 9 | 50-66 |
| HCDR3 | SGPYSYYAGGYALDY | 10 | 99-113 |
| LCDR1 | RTSENINSYLA | 3 | 24-34 |
| LCDR2 | YAKTLAE | 4 | 50-56 |
| LCDR3 | QHHYGTSLT | 5 | 89-97 |
| | A#2 | | |
| HCDR1 | TYWMN | 8 | 31-35 |
| HCDR2 | QIFPASGSAYYNQKFKG | 149 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#3 | | |
| HCDR1 | TYWIN | 142 | 31-35 |
| HCDR2 | QIFPASDSTYYNQKFKG | 153 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#4 | | |
| HCDR1 | TYWIN | 142 | 31-35 |
| HCDR2 | QIFPASGSAYYAQKFQG | 148 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#5 | | |
| HCDR1 | TYWMN | 8 | 31-35 |
| HCDR2 | QIFPASGSAYYAQKFQG | 148 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | A#6 | | |
| HCDR1 | TYWMN | 8 | 31-35 |
| HCDR2 | QIFPASGSAYYNQKFKG | 149 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | A#7 | | |
| HCDR1 | TYWIN | 142 | 31-35 |
| HCDR2 | QIFPASGSTYYNEKFKG | 151 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | KABAT CONSENSUS | | |
| | | X | SEQ ID NO: |
| HCDR1 | TYWX₁X₂ | X1 = I, M; | 146 |
| | | X2 = S, N | |
| HCDR2 | QIFPASX1SX2YYX3X4X5 | X1 = G, D; | 165 |
| | FX6X7 | X2 = A, T; | |
| | | X3 = A, N, | |
| | | X4 = Q, E; | |
| | | X5 = K, M; | |
| | | X6 = K, Q; | |
| | | X7 = G, D | |
| HCDR3 | SGPYSYYAGGYALDY | | 10 |
| LCDR1 | RX1SX2X3IX4SYLA | X1 = A, T; | 134 |
| | | X2 = Q, E; | |
| | | X3 = S, N; | |
| | | X4 = N, S | |
| LCDR2 | YAX1X2LAE | X1 = S, K; | 140 |
| | | X2 = S, T | |
| LCDR1 | QHHYGTSLT | | 5 |

CDRs 1-3 of the variable light chain and the variable heavy chain regions for antibodies A #1-A #7 are presented according to the CCG (Chemical Computing Group as illustrated in Almagro et al., Proteins 2011; 79:3050-3066 and Maier et al, Proteins 2014; 82:1599-1610) in Table 2 below.

TABLE 2
CDRS according to CCG Nomenclature

| | CCG SEQ | SEQ ID NO: | Amino Acid # |
|---|---|---|---|
| | A#1 | | |
| HCDR1 | GYIFLTYWMN | 11 | 26-35 |
| HCDR2 | QIFPASDSTYYNEMFKD | 9 | 50-66 |
| HCDR3 | SGPYSYYAGGYALDY | 10 | 99-113 |
| LCDR1 | RTSENINSYLA | 3 | 24-34 |
| LCDR2 | YAKTLAE | 4 | 50-56 |
| LCDR3 | QHHYGTSLT | 5 | 89-97 |
| | A#2 | | |
| HCDR1 | GYIFLTYWMN | 11 | 26-35 |
| HCDR2 | QIFPASGSAYYNQKFKG | 149 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#3 | | |
| HCDR1 | GYIFLTYWIN | 144 | 26-35 |
| HCDR2 | QIFPASDSTYYNQKFKG | 153 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#4 | | |
| HCDR1 | GYIFLTYWIN | 144 | 26-35 |
| HCDR2 | QIFPASGSAYYAQKFQG | 148 | 50-66 |
| HCDR3 | SGPYSYYAGGYALDY | 10 | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#5 | | |
| HCDR1 | GYIFLTYWMN | 11 | 26-35 |
| HCDR2 | QIFPASGSAYYAQKFQG | 148 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | A#6 | | |
| HCDR1 | GYIFLTYWMN | 11 | 26-35 |
| HCDR2 | QIFPASGSAYYNQKFKG | 149 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | A#7 | | |
| HCDR1 | GYIFLTYWIN | 144 | 26-35 |
| HCDR2 | QIFPASGSTYYNEKFKG | 151 | 50-66 |
| HCDR3 | SGPYSYYAGGYALDY | 10 | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | CCG CONSENSUS | | |
| | | X | SEQ ID NO: |
| | GYIFLTYWX1N | X1 = I, M | 193 [symbol] |
| | QIFPASX1SX2YYX3X4X5 | X1 = G, D; | 165 |
| | FX6X7 | X2 = A, T; | |
| | | X3 = A, N, | |
| | | X4 = Q, E; | |
| | | X5 = K, M; | |
| | | X6 = K, Q; | |
| | | X7 = G, D | |
| | SGPYSYYAGGYALDY | | 10 |
| | RX1SX2X3IX4SYLA | X1 = A,T; | 134 |
| | | X2 = Q, E; | |
| | | X3 = S, N; | |
| | | X4 = N, S | |
| | YAX1X2LAE | X1 = S, K; | 140 |
| | | X2 = S, T | |
| | QHHYGTSLT | | 5 |

An additional numbering system based on the Chothia scheme is presented for CDRs 1-3 of the variable light chain and the variable heavy chain regions for antibodies A #1-A #7 in Table 3 below.

TABLE 3
CDRS according to Chothia Nomenclature

| | CHOTHIA SEQ | SEQ ID NO: | Amino Acid # |
|---|---|---|---|
| | A#1 | | |
| HCDR1 | GYIFLTY | 12 [symbol] | 26-32 |
| HCDR2 | FPASDS | 13 [symbol] | 52-57 |
| HCDR3 | SGPYSYYAGGYALDY | 10 [symbol] | 99-113 |
| LCDR1 | RTSENINSYLA | 3 | 24-34 |
| LCDR2 | YAKTLAE | 4 | 50-56 |
| LCDR3 | QHHYGTSLT | 5 | 89-97 |
| | A#2 | | |
| HCDR1 | GYIFLTY | 12 [symbol] | 26-32 |
| HCDR2 | FPASGS | 161 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#3 | | |
| HCDR1 | GYIFLTY | 12 [symbol] | 26-32 |
| HCDR2 | FPASDS | 13 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#4 | | |
| HCDR1 | GYIFLTY | 12 [symbol] | 26-32 |
| HCDR2 | FPASGS | 161 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YAKSLAE | 136 | 50-56 |
| LCDR3 | QHHYGTSLT | 5 | 89-97 |
| | A#5 | | |
| HCDR1 | GYIFLTY | 12 [symbol] | 26-32 |
| HCDR2 | FPASGS | 161 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | A#6 | | |
| HCDR1 | GYIFLTY | 12 [symbol] | 26-32 |
| HCDR2 | FPASGS | 161 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | #A7 | | |
| HCDR1 | GYIFLTY | 12 [symbol] | 26-32 |
| HCDR2 | FPASGS | 161 [symbol] | 52-57 |
| HCDR3 | SGPYSYYAGGYALDY | 10 [symbol] | 99-113 |
| LCDR1 | RASQSISSYLA | 117 | 24-34 |
| LCDR2 | YASSLAE | 137 | 50-56 |
| LCDR3 | QHHYGTSLT | 5 | 89-97 |
| | CHOTHIA CONSENSUS | | |
| | | X | SEQ ID NO: |
| HCDR1 | GYIFLTY | | 12 [symbol] |
| HCDR2 | FPASX1S | X1 = G, D | 166 [symbol] |
| HCDR3 | SGPYSYYAGGYALDY | | 10 [symbol] |
| LCDR1 | RX1SX2X3IX4SYLA | X1 = A, T; X2 = | 134 |
| | | Q, E; X3 = S, | |
| | | N; X4 = N, S | |
| LCDR2 | YAX1X2LAE | X1 = S, K; X2 = | 140 |
| | | S, T | |
| LCDR3 | QHHYGTSLT | | 5 |

CDRs 1-3 of the variable light chain and the variable heavy chain regions for antibodies A #1-A #7 C based on the IMGT numbering scheme (Lefranc M P, et al. IMGT unique numbering for immunoglobulin and T cell receptor variable domains and Ig superfamily V-like domains and Ig superfamily V-like domains. Dev Comp Immunol (2003) 27:55-77) is presented in the Table 4 below.

TABLE 4
CDRS according to IMGT Nomenclature

| | IMGT SEQ | SEQ ID NO: | Amino Acid # |
|---|---|---|---|
| | A#1 | | |
| HCDR1 | GYIFLTYW | 14 | 26-33 |
| HCDR2 | IFPASDST | 15 | 51-58 |
| HCDR3 | ARSGPYSYYAGGYALDY | 16 | 97-113 |
| LCDR1 | ENINSY | 6 | 27-32 |
| LCDR2 | YAK | 7 | 50-52 |
| LCDR3 | QHHYGTSLT | 5 | 89-97 |
| | A#2 | | |
| HCDR1 | GYIFLTYW | 14 | 26-33 |
| HCDR2 | IFPASGSA | 162 | 51-58 |
| HCDR3 | ARSGPYSYYAGGYALDY | 16 | 97-113 |
| LCDR1 | QSISSY | 127 | 27-32 |
| LCDR2 | YAK | 7 | 50-52 |
| LCDR3 | QHHYGTSLT | 5 | 89-97 |
| | A#3 | | |
| HCDR1 | GYIFLTYW | 14 | 26-33 |
| HCDR2 | IFPASDST | 15 | 51-58 |
| HCDR3 | ARSGPYSYYAGGYALDY | 16 | 97-113 |
| LCDR1 | QSISSY | 127 | 27-32 |
| LCDR2 | YAK | 7 | 50-52 |
| LCDR3 | QHHYGTSLT | 5 | 89-97 |
| | A#4 | | |
| HCDR1 | GYIFLTYW | 14 | 26-33 |
| HCDR2 | IFPASGSA | 162 | 51-58 |
| HCDR3 | ARSGPYSYYAGGYALDY | 16 | 97-113 |
| LCDR1 | QSISSY | 127 | 27-32 |
| LCDR2 | YAK | 7 | 50-52 |
| LCDR3 | QHHYGTSLT | 5 | 89-97 |
| | A#5 | | |
| HCDR1 | GYIFLTYW | 14 | 26-33 |
| HCDR2 | IFPASGSA | 162 | 51-58 |
| HCDR3 | ARSGPYSYYAGGYALDY | 16 | 97-113 |
| LCDR1 | QSISSY | 127 | 27-32 |
| LCDR2 | YAS | 139 | 50-52 |
| LCDR3 | QHHYGTSLT | 5 | 89-97 |
| | A#6 | | |
| HCDR1 | GYIFLTYW | 14 | 26-33 |
| HCDR2 | IFPASGSA | 162 | 51-58 |
| HCDR3 | ARSGPYSYYAGGYALDY | 16 | 97-113 |
| LCDR1 | QSISSY | 127 | 27-32 |
| LCDR2 | YAS | 139 | 50-52 |
| LCDR3 | QHHYGTSLT | 5 | 89-97 |
| | #A7 | | |
| HCDR1 | GYIFLTYW | 14 | 26-33 |
| HCDR2 | IFPASGST | 163 | 51-58 |
| HCDR3 | ARSGPYSYYAGGYALDY | 16 | 97-113 |
| LCDR1 | QSISSY | 127 | 27-32 |
| LCDR2 | YAS | 139 | 50-52 |
| LCDR3 | QHHYGTSLT | 5 | 89-97 |
| | IMGT CONSENSUS | | |
| | | X | SEQ ID NO: |
| HCDR1 | GYIFLTYW | | 14 |
| HCDR2 | IFPASX1SX2 | X1 = D, G; X2 = | 167 |
| | | T, A [symbol] | |
| HCDR3 | ARSGPYSYYAGGYALDY | | 16 |
| LCDR1 | X1X2IX3SY | X1, = E, Q; | 135 [symbol] |
| | | X2 = N, S; X3 = | |
| | | N, S | |
| LCDR2 | YAX1 | X1 = K, S | 141 |
| LCDR3 | QHHYGTSLT | | 5 |

The amino acid positions indicated in this context of Kabat, CCG, Chothia, or IMGT positions (Tables 1-4) are linear, i.e. the amino acids of the respective full length molecule chain are consecutively numbered starting from number 1 at the N-terminus and end with the number that corresponds to the total number of amino acids in said molecule. For example, a heavy chain consisting of 118 amino acids in length will start with number 1 at the N-terminus and will end with number 118 at the most C-terminal amino acid. Thus, any reference to e.g. position 25 means that the amino acid number 25 as counted from the N-terminus of this molecule.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “anti-IL1RAP antibody”, “anti-IL1-RAP antibody”, “humanized anti-IL1RAP antibody”, “humanized anti-IL1RAP epitope antibody”, and “variant humanized anti-IL1RAP epitope antibody” specifically encompass monoclonal antibodies (including full length monoclonal antibodies), polyclonal antibodies, multispecific antibodies (e.g., bispecific antibodies), and antibody fragments such as variable domains and other portions of antibodies that exhibit a desired biological activity, e.g., IL1RAP binding. Humanized antibodies are for the most part human immunoglobulins (e.g., chimeric immunoglobulins, an immunoglobulin chain, or a fragment thereof (such as Fv, Fab, Fab′, F(ab′)2 or other antigen-binding subsequences of antibodies) that contains minimal sequence derived from a non-human immunoglobulin) in which residues from a complementarily determining region (CDR) of the recipient are replaced by residues from a CDR of a non-human species such as mouse, rat, or rabbit having the desired specificity, affinity, and biological activity. The term “monoclonal antibody” (mAb) refers to an antibody that is highly specific, being directed against a single antigenic determinant, an “epitope”. Therefore, the modifier “monoclonal” is indicative of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352:624-628, and Marks et al., 1991, J. Mol. Biol. 222:581-597.

The term “monomer” refers to a homogenous form of an antibody. For example, for a full-length antibody, monomer means a monomeric antibody having two identical heavy chains and two identical light chains.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81:6851-6855).

The terms, “antibody fragment”, “anti-IL1RAP antibody fragment”, “anti-IL1RAP epitope antibody fragment”, “humanized anti-IL1RAP antibody fragment”, “humanized anti-IL1RAP epitope antibody fragment”, “variant humanized anti-IL1RAP epitope antibody fragment” refer to a portion of a full length anti-IL1RAP antibody, in which a variable region or a functional capability is retained, for example, specific IL1RAP epitope binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Full length antibodies can be treated with enzymes such as papain or pepsin to generate useful antibody fragments. Papain digestion is used to produces two identical antigen-binding antibody fragments called “Fab” fragments, each with a single antigen-binding site, and a residual “Fc” fragment. The Fab fragment also contains the constant domain of the light chain and the C_{H1} domain of the heavy chain. Pepsin treatment yields a F(ab′)₂ fragment that has two antigen-binding sites and is still capable of cross-linking antigen.

Fab′ fragments differ from Fab fragments by the presence of additional residues including one or more cysteines from the antibody hinge region at the C-terminus of the C_{H1} domain. F(ab′)₂ antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

A “diabody” refers to small antibody fragments with two antigen-binding sites, which fragments comprise a heavy chain variable domain (V.sub.H) connected to a light chain variable domain (V.sub.L) in the same polypeptide chain (V.sub.H-V.sub.L or V.sub.L-V.sub.H). Diabodies are described more fully in, e.g., Holliger et al. (1993) Proc. Natl. Acad. Sci. USA 90:6444-6448.

Other recognized antibody fragments include those that comprise a pair of tandem Fd segments (V_{H}-C_{H1}-V_{H}-C_{H1}) to form a pair of antigen binding regions. These “linear antibodies” can be bispecific or monospecific as described in, for example, Zapata et al. 1995, Protein Eng. 8 (10): 1057-1062.

A “humanized antibody” or a “humanized antibody fragment” is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain. The present invention describes specific humanized anti-IL1RAP antibodies which contain CDRs derived from the mouse monoclonal antibodies or humanized CDRs inserted between the FRs of human germline sequence heavy and light chain variable domains. It will be understood that certain mouse FR residues may be important to the function of the humanized antibodies and therefore certain of the human germline sequence heavy and light chain variable domains residues are modified to be the same as those of the corresponding mouse sequence.

In another aspect, a humanized anti-IL1RAP antibody comprises substantially all of at least one, and typically two, variable domains (such as contained, for example, in Fab, Fab′, F(ab′)2, Fabc, and Fv fragments) in which all, or substantially all, of the CDRs correspond to those of a non-human immunoglobulin, and specifically herein, all of the CDRs are mouse or humanized sequences as detailed herein below and all, or substantially all, of the FRs are those of a human immunoglobulin consensus or germline sequence. In another aspect, a humanized anti-IL1RAP antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H1}, hinge, C_{H2}, C_{H3}, and/or C_{H4} regions of the heavy chain, as appropriate.

A humanized anti-IL1RAP antibody can be selected from any class of immunoglobulins, including IgM, IgG, IgD, IgA and IgE, and any isotype, including IgG1, IgG2, IgG3, IgG4, IgA₁ and IgA₂. For example, the constant domain can be a complement fixing constant domain where it is desired that the humanized antibody exhibit cytotoxic activity, and the isotype is typically IgG1. Where such cytotoxic activity is not desirable, the constant domain may be of another isotype, e.g., IgG2. An alternative humanized anti-IL1RAP antibody can comprise sequences from more than one immunoglobulin class or isotype, and selecting particular constant domains to optimize desired effector functions is within the ordinary skill in the art. In specific embodiments, the present invention provides antibodies that are IgG1 antibodies and more particularly, are IgG1 antibodies in which there is a knock-out of effector functions.

The FRs and CDRs, or HVLs, of a humanized anti-IL1RAP antibody need not correspond precisely to the parental sequences. For example, one or more residues in the import CDR, or HVL, or the consensus or germline FR sequence may be altered (e.g., mutagenized) by substitution, insertion or deletion such that the resulting amino acid residue is no longer identical to the original residue in the corresponding position in either parental sequence but the antibody nevertheless retains the function of binding to IL1RAP. Such alteration typically will not be extensive and will be conservative alterations. Usually, at least 75% of the humanized antibody residues will correspond to those of the parental consensus or germline FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof.

Human germline sequences are found naturally in the human population. A combination of those germline genes generates antibody diversity. Germline antibody sequences for the light chain of the antibody come from conserved human germline kappa or lambda v-genes and j-genes. Similarly the heavy chain sequences come from germline v-, d- and j-genes (LeFranc, M-P, and LeFranc, G, “The Immunoglobulin Facts Book” Academic Press, 2001).

As used herein, “variant”, “anti-IL1RAP variant”, “humanized anti-IL1RAP variant”, or “variant humanized anti-IL1RAP” each refers to a humanized anti-IL1RAP antibody having at least a light chain variable murine CDR. Variants include those having one or more amino acid changes in one or both light chain or heavy chain variable domains, provided that the amino acid change does not substantially impair binding of the antibody to IL1RAP.

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

The term “antibody performance” refers to factors that contribute to antibody recognition of antigen or the effectiveness of an antibody in vivo. Changes in the amino acid sequence of an antibody can affect antibody properties such as folding, and can influence physical factors such as initial rate of antibody binding to antigen (kₐ), dissociation constant of the antibody from antigen (k_{d}), affinity constant of the antibody for the antigen (Kd), conformation of the antibody, protein stability, and half-life of the antibody.

The term “epitope tagged” when used herein, refers to an anti-IL1RAP antibody fused to an “epitope tag”. An “epitope tag” is a polypeptide having a sufficient number of amino acids to provide an epitope for antibody production, yet is designed such that it does not interfere with the desired activity of the humanized anti-IL1RAP antibody. The epitope tag is usually sufficiently unique such that an antibody raised against the epitope tag does not substantially cross-react with other epitopes. Suitable tag polypeptides generally contain at least 6 amino acid residues and usually contain about 8 to 50 amino acid residues, or about 9 to 30 residues. Examples of epitope tags and the antibody that binds the epitope include the flu HA tag polypeptide and its antibody 12CA5 (Field et al., 1988 Mol. Cell. Biol. 8:2159-2165; c-myc tag and 8F9, 3C7, 6E10, G4, B7 and 9E10 antibodies thereto (Evan et al., 1985, Mol. Cell. Biol. 5 (12): 3610-3616; and Herpes simplex virus glycoprotein D (gD) tag and its antibody (Paborsky et al. 1990, Protein Engineering 3 (6): 547-553). In certain embodiments, the epitope tag is a “salvage receptor binding epitope”. As used herein, the term “salvage receptor binding epitope” refers to an epitope of the Fc region of an IgG molecule (such as IgG₁, IgG₂, IgG₃, or IgG₄) that is responsible for increasing the in vivo serum half-life of the IgG molecule.

In some embodiments, the antibodies of the present invention may be conjugated to a cytotoxic agent. This is any substance that inhibits or prevents the function of cells and/or causes destruction of cells. The term is intended to include radioactive isotopes (such as I¹³¹, I¹²⁵, Y⁹⁰, and Re¹⁸⁶), chemotherapeutic agents, and toxins such as enzymatically active toxins of bacterial, fungal, plant, or animal origin, and fragments thereof. Such cytotoxic agents can be coupled to the humanized antibodies of the present invention using standard procedures, and used, for example, to treat a patient indicated for therapy with the antibody.

A “chemotherapeutic agent” is a chemical compound useful in the treatment of cancer. There are numerous examples of chemotherapeutic agents that could be conjugated with the therapeutic antibodies of the present invention. Examples of such chemotherapeutic agents include alkylating agents such a thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphoramide, and trimethylolomelamine; acetogenins (especially bullatacin and bullatacinone); camptothecin (including the synthetic analogue topotecan); bryostatin; callystatin; CC-1065 (including its adozelesin, carzelesin, and bizelesin synthetic analogues); cryptophycines (particularly cryptophycin 1 and cryptophycin 8); dolastatin, auristatins, (including analogues monomethyl-auristatin E and monomethyl-auristatin F); duocarmycin (including the synthetic analogues, KW-2189 and CBI-TMI); eleutherobin; pancratistatin; sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine; trofosfamide, uracil mustard; nitrosureas such as carmustine, chlorozotocin, fotemustine, lomustine, nimustine, ranimustine; antibiotics such as the enediyne antibiotics (e.g., calicheamicin, especially calichemicin gamma1I and calicheamicin phil1, see for example, Agnew, Chem. Intl. Ed. Engl., 33:183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin, and deoxydoxorubicin), epirubucin, esorubicin, idarubicin, marcellomycin, mitomycins such as mitomycin C, mycophenolic acid, nogalamycin, olivomycins, peplomycin, potfiromycin, puromycine, quelamycin, rodorubicin, streptonigrin, streptozocin, tubercidin, ubenimex, zinostatin, zorubicin; anti-metabolites such a methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adranals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; democolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone, mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitabronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4 (5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids, or derivatives of any of the above. Any one or more of these agents may be conjugated to the humanized antibodies of the present invention to provide a useful therapeutic agent for the treatment of various disorders.

The antibodies also may be conjugated to prodrugs. A “prodrug” is a precursor or derivative form of a pharmaceutically active substance that is less cytotoxic to tumor cells compared to the parent drug and is capable of being enzymatically activated or converted into the more active form. See, for example, Wilman, 1986, “Prodrugs in Cancer Chemotherapy”, In Biochemical Society Transactions, 14, pp. 375-382, 615th Meeting Belfast and Stella et al., 1985, “Prodrugs: A Chemical Approach to Targeted Drug Delivery, In: “Directed Drug Delivery, Borchardt et al., (ed.), pp. 247-267, Humana Press. Useful prodrugs include, but are not limited to, phosphate-containing prodrugs, thiophosphate-containing prodrugs, sulfate-containing prodrugs peptide-containing prodrugs, D-amino acid-modified prodrugs, glycosylated prodrugs, β-lactam-containing prodrugs, optionally substituted phenoxyacetamide-containing prodrugs, and optionally substituted phenylacetamide-containing prodrugs, 5-fluorocytosine and other 5-fluorouridine prodrugs that can be converted into the more active cytotoxic free drug. Examples of cytotoxic drugs that can be derivatized into a prodrug form include, but are not limited to, those chemotherapeutic agents described above.

For diagnostic as well as therapeutic monitoring purposes, the antibodies of the invention also may be conjugated to a label, either a label alone or a label and an additional second agent (prodrug, chemotherapeutic agent and the like). A label, as distinguished from the other second agents refers to an agent that is a detectable compound or composition and it may be conjugated directly or indirectly to a humanized antibody of the present invention. The label may itself be detectable (e.g., radioisotope labels or fluorescent labels) or, in the case of an enzymatic label, may catalyze chemical alteration of a substrate compound or composition that is detectable. Labeled humanized anti-IL1RAP antibody can be prepared and used in various applications including in vitro and in vivo diagnostics.

The antibodies of the present invention may be formulated as part of a liposomal preparation in order to affect delivery thereof in vivo. A “liposome” is a small vesicle composed of various types of lipids, phospholipids, and/or surfactant. Liposomes are useful for delivery to a mammal of a compound or formulation, such as a humanized anti-IL1RAP antibody disclosed herein, optionally, coupled to or in combination with one or more pharmaceutically active agents and/or labels. The components of the liposome are commonly arranged in a bilayer formation, similar to the lipid arrangement of biological membranes.

Certain aspects of the present invention related to isolated nucleic acids that encode one or more domains of the humanized antibodies of the present invention. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is distinguished from the nucleic acid molecule as it exists in natural cells.

In various aspects of the present invention one or more domains of the humanized antibodies will be recombinantly expressed. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of humanized anti-IL1RAP antibody in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with a humanized anti-IL1RAP antibody described herein. This includes chronic and acute disorders or diseases including those pathological conditions that predispose the mammal to the disorder in question. Non-limiting examples or disorders to be treated herein include inflammatory, angiogenic, autoimmune and immunologic disorders, respiratory disorders, cancer, hematological malignancies, benign and malignant tumors, leukemias and lymphoid malignancies.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia.

An IL1RAP-associated disorder includes diseases and disorders of the immune system, such as autoimmune disorders and inflammatory disorders. Such conditions include, but are not limited to, rheumatoid arthritis (RA), systemic lupus erythematosus (SLE), scleroderma, Sjogren's syndrome, multiple sclerosis, psoriasis, psoriatic arthritis, pulmonary inflammation, asthma, idiopathic thrombocytopenia purara (ITP) epithelial inflammatory disorders, fibrosis and ankylosing spondylitis.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized anti-IL1RAP antibody composition, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Antibodies

In one aspect, described and disclosed herein are anti-IL1RAP antibodies, in particular humanized anti-IL1RAP antibodies, and compositions and articles of manufacture comprising one or more anti-IL1RAP antibody, in particular one or more humanized anti-IL1RAP antibody of the present invention.

Variable regions and CDRs of representative antibodies of the present invention are disclosed below:

### Anti-IL1RAP Mouse Antibody Sequences

Variable regions and CDRs of representative mouse lead antibodies of the present invention (mouse leads) are shown below:

Light Chain Variable Region (VK)
Amino Acid Sequences

>005-G011 vK Protein
DIQMTQSPASLSASVGETVTITCRTSENINSYLAWYQQKQGKSPQ
LLVHYAKTLAEGVPSRFSGSGSGTQFSLKINSLKPEDFGSYYCQH
HYGTSLTFGAGTKLELK (SEQ ID NO: 1)

Heavy Chain Variable Region (VH)
Amino Acid Sequences

>005-GO11 vH Protein
QVHLQQSGPELVRPGTSVKISCEASGYIFLTYWMNWVKQRPGQGLEW
IGQIFPASDSTYYNEMFKDKARFTVDKSSSTAYMQFSSLTSEDTAVY
FCARSGPYSYYAGGYALDYWGQGTSVTVSS (SEQ ID NO: 2)

### Light Chain CDR-1, CDR-2, CDR3 (L-CDR1-3) and Heavy Chain CDR-1, CDR-2, CDR3 (L-CDR1-3) Amino Acid Sequences According to Kabat, CCG, Chothia and IMGT Nomenclatures

TABLE 5
KABAT NOMENCLATURE

| | Murine 005-G011 | |
|---|---|---|
| | KABAT SEQ | SEQ ID NO: |
| LCDR1 | RTSENINSYLA | 3 |
| LCDR2 | YAKTLAE | 4 |
| LCDR3 | QHHYGTSLT | 5 |
| HCDR1 | TYWMN | 8 |
| HCDR2 | QIFPASDSTYYNEMFKD | 9 |
| HCDR3 | SGPYSYYAGGYALDY | 10 |

TABLE 6
CCG NOMENCLATURE

| | Murine 005-G011 | |
|---|---|---|
| | CCG SEQ | SEQ ID NO: |
| LCDR1 | RTSENINSYLA | 3 |
| LCDR2 | YAKTLAE | 4 |
| LCDR3 | QHHYGTSLT | 5 |
| HCDR1 | GYIFLTYWMN | 11 |
| HCDR2 | QIFPASDSTYYNEMFKD | 9 |
| HCDR3 | SGPYSYYAGGYALDY | 10 |

TABLE 7
CHOTHIA NOMENCLATURE

| | Murine 005-G011 | |
|---|---|---|
| | CHOTHIA SEQ | SEQ ID NO: |
| LCDR1 | RTSENINSYLA | 3 |
| LCDR2 | YAKTLAE | 4 |
| LCDR3 | QHHYGTSLT | 5 |
| HCDR1 | GYIFLTY | 12 |
| HCDR2 | FPASDS | 13 |
| HCDR3 | SGPYSYYAGGYALDY | 10 |

TABLE 8
IMGT NOMENCLATURE

| | Murine 005-G011 | |
|---|---|---|
| | IMGT SEQ | SEQ ID NO: |
| LCDR1 | ENINSY | 6 |
| LCDR2 | YAK | 7 |
| LCDR3 | QHHYGTSLT | 5 |
| HCDR1 | GYIFLTYW | 14 |
| HCDR2 | IFPASDST | 15 |
| HCDR3 | ARSGPYSYYAGGYALDY | 16 |

### Anti-IL1RAP Humanized Antibody Sequences

Human framework sequences were selected for the mouse leads based on the framework homology, CDR structure, conserved canonical residues, conserved interface packing residues and other parameters to produce humanized variable regions (see Examples 1-2).

Representative humanized variable regions derived from antibodies opt-27-opt 73 are shown below.

TABLE 9
Light Chain Variable Region (VK) Amino Acid Sequences

| CLONE | | |
|---|---|---|
| NAME | VL SEQUENCE | SEQ ID NO: |
| HUMANIZED/ | DIQMTQSPSSLSASVGDRVTITCRTSENINSYLAWYQQKPGKAPKLLIY | 17 |
| GRAFT 005- | YAKTLAEGVPSRFSGSGSGTDFTLTISSLQPEDFATYYCQHHYGTSLTF | |
| G011 #A1 | GQGTKLEIK | |
| 405.v10\|VK\| | DIQMTQSPSSLSASVGDRVTITCRTSENINSYLAWYQQKPGKAPKLLIH | 18 |
| (Y49H) | YAKTLAEGVPSRFSGSGSGTDFTLTISSLQPEDFATYYCQHHYGTSLTF | |
| | GQGTKLEIK | |
| 405-v12\|VK\| | DIQMTQSPSSLSASVGDRVTITCRTSENINSYLAWYQQKPGKAPKLLIH | 19 |
| (Y49H) | YAKTLAEGVPSRFSGSGSGTDFTLTISSLQPEDFATYYCQHHYGTSLTF | |
| | GQGTKLEIK | |
| opt-27\|VK\| | DIQMTQSPSSLSASVGDRVTITCRASQSISSYLAWYQQKPGKAPKLLIH | 20 |
| | YAKSLAEGVPSRFSGSGSGTDFTLTISSLQPEDFATYYCQHHYGTSLTF | |
| | GQGTKLEIK | |
| -opt-28\|VK\| | DIQMTQSPSSLSASVGDRVTITCRASENINSYLAWYQQKPGKAPKLLIH | 21 |
| | YAKTLAEGVPSRFSGSGSGTDFTLTISSLQPEDFATYYCQHHYGTSLTF | |
| | GQGTKLEIK | |
| opt-29\|VK\| | DIQMTQSPSSLSASVGDRVTITCRTSQNINSYLAWYQQKPGKAPKLLIH | 22 |
| | YAKTLAEGVPSRFSGSGSGTDFTLTISSLQPEDFATYYCQHHYGTSLTF | |
| | GQGTKLEIK | |
| opt-30\|VK\| | DIQMTQSPSSLSASVGDRVTITCRASQSISSYLAWYQQKPGKAPKLLIH | 23 |
| | YASSLAEGVPSRFSGSGSGTDFTLTISSLQPEDFATYYCQHHYGTSLTF | |
| | GQGTKLEIK | |
| opt-31\|VK\| | DIQMTQSPSSLSASVGDRVTITCRTSESINSYLAWYQQKPGKAPKLLIH | 24 |
| | YAKSLAEGVPSRFSGSGSGTDFTLTISSLQPEDFATYYCQHHYGTSLTF | |
| | GQGTKLEIK | |
| opt-32\|VK\| | DIQMTQSPSSLSASVGDRVTITCRASQSINSYLAWYQQKPGKAPKLLIH | 25 |
| | YAKTLAEGVPSRFSGSGSGTDFTLTISSLQPEDFATYYCQHHYGTSLTF | |
| | GQGTKLEIK | |
| opt-33\|VK\| | DIQMTQSPSSLSASVGDRVTITCRTSESISSYLAWYQQKPGKAPKLLIH | 26 |
| | YASTLAEGVPSRFSGSGSGTDFTLTISSLQPEDFATYYCQHHYGTSLTF | |
| | GQGTKLEIK | |
| opt-34\|VK\| | DIQMTQSPSSLSASVGDRVTITCRASQSISSYLAWYQQKPGKAPKLLIH | 27 |
| | YAKTLAEGVPSRFSGSGSGTDFTLTISSLQPEDFATYYCQHHYGTSLTF | |
| | GQGTKLEIK | |
| opt-35\|VK\| | DIQMTQSPSSLSASVGDRVTITCRASQSISSYLAWYQQKPGKAPKLLIH | 28 |
| | YAKSLAEGVPSRFSGSGSGTDFTLTISSLQPEDFATYYCQHHYGTSLTF | |
| | GQGTKLEIK | |
| opt-36\|VK\| | DIQMTQSPSSLSASVGDRVTITCRASQSINSYLAWYQQKPGKAPKLLIH | 29 |
| | YASTLAEGVPSRFSGSGSGTDFTLTISSLQPEDFATYYCQHHYGTSLTF | |
| | GQGTKLEIK | |
| -opt-37\|VK\| | DIQMTQSPSSLSASVGDRVTITCRTSESINSYLAWYQQKPGKAPKLLIH | 30 |
| | YAKSLAEGVPSRFSGSGSGTDFTLTISSLQPEDFATYYCQHHYGTSLTF | |
| | GQGTKLEIK | |
| opt-38\|VK\| | DIQMTQSPSSLSASVGDRVTITCRASENISSYLAWYQQKPGKAPKLLIH | 31 |
| | YAKSLAEGVPSRFSGSGSGTDFTLTISSLQPEDFATYYCQHHYGTSLTF | |
| | GQGTKLEIK | |
| opt-39\|VK\| | DIQMTQSPSSLSASVGDRVTITCRTSQNISSYLAWYQQKPGKAPKLLIH | 32 |
| | YAKSLAEGVPSRFSGSGSGTDFTLTISSLQPEDFATYYCQHHYGTSLTF | |
| | GQGTKLEIK | |
| opt-40\|VK\| | DIQMTQSPSSLSASVGDRVTITCRTSQSINSYLAWYQQKPGKAPKLLIH | 33 |
| | YASSLAEGVPSRFSGSGSGTDFTLTISSLQPEDFATYYCQHHYGTSLTF | |
| | GQGTKLEIK | |
| opt-41\|VK\| | DIQMTQSPSSLSASVGDRVTITCRASQSISSYLAWYQQKPGKAPKLLIH | 34 |
| | YAKTLAEGVPSRFSGSGSGTDFTLTISSLQPEDFATYYCQHHYGTSLTF | |
| | GQGTKLEIK | |
| opt-42\|VK\| | DIQMTQSPSSLSASVGDRVTITCRASQSISSYLAWYQQKPGKAPKLLIH | 35 |
| | YAKSLAEGVPSRFSGSGSGTDFTLTISSLQPEDFATYYCQHHYGTSLTF | |
| | GQGTKLEIK | |
| opt-43\|VK\| | DIQMTQSPSSLSASVGDRVTITCRASQSISSYLAWYQQKPGKAPKLLIH | 36 |
| #A2 | YAKSLAEGVPSRFSGSGSGTDFTLTISSLQPEDFATYYCQHHYGTSLTF | |
| | GQGTKLEIK | |
| opt-44\|VK\| | DIQMTQSPSSLSASVGDRVTITCRASQSISSYLAWYQQKPGKAPKLLIH | 37 |
| | YAKSLAEGVPSRFSGSGSGTDFTLTISSLQPEDFATYYCQHHYGTSLTF | |
| | GQGTKLEIK | |
| opt-45\|VK\| | DIQMTQSPSSLSASVGDRVTITCRASQSISSYLAWYQQKPGKAPKLLIH | 38 |
| | YAKSLAEGVPSRFSGSGSGTDFTLTISSLQPEDFATYYCQHHYGTSLTF | |
| | GQGTKLEIK | |
| opt-46\|VK\| | DIQMTQSPSSLSASVGDRVTITCRASQSISSYLAWYQQKPGKAPKLLIH | 39 |
| | YAKSLAEGVPSRFSGSGSGTDFTLTISSLQPEDFATYYCQHHYGTSLTF | |
| | GQGTKLEIK | |
| opt-47\|VK\| | DIQMTQSPSSLSASVGDRVTITCRASQSISSYLAWYQQKPGKAPKLLIH | 40 |
| #A3 | YAKSLAEGVPSRFSGSGSGTDFTLTISSLQPEDFATYYCQHHYGTSLTF | |
| | GQGTKLEIK | |
| opt-48\|VK\| | DIQMTQSPSSLSASVGDRVTITCRASQSISSYLAWYQQKPGKAPKLLIH | 41 |
| | YAKSLAEGVPSRFSGSGSGTDFTLTISSLQPEDFATYYCQHHYGTSLTF | |
| | GQGTKLEIK | |
| opt-49\|VK\| | DIQMTQSPSSLSASVGDRVTITCRASQSISSYLAWYQQKPGKAPKLLIH | 42 |
| | YAKSLAEGVPSRFSGSGSGTDFTLTISSLQPEDFATYYCQHHYGTSLTF | |
| | GQGTKLEIK | |
| opt-50\|VK\| | DIQMTQSPSSLSASVGDRVTITCRASQSISSYLAWYQQKPGKAPKLLIH | 43 |
| | YAKSLAEGVPSRFSGSGSGTDFTLTISSLQPEDFATYYCQHHYGTSLTF | |
| | GQGTKLEIK | |
| opt-51\|VK\| | DIQMTQSPSSLSASVGDRVTITCRASQSISSYLAWYQQKPGKAPKLLIH | 44 |
| | YAKSLAEGVPSRFSGSGSGTDFTLTISSLQPEDFATYYCQHHYGTSLTF | |
| | GQGTKLEIK | |
| opt-52\|VK\| | DIQMTQSPSSLSASVGDRVTITCRASQSISSYLAWYQQKPGKAPKLLIH | 45 |
| | YAKSLAEGVPSRFSGSGSGTDFTLTISSLQPEDFATYYCQHHYGTSLTF | |
| | GQGTKLEIK | |
| opt-53\|VK\| | DIQMTQSPSSLSASVGDRVTITCRASQSISSYLAWYQQKPGKAPKLLIH | 46 |
| | YAKSLAEGVPSRFSGSGSGTDFTLTISSLQPEDFATYYCQHHYGTSLTF | |
| | GQGTKLEIK | |
| opt-54\|VK\| | DIQMTQSPSSLSASVGDRVTITCRASQSISSYLAWYQQKPGKAPKLLIH | 47 |
| #A4 | YAKSLAEGVPSRFSGSGSGTDFTLTISSLQPEDFATYYCQHHYGTSLTF | |
| | GQGTKLEIK | |
| opt-55\|VK\| | DIQMTQSPSSLSASVGDRVTITCRASQSISSYLAWYQQKPGKAPKLLIH | 48 |
| | YAKSLAEGVPSRFSGSGSGTDFTLTISSLQPEDFATYYCQHHYGTSLTF | |
| | GQGTKLEIK | |
| opt-56\|VK\| | DIQMTQSPSSLSASVGDRVTITCRASQSISSYLAWYQQKPGKAPKLLIH | 49 |
| | YASSLAEGVPSRFSGSGSGTDFTLTISSLQPEDFATYYCQHHYGTSLTF | |
| | GQGTKLEIK | |
| opt-57\|VK\| | DIQMTQSPSSLSASVGDRVTITCRASQSISSYLAWYQQKPGKAPKLLIH | 50 |
| #A5 | YASSLAEGVPSRFSGSGSGTDFTLTISSLQPEDFATYYCQHHYGTSLTF | |
| | GQGTKLEIK | |
| opt-58\|VK\| | DIQMTQSPSSLSASVGDRVTITCRASQSISSYLAWYQQKPGKAPKLLIH | 51 |
| #A6 | YASSLAEGVPSRFSGSGSGTDFTLTISSLQPEDFATYYCQHHYGTSLTF | |
| | GQGTKLEIK | |
| opt-59\|VK\| | DIQMTQSPSSLSASVGDRVTITCRASQSISSYLAWYQQKPGKAPKLLIH | 52 |
| #A7 | YASSLAEGVPSRFSGSGSGTDFTLTISSLQPEDFATYYCQHHYGTSLTF | |
| | GQGTKLEIK | |
| opt-60\|VK\| | DIQMTQSPSSLSASVGDRVTITCRASQSISSYLAWYQQKPGKAPKLLIH | 53 |
| | YASSLAEGVPSRFSGSGSGTDFTLTISSLQPEDFATYYCQHHYGTSLTF | |
| | GQGTKLEIK | |
| opt-61\|VK\| | DIQMTQSPSSLSASVGDRVTITCRASQSISSYLAWYQQKPGKAPKLLIH | 54 |
| | YASSLAEGVPSRFSGSGSGTDFTLTISSLQPEDFATYYCQHHYGTSLTF | |
| | GQGTKLEIK | |
| opt-62\|VK\| | DIQMTQSPSSLSASVGDRVTITCRASQSISSYLAWYQQKPGKAPKLLIH | 55 |
| | YASSLAEGVPSRFSGSGSGTDFTLTISSLQPEDFATYYCQHHYGTSLTF | |
| | GQGTKLEIK | |
| opt-63\|VK\| | DIQMTQSPSSLSASVGDRVTITCRASQSISSYLAWYQQKPGKAPKLLIH | 56 |
| | YASSLAEGVPSRFSGSGSGTDFTLTISSLQPEDFATYYCQHHYGTSLTF | |
| | GQGTKLEIK | |
| opt-64\|VK\| | DIQMTQSPSSLSASVGDRVTITCRASQSISSYLAWYQQKPGKAPKLLIH | 57 |
| | YASSLAEGVPSRFSGSGSGTDFTLTISSLQPEDFATYYCQHHYGTSLTF | |
| | GQGTKLEIK | |
| opt-65\|VK\| | DIQMTQSPSSLSASVGDRVTITCRASQSISSYLAWYQQKPGKAPKLLIH | 58 |
| | YASSLAEGVPSRFSGSGSGTDFTLTISSLQPEDFATYYCQHHYGTSLTF | |
| | GQGTKLEIK | |
| opt-66\|VK\| | DIQMTQSPSSLSASVGDRVTITCRASQSISSYLAWYQQKPGKAPKLLIH | 59 |
| | YASSLAEGVPSRFSGSGSGTDFTLTISSLQPEDFATYYCQHHYGTSLTF | |
| | GQGTKLEIK | |
| opt-67\|VK\| | DIQMTQSPSSLSASVGDRVTITCRASQSISSYLAWYQQKPGKAPKLLIH | 60 |
| | YASSLAEGVPSRFSGSGSGTDFTLTISSLQPEDFATYYCQHHYGTSLTF | |
| | GQGTKLEIK | |
| opt-68\|VK\| | DIQMTQSPSSLSASVGDRVTITCRASQSISSYLAWYQQKPGKAPKLLIH | 61 |
| | YASSLAEGVPSRFSGSGSGTDFTLTISSLQPEDFATYYCQHHYGTSLTF | |
| | GQGTKLEIK | |
| opt-69\|VK\| | DIQMTQSPSSLSASVGDRVTITCRASQSISSYLAWYQQKPGKAPKLLIH | 62 |
| | YASSLAEGVPSRFSGSGSGTDFTLTISSLQPEDFATYYCQHHYGTSLTF | |
| | GQGTKLEIK | |
| opt-70\|VK\| | DIQMTQSPSSLSASVGDRVTITCRTSENISSYLAWYQQKPGKAPKLLIH | 63 |
| | YAKTLAEGVPSRFSGSGSGTDFTLTISSLQPEDFATYYCQHHYGTSLTF | |
| | GQGTKLEIK | |
| opt-71\|VK\| | DIQMTQSPSSLSASVGDRVTITCRTSENISSYLAWYQQKPGKAPKLLIH | 64 |
| | YAKTLAEGVPSRFSGSGSGTDFTLTISSLQPEDFATYYCQHHYGTSLTF | |
| | GQGTKLEIK | |
| opt-72\|VK\| | DIQMTQSPSSLSASVGDRVTITCRTSENISSYLAWYQQKPGKAPKLLIH | 65 |
| | YAKTLAEGVPSRFSGSGSGTDFTLTISSLQPEDFATYYCQHHYGTSLTF | |
| | GQGTKLEIK | |
| opt-73\|VK\| | DIQMTQSPSSLSASVGDRVTITCRTSENISSYLAWYQQKPGKAPKLLIH | 66 |
| | YAKTLAEGVPSRFSGSGSGTDFTLTISSLQPEDFATYYCQHHYGTSLTF | |
| | GQGTKLEIK | |

TABLE 10
Heavy Chain Variable Region (VH) Amino Acid Sequences

| CLONE | | |
|---|---|---|
| NAME | VH SEQUENCE | SEQ ID NO: |
| HUMANIZED/ | QVQLVQSGAEVKKPGSSVKVSCKASGYIFLTYWMNWVRQAPGQGLE | 67 |
| GRAFT \|VH\| | WMGQIFPASDSTYYNEMFKDRVTITADKSTSTAYMELSSLRSEDTAVYY | |
| 005-G011 | CARSGPYSYYAGGYALDYWGQGTLVTVSS | |
| #A1 | | |
| 406-v18 \|VH\| | QVQLVQSGAEVKKPGSSVKVSCKASGYIFLTYWMNWVRQAPGQGLE | 68 |
| (A72V) | WMGQIFPASDSTYYNEMFKDRVRITVDKSTSTAYMELSSLRSEDTAVY | |
| | YCARSGPYSYYAGGYALDYWGQGTLVTVSS | |
| 406-v20 \|VH\| | QVQLVQSGAEVKKPGSSVKVSCKASGYIFLTYWMNWVRQAPGQGLE | 69 |
| (A72V) | WMGQIFPASDSTYYNEMFKDRVTFTVDKSTSTAYMELSSLRSEDTAVY | |
| | YCARSGPYSYYAGGYALDYWGQGTLVTVSS | |
| opt-27 \|VH\| | QVQLVQSGAEVKKPGSSVKVSCKASGYIFLTYWINWVRQAPGQGLEW | 70 |
| | MGQIFPASGSAYYAQKFQGRVTITADKSTSTAYMELSSLRSEDTAVYYC | |
| | ARSGPYSYYAGGYALDYWGQGTLVTVSS | |
| -opt-28 \|VH\| | QVQLVQSGAEVKKPGSSVKVSCKASGYIFLTYWMNWVRQAPGQGLE | 71 |
| | WMGQIFPASGSAYYAQKFQGRVTITVDKSTSTAYMELSSLRSEDTAVY | |
| | YCARSGPYSYYAGGYALDYWGQGTLVTVSS | |
| opt-29 \|VH\| | QVQLVQSGAEVKKPGSSVKVSCKASGYIFLTYWMNWVRQAPGQGLE | 72 |
| | WMGQIFPASGSAYYNQKFKGRVTITADKSTSTAYMELSSLRSEDTAVY | |
| | YCARSGPYSYYAGGYALDYWGQGTLVTVSS | |
| opt-30 \|VH\| | QVQLVQSGAEVKKPGSSVKVSCKASGYIFLTYWMNWVRQAPGQGLE | 73 |
| | WMGQIFPASDSTYYAQMFQDRVTITADKSTSTAYMELSSLRSEDTAVY | |
| | YCARSGPYSYYAGGYALDYWGQGTLVTVSS | |
| opt-31 \|VH\| | QVQLVQSGAEVKKPGSSVKVSCKASGYIFLTYWINWVRQAPGQGLEW | 74 |
| | MGQIFPASGSTYYNEKFKGRVTITADKSTSTAYMELSSLRSEDTAVYYC | |
| | ARSGPYSYYAGGYALDYWGQGTLVTVSS | |
| -opt-32 \|VH\| | QVQLVQSGAEVKKPGSSVKVSCKASGYIFLTYWINWVRQAPGQGLEW | 75 |
| | MGQIFPASGSTYYAQKFQDRVTITVDKSTSTAYMELSSLRSEDTAVYYC | |
| | ARSGPYSYYAGGYALDYWGQGTLVTVSS | |
| opt-33 \|VH\| | QVQLVQSGAEVKKPGSSVKVSCKASGYIFLTYWINWVRQAPGQGLEW | 76 |
| | MGQIFPASDSTYYNQKFKGRVTITADKSTSTAYMELSSLRSEDTAVYYC | |
| | ARSGPYSYYAGGYALDYWGQGTLVTVSS | |
| opt-34 \|VH\| | QVQLVQSGAEVKKPGSSVKVSCKASGYIFLTYWMNWVRQAPGQGLE | 77 |
| | WMGQIFPASDSAYYNEKFKGRVTITVDKSTSTAYMELSSLRSEDTAVYY | |
| | CARSGPYSYYAGGYALDYWGQGTLVTVSS | |
| opt-35 \|VH\| | QVQLVQSGAEVKKPGSSVKVSCKASGYIFLTYWINWVRQAPGQGLEW | 78 |
| | MGQIFPASDSAYYAQKFQDRVTITADKSTSTAYMELSSLRSEDTAVYYC | |
| | ARSGPYSYYAGGYALDYWGQGTLVTVSS | |
| opt-36 \|VH\| | QVQLVQSGAEVKKPGSSVKVSCKASGYIFLTYWMNWVRQAPGQGLE | 79 |
| | WMGQIFPASGSAYYAQKFQGRVTITVDKSTSTAYMELSSLRSEDTAVY | |
| | YCARSGPYSYYAGGYALDYWGQGTLVTVSS | |
| opt-37 \|VH\| | QVQLVQSGAEVKKPGSSVKVSCKASGYIFLTYWMNWVRQAPGQGLE | 80 |
| | WMGQIFPASGSAYYAQKFQGRVTITADKSTSTAYMELSSLRSEDTAVY | |
| | YCARSGPYSYYAGGYALDYWGQGTLVTVSS | |
| opt-38 \|VH\| | QVQLVQSGAEVKKPGSSVKVSCKASGYIFLTYWMNWVRQAPGQGLE | 81 |
| | WMGQIFPASGSAYYNEKFKDRVTITVDKSTSTAYMELSSLRSEDTAVYY | |
| | CARSGPYSYYAGGYALDYWGQGTLVTVSS | |
| opt-39 \|VH\| | QVQLVQSGAEVKKPGSSVKVSCKASGYIFLTYWISWVRQAPGQGLEW | 82 |
| | MGQIFPASDSAYYNQMFKDRVTITVDKSTSTAYMELSSLRSEDTAVYYC | |
| | ARSGPYSYYAGGYALDYWGQGTLVTVSS | |
| opt-40 \|VH\| | QVQLVQSGAEVKKPGSSVKVSCKASGYIFLTYWINWVRQAPGQGLEW | 83 |
| | MGQIFPASGSAYYAQKFQGRVTITADKSTSTAYMELSSLRSEDTAVYYC | |
| | ARSGPYSYYAGGYALDYWGQGTLVTVSS | |
| opt-41 \|VH\| | QVQLVQSGAEVKKPGSSVKVSCKASGYIFLTYWINWVRQAPGQGLEW | 84 |
| | MGQIFPASDSAYYNQMFKDRVTITADKSTSTAYMELSSLRSEDTAVYYC | |
| | ARSGPYSYYAGGYALDYWGQGTLVTVSS | |
| opt-42 \|VH\| | QVQLVQSGAEVKKPGSSVKVSCKASGYIFLTYWMNWVRQAPGQGLE | 85 |
| | WMGQIFPASGSAYYAQKFQGRVTITVDKSTSTAYMELSSLRSEDTAVY | |
| | YCARSGPYSYYAGGYALDYWGQGTLVTVSS | |
| opt-43 \|VH\| | QVQLVQSGAEVKKPGSSVKVSCKASGYIFLTYWMNWVRQAPGQGLE | 86 |
| #A2 | WMGQIFPASGSAYYNQKFKGRVTITADKSTSTAYMELSSLRSEDTAVY | |
| | YCARSGPYSYYAGGYALDYWGQGTLVTVSS | |
| opt-44 \|VH\| | QVQLVQSGAEVKKPGSSVKVSCKASGYIFLTYWMNWVRQAPGQGLE | 87 |
| | WMGQIFPASDSTYYAQMFQDRVTITADKSTSTAYMELSSLRSEDTAVY | |
| | YCARSGPYSYYAGGYALDYWGQGTLVTVSS | |
| opt-45 \|VH\| | QVQLVQSGAEVKKPGSSVKVSCKASGYIFLTYWINWVRQAPGQGLEW | 88 |
| | MGQIFPASGSTYYNEKFKGRVTITADKSTSTAYMELSSLRSEDTAVYYC | |
| | ARSGPYSYYAGGYALDYWGQGTLVTVSS | |
| opt-46 \|VH\| | QVQLVQSGAEVKKPGSSVKVSCKASGYIFLTYWINWVRQAPGQGLEW | 89 |
| | MGQIFPASGSTYYAQKFQDRVTITVDKSTSTAYMELSSLRSEDTAVYYC | |
| | ARSGPYSYYAGGYALDYWGQGTLVTVSS | |
| opt-47 \|VH\| | QVQLVQSGAEVKKPGSSVKVSCKASGYIFLTYWINWVRQAPGQGLEW | 90 |
| #A3 | MGQIFPASDSTYYNQKFKGRVTITADKSTSTAYMELSSLRSEDTAVYYC | |
| | ARSGPYSYYAGGYALDYWGQGTLVTVSS | |
| opt-48 \|VH\| | QVQLVQSGAEVKKPGSSVKVSCKASGYIFLTYWMNWVRQAPGQGLE | 91 |
| | WMGQIFPASDSAYYNEKFKGRVTITVDKSTSTAYMELSSLRSEDTAVYY | |
| | CARSGPYSYYAGGYALDYWGQGTLVTVSS | |
| opt-49 \|VH\| | QVQLVQSGAEVKKPGSSVKVSCKASGYIFLTYWINWVRQAPGQGLEW | 92 |
| | MGQIFPASDSAYYAQKFQDRVTITADKSTSTAYMELSSLRSEDTAVYYC | |
| | ARSGPYSYYAGGYALDYWGQGTLVTVSS | |
| opt-50 \|VH\| | QVQLVQSGAEVKKPGSSVKVSCKASGYIFLTYWMNWVRQAPGQGLE | 93 |
| | WMGQIFPASGSAYYAQKFQGRVTITVDKSTSTAYMELSSLRSEDTAVY | |
| | YCARSGPYSYYAGGYALDYWGQGTLVTVSS | |
| opt-51 \|VH\| | QVQLVQSGAEVKKPGSSVKVSCKASGYIFLTYWMNWVRQAPGQGLE | 94 |
| | WMGQIFPASGSAYYAQKFQGRVTITADKSTSTAYMELSSLRSEDTAVY | |
| | YCARSGPYSYYAGGYALDYWGQGTLVTVSS | |
| opt-52 \|VH\| | QVQLVQSGAEVKKPGSSVKVSCKASGYIFLTYWMNWVRQAPGQGLE | 95 |
| | WMGQIFPASGSAYYNEKFKDRVTITVDKSTSTAYMELSSLRSEDTAVYY | |
| | CARSGPYSYYAGGYALDYWGQGTLVTVSS | |
| opt-53 \|VH\| | QVQLVQSGAEVKKPGSSVKVSCKASGYIFLTYWISWVRQAPGQGLEW | 96 |
| | MGQIFPASDSAYYNQMFKDRVTITVDKSTSTAYMELSSLRSEDTAVYYC | |
| | ARSGPYSYYAGGYALDYWGQGTLVTVSS | |
| opt-54 \|VH\| | QVQLVQSGAEVKKPGSSVKVSCKASGYIFLTYWINWVRQAPGQGLEW | 97 |
| #A4 | MGQIFPASGSAYYAQKFQGRVTITADKSTSTAYMELSSLRSEDTAVYYC | |
| | ARSGPYSYYAGGYALDYWGQGTLVTVSS | |
| opt-55 \|VH\| | QVQLVQSGAEVKKPGSSVKVSCKASGYIFLTYWINWVRQAPGQGLEW | 98 |
| | MGQIFPASDSAYYNQMFKDRVTITADKSTSTAYMELSSLRSEDTAVYYC | |
| | ARSGPYSYYAGGYALDYWGQGTLVTVSS | |
| opt-56 \|VH\| | QVQLVQSGAEVKKPGSSVKVSCKASGYIFLTYWINWVRQAPGQGLEW | 99 |
| | MGQIFPASGSAYYAQKFQGRVTITADKSTSTAYMELSSLRSEDTAVYYC | |
| | ARSGPYSYYAGGYALDYWGQGTLVTVSS | |
| opt-57 \|VH\| | QVQLVQSGAEVKKPGSSVKVSCKASGYIFLTYWMNWVRQAPGQGLE | 100 |
| #A5 | WMGQIFPASGSAYYAQKFQGRVTITVDKSTSTAYMELSSLRSEDTAVY | |
| | YCARSGPYSYYAGGYALDYWGQGTLVTVSS | |
| opt-58 \|VH\| | QVQLVQSGAEVKKPGSSVKVSCKASGYIFLTYWMNWVRQAPGQGLE | 101 |
| #A6 | WMGQIFPASGSAYYNQKFKGRVTITADKSTSTAYMELSSLRSEDTAVY | |
| | YCARSGPYSYYAGGYALDYWGQGTLVTVSS | |
| opt-59 \|VH\| | QVQLVQSGAEVKKPGSSVKVSCKASGYIFLTYWINWVRQAPGQGLEW | 102 |
| #A7 | MGQIFPASGSTYYNEKFKGRVTITADKSTSTAYMELSSLRSEDTAVYYC | |
| | ARSGPYSYYAGGYALDYWGQGTLVTVSS | |
| opt-60 \|VH\| | QVQLVQSGAEVKKPGSSVKVSCKASGYIFLTYWINWVRQAPGQGLEW | 103 |
| | MGQIFPASGSTYYAQKFQDRVTITVDKSTSTAYMELSSLRSEDTAVYYC | |
| | ARSGPYSYYAGGYALDYWGQGTLVTVSS | |
| opt-61 \|VH\| | QVQLVQSGAEVKKPGSSVKVSCKASGYIFLTYWINWVRQAPGQGLEW | 104 |
| | MGQIFPASDSTYYNQKFKGRVTITADKSTSTAYMELSSLRSEDTAVYYC | |
| | ARSGPYSYYAGGYALDYWGQGTLVTVSS | |
| opt-62 \|VH\| | QVQLVQSGAEVKKPGSSVKVSCKASGYIFLTYWMNWVRQAPGQGLE | 105 |
| | WMGQIFPASDSAYYNEKFKGRVTITVDKSTSTAYMELSSLRSEDTAVYY | |
| | CARSGPYSYYAGGYALDYWGQGTLVTVSS | |
| opt-63 \|VH\| | QVQLVQSGAEVKKPGSSVKVSCKASGYIFLTYWINWVRQAPGQGLEW | 106 |
| | MGQIFPASDSAYYAQKFQDRVTITADKSTSTAYMELSSLRSEDTAVYYC | |
| | ARSGPYSYYAGGYALDYWGQGTLVTVSS | |
| opt-64 \|VH\| | QVQLVQSGAEVKKPGSSVKVSCKASGYIFLTYWMNWVRQAPGQGLE | 107 |
| | WMGQIFPASGSAYYAQKFQGRVTITVDKSTSTAYMELSSLRSEDTAVY | |
| | YCARSGPYSYYAGGYALDYWGQGTLVTVSS | |
| opt-65 \|VH\| | QVQLVQSGAEVKKPGSSVKVSCKASGYIFLTYWMNWVRQAPGQGLE | 108 |
| | WMGQIFPASGSAYYAQKFQGRVTITADKSTSTAYMELSSLRSEDTAVY | |
| | YCARSGPYSYYAGGYALDYWGQGTLVTVSS | |
| opt-66 \|VH\| | QVQLVQSGAEVKKPGSSVKVSCKASGYIFLTYWMNWVRQAPGQGLE | 109 |
| | WMGQIFPASGSAYYNEKFKDRVTITVDKSTSTAYMELSSLRSEDTAVYY | |
| | CARSGPYSYYAGGYALDYWGQTLVTVSS | |
| opt-67 \|VH\| | QVQLVQSGAEVKKPGSSVKVSCKASGYIFLTYWISWVRQAPGQGLEW | 110 |
| | MGQIFPASDSAYYNQMFKDRVTITVDKSTSTAYMELSSLRSEDTAVYYC | |
| | ARSGPYSYYAGGYALDYWGQGTLVTVSS | |
| opt-68 \|VH\| | QVQLVQSGAEVKKPGSSVKVSCKASGYIFLTYWINWVRQAPGQGLEW | 111 |
| | MGQIFPASGSAYYAQKFQGRVTITADKSTSTAYMELSSLRSEDTAVYYC | |
| | ARSGPYSYYAGGYALDYWGQGTLVTVSS | |
| opt-6 \|VH\| | QVQLVQSGAEVKKPGSSVKVSCKASGYIFLTYWINWVRQAPGQGLEW | 112 |
| | MGQIFPASDSAYYNQMFKDRVTITADKSTSTAYMELSSLRSEDTAVYYC | |
| | ARSGPYSYYAGGYALDYWGQGTLVTVSS | |
| opt-70 \|VH\| | QVQLVQSGAEVKKPGSSVKVSCKASGYIFLTYWINWVRQAPGQGLEW | 113 |
| | MGQIFPASGSTYYNEKFKDRVRITVDKSTSTAYMELSSLRSEDTAVYYC | |
| | ARSGPYSYYAGGYALDYWGQGTLVTVSS | |
| opt-71 \|VH\| | QVQLVQSGAEVKKPGSSVKVSCKASGYIFLTYWINWVRQAPGQGLEW | 114 |
| | MGQIFPASGSTYYNEMFKDRVRITVDKSTSTAYMELSSLRSEDTAVYYC | |
| | ARSGPYSYYAGGYALDYWGQGTLVTVSS | |
| opt-72 \|VH\| | QVQLVQSGAEVKKPGSSVKVSCKASGYIFLTYWMNWVRQAPGQGLE | 115 |
| | WMGQIFPASGSTYYNEKFKDRVRITVDKSTSTAYMELSSLRSEDTAVYY | |
| | CARSGPYSYYAGGYALDYWGQGTLVTVSS | |
| opt-73 \|VH\| | QVQLVQSGAEVKKPGSSVKVSCKASGYIFLTYWMNWVRQAPGQGLE | 116 |
| | WMGQIFPASGSTYYNEMFKDRVRITVDKSTSTAYMELSSLRSEDTAVY | |
| | YCARSGPYSYYAGGYALDYWGQGTLVTVSS | |

The CDR sequences from the humanized variable regions, VL/VH from clones OPT 27-73 derived from murine antibody 005-GO11, including Opt-43 (#A2), Opt 47 (#A3), Opt-54 (#A4), Opt-57 (#A5), Opt-58 (#A6), and Opt-59 (#A7) are depicted below. The CDRs are indicated according to the naming conventions KABAT, CCG, CHOTHIA, and IMGT.

TABLE 11
L-CDR1 Amino Acid Sequences-KABAT, CCG, & CHOTHIA

| | KABAT/CCG/ | | T25A | | E27Q | N28S | | N30S | | | | | |
|---|---|---|---|---|---|---|---|---|---|---|---|---|---|
| | CHOTHIA | AMINO ACID NO. | | | | | | | | | | | SEQ ID |
| | LCDR1 | 24 | 25 | 26 | 27 | 28 | 29 | 30 | 31 | 32 | 33 | 34 | NO. |
| PARENT GRAFT | RTSENINSYLA | R | T | S | E | N | I | N | S | Y | L | A | 3 |
| 005-G011 (#A1) | | | | | | | | | | | | | |
| OPT-27 | RASQSISSYLA | R | A | S | Q | S | I | S | S | Y | L | A | 117 |
| OPT28 | RASENINSYLA | R | A | S | E | N | I | N | S | Y | L | A | 118 |
| OPT 29 | RTSQNINSYLA | R | T | S | Q | N | I | N | S | Y | L | A | 119 |
| OPT 30 | RASQSISSYLA | R | A | S | Q | S | I | S | S | Y | L | A | 117 |
| OPT 31 | RTSESINSYLA | R | T | S | E | S | I | N | S | Y | L | A | 120 |
| OPT-32 | RASQSINSYLA | R | A | S | Q | S | I | N | S | Y | L | A | 121 |
| OPT-33 | RTSESISSYLA | R | T | S | E | S | I | S | S | Y | L | A | 122 |
| OPT-34 | RASQSISSYLA | R | A | S | Q | S | I | S | S | Y | L | A | 117 |
| OPT-35 | RASQSISSYLA | R | A | S | Q | S | I | S | S | Y | L | A | 117 |
| OPT-36 | RASQSINSYLA | R | A | S | Q | S | I | N | S | Y | L | A | 121 |
| OPT-37 | RTSESINSYLA | R | T | S | E | S | I | N | S | Y | L | A | 120 |
| OPT-38 | RASENISSYLA | R | A | S | E | N | I | S | S | Y | L | A | 123 |
| OPT-39 | RTSQNISSYLA | R | T | S | Q | N | I | S | S | Y | L | A | 124 |
| OPT-40 | RTSQSINSYLA | R | T | S | Q | S | I | N | S | Y | L | A | 125 |
| OPT-41 | RASQSISSYLA | R | A | S | Q | S | I | S | S | Y | L | A | 117 |
| OPT-42 | RASQSISSYLA | R | A | S | Q | S | I | S | S | Y | L | A | 117 |
| OPT-43 #A2 | RASQSISSYLA | R | A | S | Q | S | I | S | S | Y | L | A | 117 |
| OPT-44 | RASQSISSYLA | R | A | S | Q | S | I | S | S | Y | L | A | 117 |
| OPT-45 | RASQSISSYLA | R | A | S | Q | S | I | S | S | Y | L | A | 117 |
| OPT-46 | RASQSISSYLA | R | A | S | Q | S | I | S | S | Y | L | A | 117 |
| OPT-47 #A3 | RASQSISSYLA | R | A | S | Q | S | I | S | S | Y | L | A | 117 |
| OPT-48 | RASQSISSYLA | R | A | S | Q | S | I | S | S | Y | L | A | 117 |
| OPT-49 | RASQSISSYLA | R | A | S | Q | S | I | S | S | Y | L | A | 117 |
| OPT-50 | RASQSISSYLA | R | A | S | Q | S | I | S | S | Y | L | A | 117 |
| OPT-51 | RASQSISSYLA | R | A | S | Q | S | I | S | S | Y | L | A | 117 |
| OPT-52 | RASQSISSYLA | R | A | S | Q | S | I | S | S | Y | L | A | 117 |
| OPT-53 | RASQSISSYLA | R | A | S | Q | S | I | S | S | Y | L | A | 117 |
| Opt-54 #A4 | RASQSISSYLA | R | A | S | Q | S | I | S | S | Y | L | A | 117 |
| OPT-55 | RASQSISSYLA | R | A | S | Q | S | I | S | S | Y | L | A | 117 |
| OPT-56 | RASQSISSYLA | R | A | S | Q | S | I | S | S | Y | L | A | 117 |
| Opt-57 #A5 | RASQSISSYLA | R | A | S | Q | S | I | S | S | Y | L | A | 117 |
| Opt-58 #A6 | RASQSISSYLA | R | A | S | Q | S | I | S | S | Y | L | A | 117 |
| Opt-59 #A7 | RASQSISSYLA | R | A | S | Q | S | I | S | S | Y | L | A | 117 |
| OPT-60 | RASQSISSYLA | R | A | S | Q | S | I | S | S | Y | L | A | 117 |
| OPT-61 | RASQSISSYLA | R | A | S | Q | S | I | S | S | Y | L | A | 117 |
| OPT-62 | RASQSISSYLA | R | A | S | Q | S | I | S | S | Y | L | A | 117 |
| OPT-63 | RASQSISSYLA | R | A | S | Q | S | I | S | S | Y | L | A | 117 |
| OPT-64 | RASQSISSYLA | R | A | S | Q | S | I | S | S | Y | L | A | 117 |
| OPT-65 | RASQSISSYLA | R | A | S | Q | S | I | S | S | Y | L | A | 117 |
| OPT-66 | RASQSISSYLA | R | A | S | Q | S | I | S | S | Y | L | A | 117 |
| OPT-67 | RASQSISSYLA | R | A | S | Q | S | I | S | S | Y | L | A | 117 |
| OPT-68 | RASQSISSYLA | R | A | S | Q | S | I | S | S | Y | L | A | 117 |
| OPT-69 | RASQSISSYLA | R | A | S | Q | S | I | S | S | Y | L | A | 117 |
| OPT-70 | RTSENISSYLA | R | T | S | E | N | I | S | S | Y | L | A | 126 |
| OPT-71 | RTSENISSYLA | R | T | S | E | N | I | S | S | Y | L | A | 126 |
| OPT-72 | RTSENISSYLA | R | T | S | E | N | I | S | S | Y | L | A | 126 |
| OPT 73 | RTSENISSYLA | R | T | S | E | N | I | S | S | Y | L | A | 126 |
| | CONSENSUS | R | X1 | S | X2 | X3 | I | X4 | S | Y | L | A | 134 |

TABLE 12
L-CDR1 Amino Acid Sequences-IMGT

| | | E27Q | N28S | | N30S | | | |
|---|---|---|---|---|---|---|---|---|
| | IMGT | AMINO ACID NO. | | | | | | SEQ ID |
| | LCDR1 | 27 | 28 | 29 | 30 | 31 | 32 | NO. |
| PARENT | ENINSY | E | N | I | N | S | Y | 6 |
| GRAFT 005- | | | | | | | | |
| G011 (#A1) | | | | | | | | |
| OPT-27 | QSISSY | Q | S | I | S | S | Y | 127 |
| OPT28 | ENINSY | E | N | I | N | S | Y | 6 |
| OPT 29 | QNINSY | Q | N | I | N | S | Y | 128 |
| OPT 30 | QSISSY | Q | S | I | S | S | Y | 127 |
| OPT 31 | ESINSY | E | S | I | N | S | Y | 129 |
| OPT-32 | QSINSY | Q | S | I | N | S | Y | 130 |
| OPT-33 | ESISSY | E | S | I | S | S | Y | 131 |
| OPT-34 | QSISSY | Q | S | I | S | S | Y | 127 |
| OPT-35 | QSISSY | Q | S | I | S | S | Y | 127 |
| OPT-36 | QSINSY | Q | S | I | N | S | Y | 130 |
| OPT-37 | ESINSY | E | S | I | N | S | Y | 129 |
| OPT-38 | ENISSY | E | N | I | S | S | Y | 132 |
| OPT-39 | QNISSY | Q | N | I | S | S | Y | 133 |
| OPT-40 | QSINSY | Q | S | I | N | S | Y | 130 |
| OPT-41 | QSISSY | Q | S | I | S | S | Y | 127 |
| OPT-42 | QSISSY | Q | S | I | S | S | Y | 127 |
| OPT-43 #A2 | QSISSY | Q | S | I | S | S | Y | 127 |
| OPT-44- | QSISSY | Q | S | I | S | S | Y | 127 |
| OPT-45 | QSISSY | Q | S | I | S | S | Y | 127 |
| OPT-46 | QSISSY | Q | S | I | S | S | Y | 127 |
| OPT-47 #A3 | QSISSY | Q | S | I | S | S | Y | 127 |
| OPT-48 | QSISSY | Q | S | I | S | S | Y | 127 |
| OPT-49 | QSISSY | Q | S | I | S | S | Y | 127 |
| OPT-50 | QSISSY | Q | S | I | S | S | Y | 127 |
| OPT-51 | QSISSY | Q | S | I | S | S | Y | 127 |
| OPT-52 | QSISSY | Q | S | I | S | S | Y | 127 |
| OPT-53 | QSISSY | Q | S | I | S | S | Y | 127 |
| Opt-54 #A4 | QSISSY | Q | S | I | S | S | Y | 127 |
| OPT-55 | QSISSY | Q | S | I | S | S | Y | 127 |
| OPT-56 | QSISSY | Q | S | I | S | S | Y | 127 |
| Opt-57 #A5 | QSISSY | Q | S | I | S | S | Y | 127 |
| Opt-58 #A6 | QSISSY | Q | S | I | S | S | Y | 127 |
| Opt-59 #A7 | QSISSY | Q | S | I | S | S | Y | 127 |
| OPT-60 | QSISSY | Q | S | I | S | S | Y | 127 |
| OPT-61 | QSISSY | Q | S | I | S | S | Y | 127 |
| OPT-62 | QSISSY | Q | S | I | S | S | Y | 127 |
| OPT-63 | QSISSY | Q | S | I | S | S | Y | 127 |
| OPT-64 | QSISSY | Q | S | I | S | S | Y | 127 |
| OPT-65 | QSISSY | Q | S | I | S | S | Y | 127 |
| OPT-66 | QSISSY | Q | S | I | S | S | Y | 127 |
| OPT-67 | QSISSY | Q | S | I | S | S | Y | 127 |
| OPT-68 | QSISSY | Q | S | I | S | S | Y | 127 |
| OPT-69 | QSISSY | Q | S | I | S | S | Y | 127 |
| OPT-70 | ENISSY | E | N | I | S | S | Y | 132 |
| OPT-71 | ENISSY | E | N | I | S | S | Y | 132 |
| OPT-72 | ENISSY | E | N | I | S | S | Y | 132 |
| OPT 73 | ENISSY | E | N | I | S | S | Y | 132 |
| | CONSENSUS | X1 | X2 | I | X3 | S | Y | 135 |

TABLE 13
L-CDR2 Amino Acid Sequences-KABAT, CCG, & CHOTHIA

| | KABAT/CCG/ | Y50A | | K52S | T53Y | | A55Q | E56S | |
|---|---|---|---|---|---|---|---|---|---|
| | CHOTHIA | AMINO ACID NO. | | | | | | | SEQ ID |
| | LCDR2 | 50 | 51 | 52 | 53 | 54 | 55 | 56 | NO. |
| PARENT GRAFT | YAKTLAE | Y | A | K | T | L | A | E | 4 |
| 005-G011 (#A1) | | | | | | | | | |
| OPT-27 | YAKSLAE | Y | A | K | S | L | A | E | 136 |
| OPT28 | YAKTLAE | Y | A | K | T | L | A | E | 4 |
| OPT 29 | YAKTLAE | Y | A | K | T | L | A | E | 4 |
| OPT 30 | YASSLAE | Y | A | S | S | L | A | E | 137 |
| OPT 31 | YAKSLAE | Y | A | K | S | L | A | E | 136 |
| OPT-32 | YAKTLAE | Y | A | K | T | L | A | E | 4 |
| OPT-33 | YASTLAE | Y | A | S | T | L | A | E | 138 |
| OPT-34 | YAKTLAE | Y | A | K | T | L | A | E | 4 |
| OPT-35 | YAKSLAE | Y | A | K | S | L | A | E | 136 |
| OPT-36 | YASTLAE | Y | A | S | T | L | A | E | 138 |
| OPT-37 | YAKSLAE | Y | A | K | S | L | A | E | 136 |
| OPT-38 | YAKSLAE | Y | A | K | S | L | A | E | 136 |
| OPT-39 | YAKSLAE | Y | A | K | S | L | A | E | 136 |
| OPT-40 | YASSLAE | Y | A | S | S | L | A | E | 137 |
| OPT-41 | YAKTLAE | Y | A | K | T | L | A | E | 4 |
| OPT-42 | YAKSLAE | Y | A | K | S | L | A | E | 136 |
| OPT-43 #A2 | YAKSLAE | Y | A | K | S | L | A | E | 136 |
| OPT-44 | YAKSLAE | Y | A | K | S | L | A | E | 136 |
| OPT-45 | YAKSLAE | Y | A | K | S | L | A | E | 136 |
| OPT-46 | YAKSLAE | Y | A | K | S | L | A | E | 136 |
| OPT-47 #A3 | YAKSLAE | Y | A | K | S | L | A | E | 136 |
| OPT-48 | YAKSLAE | Y | A | K | S | L | A | E | 136 |
| OPT-49 | YAKSLAE | Y | A | K | S | L | A | E | 136 |
| OPT-50 | YAKSLAE | Y | A | K | S | L | A | E | 136 |
| OPT-51 | YAKSLAE | Y | A | K | S | L | A | E | 136 |
| OPT-52 | YAKSLAE | Y | A | K | S | L | A | E | 136 |
| OPT-53 | YAKSLAE | Y | A | K | S | L | A | E | 136 |
| Opt-54 #A4 | YAKSLAE | Y | A | K | S | L | A | E | 136 |
| OPT-55 | YAKSLAE | Y | A | K | S | L | A | E | 136 |
| OPT-56 | YASSLAE | Y | A | S | S | L | A | E | 137 |
| Opt-57 #A5 | YASSLAE | Y | A | S | S | L | A | E | 137 |
| Opt-58 #A6 | YASSLAE | Y | A | S | S | L | A | E | 137 |
| Opt-59 #A7 | YASSLAE | Y | A | S | S | L | A | E | 137 |
| OPT-60 | YASSLAE | Y | A | S | S | L | A | E | 137 |
| OPT-61 | YASSLAE | Y | A | S | S | L | A | E | 137 |
| OPT-62 | YASSLAE | Y | A | S | S | L | A | E | 137 |
| OPT-63 | YASSLAE | Y | A | S | S | L | A | E | 137 |
| OPT-64 | YASSLAE | Y | A | S | S | L | A | E | 137 |
| OPT-65 | YASSLAE | Y | A | S | S | L | A | E | 137 |
| OPT-66 | YASSLAE | Y | A | S | S | L | A | E | 137 |
| OPT-67 | YASSLAE | Y | A | S | S | L | A | E | 137 |
| OPT-68 | YASSLAE | Y | A | S | S | L | A | E | 137 |
| OPT-69 | YASSLAE | Y | A | S | S | L | A | E | 137 |
| OPT-70 | YAKTLAE | Y | A | K | T | L | A | E | 4 |
| OPT-71 | YAKTLAE | Y | A | K | T | L | A | E | 4 |
| OPT-72 | YAKTLAE | Y | A | K | T | L | A | E | 4 |
| OPT 73 | YAKTLAE | Y | A | K | T | L | A | E | 4 |
| | CONSENSUS | Y | A | X1 | X2 | L | A | E | 140 |

TABLE 14
L-CDR2 Amino Acid Sequences-IMGT

| | | Y50A | K52S | | |
|---|---|---|---|---|---|
| | IMGT | AMINO ACID NO. | | | SEQ ID |
| | LCDR2 | 50 | 51 | 52 | NO. |
| PARENT GRAFT | YAKTLAE | Y | A | K | 7 |
| 005-G011 (#A1) | (SEQ ID | | | | |
| | NO: 4) | | | | |
| OPT-27 | YAK | Y | A | K | 7 |
| OPT28 | YAK | Y | A | K | 7 |
| OPT 29 | YAK | Y | A | K | 7 |
| OPT 30 | YAS | Y | A | S | 139 |
| OPT 31 | YAK | Y | A | K | 7 |
| OPT-32 | YAK | Y | A | K | 7 |
| OPT-33 | YAS | Y | A | S | 139 |
| OPT-34 | YAK | Y | A | K | 7 |
| OPT-35 | YAK | Y | A | K | 7 |
| OPT-36 | YAS | Y | A | S | 139 |
| OPT-37 | YAK | Y | A | K | 7 |
| OPT-38 | YAK | Y | A | K | 7 |
| OPT-39 | YAK | Y | A | K | 7 |
| OPT-40 | YAS | Y | A | S | 139 |
| OPT-41 | YAK | Y | A | K | 7 |
| OPT-42 | YAK | Y | A | K | 7 |
| OPT-43 #A2 | YAK | Y | A | K | 7 |
| OPT-44 | YAK | Y | A | K | 7 |
| OPT-45 | YAK | Y | A | K | 7 |
| OPT-46 | YAK | Y | A | K | 7 |
| OPT-47 #A3 | YAK | Y | A | K | 7 |
| OPT-48 | YAK | Y | A | K | 7 |
| OPT-49 | YAK | Y | A | K | 7 |
| OPT-50 | YAK | Y | A | K | 7 |
| OPT-51 | YAK | Y | A | K | 7 |
| OPT-52 | YAK | Y | A | K | 7 |
| OPT-53 | YAK | Y | A | K | 7 |
| Opt-54 #A4 | YAK | Y | A | K | 7 |
| OPT-55 | YAK | Y | A | K | 7 |
| OPT-56 | YAS | Y | A | S | 139 |
| Opt-57 #A5 | YAS | Y | A | S | 139 |
| Opt-58 #A6 | YAS | Y | A | S | 139 |
| Opt-59 #A7 | YAS | Y | A | S | 139 |
| OPT-60 | YAS | Y | A | S | 139 |
| OPT-61 | YAS | Y | A | S | 139 |
| OPT-62 | YAS | Y | A | S | 139 |
| OPT-63 | YAS | Y | A | S | 139 |
| OPT-64 | YAS | Y | A | S | 139 |
| OPT-65 | YAS | Y | A | S | 139 |
| OPT-66 | YAS | Y | A | S | 139 |
| OPT-67 | YAS | Y | A | S | 139 |
| OPT-68 | YAS | Y | A | S | 139 |
| OPT-69 | YAS | Y | A | S | 139 |
| OPT-70 | YAK | Y | A | K | 7 |
| OPT-71 | YAK | Y | A | K | 7 |
| OPT-72 | YAK | Y | A | K | 7 |
| OPT 73 | YAK | Y | A | K | 7 |
| | CONSENSUS | Y | A | X1 | 141 |

TABLE 15
L-CDR3 Amino Acid Sequences-KABAT, CCG, Chothia, and IMGT

| | KABAT/CCG/ | | H90Q | H91S | | G933 | | S95P | | | |
|---|---|---|---|---|---|---|---|---|---|---|---|
| | CHOTHIA/IMGT | AMINO ACID NO. | | | | | | | | | SEQ ID |
| | LCDR3 | 89 | 90 | 91 | 92 | 93 | 94 | 95 | 96 | 97 | NO. |
| PARENT | QHHYGTSLT | Q | H | H | Y | G | T | S | L | T | 5 |
| GRAFT 005- | | | | | | | | | | | |
| G011 (#A1) | | | | | | | | | | | |
| OPT-27 | QHHYGTSLT | Q | H | H | Y | G | T | S | L | T | 5 |
| OPT28 | QHHYGTSLT | Q | H | H | Y | G | T | S | L | T | 5 |
| OPT 29 | QHHYGTSLT | Q | H | H | Y | G | T | S | L | T | 5 |
| OPT 30 | QHHYGTSLT | Q | H | H | Y | G | T | S | L | T | 5 |
| OPT 31 | QHHYGTSLT | Q | H | H | Y | G | T | S | L | T | 5 |
| OPT-32 | QHHYGTSLT | Q | H | H | Y | G | T | S | L | T | 5 |
| OPT-33 | QHHYGTSLT | Q | H | H | Y | G | T | S | L | T | 5 |
| OPT-34 | QHHYGTSLT | Q | H | H | Y | G | T | S | L | T | 5 |
| OPT-35 | QHHYGTSLT | Q | H | H | Y | G | T | S | L | T | 5 |
| OPT-36 | QHHYGTSLT | Q | H | H | Y | G | T | S | L | T | 5 |
| OPT-37 | QHHYGTSLT | Q | H | H | Y | G | T | S | L | T | 5 |
| OPT-38 | QHHYGTSLT | Q | H | H | Y | G | T | S | L | T | 5 |
| OPT-39 | QHHYGTSLT | Q | H | H | Y | G | T | S | L | T | 5 |
| OPT-40 | QHHYGTSLT | Q | H | H | Y | G | T | S | L | T | 5 |
| OPT-41 | QHHYGTSLT | Q | H | H | Y | G | T | S | L | T | 5 |
| OPT-42 | QHHYGTSLT | Q | H | H | Y | G | T | S | L | T | 5 |
| OPT-43 #A2 | QHHYGTSLT | Q | H | H | Y | G | T | S | L | T | 5 |
| OPT-44 | QHHYGTSLT | Q | H | H | Y | G | T | S | L | T | 5 |
| OPT-45 | QHHYGTSLT | Q | H | H | Y | G | T | S | L | T | 5 |
| OPT-46 | QHHYGTSLT | Q | H | H | Y | G | T | S | L | T | 5 |
| OPT-47 #A3 | QHHYGTSLT | Q | H | H | Y | G | T | S | L | T | 5 |
| OPT-48 | QHHYGTSLT | Q | H | H | Y | G | T | S | L | T | 5 |
| OPT-49 | QHHYGTSLT | Q | H | H | Y | G | T | S | L | T | 5 |
| OPT-50 | QHHYGTSLT | Q | H | H | Y | G | T | S | L | T | 5 |
| OPT-51 | QHHYGTSLT | Q | H | H | Y | G | T | S | L | T | 5 |
| OPT-52 | QHHYGTSLT | Q | H | H | Y | G | T | S | L | T | 5 |
| OPT-53 | QHHYGTSLT | Q | H | H | Y | G | T | S | L | T | 5 |
| Opt-54 #A4 | QHHYGTSLT | Q | H | H | Y | G | T | S | L | T | 5 |
| OPT-55 | QHHYGTSLT | Q | H | H | Y | G | T | S | L | T | 5 |
| OPT-56 | QHHYGTSLT | Q | H | H | Y | G | T | S | L | T | 5 |
| Opt-57 #A5 | QHHYGTSLT | Q | H | H | Y | G | T | S | L | T | 5 |
| Opt-58 #A6 | QHHYGTSLT | Q | H | H | Y | G | T | S | L | T | 5 |
| Opt-59 #A7 | QHHYGTSLT | Q | H | H | Y | G | T | S | L | T | 5 |
| OPT-60 | QHHYGTSLT | Q | H | H | Y | G | T | S | L | T | 5 |
| OPT-61 | QHHYGTSLT | Q | H | H | Y | G | T | S | L | T | 5 |
| OPT-62 | QHHYGTSLT | Q | H | H | Y | G | T | S | L | T | 5 |
| OPT-63 | QHHYGTSLT | Q | H | H | Y | G | T | S | L | T | 5 |
| OPT-64 | QHHYGTSLT | Q | H | H | Y | G | T | S | L | T | 5 |
| OPT-65 | QHHYGTSLT | Q | H | H | Y | G | T | S | L | T | 5 |
| OPT-66 | QHHYGTSLT | Q | H | H | Y | G | T | S | L | T | 5 |
| OPT-67 | QHHYGTSLT | Q | H | H | Y | G | T | S | L | T | 5 |
| OPT-68 | QHHYGTSLT | Q | H | H | Y | G | T | S | L | T | 5 |
| OPT-69 | QHHYGTSLT | Q | H | H | Y | G | T | S | L | T | 5 |
| OPT-70 | QHHYGTSLT | Q | H | H | Y | G | T | S | L | T | 5 |
| OPT-71 | QHHYGTSLT | Q | H | H | Y | G | T | S | L | T | 5 |
| OPT-72 | QHHYGTSLT | Q | H | H | Y | G | T | S | L | T | 5 |
| OPT 73 | QHHYGTSLT | Q | H | H | Y | G | T | S | L | T | 5 |
| | CONSENSUS | Q | H | H | Y | G | T | S | L | T | 5 |

TABLE 16
H-CDR1 Amino Acid Sequences-KABAT

| | | | | | M43I | N35S | |
|---|---|---|---|---|---|---|---|
| | KABAT | AMINO ACID NO. | | | | | SEQ ID |
| | HCDR1 | 31 | 32 | 33 | 34 | 35 | NO. |
| PARENTGRAFT | TYWMN | T | Y | W | M | N | 8 |
| 005-G011 (#A1) | | | | | | | |
| OPT-27 | TYWIN | T | Y | W | I | N | 142 |
| OPT28 | TYWMN | T | Y | W | M | N | 8 |
| OPT 29 | TYWMN | T | Y | W | M | N | 8 |
| OPT 30 | TYWMN | T | Y | W | M | N | 8 |
| OPT 31 | TYWIN | T | Y | W | I | N | 142 |
| OPT-32 | TYWIN | T | Y | W | I | N | 142 |
| OPT-33 | TYWIN | T | Y | W | I | N | 142 |
| OPT-34 | TYWMN | T | Y | W | M | N | 8 |
| OPT-35 | TYWIN | T | Y | W | I | N | 142 |
| OPT-36 | TYWMN | T | Y | W | M | N | 8 |
| OPT-37 | TYWMN | T | Y | W | M | N | 8 |
| OPT-38 | TYWMN | T | Y | W | M | N | 8 |
| OPT-39 | TYWIS | T | Y | W | I | S | 143 |
| OPT-40 | TYWIN | T | Y | W | I | N | 142 |
| OPT-41 | TYWIN | T | Y | W | I | N | 142 |
| OPT-42 | TYWMN | T | Y | W | M | N | 8 |
| OPT-43 #A2 | TYWMN | T | Y | W | M | N | 8 |
| OPT-44 | TYWMN | T | Y | W | M | N | 8 |
| OPT-45 | TYWIN | T | Y | W | I | N | 142 |
| OPT-46 | TYWIN | T | Y | W | I | N | 142 |
| OPT-47 #A3 | TYWIN | T | Y | W | I | N | 142 |
| OPT-48 | TYWMN | T | Y | W | M | N | 8 |
| OPT-49 | TYWIN | T | Y | W | I | N | 142 |
| OPT-50 | TYWMN | T | Y | W | M | N | 8 |
| OPT-51 | TYWMN | T | Y | W | M | N | 8 |
| OPT-52 | TYWMN | T | Y | W | M | N | 8 |
| OPT-53 | TYWIS | T | Y | W | I | S | 143 |
| Opt-54 #A4 | TYWIN | T | Y | W | I | N | 142 |
| OPT-55 | TYWIN | T | Y | W | I | N | 142 |
| OPT-56 | TYWIN | T | Y | W | I | N | 142 |
| Opt-57 #A5 | TYWMN | T | Y | W | M | N | 8 |
| Opt-58 #A6 | TYWMN | T | Y | W | M | N | 8 |
| Opt-59 #A7 | TYWIN | T | Y | W | I | N | 142 |
| OPT-60 | TYWIN | T | Y | W | I | N | 142 |
| OPT-61 | TYWIN | T | Y | W | I | N | 142 |
| OPT-62 | TYWMN | T | Y | W | M | N | 8 |
| OPT-63 | TYWIN | T | Y | W | I | N | 142 |
| OPT-64 | TYWMN | T | Y | W | M | N | 8 |
| OPT-65 | TYWMN | T | Y | W | M | N | 8 |
| OPT-66 | TYWMN | T | Y | W | M | N | 8 |
| OPT-67 | TYWIS | T | Y | W | I | S | 143 |
| OPT-68 | TYWIN | T | Y | W | I | N | 142 |
| OPT-69 | TYWIN | T | Y | W | I | N | 142 |
| OPT-70 | TYWIN | T | Y | W | I | N | 142 |
| OPT-71 | TYWIN | T | Y | W | I | N | 142 |
| OPT-72 | TYWMN | T | Y | W | M | N | 8 |
| OPT 73 | TYWMN | T | Y | W | M | N | 8 |
| | | T | Y | W | X1 | X2 | 146 |

TABLE 17
H-CDR1 Amino Acid Sequences-CCG

| | | | | | | | | | | M34I | N35S | |
|---|---|---|---|---|---|---|---|---|---|---|---|---|
| | CCG | AMINO ACID NO. | | | | | | | | | | SEQ ID |
| | HCDR1 | 26 | 27 | 28 | 29 | 30 | 31 | 32 | 33 | 34 | 35 | NO. |
| PARENT GRAFT | GYIFLTYWMN | G | Y | I | F | L | T | Y | W | M | N | 11 |
| 005-G011 (#A1) | | | | | | | | | | | | |
| OPT-27 | GYIFLTYWIN | G | Y | I | F | L | T | Y | W | I | N | 144 |
| OPT28 | GYIFLTYWMN | G | Y | I | F | L | T | Y | W | M | N | 11 |
| OPT 29 | GYIFLTYWMN | G | Y | I | F | L | T | Y | W | M | N | 11 |
| OPT 30 | GYIFLTYWMN | G | Y | I | F | L | T | Y | W | M | N | 11 |
| OPT 31 | GYIFLTYWIN | G | Y | I | F | L | T | Y | W | I | N | 144 |
| OPT-32 | GYIFLTYWIN | G | Y | I | F | L | T | Y | W | I | N | 144 |
| OPT-33 | GYIFLTYWIN | G | Y | I | F | L | T | Y | W | I | N | 144 |
| OPT-34 | GYIFLTYWMN | G | Y | I | F | L | T | Y | W | M | N | 11 |
| OPT-35 | GYIFLTYWIN | G | Y | I | F | L | T | Y | W | I | N | 144 |
| OPT-36 | GYIFLTYWMN | G | Y | I | F | L | T | Y | W | M | N | 11 |
| OPT-37 | GYIFLTYWMN | G | Y | I | F | L | T | Y | W | M | N | 11 |
| OPT-38 | GYIFLTYWMN | G | Y | I | F | L | T | Y | W | M | N | 11 |
| OPT-39 | GYIFLTYWIS | G | Y | I | F | L | T | Y | W | I | S | 145 |
| OPT-40 | GYIFLTYWIN | G | Y | I | F | L | T | Y | W | I | N | 144 |
| OPT-41 | GYIFLTYWIN | G | Y | I | F | L | T | Y | W | I | N | 144 |
| OPT-42 | GYIFLTYWMN | G | Y | I | F | L | T | Y | W | M | N | 11 |
| OPT-43 #A2 | GYIFLTYWMN | G | Y | I | F | L | T | Y | W | M | N | 11 |
| OPT-44 | GYIFLTYWMN | G | Y | I | F | L | T | Y | W | M | N | 11 |
| OPT-45 | GYIFLTYWIN | G | Y | I | F | L | T | Y | W | I | N | 144 |
| OPT-46 | GYIFLTYWIN | G | Y | I | F | L | T | Y | W | I | N | 144 |
| OPT-47 #A3 | GYIFLTYWIN | G | Y | I | F | L | T | Y | W | I | N | 144 |
| OPT-48 | GYIFLTYWMN | G | Y | I | F | L | T | Y | W | M | N | 11 |
| OPT-49 | GYIFLTYWIN | G | Y | I | F | L | T | Y | W | I | N | 144 |
| OPT-50 | GYIFLTYWMN | G | Y | I | F | L | T | Y | W | M | N | 11 |
| OPT-51 | GYIFLTYWMN | G | Y | I | F | L | T | Y | W | M | N | 11 |
| OPT-52 | GYIFLTYWMN | G | Y | I | F | L | T | Y | W | M | N | 11 |
| OPT-53 | GYIFLTYWIS | G | Y | I | F | L | T | Y | W | I | S | 145 |
| Opt-54 #A4 | GYIFLTYWIN | G | Y | I | F | L | T | Y | W | I | N | 144 |
| OPT-55 | GYIFLTYWIN | G | Y | I | F | L | T | Y | W | I | N | 144 |
| OPT-56 | GYIFLTYWIN | G | Y | I | F | L | T | Y | W | I | N | 144 |
| Opt-57 #A5 | GYIFLTYWMN | G | Y | I | F | L | T | Y | W | M | N | 11 |
| Opt-58 #A6 | GYIFLTYWMN | G | Y | I | F | L | T | Y | W | M | N | 11 |
| Opt-59 #A7 | GYIFLTYWIN | G | Y | I | F | L | T | Y | W | I | N | 144 |
| OPT-60 | GYIFLTYWIN | G | Y | I | F | L | T | Y | W | I | N | 144 |
| OPT-61 | GYIFLTYWIN | G | Y | I | F | L | T | Y | W | I | N | 144 |
| OPT-62 | GYIFLTYWMN | G | Y | I | F | L | T | Y | W | M | N | 11 |
| OPT-63 | GYIFLTYWIN | G | Y | I | F | L | T | Y | W | I | N | 144 |
| OPT-64 | GYIFLTYWMN | G | Y | I | F | L | T | Y | W | M | N | 11 |
| OPT-65 | GYIFLTYWMN | G | Y | I | F | L | T | Y | W | M | N | 11 |
| OPT-66 | GYIFLTYWMN | G | Y | I | F | L | T | Y | W | M | N | 11 |
| OPT-67 | GYIFLTYWIS | G | Y | I | F | L | T | Y | W | I | S | 145 |
| OPT-68 | GYIFLTYWIN | G | Y | I | F | L | T | Y | W | I | N | 144 |
| OPT-69 | GYIFLTYWIN | G | Y | I | F | L | T | Y | W | I | N | 144 |
| OPT-70 | GYIFLTYWIN | G | Y | I | F | L | T | Y | W | I | N | 144 |
| OPT-71 | GYIFLTYWIN | G | Y | I | F | L | T | Y | W | I | N | 144 |
| OPT-72 | GYIFLTYWMN | G | Y | I | F | L | T | Y | W | M | N | 11 |
| OPT 73 | GYIFLTYWMN | G | Y | I | F | L | T | Y | W | M | N | 11 |
| | | G | Y | I | F | L | T | Y | W | X1 | X2 | 194 |

TABLE 18
H-CDR1 Amino Acid Sequences-CHOTHIA

| | CHOTHIA | AMINO ACID NO. | | | | | | | SEQ ID |
|---|---|---|---|---|---|---|---|---|---|
| | HCDR1 | 26 | 27 | 28 | 29 | 30 | 31 | 32 | NO. |
| PARENT GRAFT | GYIFLTY | G | Y | I | F | L | T | Y | 12 |
| 005-G011 (#A1) | | | | | | | | | |
| OPT-27 | GYIFLTY | G | Y | I | F | L | T | Y | 12 |
| OPT28 | GYIFLTY | G | Y | I | F | L | T | Y | 12 |
| OPT 29 | GYIFLTY | G | Y | I | F | L | T | Y | 12 |
| OPT 30 | GYIFLTY | G | Y | I | F | L | T | Y | 12 |
| OPT 31 | GYIFLTY | G | Y | I | F | L | T | Y | 12 |
| OPT-32 | GYIFLTY | G | Y | I | F | L | T | Y | 12 |
| OPT-33 | GYIFLTY | G | Y | I | F | L | T | Y | 12 |
| OPT-34 | GYIFLTY | G | Y | I | F | L | T | Y | 12 |
| OPT-35 | GYIFLTY | G | Y | I | F | L | T | Y | 12 |
| OPT-36 | GYIFLTY | G | Y | I | F | L | T | Y | 12 |
| OPT-37 | GYIFLTY | G | Y | I | F | L | T | Y | 12 |
| OPT-38 | GYIFLTY | G | Y | I | F | L | T | Y | 12 |
| OPT-39 | GYIFLTY | G | Y | I | F | L | T | Y | 12 |
| OPT-40 | GYIFLTY | G | Y | I | F | L | T | Y | 12 |
| OPT-41 | GYIFLTY | G | Y | I | F | L | T | Y | 12 |
| OPT-42 | GYIFLTY | G | Y | I | F | L | T | Y | 12 |
| OPT-43 #A2 | GYIFLTY | G | Y | I | F | L | T | Y | 12 |
| OPT-44 | GYIFLTY | G | Y | I | F | L | T | Y | 12 |
| OPT-45 | GYIFLTY | G | Y | I | F | L | T | Y | 12 |
| OPT-46 | GYIFLTY | G | Y | I | F | L | T | Y | 12 |
| OPT-47 #A3 | GYIFLTY | G | Y | I | F | L | T | Y | 12 |
| OPT-48 | GYIFLTY | G | Y | I | F | L | T | Y | 12 |
| OPT-49 | GYIFLTY | G | Y | I | F | L | T | Y | 12 |
| OPT-50 | GYIFLTY | G | Y | I | F | L | T | Y | 12 |
| OPT-51 | GYIFLTY | G | Y | I | F | L | T | Y | 12 |
| OPT-52 | GYIFLTY | G | Y | I | F | L | T | Y | 12 |
| OPT-53 | GYIFLTY | G | Y | I | F | L | T | Y | 12 |
| Opt-54 #A4 | GYIFLTY | G | Y | I | F | L | T | Y | 12 |
| OPT-55 | GYIFLTY | G | Y | I | F | L | T | Y | 12 |
| OPT-56 | GYIFLTY | G | Y | I | F | L | T | Y | 12 |
| Opt-57 #A5 | GYIFLTY | G | Y | I | F | L | T | Y | 12 |
| Opt-58 #A6 | GYIFLTY | G | Y | I | F | L | T | Y | 12 |
| Opt-59 #A7 | GYIFLTY | G | Y | I | F | L | T | Y | 12 |
| OPT-60 | GYIFLTY | G | Y | I | F | L | T | Y | 12 |
| OPT-61 | GYIFLTY | G | Y | I | F | L | T | Y | 12 |
| OPT-62 | GYIFLTY | G | Y | I | F | L | T | Y | 12 |
| OPT-63 | GYIFLTY | G | Y | I | F | L | T | Y | 12 |
| OPT-64 | GYIFLTY | G | Y | I | F | L | T | Y | 12 |
| OPT-65 | GYIFLTY | G | Y | I | F | L | T | Y | 12 |
| OPT-66 | GYIFLTY | G | Y | I | F | L | T | Y | 12 |
| OPT-67 | GYIFLTY | G | Y | I | F | L | T | Y | 12 |
| OPT-68 | GYIFLTY | G | Y | I | F | L | T | Y | 12 |
| OPT-69 | GYIFLTY | G | Y | I | F | L | T | Y | 12 |
| OPT-70 | GYIFLTY | G | Y | I | F | L | T | Y | 12 |
| OPT-71 | GYIFLTY | G | Y | I | F | L | T | Y | 12 |
| OPT-72 | GYIFLTY | G | Y | I | F | L | T | Y | 12 |
| OPT 73 | GYIFLTY | G | Y | I | F | L | T | Y | 12 |
| | | G | Y | I | F | L | T | Y | 12 |

TABLE 19
H-CDR1 Amino Acid Sequences-IMGT

| | IMGT | AMINO ACID NO. | | | | | | | | SEQ ID |
|---|---|---|---|---|---|---|---|---|---|---|
| | HCDR1 | 26 | 27 | 28 | 29 | 30 | 31 | 32 | 33 | NO. |
| PARENTGRAFT | GYIFLTYW | G | Y | I | F | L | T | Y | W | 14 |
| 005-G011 | | | | | | | | | | |
| (#A1) | | | | | | | | | | |
| OPT-27 | GYIFLTYW | G | Y | I | F | L | T | Y | W | 14 |
| OPT 28 | GYIFLTYW | G | Y | I | F | L | T | Y | W | 14 |
| OPT 29 | GYIFLTYW | G | Y | I | F | L | T | Y | W | 14 |
| OPT 30 | GYIFLTYW | G | Y | I | F | L | T | Y | W | 14 |
| OPT 31 | GYIFLTYW | G | Y | I | F | L | T | Y | W | 14 |
| OPT-32 | GYIFLTYW | G | Y | I | F | L | T | Y | W | 14 |
| OPT-33 | GYIFLTYW | G | Y | I | F | L | T | Y | W | 14 |
| OPT-34 | GYIFLTYW | G | Y | I | F | L | T | Y | W | 14 |
| OPT-35 | GYIFLTYW | G | Y | I | F | L | T | Y | W | 14 |
| OPT-36 | GYIFLTYW | G | Y | I | F | L | T | Y | W | 14 |
| OPT-37 | GYIFLTYW | G | Y | I | F | L | T | Y | W | 14 |
| OPT-38 | GYIFLTYW | G | Y | I | F | L | T | Y | W | 14 |
| OPT-39 | GYIFLTYW | G | Y | I | F | L | T | Y | W | 14 |
| OPT-40 | GYIFLTYW | G | Y | I | F | L | T | Y | W | 14 |
| OPT-41 | GYIFLTYW | G | Y | I | F | L | T | Y | W | 14 |
| OPT-42 | GYIFLTYW | G | Y | I | F | L | T | Y | W | 14 |
| OPT-43 #A2 | GYIFLTYW | G | Y | I | F | L | T | Y | W | 14 |
| OPT-44 | GYIFLTYW | G | Y | I | F | L | T | Y | W | 14 |
| OPT-45 | GYIFLTYW | G | Y | I | F | L | T | Y | W | 14 |
| OPT-46 | GYIFLTYW | G | Y | I | F | L | T | Y | W | 14 |
| OPT-47 #A3 | GYIFLTYW | G | Y | I | F | L | T | Y | W | 14 |
| OPT-48 | GYIFLTYW | G | Y | I | F | L | T | Y | W | 14 |
| OPT-49 | GYIFLTYW | G | Y | I | F | L | T | Y | W | 14 |
| OPT-50 | GYIFLTYW | G | Y | I | F | L | T | Y | W | 14 |
| OPT-51 | GYIFLTYW | G | Y | I | F | L | T | Y | W | 14 |
| OPT-52 | GYIFLTYW | G | Y | I | F | L | T | Y | W | 14 |
| OPT-53 | GYIFLTYW | G | Y | I | F | L | T | Y | W | 14 |
| Opt-54 #A4 | GYIFLTYW | G | Y | I | F | L | T | Y | W | 14 |
| OPT-55 | GYIFLTYW | G | Y | I | F | L | T | Y | W | 14 |
| OPT-56 | GYIFLTYW | G | Y | I | F | L | T | Y | W | 14 |
| Opt-57 #A5 | GYIFLTYW | G | Y | I | F | L | T | Y | W | 14 |
| Opt-58 #A6 | GYIFLTYW | G | Y | I | F | L | T | Y | W | 14 |
| Opt-59 #A7 | GYIFLTYW | G | Y | I | F | L | T | Y | W | 14 |
| OPT-60 | GYIFLTYW | G | Y | I | F | L | T | Y | W | 14 |
| OPT-61 | GYIFLTYW | G | Y | I | F | L | T | Y | W | 14 |
| OPT-62 | GYIFLTYW | G | Y | I | F | L | T | Y | W | 14 |
| OPT-63 | GYIFLTYW | G | Y | I | F | L | T | Y | W | 14 |
| OPT-64 | GYIFLTYW | G | Y | I | F | L | T | Y | W | 14 |
| OPT-65 | GYIFLTYW | G | Y | I | F | L | T | Y | W | 14 |
| OPT-66 | GYIFLTYW | G | Y | I | F | L | T | Y | W | 14 |
| OPT-67 | GYIFLTYW | G | Y | I | F | L | T | Y | W | 14 |
| OPT-68 | GYIFLTYW | G | Y | I | F | L | T | Y | W | 14 |
| OPT-69 | GYIFLTYW | G | Y | I | F | L | T | Y | W | 14 |
| OPT-70 | GYIFLTYW | G | Y | I | F | L | T | Y | W | 14 |
| OPT-71 | GYIFLTYW | G | Y | I | F | L | T | Y | W | 14 |
| OPT-72 | GYIFLTYW | G | Y | I | F | L | T | Y | W | 14 |
| OPT 73 | GYIFLTYW | G | Y | I | F | L | T | Y | W | 14 |
| | | G | Y | I | F | L | T | Y | W | 14 |

TABLE 20
H-CDR2 Amino Acid Sequences-KABAT, CCG

| | | | | | | | | D56G | | T58A | | | N61A | E62Q | M63K | | K65Q | D66G | SEQ |
|---|---|---|---|---|---|---|---|---|---|---|---|---|---|---|---|---|---|---|---|
| | KABAT/CCG | AMINO ACID NO. | | | | | | | | | | | | | | | | | ID |
| | HCDR2 | 50 | 51 | 52 | 53 | 54 | 55 | 56 | 57 | 58 | 59 | 60 | 61 | 62 | 63 | 64 | 65 | 66 | NO. |
| PARENT | QIFPASDSTYYNEMFKD | Q | I | F | P | A | S | D | S | T | Y | Y | N | E | M | F | K | D | 9 |
| GRAFT | | | | | | | | | | | | | | | | | | | |
| 005-G011 | | | | | | | | | | | | | | | | | | | |
| (#A1) | | | | | | | | | | | | | | | | | | | |
| OPT-27 | QIFPASGSAYYAQKFQG | Q | I | F | P | A | S | G | S | A | Y | Y | A | Q | K | F | Q | G | 148 |
| OPT-28 | QIFPASGSAYYAQKFQG | Q | I | F | P | A | S | G | S | A | Y | Y | A | Q | K | F | Q | G | 148 |
| OPT 29 | QIFPASGSAYYNQKFKG | Q | I | F | P | A | S | G | S | A | Y | Y | N | Q | K | F | K | G | 149 |
| OPT 30 | QIFPASDSTYYAQMFQD | Q | I | F | P | A | S | D | S | T | Y | Y | A | Q | M | F | Q | D | 150 |
| OPT 31 | QIFPASGSTYYNEKFKG | Q | I | F | P | A | S | G | S | T | Y | Y | N | E | K | F | K | G | 151 |
| OPT-32 | QIFPASGSTYYAQKFQD | Q | I | F | P | A | S | G | S | T | Y | Y | A | Q | K | F | Q | D | 152 |
| OPT-33 | QIFPASDSTYYNQKFKG | Q | I | F | P | A | S | D | S | T | Y | Y | N | Q | K | F | K | G | 153 |
| OPT-34 | QIFPASDSAYYNEKFKG | Q | I | F | P | A | S | D | S | A | Y | Y | N | E | K | F | K | G | 154 |
| OPT-35 | QIFPASDSAYYAQKFQD | Q | I | F | P | A | S | D | S | A | Y | Y | A | Q | K | F | Q | D | 155 |
| OPT-36 | QIFPASGSAYYAQKFQG | Q | I | F | P | A | S | G | S | A | Y | Y | A | Q | K | F | Q | G | 148 |
| OPT-37 | QIFPASGSAYYAQKFQG | Q | I | F | P | A | S | G | S | A | Y | Y | A | Q | K | F | Q | G | 148 |
| OPT-38 | QIFPASGSAYYNEKFKD | Q | I | F | P | A | S | G | S | A | Y | Y | N | E | K | F | K | D | 156 |
| OPT-39 | QIFPASDSAYYNQMFKD | Q | I | F | P | A | S | D | S | A | Y | Y | N | Q | M | F | K | D | 157 |
| OPT-40 | QIFPASGSAYYAQKFQG | Q | I | F | P | A | S | G | S | A | Y | Y | A | Q | K | F | Q | G | 148 |
| OPT-41 | QIFPASDSAYYNQMFKD | Q | I | F | P | A | S | D | S | A | Y | Y | N | Q | M | F | K | D | 157 |
| OPT-42 | QIFPASGSAYYAQKFQG | Q | I | F | P | A | S | G | S | A | Y | Y | A | Q | K | F | Q | G | 148 |
| OPT-43 | QIFPASGSAYYNQKFKG | Q | I | F | P | A | S | G | S | A | Y | Y | N | Q | K | F | K | G | 149 |
| #A2 | | | | | | | | | | | | | | | | | | | |
| OPT-44 | QIFPASDSTYYAQMFQD | Q | I | F | P | A | S | D | S | T | Y | Y | A | Q | M | F | Q | D | 158 |
| OPT-45 | QIFPASGSTYYNEKFKG | Q | I | F | P | A | S | G | S | T | Y | Y | N | E | K | F | K | G | 151 |
| OPT-46 | QIFPASGSTYYAQKFQD | Q | I | F | P | A | S | G | S | T | Y | Y | A | Q | K | F | Q | D | 152 |
| OPT-47 | QIFPASDSTYYNQKFKG | Q | I | F | P | A | S | D | S | T | Y | Y | N | Q | K | F | K | G | 153 |
| #A3 | | | | | | | | | | | | | | | | | | | |
| OPT-48 | QIFPASDSAYYNEKFKG | Q | I | F | P | A | S | D | S | A | Y | Y | N | E | K | F | K | G | 154 |
| OPT-49 | QIFPASDSAYYAQKFQD | Q | I | F | P | A | S | D | S | A | Y | Y | A | Q | K | F | Q | D | 155 |
| OPT-50 | QIFPASGSAYYAQKFQG | Q | I | F | P | A | S | G | S | A | Y | Y | A | Q | K | F | Q | G | 148 |
| OPT-51 | QIFPASGSAYYAQKFQG | Q | I | F | P | A | S | G | S | A | Y | Y | A | Q | K | F | Q | G | 148 |
| OPT-52 | QIFPASGSAYYNEKFKD | Q | I | F | P | A | S | G | S | A | Y | Y | N | E | K | F | K | D | 156 |
| OPT-53 | QIFPASDSAYYNQMFKD | Q | I | F | P | A | S | D | S | A | Y | Y | N | Q | M | F | K | D | 157 |
| Opt-54 | QIFPASGSAYYAQKFQG | Q | I | F | P | A | S | G | S | A | Y | Y | A | Q | K | F | Q | G | 148 |
| #A4 | | | | | | | | | | | | | | | | | | | |
| OPT-55 | QIFPASDSAYYNQMFKD | Q | I | F | P | A | S | D | S | A | Y | Y | N | Q | M | F | K | D | 157 |
| OPT-56 | QIFPASGSAYYAQKFQG | Q | I | F | P | A | S | G | S | A | Y | Y | A | Q | K | F | Q | G | 148 |
| Opt-57 | QIFPASGSAYYAQKFQG | Q | I | F | P | A | S | G | S | A | Y | Y | A | Q | K | F | Q | G | 148 |
| #A5 | | | | | | | | | | | | | | | | | | | |
| Opt-58 | QIFPASGSAYYNQKFKG | Q | I | F | P | A | S | G | S | A | Y | Y | N | Q | K | F | K | G | 149 |
| #A6 | | | | | | | | | | | | | | | | | | | |
| Opt-59 | QIFPASGSTYYNEKFKG | Q | I | F | P | A | S | G | S | T | Y | Y | N | E | K | F | K | G | 151 |
| #A7 | | | | | | | | | | | | | | | | | | | |
| OPT-60 | QIFPASGSTYYAQKFQD | Q | I | F | P | A | S | G | S | T | Y | Y | A | Q | K | F | Q | D | 152 |
| OPT-61 | QIFPASDSTYYNQKFKG | Q | I | F | P | A | S | D | S | T | Y | Y | N | Q | K | F | K | G | 153 |
| OPT-62 | QIFPASDSAYYNEKFKG | Q | I | F | P | A | S | D | S | A | Y | Y | N | E | K | F | K | G | 154 |
| OPT-63 | QIFPASDSAYYAQKFQD | Q | I | F | P | A | S | D | S | A | Y | Y | A | Q | K | F | Q | D | 155 |
| OPT-64 | QIFPASGSAYYAQKFQG | Q | I | F | P | A | S | G | S | A | Y | Y | A | Q | K | F | Q | G | 148 |
| OPT-65 | QIFPASGSAYYAQKFQG | Q | I | F | P | A | S | G | S | A | Y | Y | A | Q | K | F | Q | G | 148 |
| OPT-66 | QIFPASGSAYYNEKFKD | Q | I | F | P | A | S | G | S | A | Y | Y | N | E | K | F | K | D | 156 |
| OPT-67 | QIFPASDSAYYNQMFKD | Q | I | F | P | A | S | D | S | A | Y | Y | N | Q | M | F | K | D | 157 |
| OPT-68 | QIFPASGSAYYAQKFQG | Q | I | F | P | A | S | G | S | A | Y | Y | A | Q | K | F | Q | G | 148 |
| OPT-69 | QIFPASDSAYYNQMFKD | Q | I | F | P | A | S | D | S | A | Y | Y | N | Q | M | F | K | D | 157 |
| OPT-70 | QIFPASGSTYYNEKFKD | Q | I | F | P | A | S | G | S | T | Y | Y | N | E | K | F | K | D | 159 |
| OPT-71 | QIFPASGSTYYNEMFKD | Q | I | F | P | A | S | G | S | T | Y | Y | N | E | M | F | K | D | 160 |
| OPT-72 | QIFPASGSTYYNEKFKD | Q | I | F | P | A | S | G | S | T | Y | Y | N | E | K | F | K | D | 159 |
| OPT 73 | QIFPASGSTYYNEMFKD | Q | I | F | P | A | S | G | S | T | Y | Y | N | E | M | F | K | D | 160 |
| | CONSENSUS | Q | I | F | P | A | S | X1 | S | X2 | Y | Y | X3 | X4 | X5 | F | X6 | X7 | 165 |

TABLE 21
H-CDR2 Amino Acid Sequences-CHOTHIA

| | | | | | | D56G | | |
|---|---|---|---|---|---|---|---|---|
| | | AMINO ACID NO. | | | | | | |
| | CHOTHIA | | | | | | | SEQ ID |
| | HCDR2 | 52 | 53 | 54 | 55 | 56 | 57 | NO. |
| PARENT | FPASDS | F | P | A | S | D | S | 13 |
| GRAFT 005- | | | | | | | | |
| G011 (#A1) | | | | | | | | |
| OPT-27 | FPASGS | F | P | A | S | G | S | 161 |
| OPT-28 | FPASGS | F | P | A | S | G | S | 161 |
| OPT 29 | FPASGS | F | P | A | S | G | S | 161 |
| OPT 30 | FPASDS | F | P | A | S | D | S | 13 |
| OPT 31 | FPASGS | F | P | A | S | G | S | 161 |
| OPT-32 | FPASGS | F | P | A | S | G | S | 161 |
| OPT-33 | FPASDS | F | P | A | S | D | S | 13 |
| OPT-34 | FPASDS | F | P | A | S | D | S | 13 |
| OPT-35 | FPASDS | F | P | A | S | D | S | 13 |
| OPT-36 | FPASGS | F | P | A | S | G | S | 161 |
| OPT-37 | FPASGS | F | P | A | S | G | S | 161 |
| OPT-38 | FPASGS | F | P | A | S | G | S | 161 |
| OPT-39 | FPASDS | F | P | A | S | D | S | 13 |
| OPT-40 | FPASGS | F | P | A | S | G | S | 161 |
| OPT-41 | FPASDS | F | P | A | S | D | S | 13 |
| OPT-42 | FPASGS | F | P | A | S | G | S | 161 |
| OPT-43 #A2 | FPASGS | F | P | A | S | G | S | 161 |
| OPT-44 | FPASDS | F | P | A | S | D | S | 13 |
| OPT-45 | FPASGS | F | P | A | S | G | S | 161 |
| OPT-46 | FPASGS | F | P | A | S | G | S | 161 |
| OPT-47 #A3 | FPASDS | F | P | A | S | D | S | 13 |
| OPT-48 | FPASDS | F | P | A | S | D | S | 13 |
| OPT-49 | FPASDS | F | P | A | S | D | S | 13 |
| OPT-50 | FPASGS | F | P | A | S | G | S | 161 |
| OPT-51 | FPASGS | F | P | A | S | G | S | 161 |
| OPT-52 | FPASGS | F | P | A | S | G | S | 161 |
| OPT-53 | FPASDS | F | P | A | S | D | S | 13 |
| Opt-54 #A4 | FPASGS | F | P | A | S | G | S | 161 |
| OPT-55 | FPASDS | F | P | A | S | D | S | 13 |
| OPT-56 | FPASGS | F | P | A | S | G | S | 161 |
| Opt-57 #A5 | FPASGS | F | P | A | S | G | S | 161 |
| Opt-58 #A6 | FPASGS | F | P | A | S | G | S | 161 |
| Opt-59 #A7 | FPASGS | F | P | A | S | G | S | 161 |
| OPT-60 | FPASGS | F | P | A | S | G | S | 161 |
| OPT-61 | FPASDS | F | P | A | S | D | S | 13 |
| OPT-62 | FPASDS | F | P | A | S | D | S | 13 |
| OPT-63 | FPASDS | F | P | A | S | D | S | 13 |
| OPT-64 | FPASGS | F | P | A | S | G | S | 161 |
| OPT-65 | FPASGS | F | P | A | S | G | S | 161 |
| OPT-66 | FPASGS | F | P | A | S | G | S | 161 |
| OPT-67 | FPASDS | F | P | A | S | D | S | 13 |
| OPT-68 | FPASGS | F | P | A | S | G | S | 161 |
| OPT-69 | FPASDS | F | P | A | S | D | S | 13 |
| OPT-70 | FPASGS | F | P | A | S | G | S | 161 |
| OPT-71 | FPASGS | F | P | A | S | G | S | 161 |
| OPT-72 | FPASGS | F | P | A | S | G | S | 161 |
| OPT 73 | FPASGS | F | P | A | S | G | S | 161 |
| | CONSENSUS | F | P | A | S | X1 | S | 166 |

TABLE 22
H-CDR2 Amino Acid Sequences-IMGT

| | | | | | | | D56G | | T58A | |
|---|---|---|---|---|---|---|---|---|---|---|
| | | AMINO ACID NO. | | | | | | | | |
| | IMGT | | | | | | | | | SEQ |
| | HCDR2 | 51 | 52 | 53 | 54 | 55 | 56 | 57 | 58 | ID NO. |
| PARENT | IFPASDST | I | F | P | A | S | D | S | T | 15 |
| GRAFT 005- | | | | | | | | | | |
| G011 (#A1) | | | | | | | | | | |
| OPT-27 | IFPASGSA | I | F | P | A | S | G | S | A | 162 |
| OPT-28 | IFPASGSA | I | F | P | A | S | G | S | A | 162 |
| OPT 29 | IFPASGSA | I | F | P | A | S | G | S | A | 162 |
| OPT 30 | IFPASDST | I | F | P | A | S | D | S | T | 15 |
| OPT 31 | IFPASGST | I | F | P | A | S | G | S | T | 163 |
| OPT-32 | IFPASGST | I | F | P | A | S | G | S | T | 163 |
| OPT-33 | IFPASDST | I | F | P | A | S | D | S | T | 15 |
| OPT-34 | IFPASDSA | I | F | P | A | S | D | S | A | 164 |
| OPT-35 | IFPASDSA | I | F | P | A | S | D | S | A | 164 |
| OPT-36 | IFPASGSA | I | F | P | A | S | G | S | A | 162 |
| OPT-37 | IFPASGSA | I | F | P | A | S | G | S | A | 162 |
| OPT-38 | IFPASGSA | I | F | P | A | S | G | S | A | 162 |
| OPT-39 | IFPASDSA | I | F | P | A | S | D | S | A | 164 |
| OPT-40 | IFPASGSA | I | F | P | A | S | G | S | A | 162 |
| OPT-41 | IFPASDSA | I | F | P | A | S | D | S | A | 164 |
| OPT-42 | IFPASGSA | I | F | P | A | S | G | S | A | 162 |
| OPT-43 #A2 | IFPASGSA | I | F | P | A | S | G | S | A | 162 |
| OPT-44 | IFPASDST | I | F | P | A | S | D | S | T | 15 |
| OPT-45 | IFPASGST | I | F | P | A | S | G | S | T | 163 |
| OPT-46 | IFPASGST | I | F | P | A | S | G | S | T | 163 |
| OPT-47 #A3 | IFPASDST | I | F | P | A | S | D | S | T | 15 |
| OPT-48 | IFPASDSA | I | F | P | A | S | D | S | A | 164 |
| OPT-49 | IFPASDSA | I | F | P | A | S | D | S | A | 164 |
| OPT-50 | IFPASGSA | I | F | P | A | S | G | S | A | 162 |
| OPT-51 | IFPASGSA | I | F | P | A | S | G | S | A | 162 |
| OPT-52 | IFPASGSA | I | F | P | A | S | G | S | A | 162 |
| OPT-53 | IFPASDSA | I | F | P | A | S | D | S | A | 164 |
| Opt-54 #A4 | IFPASGSA | I | F | P | A | S | G | S | A | 162 |
| OPT-55 | IFPASDSA | I | F | P | A | S | D | S | A | 164 |
| OPT-56 | IFPASGSA | I | F | P | A | S | G | S | A | 162 |
| Opt-57 #A5 | IFPASGSA | I | F | P | A | S | G | S | A | 162 |
| Opt-58 #A6 | IFPASGSA | I | F | P | A | S | G | S | A | 162 |
| Opt-59 #A7 | IFPASGST | I | F | P | A | S | G | S | T | 163 |
| OPT-60 | IFPASGST | I | F | P | A | S | G | S | T | 163 |
| OPT-61 | IFPASDST | I | F | P | A | S | D | S | T | 15 |
| OPT-62 | IFPASDSA | I | F | P | A | S | D | S | A | 164 |
| OPT-63 | IFPASDSA | I | F | P | A | S | D | S | A | 164 |
| OPT-64 | IFPASGSA | I | F | P | A | S | G | S | A | 162 |
| OPT-65 | IFPASGSA | I | F | P | A | S | G | S | A | 162 |
| OPT-66 | IFPASGSA | I | F | P | A | S | G | S | A | 162 |
| OPT-67 | IFPASDSA | I | F | P | A | S | D | S | A | 164 |
| OPT-68 | IFPASGSA | I | F | P | A | S | G | S | A | 162 |
| OPT-69 | IFPASDSA | I | F | P | A | S | D | S | A | 164 |
| OPT-70 | IFPASGST | I | F | P | A | S | G | S | T | 163 |
| OPT-71 | IFPASGST | I | F | P | A | S | G | S | T | 163 |
| OPT-72 | IFPASGST | I | F | P | A | S | G | S | T | 163 |
| OPT 73 | IFPASGST | I | F | P | A | S | G | S | T | 163 |
| | CONSENSUS | I | F | P | A | S | X1 | S | X2 | 167 |

TABLE 23
H-CDR3 Amino Acid Sequences-KABAT, CCG, CHOTHIA

| | | AMINO ACID NO. | | | | | | | | | | | | | | | |
|---|---|---|---|---|---|---|---|---|---|---|---|---|---|---|---|---|---|
| | KABAT/CCG/ | | | | | | | | | | | | | | | | SEQ |
| | CHOTHIA | | | | | | | | | | | | | | | | ID |
| | HCDR3 | 99 | 100 | 101 | 102 | 103 | 104 | 105 | 106 | 107 | 108 | 109 | 110 | 111 | 112 | 113 | NO. |
| PARENT | SGPYSYYAGGYALDY | S | G | P | Y | S | Y | Y | A | G | G | Y | A | L | D | Y | 10 |
| (#A1) | | | | | | | | | | | | | | | | | |
| GRAFT | | | | | | | | | | | | | | | | | |
| 005-G011 | | | | | | | | | | | | | | | | | |
| OPT-27 | SGPYSYYAGGYALDY | S | G | P | Y | S | Y | Y | A | G | G | Y | A | L | D | Y | 10 |
| OPT-28 | SGPYSYYAGGYALDY | S | G | P | Y | S | Y | Y | A | G | G | Y | A | L | D | Y | 10 |
| OPT 29 | SGPYSYYAGGYALDY | S | G | P | Y | S | Y | Y | A | G | G | Y | A | L | D | Y | 10 |
| OPT 30 | SGPYSYYAGGYALDY | S | G | P | Y | S | Y | Y | A | G | G | Y | A | L | D | Y | 10 |
| OPT 31 | SGPYSYYAGGYALDY | S | G | P | Y | S | Y | Y | A | G | G | Y | A | L | D | Y | 10 |
| OPT-32 | SGPYSYYAGGYALDY | S | G | P | Y | S | Y | Y | A | G | G | Y | A | L | D | Y | 10 |
| OPT-33 | SGPYSYYAGGYALDY | S | G | P | Y | S | Y | Y | A | G | G | Y | A | L | D | Y | 10 |
| OPT-34 | SGPYSYYAGGYALDY | S | G | P | Y | S | Y | Y | A | G | G | Y | A | L | D | Y | 10 |
| OPT-35 | SGPYSYYAGGYALDY | S | G | P | Y | S | Y | Y | A | G | G | Y | A | L | D | Y | 10 |
| OPT-36 | SGPYSYYAGGYALDY | S | G | P | Y | S | Y | Y | A | G | G | Y | A | L | D | Y | 10 |
| OPT-37 | SGPYSYYAGGYALDY | S | G | P | Y | S | Y | Y | A | G | G | Y | A | L | D | Y | 10 |
| OPT-38 | SGPYSYYAGGYALDY | S | G | P | Y | S | Y | Y | A | G | G | Y | A | L | D | Y | 10 |
| OPT-39 | SGPYSYYAGGYALDY | S | G | P | Y | S | Y | Y | A | G | G | Y | A | L | D | Y | 10 |
| OPT-40 | SGPYSYYAGGYALDY | S | G | P | Y | S | Y | Y | A | G | G | Y | A | L | D | Y | 10 |
| OPT-41 | SGPYSYYAGGYALDY | S | G | P | Y | S | Y | Y | A | G | G | Y | A | L | D | Y | 10 |
| OPT-42 | SGPYSYYAGGYALDY | S | G | P | Y | S | Y | Y | A | G | G | Y | A | L | D | Y | 10 |
| OPT-43 | SGPYSYYAGGYALDY | S | G | P | Y | S | Y | Y | A | G | G | Y | A | L | D | Y | 10 |
| #A2 | | | | | | | | | | | | | | | | | |
| OPT-44 | SGPYSYYAGGYALDY | S | G | P | Y | S | Y | Y | A | G | G | Y | A | L | D | Y | 10 |
| OPT-45 | SGPYSYYAGGYALDY | S | G | P | Y | S | Y | Y | A | G | G | Y | A | L | D | Y | 10 |
| OPT-46 | SGPYSYYAGGYALDY | S | G | P | Y | S | Y | Y | A | G | G | Y | A | L | D | Y | 10 |
| OPT-47 | SGPYSYYAGGYALDY | S | G | P | Y | S | Y | Y | A | G | G | Y | A | L | D | Y | 10 |
| #A3 | | | | | | | | | | | | | | | | | |
| OPT-48 | SGPYSYYAGGYALDY | S | G | P | Y | S | Y | Y | A | G | G | Y | A | L | D | Y | 10 |
| OPT-49 | SGPYSYYAGGYALDY | S | G | P | Y | S | Y | Y | A | G | G | Y | A | L | D | Y | 10 |
| OPT-50 | SGPYSYYAGGYALDY | S | G | P | Y | S | Y | Y | A | G | G | Y | A | L | D | Y | 10 |
| OPT-51 | SGPYSYYAGGYALDY | S | G | P | Y | S | Y | Y | A | G | G | Y | A | L | D | Y | 10 |
| OPT-52 | SGPYSYYAGGYALDY | S | G | P | Y | S | Y | Y | A | G | G | Y | A | L | D | Y | 10 |
| OPT-53 | SGPYSYYAGGYALDY | S | G | P | Y | S | Y | Y | A | G | G | Y | A | L | D | Y | 10 |
| Opt-54 | SGPYSYYAGGYALDY | S | G | P | Y | S | Y | Y | A | G | G | Y | A | L | D | Y | 10 |
| #A4 | | | | | | | | | | | | | | | | | |
| OPT-55 | SGPYSYYAGGYALDY | S | G | P | Y | S | Y | Y | A | G | G | Y | A | L | D | Y | 10 |
| OPT-56 | SGPYSYYAGGYALDY | S | G | P | Y | S | Y | Y | A | G | G | Y | A | L | D | Y | 10 |
| Opt-57 | SGPYSYYAGGYALDY | S | G | P | Y | S | Y | Y | A | G | G | Y | A | L | D | Y | 10 |
| #A5 | | | | | | | | | | | | | | | | | |
| Opt-58 | SGPYSYYAGGYALDY | S | G | P | Y | S | Y | Y | A | G | G | Y | A | L | D | Y | 10 |
| #A6 | | | | | | | | | | | | | | | | | |
| Opt-59 | SGPYSYYAGGYALDY | S | G | P | Y | S | Y | Y | A | G | G | Y | A | L | D | Y | 10 |
| #A7 | | | | | | | | | | | | | | | | | |
| OPT-60 | SGPYSYYAGGYALDY | S | G | P | Y | S | Y | Y | A | G | G | Y | A | L | D | Y | 10 |
| OPT-61 | SGPYSYYAGGYALDY | S | G | P | Y | S | Y | Y | A | G | G | Y | A | L | D | Y | 10 |
| OPT-62 | SGPYSYYAGGYALDY | S | G | P | Y | S | Y | Y | A | G | G | Y | A | L | D | Y | 10 |
| OPT-63 | SGPYSYYAGGYALDY | S | G | P | Y | S | Y | Y | A | G | G | Y | A | L | D | Y | 10 |
| OPT-64 | SGPYSYYAGGYALDY | S | G | P | Y | S | Y | Y | A | G | G | Y | A | L | D | Y | 10 |
| OPT-65 | SGPYSYYAGGYALDY | S | G | P | Y | S | Y | Y | A | G | G | Y | A | L | D | Y | 10 |
| OPT-66 | SGPYSYYAGGYALDY | S | G | P | Y | S | Y | Y | A | G | G | Y | A | L | D | Y | 10 |
| OPT-67 | SGPYSYYAGGYALDY | S | G | P | Y | S | Y | Y | A | G | G | Y | A | L | D | Y | 10 |
| OPT-68 | SGPYSYYAGGYALDY | S | G | P | Y | S | Y | Y | A | G | G | Y | A | L | D | Y | 10 |
| OPT-69 | SGPYSYYAGGYALDY | S | G | P | Y | S | Y | Y | A | G | G | Y | A | L | D | Y | 10 |
| OPT-70 | SGPYSYYAGGYALDY | S | G | P | Y | S | Y | Y | A | G | G | Y | A | L | D | Y | 10 |
| OPT-71 | SGPYSYYAGGYALDY | S | G | P | Y | S | Y | Y | A | G | G | Y | A | L | D | Y | 10 |
| OPT-72 | SGPYSYYAGGYALDY | S | G | P | Y | S | Y | Y | A | G | G | Y | A | L | D | Y | 10 |
| OPT 73 | SGPYSYYAGGYALDY | S | G | P | Y | S | Y | Y | A | G | G | Y | A | L | D | Y | 10 |
| | CONSENSUS | S | G | P | Y | S | Y | Y | A | G | G | Y | A | L | D | Y | 10 |

TABLE 24
H-CDR3 Amino Acid Sequences-IMGT

| | IMGT | AMINO ACID NO. | | | | | | | | | | | | | | | | | SEQ |
|---|---|---|---|---|---|---|---|---|---|---|---|---|---|---|---|---|---|---|---|
| | HCDR3 | 97 | 98 | 99 | 100 | 101 | 102 | 103 | 104 | 105 | 106 | 107 | 108 | 109 | 110 | 111 | 112 | 113 | ID NO. |
| PARENT | ARSGPYSYYAGGYALDY | A | R | S | G | P | Y | S | Y | Y | A | G | G | Y | A | L | D | Y | 16 |
| (#A1) | | | | | | | | | | | | | | | | | | | |
| GRAFT | | | | | | | | | | | | | | | | | | | |
| 005-G011 | | | | | | | | | | | | | | | | | | | |
| OPT-27 | ARSGPYSYYAGGYALDY | A | R | S | G | P | Y | S | Y | Y | A | G | G | Y | A | L | D | Y | 16 |
| OPT-28 | ARSGPYSYYAGGYALDY | A | R | S | G | P | Y | S | Y | Y | A | G | G | Y | A | L | D | Y | 16 |
| OPT 29 | ARSGPYSYYAGGYALDY | A | R | S | G | P | Y | S | Y | Y | A | G | G | Y | A | L | D | Y | 16 |
| OPT 30 | ARSGPYSYYAGGYALDY | A | R | S | G | P | Y | S | Y | Y | A | G | G | Y | A | L | D | Y | 16 |
| OPT 31 | ARSGPYSYYAGGYALDY | A | R | S | G | P | Y | S | Y | Y | A | G | G | Y | A | L | D | Y | 16 |
| OPT-32 | ARSGPYSYYAGGYALDY | A | R | S | G | P | Y | S | Y | Y | A | G | G | Y | A | L | D | Y | 16 |
| OPT-33 | ARSGPYSYYAGGYALDY | A | R | S | G | P | Y | S | Y | Y | A | G | G | Y | A | L | D | Y | 16 |
| OPT-34 | ARSGPYSYYAGGYALDY | A | R | S | G | P | Y | S | Y | Y | A | G | G | Y | A | L | D | Y | 16 |
| OPT-35 | ARSGPYSYYAGGYALDY | A | R | S | G | P | Y | S | Y | Y | A | G | G | Y | A | L | D | Y | 16 |
| OPT-36 | ARSGPYSYYAGGYALDY | A | R | S | G | P | Y | S | Y | Y | A | G | G | Y | A | L | D | Y | 16 |
| OPT-37 | ARSGPYSYYAGGYALDY | A | R | S | G | P | Y | S | Y | Y | A | G | G | Y | A | L | D | Y | 16 |
| OPT-38 | ARSGPYSYYAGGYALDY | A | R | S | G | P | Y | S | Y | Y | A | G | G | Y | A | L | D | Y | 16 |
| OPT-39 | ARSGPYSYYAGGYALDY | A | R | S | G | P | Y | S | Y | Y | A | G | G | Y | A | L | D | Y | 16 |
| OPT-40 | ARSGPYSYYAGGYALDY | A | R | S | G | P | Y | S | Y | Y | A | G | G | Y | A | L | D | Y | 16 |
| OPT-41 | ARSGPYSYYAGGYALDY | A | R | S | G | P | Y | S | Y | Y | A | G | G | Y | A | L | D | Y | 16 |
| OPT-42 | ARSGPYSYYAGGYALDY | A | R | S | G | P | Y | S | Y | Y | A | G | G | Y | A | L | D | Y | 16 |
| OPT-43 | ARSGPYSYYAGGYALDY | A | R | S | G | P | Y | S | Y | Y | A | G | G | Y | A | L | D | Y | 16 |
| #A2 | | | | | | | | | | | | | | | | | | | |
| OPT-44 | ARSGPYSYYAGGYALDY | A | R | S | G | P | Y | S | Y | Y | A | G | G | Y | A | L | D | Y | 16 |
| OPT-45 | ARSGPYSYYAGGYALDY | A | R | S | G | P | Y | S | Y | Y | A | G | G | Y | A | L | D | Y | 16 |
| OPT-46 | ARSGPYSYYAGGYALDY | A | R | S | G | P | Y | S | Y | Y | A | G | G | Y | A | L | D | Y | 16 |
| OPT-47 | ARSGPYSYYAGGYALDY | A | R | S | G | P | Y | S | Y | Y | A | G | G | Y | A | L | D | Y | 16 |
| #A3 | | | | | | | | | | | | | | | | | | | |
| OPT-48 | ARSGPYSYYAGGYALDY | A | R | S | G | P | Y | S | Y | Y | A | G | G | Y | A | L | D | Y | 16 |
| OPT-49 | ARSGPYSYYAGGYALDY | A | R | S | G | P | Y | S | Y | Y | A | G | G | Y | A | L | D | Y | 16 |
| OPT-50 | ARSGPYSYYAGGYALDY | A | R | S | G | P | Y | S | Y | Y | A | G | G | Y | A | L | D | Y | 16 |
| OPT-51 | ARSGPYSYYAGGYALDY | A | R | S | G | P | Y | S | Y | Y | A | G | G | Y | A | L | D | Y | 16 |
| OPT-52 | ARSGPYSYYAGGYALDY | A | R | S | G | P | Y | S | Y | Y | A | G | G | Y | A | L | D | Y | 16 |
| OPT-53 | ARSGPYSYYAGGYALDY | A | R | S | G | P | Y | S | Y | Y | A | G | G | Y | A | L | D | Y | 16 |
| Opt-54 | ARSGPYSYYAGGYALDY | A | R | S | G | P | Y | S | Y | Y | A | G | G | Y | A | L | D | Y | 16 |
| #A4 | | | | | | | | | | | | | | | | | | | |
| OPT-55 | ARSGPYSYYAGGYALDY | A | R | S | G | P | Y | S | Y | Y | A | G | G | Y | A | L | D | Y | 16 |
| OPT-56 | ARSGPYSYYAGGYALDY | A | R | S | G | P | Y | S | Y | Y | A | G | G | Y | A | L | D | Y | 16 |
| Opt-57 | ARSGPYSYYAGGYALDY | A | R | S | G | P | Y | S | Y | Y | A | G | G | Y | A | L | D | Y | 16 |
| #A5 | | | | | | | | | | | | | | | | | | | |
| Opt-58 | ARSGPYSYYAGGYALDY | A | R | S | G | P | Y | S | Y | Y | A | G | G | Y | A | L | D | Y | 16 |
| #A6 | | | | | | | | | | | | | | | | | | | |
| Opt-59 | ARSGPYSYYAGGYALDY | A | R | S | G | P | Y | S | Y | Y | A | G | G | Y | A | L | D | Y | 16 |
| #A7 | | | | | | | | | | | | | | | | | | | |
| OPT-60 | ARSGPYSYYAGGYALDY | A | R | S | G | P | Y | S | Y | Y | A | G | G | Y | A | L | D | Y | 16 |
| OPT-61 | ARSGPYSYYAGGYALDY | A | R | S | G | P | Y | S | Y | Y | A | G | G | Y | A | L | D | Y | 16 |
| OPT-62 | ARSGPYSYYAGGYALDY | A | R | S | G | P | Y | S | Y | Y | A | G | G | Y | A | L | D | Y | 16 |
| OPT-63 | ARSGPYSYYAGGYALDY | A | R | S | G | P | Y | S | Y | Y | A | G | G | Y | A | L | D | Y | 16 |
| OPT-64 | ARSGPYSYYAGGYALDY | A | R | S | G | P | Y | S | Y | Y | A | G | G | Y | A | L | D | Y | 16 |
| OPT-65 | ARSGPYSYYAGGYALDY | A | R | S | G | P | Y | S | Y | Y | A | G | G | Y | A | L | D | Y | 16 |
| OPT-66 | ARSGPYSYYAGGYALDY | A | R | S | G | P | Y | S | Y | Y | A | G | G | Y | A | L | D | Y | 16 |
| OPT-67 | ARSGPYSYYAGGYALDY | A | R | S | G | P | Y | S | Y | Y | A | G | G | Y | A | L | D | Y | 16 |
| OPT-68 | ARSGPYSYYAGGYALDY | A | R | S | G | P | Y | S | Y | Y | A | G | G | Y | A | L | D | Y | 16 |
| OPT-69 | ARSGPYSYYAGGYALDY | A | R | S | G | P | Y | S | Y | Y | A | G | G | Y | A | L | D | Y | 16 |
| OPT-70 | ARSGPYSYYAGGYALDY | A | R | S | G | P | Y | S | Y | Y | A | G | G | Y | A | L | D | Y | 16 |
| OPT-71 | ARSGPYSYYAGGYALDY | A | R | S | G | P | Y | S | Y | Y | A | G | G | Y | A | L | D | Y | 16 |
| OPT-72 | ARSGPYSYYAGGYALDY | A | R | S | G | P | Y | S | Y | Y | A | G | G | Y | A | L | D | Y | 16 |
| OPT 73 | ARSGPYSYYAGGYALDY | A | R | S | G | P | Y | S | Y | Y | A | G | G | Y | A | L | D | Y | 16 |
| | CONSENSUS | A | R | S | G | P | Y | S | Y | Y | A | G | G | Y | A | L | D | Y | 16 |

In one aspect, a variable region of the present invention is linked to a constant region. For example, a variable region of the present invention is linked to a constant region shown below to form a heavy chain or a light chain of an antibody.

Heavy Chain Constant region linked downstream
of a humanized variable heavy region:
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK (SEQ ID NO: 168)
Light Chain Constant region linked downstream
of a humanized variable light region:
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC (SEQ ID NO: 169)

Representative light chain and heavy chain sequences of the present invention are shown below (humanized variable regions derived from 005-GO11 parent (A1): opt-43 (A2), opt 47 (A3), opt-54 (A4), opt-57 (A5), opt-58 (A6), and opt-59 (A7) linked to constant regions).

Light Chain Amino Acid Sequences

A1 Light Chain
DIQMTQSPASLSASVGETVTITCRTSENINSYLAWYQQKQGKSPQLL
VHYAKTLAEGVPSRFSGSGSGTQFSLKINSLKPEDFGSYYCQHHYGT
SLTFGAGTKLELKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 170)
A2 Light Chain
DIQMTQSPSSLSASVGDRVTITCRASQSISSYLAWYQQKPGKAPKLL
IHYAKSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 171)
A3 Light Chain
DIQMTQSPSSLSASVGDRVTITCRASQSISSYLAWYQQKPGKAPKLL
IHYAKSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 172)
A4 Light Chain
DIQMTQSPSSLSASVGDRVTITCRASQSISSYLAWYQQKPGKAPKLL
IHYAKSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 173)
A5 Light Chain
DIQMTQSPSSLSASVGDRVTITCRASQSISSYLAWYQQKPGKAPKLL
IHYASSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 175)
A6 Light Chain
DIQMTQSPSSLSASVGDRVTITCRASQSISSYLAWYQQKPGKAPKLL
IHYASSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 175)
A7 Light Chain
DIQMTQSPSSLSASVGDRVTITCRASQSISSYLAWYQQKPGKAPKLL
IHYASSLAEGVPSRFSGSGSGTDFTLTISSLQPEDFATYYCQHHYGT
SLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC (SEQ ID NO: 176)

Heavy Chain Amino Acid Sequences

A1 Heavy Chain
QVHLQQSGPELVRPGTSVKISCEASGYIFLTYWMNWVKQRPGQGLEWI
GQIFPASDSTYYNEMFKDKARFTVDKSSSTAYMQFSSLTSEDTAVYFC
ARSGPYSYYAGGYALDYWGQGTSVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 177)
A2 Heavy Chain
QVQLVQSGAEVKKPGSSVKVSCKASGYIFLTYWMNWVRQAPGQGLEWM
GQIFPASGSAYYNQKFKGRVTITADKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 178)
A3 Heavy Chain
QVQLVQSGAEVKKPGSSVKVSCKASGYIFLTYWINWVRQAPGQGLEWM
GQIFPASDSTYYNQKFKGRVTITADKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 179)
A4 Heavy Chain
QVQLVQSGAEVKKPGSSVKVSCKASGYIFLTYWINWVRQAPGQGLEWM
GQIFPASGSAYYAQKFQGRVTITADKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 180)
A5 Heavy Chain
QVQLVQSGAEVKKPGSSVKVSCKASGYIFLTYWMNWVRQAPGQGLEWM
GQIFPASGSAYYAQKFQGRVTITVDKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 181)
A6 Heavy Chain
QVQLVQSGAEVKKPGSSVKVSCKASGYIFLTYWMNWVRQAPGQGLEWM
GQIFPASGSAYYNQKFKGRVTITADKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 182)
A7 Heavy Chain
QVQLVQSGAEVKKPGSSVKVSCKASGYIFLTYWINWVRQAPGQGLEWM
GQIFPASGSTYYNEKFKGRVTITADKSTSTAYMELSSLRSEDTAVYYC
ARSGPYSYYAGGYALDYWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPG (SEQ ID NO: 183)

In one aspect, an antibody of the present invention comprises 3 light chain CDRs and 3 heavy chain CDRs, for example as set forth in the Tables described above.

In one aspect, an antibody of the present invention comprises a light chain and a heavy chain variable region as set forth above. In one aspect, a light chain variable region of the invention is fused to a light chain constant region, for example a kappa or lambda constant region. In one aspect, a heavy chain variable region of the invention is fused to a heavy chain constant region, for example IgA, IgD, IgE, IgG or IgM, in particular, IgG₁, IgG₂, IgG₃ or IgG₄.

The present invention provides an anti-IL1RAP antibody comprising a light chain comprising the amino acid sequence of SEQ ID NO: 170; and a heavy chain comprising the amino acid sequence of SEQ ID NO: 177 (Antibody A1).

The present invention provides an anti-IL1RAP antibody comprising a light chain comprising the amino acid sequence of SEQ ID NO: 171; and a heavy chain comprising the amino acid sequence of SEQ ID NO: 178 (Antibody A2).

The present invention provides an anti-IL1RAP antibody comprising a light chain comprising the amino acid sequence of SEQ ID NO: 172; and a heavy chain comprising the amino acid sequence of SEQ ID NO: 179 (Antibody A3).

The present invention provides an anti-IL1RAP antibody comprising a light chain comprising the amino acid sequence of SEQ ID NO: 173; and a heavy chain comprising the amino acid sequence of SEQ ID NO: 180 (Antibody A4).

The present invention provides an anti-IL1RAP antibody comprising a light chain comprising the amino acid sequence of SEQ ID NO: 174; and a heavy chain comprising the amino acid sequence of SEQ ID NO: 181 (Antibody A5).

The present invention provides an anti-IL1RAP antibody comprising a light chain comprising the amino acid sequence of SEQ ID NO: 175; and a heavy chain comprising the amino acid sequence of SEQ ID NO: 182 Antibody A6).

The present invention provides an anti-IL1RAP antibody comprising a light chain comprising the amino acid sequence of SEQ ID NO: 176; and a heavy chain comprising the amino acid sequence of SEQ ID NO: 183 (Antibody A7).

Representative antibodies of the present invention are shown below.

TABLE 25

| Antibody | Light Chain Sequences | Heavy Chain Sequences |
|---|---|---|
| A1 | DIQMTQSPASLSASVGETVTITCR | QVHLQQSGPELVRPGTSVKISCEASGYI |
| | TSENINSYLAWYQQKQGKSPQLL | FLTYWMNWVKQRPGQGLEWIGQIFPAS |
| | VHYAKTLAEGVPSRFSGSGSGTQ | DSTYYNEMFKDKARFTVDKSSSTAYMQ |
| | FSLKINSLKPEDFGSYYCQHHYGT | FSSLTSEDTAVYFCARSGPYSYYAGGYA |
| | SLTFGAGTKLELKRTVAAPSVFIFP | LDYWGQGTSVTVSSASTKGPSVFPLAPS |
| | PSDEQLKSGTASVVCLLNNFYPR | SKSTSGGTAALGCLVKDYFPEPVTVSWN |
| | EAKVQWKVDNALQSGNSQESVT | SGALTSGVHTFPAVLQSSGLYSLSSVVT |
| | EQDSKDSTYSLSSTLTLSKADYEK | VPSSSLGTQTYICNVNHKPSNTKVDKRV |
| | HKVYACEVTHQGLSSPVTKSFNR | EPKSCDKTHTCPPCPAPEAAGGPSVFLF |
| | GEC (SEQ ID NO: 170) | PPKPKDTLMISRTPEVTCVVVDVSHEDP |
| | | EVKFNWYVDGVEVHNAKTKPREEQYNS |
| | | TYRVVSVLTVLHQDWLNGKEYKCKVSN |
| | | KALPAPIEKTISKAKGQPREPQVYTLPPS |
| | | REEMTKNQVSLTCLVKGFYPSDIAVEWE |
| | | SNGQPENNYKTTPPVLDSDGSFFLYSKL |
| | | TVDKSRWQQGNVFSCSVMHEALHNHYT |
| | | QKSLSLSPG (SEQ ID NO: 177) |
| A2 | DIQMTQSPSSLSASVGDRVTITCR | QVQLVQSGAEVKKPGSSVKVSCKASGYI |
| | ASQSISSYLAWYQQKPGKAPKLLI | FLTYWMNWVRQAPGQGLEWMGQIFPA |
| | HYAKSLAEGVPSRFSGSGSGTDF | SGSAYYNQKFKGRVTITADKSTSTAYME |
| | TLTISSLQPEDFATYYCQHHYGTS | LSSLRSEDTAVYYCARSGPYSYYAGGYA |
| | LTFGQGTKLEIKRTVAAPSVFIFPP | LDYWGQGTLVTVSSASTKGPSVFPLAPS |
| | SDEQLKSGTASVVCLLNNFYPRE | SKSTSGGTAALGCLVKDYFPEPVTVSWN |
| | AKVQWKVDNALQSGNSQESVTE | SGALTSGVHTFPAVLQSSGLYSLSSVVT |
| | QDSKDSTYSLSSTLTLSKADYEKH | VPSSSLGTQTYICNVNHKPSNTKVDKRV |
| | KVYACEVTHQGLSSPVTKSFNRG | EPKSCDKTHTCPPCPAPEAAGGPSVFLF |
| | EC (SEQ ID NO: 171) | PPKPKDTLMISRTPEVTCVVVDVSHEDP |
| | | EVKFNWYVDGVEVHNAKTKPREEQYNS |
| | | TYRVVSVLTVLHQDWLNGKEYKCKVSN |
| | | KALPAPIEKTISKAKGQPREPQVYTLPPS |
| | | REEMTKNQVSLTCLVKGFYPSDIAVEWE |
| | | SNGQPENNYKTTPPVLDSDGSFFLYSKL |
| | | TVDKSRWQQGNVFSCSVMHEALHNHYT |
| | | QKSLSLSPG (SEQ ID NO: 178) |
| A3 | DIQMTQSPSSLSASVGDRVTITCR | QVQLVQSGAEVKKPGSSVKVSCKASGYI |
| | ASQSISSYLAWYQQKPGKAPKLLI | FLTYWINWVRQAPGQGLEWMGQIFPAS |
| | HYAKSLAEGVPSRFSGSGSGTDF | DSTYYNQKFKGRVTITADKSTSTAYMEL |
| | TLTISSLQPEDFATYYCQHHYGTS | SSLRSEDTAVYYCARSGPYSYYAGGYAL |
| | LTFGQGTKLEIKRTVAAPSVFIFPP | DYWGQGTLVTVSSASTKGPSVFPLAPSS |
| | SDEQLKSGTASVVCLLNNFYPRE | KSTSGGTAALGCLVKDYFPEPVTVSWNS |
| | AKVQWKVDNALQSGNSQESVTE | GALTSGVHTFPAVLQSSGLYSLSSVVTV |
| | QDSKDSTYSLSSTLTLSKADYEKH | PSSSLGTQTYICNVNHKPSNTKVDKRVE |
| | KVYACEVTHQGLSSPVTKSFNRG | PKSCDKTHTCPPCPAPEAAGGPSVFLFP |
| | EC (SEQ ID NO: 172) | PKPKDTLMISRTPEVTCVVVDVSHEDPE |
| | | VKFNWYVDGVEVHNAKTKPREEQYNST |
| | | YRVVSVLTVLHQDWLNGKEYKCKVSNK |
| | | ALPAPIEKTISKAKGQPREPQVYTLPPSR |
| | | EEMTKNQVSLTCLVKGFYPSDIAVEWES |
| | | NGQPENNYKTTPPVLDSDGSFFLYSKLT |
| | | VDKSRWQQGNVFSCSVMHEALHNHYT |
| | | QKSLSLSPG (SEQ ID NO: 179) |
| A4 | DIQMTQSPSSLSASVGDRVTITCR | QVQLVQSGAEVKKPGSSVKVSCKASGYI |
| | ASQSISSYLAWYQQKPGKAPKLLI | FLTYWINWVRQAPGQGLEWMGQIFPAS |
| | HYAKSLAEGVPSRFSGSGSGTDF | GSAYYAQKFQGRVTITADKSTSTAYMEL |
| | TLTISSLQPEDFATYYCQHHYGTS | SSLRSEDTAVYYCARSGPYSYYAGGYAL |
| | LTFGQGTKLEIKRTVAAPSVFIFPP | DYWGQGTLVTVSSASTKGPSVFPLAPSS |
| | SDEQLKSGTASVVCLLNNFYPRE | KSTSGGTAALGCLVKDYFPEPVTVSWNS |
| | AKVQWKVDNALQSGNSQESVTE | GALTSGVHTFPAVLQSSGLYSLSSVVTV |
| | QDSKDSTYSLSSTLTLSKADYEKH | PSSSLGTQTYICNVNHKPSNTKVDKRVE |
| | KVYACEVTHQGLSSPVTKSFNRG | PKSCDKTHTCPPCPAPEAAGGPSVFLFP |
| | EC (SEQ ID NO: 173) | PKPKDTLMISRTPEVTCVVVDVSHEDPE |
| | | VKFNWYVDGVEVHNAKTKPREEQYNST |
| | | YRVVSVLTVLHQDWLNGKEYKCKVSNK |
| | | ALPAPIEKTISKAKGQPREPQVYTLPPSR |
| | | EEMTKNQVSLTCLVKGFYPSDIAVEWES |
| | | NGQPENNYKTTPPVLDSDGSFFLYSKLT |
| | | VDKSRWQQGNVFSCSVMHEALHNHYT |
| | | QKSLSLSPG (SEQ ID NO: 180) |
| A5 | DIQMTQSPSSLSASVGDRVTITCR | QVQLVQSGAEVKKPGSSVKVSCKASGYI |
| | ASQSISSYLAWYQQKPGKAPKLLI | FLTYWMNWVRQAPGQGLEWMGQIFPA |
| | HYASSLAEGVPSRFSGSGSGTDF | SGSAYYAQKFQGRVTITVDKSTSTAYME |
| | TLTISSLQPEDFATYYCQHHYGTS | LSSLRSEDTAVYYCARSGPYSYYAGGYA |
| | LTFGQGTKLEIKRTVAAPSVFIFPP | LDYWGQGTLVTVSSASTKGPSVFPLAPS |
| | SDEQLKSGTASVVCLLNNFYPRE | SKSTSGGTAALGCLVKDYFPEPVTVSWN |
| | AKVQWKVDNALQSGNSQESVTE | SGALTSGVHTFPAVLQSSGLYSLSSVVT |
| | QDSKDSTYSLSSTLTLSKADYEKH | VPSSSLGTQTYICNVNHKPSNTKVDKRV |
| | KVYACEVTHQGLSSPVTKSFNRG | EPKSCDKTHTCPPCPAPEAAGGPSVFLF |
| | EC (SEQ ID NO: 174) | PPKPKDTLMISRTPEVTCVVVDVSHEDP |
| | | EVKFNWYVDGVEVHNAKTKPREEQYNS |
| | | TYRVVSVLTVLHQDWLNGKEYKCKVSN |
| | | KALPAPIEKTISKAKGQPREPQVYTLPPS |
| | | REEMTKNQVSLTCLVKGFYPSDIAVEWE |
| | | SNGQPENNYKTTPPVLDSDGSFFLYSKL |
| | | TVDKSRWQQGNVFSCSVMHEALHNHYT |
| | | QKSLSLSPG (SEQ ID NO: 181) |
| A6 | DIQMTQSPSSLSASVGDRVTITCR | QVQLVQSGAEVKKPGSSVKVSCKASGYI |
| | ASQSISSYLAWYQQKPGKAPKLLI | FLTYWMNWVRQAPGQGLEWMGQIFPA |
| | HYASSLAEGVPSRFSGSGSGTDF | SGSAYYNQKFKGRVTITADKSTSTAYME |
| | TLTISSLQPEDFATYYCQHHYGTS | LSSLRSEDTAVYYCARSGPYSYYAGGYA |
| | LTFGQGTKLEIKRTVAAPSVFIFPP | LDYWGQGTLVTVSSASTKGPSVFPLAPS |
| | SDEQLKSGTASVVCLLNNFYPRE | SKSTSGGTAALGCLVKDYFPEPVTVSWN |
| | AKVQWKVDNALQSGNSQESVTE | SGALTSGVHTFPAVLQSSGLYSLSSVVT |
| | QDSKDSTYSLSSTLTLSKADYEKH | VPSSSLGTQTYICNVNHKPSNTKVDKRV |
| | KVYACEVTHQGLSSPVTKSFNRG | EPKSCDKTHTCPPCPAPEAAGGPSVFLF |
| | EC (SEQ ID NO: 175) | PPKPKDTLMISRTPEVTCVVVDVSHEDP |
| | | EVKFNWYVDGVEVHNAKTKPREEQYNS |
| | | TYRVVSVLTVLHQDWLNGKEYKCKVSN |
| | | KALPAPIEKTISKAKGQPREPQVYTLPPS |
| | | REEMTKNQVSLTCLVKGFYPSDIAVEWE |
| | | SNGQPENNYKTTPPVLDSDGSFFLYSKL |
| | | TVDKSRWQQGNVFSCSVMHEALHNHYT |
| | | QKSLSLSPG (SEQ ID NO: 182) |
| A7 | DIQMTQSPSSLSASVGDRVTITCR | QVQLVQSGAEVKKPGSSVKVSCKASGYI |
| | ASQSISSYLAWYQQKPGKAPKLLI | FLTYWINWVRQAPGQGLEWMGQIFPAS |
| | HYASSLAEGVPSRFSGSGSGTDF | GSTYYNEKFKGRVTITADKSTSTAYMEL |
| | TLTISSLQPEDFATYYCQHHYGTS | SSLRSEDTAVYYCARSGPYSYYAGGYAL |
| | LTFGQGTKLEIKRTVAAPSVFIFPP | DYWGQGTLVTVSSASTKGPSVFPLAPSS |
| | SDEQLKSGTASVVCLLNNFYPRE | KSTSGGTAALGCLVKDYFPEPVTVSWNS |
| | AKVQWKVDNALQSGNSQESVTE | GALTSGVHTFPAVLQSSGLYSLSSVVTV |
| | QDSKDSTYSLSSTLTLSKADYEKH | PSSSLGTQTYICNVNHKPSNTKVDKRVE |
| | KVYACEVTHQGLSSPVTKSFNRG | PKSCDKTHTCPPCPAPEAAGGPSVFLFP |
| | EC (SEQ ID NO: 176) | PKPKDTLMISRTPEVTCVVVDVSHEDPE |
| | | VKFNWYVDGVEVHNAKTKPREEQYNST |
| | | YRVVSVLTVLHQDWLNGKEYKCKVSNK |
| | | ALPAPIEKTISKAKGQPREPQVYTLPPSR |
| | | EEMTKNQVSLTCLVKGFYPSDIAVEWES |
| | | NGQPENNYKTTPPVLDSDGSFFLYSKLT |
| | | VDKSRWQQGNVFSCSVMHEALHNHYT |
| | | QKSLSLSPG (SEQ ID NO: 183) |

In some aspects, the invention comprises a neutralizing antibody that binds to IL1RAP. In some embodiments, the antibody specifically binds to IL1RAP. In some embodiments, the antibody binds to the domain 3 comprising amino acids 235-367 of IL1RAP (SEQ ID NO: 187). In some embodiments, the antibody binds to an epitope within amino acid residues corresponding to positions 235-315 of IL1RAP extracellular domain (ECD) 3 (SEQ ID NO: 188). In some embodiments, the antibody binds to IL1RAP having an amino acid sequence that is at least 90% identical to SEQ ID NO:187.

In some aspects, the invention comprises an antigen binding protein that binds to IL1RAP, wherein the antigen binding protein binds to IL1RAP at a location within residues corresponding to positions 235-315 of SEQ ID NO:188. In some embodiments, when the antigen binding protein is bound to IL1RAP, the antibody is positioned 8 angstroms or less from at least one of the following residues of IL1RAP 238, 239, 244-247, 249, 251-256, 261, 263, 265, 267, 269, 271, 301, 303, 305-306, 311, 313, or 315.

In some aspects, the invention comprises an antigen binding protein that binds to IL1RAP, wherein the antigen binding protein binds to IL1RAP at a location within residues corresponding to positions 235-273 of SEQ ID NO:189. In some embodiments, when the antigen binding protein is bound to IL1RAP, the antibody is positioned 8 angstroms or less from at least one of the following residues of IL1RAP 238, 239, 244-247, 249, 251-256, 261, 263, 265, 267, 269, or 271.

In some aspects, the invention comprises an antigen binding protein that binds to IL1RAP, wherein the antigen binding protein binds to IL1RAP at a location within residues corresponding to positions 300-315 of SEQ ID NO:190. In some embodiments, when the antigen binding protein is bound to IL1RAP, the antibody is positioned 8 angstroms or less from at least one of the following residues of IL1RAP 301, 303, 305-306, 311, 313, or 315.

In some aspects, the invention comprises an antigen binding protein that binds to IL1RAP, wherein the antigen binding protein binds to IL1RAP at a location within residues corresponding to positions 226-262 of SEQ ID NO:192. In some embodiments, when the antigen binding protein is bound to IL1RAP, the antibody is positioned 8 angstroms or less from at least one of the following residues of IL1RAP 238, 239, 244-247, 249, 251-256, or 261.

In some aspects, the invention comprises an antigen binding protein that binds to IL1RAP, wherein the antigen binding protein binds to IL1RAP at a location within residues corresponding to positions 226-273 of SEQ ID NO:192. In some embodiments, when the antigen binding protein is bound to IL1RAP, the antibody is positioned 8 angstroms or less from at least one of the following residues of IL1RAP 238, 239, 244-247, 249, 251-256, 261, 263, 265, 267, 269, or 271.

The antibodies of the present invention are useful in methods for the treatment of various diseases or disorders, for example immunological, inflammatory, autoimmune diseases and respiratory diseases in humans. For example, the antibodies of the present invention are useful in methods for the treatment of psoriasis, rheumatoid arthritis, or psoriatic arthritis. For example, the antibodies of the present invention are useful in methods for the treatment of chronic obstructive pulmonary disorder (COPD) or asthma. For example, the antibodies of the present invention are useful in methods for the treatment of scleroderma, palmoplantar pustulosis, generalized pustular psoriasis, diabetic nephropathy, lupus nephritis, scleroderma, ankylosing spondylitis, deficiency in the IL-36 receptor antagonist autoimmune disease (DITRA), deficiency in the IL-1 receptor antagonist autoimmune disease (DIRA) or cryopyrin associated periodic syndromes (CAPS).

In some aspects, the humanized antibody displays blocking activity, whereby it decreases IL-33, IL-36, or IL-1 mediated activation by at least 45%, by at least 50%, by at least 55%, by at least 60%, by at least 65%, by at least 70%, by at least 75%, by at least 80%, by at least 85%, by at least 90%, or by at least 95% when compared with a comparitor antibody control or in the absence of an anti-IL1RAP antibody or antibody fragment of the invention. The ability of an antibody to block binding of IL-33, IL-36, and IL-1 can be measured using binding assays known in the art. Alternatively, the blocking activity of an antibody can be measured by assessing the biological effects of IL-33, IL-36, and IL-1, such as the production of IL-8, IL-6, or IL-12 to determine if signaling mediated by IL1RAP is inhibited.

In a further aspect, the present invention provides a humanized anti-IL1RAP antibody having favorable biophysical properties. In one aspect, a humanized anti-IL1RAP antibody of the present invention is present in at least 90% monomer form, or in at least 92% monomer form, or in at least 95% monomer form in a buffer. In a further aspect, a humanized anti-IL1RAP antibody of the present invention remains in at least 90% monomer form, or in at least 92% monomer form, or in at least 95% monomer form in a buffer for one month or for four months.

In a further embodiment, a humanized antibody of the present invention consists of the light chain sequence of SEQ ID NO:170 and the heavy chain sequence of SEQ ID NO: 177 (Antibody A1). In another embodiment, a humanized antibody of the present invention consists of the light chain sequence of SEQ ID NO:171 and the heavy chain sequence of SEQ ID NO:178 (Antibody A2). In another embodiment, a humanized antibody of the present invention consists of the light chain sequence of SEQ ID NO:172 and the heavy chain sequence of SEQ ID NO:179 (Antibody A3). In another embodiment, a humanized antibody of the present invention consists of the light chain sequence of SEQ ID NO:173 and the heavy chain sequence of SEQ ID NO:180 (Antibody A4). In another embodiment, a humanized antibody of the present invention consists of the light chain sequence of SEQ ID NO:174 and the heavy chain sequence of SEQ ID NO:181 (Antibody A5). In another embodiment, a humanized antibody of the present invention consists of the light chain sequence of SEQ ID NO:175 and the heavy chain sequence of SEQ ID NO:182 (Antibody A6). In another embodiment, a humanized antibody of the present invention consists of the light chain sequence of SEQ ID NO:176 and the heavy chain sequence of SEQ ID NO:183 (Antibody A7).

In some embodiments, the humanized anti-IL1RAP antibodies, including antigen-binding fragments thereof, such as heavy and light chain variable regions, comprise an amino acid sequence of the residues derived from Antibody A1, Antibody A2, Antibody A3, Antibody A4, Antibody A5, Antibody A6, or Antibody A7.

In a further embodiment, the present invention provides an anti-IL1RAP antibody or antigen-binding fragment thereof that competitively binds to human IL1RAP with an antibody of the present invention, for example Antibody A1, Antibody A2, Antibody A3, Antibody A4, Antibody A5, Antibody A6, or Antibody A7 described herein. The ability of an antibody or antigen-binding fragment to competitively bind to IL1RAP can be measured using competitive binding assays known in the art.

The humanized anti-IL1RAP antibodies optionally include specific amino acid substitutions in the consensus or germline framework regions. The specific substitution of amino acid residues in these framework positions can improve various aspects of antibody performance including binding affinity and/or stability, over that demonstrated in humanized antibodies formed by “direct swap” of CDRs or HVLs into the human germline framework regions.

In some embodiments, the present invention describes a monoclonal antibody with a light chain variable region having the amino acid sequence set forth in SEQ ID NO: 1. In some embodiments, the present invention describes other monoclonal antibodies with a heavy chain variable region having the amino acid sequence set forth in SEQ ID NO:2. Placing such CDRs into FRs of the human consensus heavy and light chain variable domains will yield useful humanized antibodies of the present invention.

In particular, the present invention provides a monoclonal antibody with the combination of light chain variable and heavy chain variable region of SEQ ID NO:1 and SEQ ID NO:2, respectively. Such variable regions can be combined with human constant regions.

In some embodiments, the present invention describes other humanized antibodies with light chain variable region sequences having the amino acid sequence set forth in any one of SEQ ID NO:17-66. In some embodiments, the present invention describes other humanized antibodies with heavy chain variable region sequences having the amino acid sequence set forth in any one of SEQ ID NO:67-116. In particular, the present invention provides monoclonal antibodies with the combinations of light chain variable and heavy chain variable regions of SEQ ID NO: 17/67, 18/68, 19/69, 20/70, 21/71, 22/72, 23/73, 24/74, 25/75, 26/76, 27/77, 28/78, 29/79, 30/80, 31/81, 32/82, 33/83, 34/84, 35/85, 36/86, 37/87, 38/88, 39/89, 40/90, 41/91, 42/92, 43/93, 44/99, 45/95, 46/96, 47/97, 48/98, 49/99, 50/100, 51/101, 52/102, 53/103, 54/104, 55/105, 56/106, 57/107, 58/108, 59/109, 60/110, 61/111, 62/112, 63/113, 64/114, 65/115, and 66/116. Such variable regions can be combined with human constant regions.

In a further embodiment, the present invention relates to an anti-IL1RAP antibody or antigen-binding fragment thereof comprising a humanized light chain variable domain comprising the CDRs of SEQ ID NO:17 and framework regions having an amino acid sequence at least 90% identical, at least 93% identical or at least 95% identical to the amino acid sequence of the framework regions of the variable domain light chain amino acid sequence of SEQ ID NO:17 and a humanized heavy chain variable domain comprising the CDRs of SEQ ID NO:67 and framework regions having an amino acid sequence at least 90% identical, at least 93% identical or at least 95% identical to the amino acid sequence of the framework regions of the variable domain heavy chain amino acid sequence of SEQ ID NO:67. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:36 and framework regions having an amino acid sequence at least 90% identical, at least 93% identical or at least 95% identical to the amino acid sequence of the framework regions of the variable domain light chain amino acid sequence of SEQ ID NO:36 and a humanized heavy chain variable domain comprising the CDRs of SEQ ID NO:86 and framework regions having an amino acid sequence at least 90% identical, at least 93% identical or at least 95% identical to the amino acid sequence of the framework regions of the variable domain heavy chain amino acid sequence of SEQ ID NO:86. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:40 and framework regions having an amino acid sequence at least 90% identical, at least 93% identical or at least 95% identical to the amino acid sequence of the framework regions of the variable domain light chain amino acid sequence of SEQ ID NO:40 and a humanized heavy chain variable domain comprising the CDRs of SEQ ID NO:90 and framework regions having an amino acid sequence at least 90% identical, at least 93% identical or at least 95% identical to the amino acid sequence of the framework regions of the variable domain heavy chain amino acid sequence of SEQ ID NO:90. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:47 and framework regions having an amino acid sequence at least 90% identical, at least 93% identical or at least 95% identical to the amino acid sequence of the framework regions of the variable domain light chain amino acid sequence of SEQ ID NO:47 and a humanized heavy chain variable domain comprising the CDRs of SEQ ID NO:97 and framework regions having an amino acid sequence at least 90% identical, at least 93% identical or at least 95% identical to the amino acid sequence of the framework regions of the variable domain heavy chain amino acid sequence of SEQ ID NO:97. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:50 and framework regions having an amino acid sequence at least 90% identical, at least 93% identical or at least 95% identical to the amino acid sequence of the framework regions of the variable domain light chain amino acid sequence of SEQ ID NO:50 and a humanized heavy chain variable domain comprising the CDRs of SEQ ID NO:100 and framework regions having an amino acid sequence at least 90% identical, at least 93% identical or at least 95% identical to the amino acid sequence of the framework regions of the variable domain heavy chain amino acid sequence of SEQ ID NO:100. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:51 and framework regions having an amino acid sequence at least 90% identical, at least 93% identical or at least 95% identical to the amino acid sequence of the framework regions of the variable domain light chain amino acid sequence of SEQ ID NO:51 and a humanized heavy chain variable domain comprising the CDRs of SEQ ID NO:101 and framework regions having an amino acid sequence at least 90% identical, at least 93% identical or at least 95% identical to the amino acid sequence of the framework regions of the variable domain heavy chain amino acid sequence of SEQ ID NO:101. In one embodiment, the anti-IL1RAP antibody is a humanized monoclonal antibody.

In a further embodiment, the present invention relates to an anti-IL1RAP antibody or antigen-binding fragment thereof comprising a humanized light chain variable domain comprising the CDRs of SEQ ID NO:52 and framework regions having an amino acid sequence at least 90% identical, at least 93% identical or at least 95% identical to the amino acid sequence of the framework regions of the variable domain light chain amino acid sequence of SEQ ID NO:52 and a humanized heavy chain variable domain comprising the CDRs of SEQ ID NO:102 and framework regions having an amino acid sequence at least 90% identical, at least 93% identical or at least 95% identical to the amino acid sequence of the framework regions of the variable domain heavy chain amino acid sequence of SEQ ID NO:102. In one embodiment, the anti-IL1RAP antibody is a humanized monoclonal antibody.

In one further aspect, the present invention provides an anti-IL1RAP antibody or antigen-binding fragment thereof comprising a light chain CDR1 (L-CDR1) sequence of any one of SEQ ID NO:3 or 6; a light chain CDR2 (L-CDR2) sequence of any one of SEQ ID NO:4 or 7; a light chain CDR3 (L-CDR3) sequence of SEQ ID NO:5; a heavy chain CDR1 (H-CDR1) sequence of any one of SEQ ID NO:8, 11, 12, 14; a heavy chain CDR2 (H-CDR2) sequence of any one of SEQ ID NO:9, 13, or 15; and a heavy chain CDR3 (H-CDR3) sequence of any one of SEQ ID NO:10 or 16. In one aspect, the anti-IL1RAP antibody or antigen-binding fragment thereof comprises a light chain variable region comprising a L-CDR1 listed above, a L-CDR2 listed above and a L-CDR3 listed above, and a heavy chain variable region comprising a H-CDR1 listed above, a H-CDR2 listed above and a H-CDR3 listed above.

In one further aspect, the present invention provides an anti-IL1RAP antibody or antigen-binding fragment thereof comprising a light chain CDR1 (L-CDR1) sequence of any one of SEQ ID NO:3, 6, 134, where X1=A or T; X2=Q or E; X3=S or N; X4=S or N) or 135, where X1=A or T; X2=Q or E; X3=S or N; X4=S or N); a light chain CDR2 (L-CDR2) sequence of any one of SEQ ID NO:4, 7, 140, where X1=S or K and X2=S or T, or 141, where X1=S or K); a light chain CDR3 (L-CDR3) sequence of SEQ ID NO:5; a heavy chain CDR1 (H-CDR1) sequence of any one of SEQ ID NO:8, 11, 12, 14, 146 where X1=M or I and X2=N or S), or 147 where X1=M or I and X2=N or S; a heavy chain CDR2 (H-CDR2) sequence of any one of SEQ ID NO:9, 13, or 15, 165 where X1=D or G, X2=A or T, X3=N or A, X4=Q or E, X5=M or K, X6=Q or K, and X7=D or G), 166 where X1=D or G), or 167 where X1=D or G; X2=A or T; and a heavy chain CDR3 (H-CDR3) sequence of any one of SEQ ID NO:10 or 16. In one aspect, the anti-IL1RAP antibody or antigen-binding fragment thereof comprises a light chain variable region comprising a L-CDR1 listed above, a L-CDR2 listed above and a L-CDR3 listed above, and a heavy chain variable region comprising a H-CDR1 listed above, a H-CDR2 listed above and a H-CDR3 listed above.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:3, 4, 5, 8, 9 and 10, (KABAT) respectively; or
b) a L-CDR1, a L-CDR2, a L-CDR3, a H-CDR1, a H-CDR2 and a H-CDR3 sequence of SEQ ID NO:3, 4, 5, 11, 9 and 10, (CCG) respectively; or
c) a L-CDR1, a L-CDR2, a L-CDR3, a H-CDR1, a H-CDR2 and a H-CDR3 sequence of SEQ ID NO:3, 4, 5, 12, 13 and 10, (CHOTHIA) respectively; or
d) a L-CDR1, a L-CDR2, a L-CDR3, a H-CDR1, a H-CDR2 and a H-CDR3 sequence of SEQ ID NO:6, 7, 5, 14, 15 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 136, 5, 8, 149 and 10, (KABAT) respectively; or
b) a L-CDR1, a L-CDR2, a L-CDR3, a H-CDR1, a H-CDR2 and a H-CDR3 sequence of SEQ ID NO:117, 136, 5, 11, 149 and 10, (CCG) respectively; or
c) a L-CDR1, a L-CDR2, a L-CDR3, a H-CDR1, a H-CDR2 and a H-CDR3 sequence of SEQ ID NO:117, 136, 5, 12, 161 and 10, (CHOTHIA) respectively; or
d) a L-CDR1, a L-CDR2, a L-CDR3, a H-CDR1, a H-CDR2 and a H-CDR3 sequence of SEQ ID NO:127, 7, 5, 14, 162 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 136, 5, 142, 153 and 10, (KABAT) respectively; or
b) a L-CDR1, a L-CDR2, a L-CDR3, a H-CDR1, a H-CDR2 and a H-CDR3 sequence of SEQ ID NO:117, 136, 5, 144, 153 and 10, (CCG) respectively; or
c) a L-CDR1, a L-CDR2, a L-CDR3, a H-CDR1, a H-CDR2 and a H-CDR3 sequence of SEQ ID NO:117, 136, 5, 12, 13 and 10, (CHOTHIA) respectively; or
d) a L-CDR1, a L-CDR2, a L-CDR3, a H-CDR1, a H-CDR2 and a H-CDR3 sequence of SEQ ID NO:127, 7, 5, 14, 15 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 136, 5, 142, 148 and 10, (KABAT) respectively; or
b) a L-CDR1, a L-CDR2, a L-CDR3, a H-CDR1, a H-CDR2 and a H-CDR3 sequence of SEQ ID NO:117, 136, 5, 144, 148 and 10, (CCG) respectively; or
c) a L-CDR1, a L-CDR2, a L-CDR3, a H-CDR1, a H-CDR2 and a H-CDR3 sequence of SEQ ID NO:117, 136, 5, 12, 161 and 10, (CHOTHIA) respectively; or
d) a L-CDR1, a L-CDR2, a L-CDR3, a H-CDR1, a H-CDR2 and a H-CDR3 sequence of SEQ ID NO:127, 7, 5, 14, 162 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 137, 5, 8, 148 and 10, (KABAT) respectively; or
b) a L-CDR1, a L-CDR2, a L-CDR3, a H-CDR1, a H-CDR2 and a H-CDR3 sequence of SEQ ID NO:117, 137, 5, 11, 148 and 10, (CCG) respectively; or
c) a L-CDR1, a L-CDR2, a L-CDR3, a H-CDR1, a H-CDR2 and a H-CDR3 sequence of SEQ ID NO:117, 137, 5, 12, 161 and 10, (CHOTHIA) respectively; or
d) a L-CDR1, a L-CDR2, a L-CDR3, a H-CDR1, a H-CDR2 and a H-CDR3 sequence of SEQ ID NO:127, 139, 5, 14, 162 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 137, 5, 8, 149 and 10, (KABAT) respectively; or
b) a L-CDR1, a L-CDR2, a L-CDR3, a H-CDR1, a H-CDR2 and a H-CDR3 sequence of SEQ ID NO:117, 137, 5, 11, 149 and 10, (CCG) respectively; or
c) a L-CDR1, a L-CDR2, a L-CDR3, a H-CDR1, a H-CDR2 and a H-CDR3 sequence of SEQ ID NO:117, 137, 5, 12, 161 and 10, (CHOTHIA) respectively; or
d) a L-CDR1, a L-CDR2, a L-CDR3, a H-CDR1, a H-CDR2 and a H-CDR3 sequence of SEQ ID NO:127, 139, 5, 14, 162 and 16, (IMGT) respectively

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:117, 137, 5, 142, 151 and 10, (KABAT) respectively; or
b) a L-CDR1, a L-CDR2, a L-CDR3, a H-CDR1, a H-CDR2 and a H-CDR3 sequence of SEQ ID NO:117, 137, 5, 144, 151 and 10, (CCG) respectively; or
c) a L-CDR1, a L-CDR2, a L-CDR3, a H-CDR1, a H-CDR2 and a H-CDR3 sequence of SEQ ID NO:117, 137, 5, 12, 161 and 10, (CHOTHIA) respectively; or
d) a L-CDR1, a L-CDR2, a L-CDR3, a H-CDR1, a H-CDR2 and a H-CDR3 sequence of SEQ ID NO:127, 139, 5, 14, 163 and 16, (IMGT) respectively.

In a further aspect, the present invention provides an anti-IL1RAP antibody or antigen-binding fragment thereof comprising:
a) a L-CDR1, a L-CDR2, a L-CDR3, a H-CDR1, a H-CDR2 and a H-CDR3 sequence of SEQ ID NO:135, 140, 5, 146, 165 and 10, (KABAT/CCG) respectively; or
b) a L-CDR1, a L-CDR2, a L-CDR3, a H-CDR1, a H-CDR2 and a H-CDR3 sequence of SEQ ID NO:135, 140, 5, 12, 166 and 10, (CHOTHIA) respectively; or
c) a L-CDR1, a L-CDR2, a L-CDR3, a H-CDR1, a H-CDR2 and a H-CDR3 sequence of SEQ ID NO:136, 141, 5, 14, 167 and 16, (IMGT) respectively.

In one aspect, the anti-IL1RAP antibody or antigen-binding fragment thereof comprises a light chain variable region comprising a L-CDR1, L-CDR2 and L-CDR3 combination listed above, and a heavy chain variable region comprising a H-CDR1, H-CDR2 and H-CDR3 combination listed above.

In specific embodiments, it is contemplated that chimeric antibodies with switched CDR regions (i.e., for example switching one or two CDRs of one of the mouse antibodies or humanized antibody derived therefrom with the analogous CDR from another mouse antibody or humanized antibody derived therefrom) between these exemplary immunoglobulins may yield useful antibodies.

In certain embodiments, the humanized anti-IL1RAP antibody is an antibody fragment. Various antibody fragments have been generally discussed above and there are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to the skilled practitioner. Accordingly, in one aspect, the present invention provides antibody fragments comprising the CDRs described herein, in particular one of the combinations of L-CDR1, L-CDR2, L-CDR3, H-CDR1, H-CDR2 and H-CDR3 described herein. In a further aspect, the present invention provides antibody fragments comprising the variable regions described herein, for example one of the combinations of light chain variable regions and heavy chain variable regions described herein.

Certain embodiments include an F(ab′)₂ fragment of a humanized anti-IL1RAP antibody comprise a light chain sequence of any of SEQ ID NO: 170, 171, 172, 173, 174, 176, or 176 in combination with a heavy chain sequence of SEQ ID NO: 177, 178, 179, 180, 181, 182, or 183. Such embodiments can include an intact antibody comprising such an F(ab′)₂.

In some embodiments, the antibody or antibody fragment includes a constant region that mediates effector function. The constant region can provide antibody-dependent cellular cytotoxicity (ADCC), antibody-dependent cellular phagocytosis (ADCP) and/or complement-dependent cytotoxicity (CDC) responses. The effector domain(s) can be, for example, an Fc region of an Ig molecule.

The effector domain of an antibody can be from any suitable vertebrate animal species and isotypes. The isotypes from different animal species differ in the abilities to mediate effector functions. For example, the ability of human immunoglobulin to mediate CDC and ADCC/ADCP is generally in the order of IgM≈IgG₁≈IgG₃>IgG₂>IgG₄ and IgG₁˜IgG₃>IgG₂/IgM/IgG₄, respectively. Murine immunoglobulins mediate CDC and ADCC/ADCP generally in the order of murine IgM˜IgG₃>>IgG_{2b}>IgG₂ₐ>>IgG₁ and IgG_{2b}>IgG₂ₐ>IgG₁>>IgG₃, respectively. In another example, murine IgG₂ₐ mediates ADCC while both murine IgG₂ₐ and IgM mediate CDC.

### Antibody Modifications

The humanized anti-IL1RAP antibodies and agents can include modifications of the humanized anti-IL1RAP antibody or antigen-binding fragment thereof. For example, it may be desirable to modify the antibody with respect to effector function, so as to enhance the effectiveness of the antibody in treating cancer. One such modification is the introduction of cysteine residue(s) into the Fc region, thereby allowing interchain disulfide bond formation in this region. The homodimeric antibody thus generated can have improved internalization capability and/or increased complement-mediated cell killing and/or antibody-dependent cellular cytotoxicity (ADCC). See, for example, Caron et al., 1992, J. Exp Med. 176:1191-1195; and Shopes, 1992, J. Immunol. 148:2918-2922. Homodimeric antibodies having enhanced anti-tumor activity can also be prepared using heterobifunctional cross-linkers as described in Wolff et al., 1993, Cancer Research 53:2560-2565. Alternatively, an antibody can be engineered to contain dual Fc regions, enhancing complement lysis and ADCC capabilities of the antibody. See Stevenson et al., 1989, Anti-Cancer Drug Design 3:219-230.

Antibodies with improved ability to support ADCC have been generated by modifying the glycosylation pattern of their Fc region. This is possible since antibody glycosylation at the asparagine residue, N297, in the C_{H2} domain is involved in the interaction between IgG and Fcγ receptors prerequisite to ADCC. Host cell lines have been engineered to express antibodies with altered glycosylation, such as increased bisecting N-acetylglucosamine or reduced fucose. Fucose reduction provides greater enhancement to ADCC activity than does increasing the presence of bisecting N-acetylglucosamine. Moreover, enhancement of ADCC by low fucose antibodies is independent of the FcγRIIIa V/F polymorphism.

Modifying the amino acid sequence of the Fc region of antibodies is an alternative to glycosylation engineering to enhance ADCC. The binding site on human IgG1 for Fcγ receptors has been determined by extensive mutational analysis. This led to the generation of humanized IgG1 antibodies with Fc mutations that increase the binding affinity for FcγRIIIa and enhance ADCC in vitro. Additionally, Fc variants have been obtained with many different permutations of binding properties, e.g., improved binding to specific FcγR receptors with unchanged or diminished binding to other FcγR receptors.

Another aspect includes immunoconjugates comprising the humanized antibody or fragments thereof conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used to form useful immunoconjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, the tricothecenes, and the like. A variety of radionuclides are available for the production of radioconjugated humanized anti-IL1RAP antibodies. Examples include ²¹²Bi, ¹³¹I, ¹³¹In, ⁹⁰Y, and ¹⁸⁶Re.

Conjugates of the humanized anti-IL1RAP antibody and cytotoxic or chemotherapeutic agent can be made by known methods, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., 1987, Science 238:1098. Carbon-14-labeled 1-isothiocyanatobenzyl-3-methyldiethylene triaminepentaacetic acid (MX-DTPA) is an exemplary chelating agent for conjugation of radionucleotide to the antibody. Conjugates also can be formed with a cleavable linker.

The humanized anti-IL1RAP antibodies disclosed herein can also be formulated as immunoliposomes. Liposomes containing the antibody are prepared by methods known in the art, such as described in Epstein et al., 1985, Proc. Natl. Acad. Sci. USA 82:3688; Hwang et al., 1980, Proc. Natl. Acad. Sci. USA 77:4030; and U.S. Pat. Nos. 4,485,045 and 4,544,545. Liposomes having enhanced circulation time are disclosed, for example, in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody disclosed herein can be conjugated to the liposomes as described in Martin et al., 1982, J. Biol. Chem. 257:286-288 via a disulfide interchange reaction. A chemotherapeutic agent (such as doxorubicin) is optionally contained within the liposome. See, e.g., Gabizon et al., 1989, J. National Cancer Inst. 81 (19): 1484.

The antibodies described and disclosed herein can also be used in ADEPT (Antibody-Directed Enzyme Prodrug Therapy) procedures by conjugating the antibody to a prodrug-activating enzyme that converts a prodrug (e.g., a peptidyl chemotherapeutic agent), to an active anti-cancer drug. See, for example, WO 81/01145, WO 88/07378, and U.S. Pat. No. 4,975,278. The enzyme component of the immunoconjugate useful for ADEPT is an enzyme capable of acting on a prodrug in such a way so as to covert it into its more active, cytotoxic form. Specific enzymes that are useful in ADEPT include, but are not limited to, alkaline phosphatase for converting phosphate-containing prodrugs into free drugs; arylsulfatase for converting sulfate-containing prodrugs into free drugs; cytosine deaminase for converting non-toxic 5-fluorocytosine into the anti-cancer drug, 5-fluorouracil; proteases, such as Serratia protease, thermolysin, subtilisin, carboxypeptidases, and cathepsins (such as cathepsins B and L), for converting peptide-containing prodrugs into free drugs; D-alanylcarboxypeptidases, for converting prodrugs containing D-amino acid substituents; carbohydrate-cleaving enzymes such as β-galactosidase and neuraminidase for converting glycosylated prodrugs into free drugs; β-lactamase for converting drugs derivatized with β-lactams into free drugs; and penicillin amidases, such as penicillin V amidase or penicillin G amidase, for converting drugs derivatized at their amine nitrogens with phenoxyacetyl or phenylacetyl groups, respectively, into free drugs. Alternatively, antibodies having enzymatic activity (“abzymes”) can be used to convert the prodrugs into free active drugs (see, for example, Massey, 1987, Nature 328:457-458). Antibody-abzyme conjugates can be prepared by known methods for delivery of the abzyme to a tumor cell population, for example, by covalently binding the enzyme to the humanized anti-IL1RAP antibody/heterobifunctional crosslinking reagents discussed above. Alternatively, fusion proteins comprising at least the antigen binding region of an antibody disclosed herein linked to at least a functionally active portion of an enzyme as described above can be constructed using recombinant DNA techniques (see, e.g., Neuberger et al., 1984, Nature 312:604-608).

In certain embodiments, it may be desirable to use a humanized anti-IL1RAP antibody fragment, rather than an intact antibody, to increase tissue penetration, for example. It may be desirable to modify the antibody fragment in order to increase its serum half life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis. See, e.g., WO 96/32478.

In other embodiments, covalent modifications of the humanized anti-IL1RAP antibody are also included. Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody. Such modifications may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

### Humanization and Amino Acid Sequence Variants

Amino acid sequence variants of the anti-IL1RAP antibody can be prepared by introducing appropriate nucleotide changes into the anti-IL1RAP antibody DNA, or by peptide synthesis. Such variants include, for example, deletions from, and/or insertions into and/or substitutions of, residues within the amino acid sequences of the anti-IL1RAP antibodies of the examples herein. Any combination of deletions, insertions, and substitutions is made to arrive at the final construct, provided that the final construct possesses the desired characteristics. The amino acid changes also may alter post-translational processes of the humanized or variant anti-IL1RAP antibody, such as changing the number or position of glycosylation sites.

A useful method for identification of certain residues or regions of the anti-IL1RAP antibody that are preferred locations for mutagenesis is called “alanine scanning mutagenesis,” as described by Cunningham and Wells (Science, 244:1081-1085 (1989)). Here, a residue or group of target residues are identified (e.g., charged residues such as arg, asp, his, lys, and glu) and replaced by a neutral or negatively charged amino acid (typically alanine) to affect the interaction of the amino acids with IL1RAP antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed anti-IL1RAP antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-IL1RAP antibody fused to an epitope tag. Other insertional variants of the anti-IL1RAP antibody molecule include a fusion to the N- or C-terminus of the anti-IL1RAP antibody of an enzyme or a polypeptide which increases the serum half-life of the antibody.

Another type of variant is an amino acid substitution variant. These variants have at least one amino acid residue in the anti-IL1RAP antibody molecule removed and a different residue inserted in its place. The sites of greatest interest for substitutional mutagenesis include the hypervariable regions, but FR alterations are also contemplated. Conservative substitutions are shown in Table 26 under the heading of “preferred substitutions”. If such substitutions result in a change in biological activity, then more substantial changes, denominated “exemplary substitutions”, or as further described below in reference to amino acid classes, may be introduced and the products screened.

TABLE 26

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | arg; asn; gln; lys; | arg |
| Ile (I) | leu; val; met; ala; phe; norleucine | leu |
| Leu (L) | ile; norleucine; val; met; ala; phe | ile |
| Lys (K) | arg; gln; asn | arg |
| Met (M) | leu; phe; ile | leu |
| Phe (F) | tyr; leu; val; ile; ala; | tyr |
| Pro (P) | ala | ala |
| Ser (S) | thr | thr |
| Thr (T) | ser | ser |
| Trp (W) | tyr; phe | tyr |
| Tyr (Y) | phe; trp; thr; ser | phe |
| Val (V) | leu; ile; met; phe ala; norleucine; | leu |

In protein chemistry, it is generally accepted that the biological properties of the antibody can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the humanized or variant anti-IL1RAP antibody also may be substituted, generally with serine, to improve the oxidative stability of the molecule, prevent aberrant crosslinking, or provide for established points of conjugation to a cytotoxic or cytostatic compound. Conversely, cysteine bond(s) may be added to the antibody to improve its stability (particularly where the antibody is an antibody fragment such as an Fv fragment).

A type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity). In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and human IL1RAP. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By “altering” is meant deleting one or more carbohydrate moieties found in the antibody, and/or adding one or more glycosylation sites that are not present in the antibody.

In some embodiments, it may be desirable to modify the antibodies of the invention to add glycosylations sites. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Thus, in order to glycosylate a given protein, e.g., an antibody, the amino acid sequence of the protein is engineered to contain one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IL1RAP antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-IL1RAP antibody.

### Polynucleotides, Vectors, Host Cells, and Recombinant Methods

Other embodiments encompass isolated polynucleotides that comprise a sequence encoding a humanized anti-IL1RAP antibody, vectors, and host cells comprising the polynucleotides, and recombinant techniques for production of the humanized antibody. The isolated polynucleotides can encode any desired form of the anti-IL1RAP antibody including, for example, full length monoclonal antibodies, Fab, Fab′, F(ab′)₂, and Fv fragments, diabodies, linear antibodies, single-chain antibody molecules, and multispecific antibodies formed from antibody fragments.

Some embodiments include isolated polynucleotides comprising sequences that encode the light chain variable region of an antibody or antibody fragment having the amino acid sequence of any of SEQ ID NO: SEQ ID NO:17-66. Some embodiments include isolated polynucleotides comprising sequences that encode the heavy chain variable region of an antibody or antibody fragment having the amino acid sequence of SEQ ID NO:67-116.

Some embodiments include isolated polynucleotides comprising sequences that encode the light chain variable region of an antibody or antibody fragment having the amino acid sequence of any of SEQ ID NO:17, 36, 40, 47, 50, 51, and 52. Some embodiments include isolated polynucleotides comprising sequences that encode the heavy chain variable region of an antibody or antibody fragment having the amino acid sequence of SEQ ID NO: 67, 86, 90, 97, 100, 101, and 102.

In one aspect, the isolated polynucleotide sequence(s) encodes an antibody or antibody fragment having a light chain and a heavy chain variable region comprising the amino acid sequences of SEQ ID NO:17 and SEQ ID NO:167, respectively; SEQ ID NO:36 and SEQ ID NO:86, respectively; SEQ ID NO:40 and SEQ ID NO:90, respectively; SEQ ID NO: 47 and SEQ ID NO:97, respectively; SEQ ID NO:50 and SEQ ID NO:100, respectively; SEQ ID NO:51 and SEQ ID NO:101, respectively; and SEQ ID NO:52 and SEQ ID NO:102, respectively.

The polynucleotide(s) that comprise a sequence encoding a humanized anti-IL1RAP antibody or a fragment or chain thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The humanized anti-IL1RAP antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the humanized anti-IL1RAP antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO90/13646. In mammalian cells, mammalian signal sequences as well as viral secretory leaders, for example, the herpes simplex gD signal, can be used. The DNA for such precursor region is ligated in reading frame to DNA encoding the humanized anti-IL 1RAP antibody.

Expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2-ν. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, and hygromycin. Common selectable markers for mammalian cells are those that enable the identification of cells competent to take up a nucleic acid encoding a humanized anti-IL1RAP antibody, such as DHFR (dihydrofolate reductase), thymidine kinase, metallothionein-I and -II (such as primate metallothionein genes), adenosine deaminase, ornithine decarboxylase, and the like. Cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., DG44).

Alternatively, host cells (particularly wild-type hosts that contain endogenous DHFR) transformed or co-transformed with DNA sequences encoding anti-IL1RAP antibody, wild-type DHFR protein, and another selectable marker such as aminoglycoside 3′-phosphotransferase (APH), can be selected by cell growth in medium containing a selection agent for the selectable marker such as an aminoglycosidic antibiotic, e.g., kanamycin, neomycin, or G418. See, e.g., U.S. Pat. No. 4,965,199.

Where the recombinant production is performed in a yeast cell as a host cell, the TRP1 gene present in the yeast plasmid YRp7 (Stinchcomb et al., 1979, Nature 282:39) can be used as a selectable marker. The TRP1 gene provides a selection marker for a mutant strain of yeast lacking the ability to grow in tryptophan, for example, ATCC No. 44076 or PEP4-1 (Jones, 1977, Genetics 85:12). The presence of the trp1 lesion in the yeast host cell genome then provides an effective environment for detecting transformation by growth in the absence of tryptophan. Similarly, Leu2p-deficient yeast strains such as ATCC 20,622 and 38,626 are complemented by known plasmids bearing the LEU2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis (Van den Berg, 1990, Bio/Technology 8:135). Stable multi-copy expression vectors for secretion of mature recombinant human serum albumin by industrial strains of Kluyveromyces have also been disclosed (Fleer et al., 1991, Bio/Technology 9:968-975).

Expression and cloning vectors usually contain a promoter that is recognized by the host organism and is operably linked to the nucleic acid molecule encoding an anti-IL1RAP antibody or polypeptide chain thereof. Promoters suitable for use with prokaryotic hosts include phoA promoter, β-lactamase and lactose promoter systems, alkaline phosphatase, tryptophan (trp) promoter system, and hybrid promoters such as the tac promoter. Other known bacterial promoters are also suitable. Promoters for use in bacterial systems also will contain a Shine-Dalgarno (S.D.) sequence operably linked to the DNA encoding the humanized anti-IL1RAP antibody.

Many eukaryotic promoter sequences are known. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Inducible promoters have the additional advantage of transcription controlled by growth conditions. These include yeast promoter regions for alcohol dehydrogenase 2, isocytochrome C, acid phosphatase, derivative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Humanized anti-IL1RAP antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601, disclosing expression of human p-interferon cDNA in mouse cells under the control of a thymidine kinase promoter from herpes simplex virus. Alternatively, the Rous sarcoma virus long terminal repeat can be used as the promoter.

Another useful element that can be used in a recombinant expression vector is an enhancer sequence, which is used to increase the transcription of a DNA encoding a humanized anti-IL1RAP antibody by higher eukaryotes. Many enhancer sequences are now known from mammalian genes (e.g., globin, elastase, albumin, α-fetoprotein, and insulin). Typically, however, an enhancer from a eukaryotic cell virus is used. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 for a description of enhancing elements for activation of eukaryotic promoters. The enhancer may be spliced into the vector at a position 5′ or 3′ to the humanized anti-IL1RAP antibody-encoding sequence, but is preferably located at a site 5′ from the promoter.

Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-IL1RAP antibody. One useful transcription termination component is the bovine growth hormone polyadenylation region. See WO94/11026 and the expression vector disclosed therein. In some embodiments, humanized anti-IL1RAP antibodies can be expressed using the CHEF system. (See, e.g., U.S. Pat. No. 5,888,809; the disclosure of which is incorporated by reference herein.)

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for humanized anti-IL1RAP antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated humanized anti-IL1RAP antibody are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk worm). A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

In another aspect, expression of humanized anti-IL1RAP is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36:59), baby hamster kidney cells (BHK, ATCC CCL 10), Chinese hamster ovary cells/−DHFR1 (CHO, Urlaub et al., 1980, Proc. Natl. Acad. Sci. USA 77:4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383:44-68), MRC 5 cells, FS4 cells, and human hepatoma line (Hep G2).

Host cells are transformed with the above-described expression or cloning vectors for humanized anti-IL1RAP antibody production and cultured in conventional nutrient media modified as appropriate for inducing promoters, selecting transformants, or amplifying the genes encoding the desired sequences.

The host cells used to produce a humanized anti-IL1RAP antibody described herein may be cultured in a variety of media. Commercially available media such as Ham's F10 (Sigma-Aldrich Co., St. Louis, Mo.), Minimal Essential Medium ((MEM), (Sigma-Aldrich Co.), RPMI-1640 (Sigma-Aldrich Co.), and Dulbecco's Modified Eagle's Medium ((DMEM), Sigma-Aldrich Co.) are suitable for culturing the host cells. In addition, any of the media described in one or more of Ham et al., 1979, Meth. Enz. 58:44, Barnes et al., 1980, Anal. Biochem. 102:255, U.S. Pat. Nos. 4,767,704, 4,657,866, 4,927,762, 4,560,655, 5,122,469, WO 90/103430, and WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamicin), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, the cells may be disrupted to release protein as a first step. Particulate debris, either host cells or lysed fragments, can be removed, for example, by centrifugation or ultrafiltration. Carter et al., 1992, Bio/Technology 10:163-167 describes a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. A variety of methods can be used to isolate the antibody from the host cell.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSET chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antibody fragment of the present invention. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding an anti-IL1RAP polypeptide (e.g., a heavy chain or light chain variable region), or its complement. Hybridizing nucleic acids of the type described herein can be used, for example, as a cloning probe, a primer, e.g., a PCR primer, or a diagnostic probe.

### Non-Therapeutic Uses

The antibodies described herein are useful as affinity purification agents. In this process, the antibodies are immobilized on a solid phase such a Protein A resin, using methods well known in the art. The immobilized antibody is contacted with a sample containing the IL1RAP protein (or fragment thereof) to be purified, and thereafter the support is washed with a suitable solvent that will remove substantially all the material in the sample except the IL1RAP protein, which is bound to the immobilized antibody. Finally, the support is washed with another suitable solvent that will release the IL1RAP protein from the antibody.

Anti-IL1RAP antibodies, for example humanized anti-IL1RAP antibodies, are also useful in diagnostic assays to detect and/or quantify IL1RAP protein, for example, detecting IL1RAP expression in specific cells, tissues, or serum. The anti-IL1RAP antibodies can be used diagnostically to, for example, monitor the development or progression of a disease as part of a clinical testing procedure to, e.g., determine the efficacy of a given treatment and/or prevention regimen. Detection can be facilitated by coupling the anti-IL1RAP antibody. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention.

The anti-IL1RAP antibodies can be used in methods for diagnosing an IL1RAP-associated disorder (e.g., a disorder characterized by abnormal expression of IL1RAP) or to determine if a subject has an increased risk of developing an IL1RAP-associated disorder. Such methods include contacting a biological sample from a subject with an IL1RAP antibody and detecting binding of the antibody to IL1RAP. By “biological sample” is intended any biological sample obtained from an individual, cell line, tissue culture, or other source of cells potentially expressing IL1RAP. Methods for obtaining tissue biopsies and body fluids from mammals are well known in the art.

In some embodiments, the method can further comprise comparing the level of IL1RAP in a patient sample to a control sample (e.g., a subject that does not have an IL1RAP-associated disorder) to determine if the patient has an IL1RAP-associated disorder or is at risk of developing an IL1RAP-associated disorder.

It will be advantageous in some embodiments, for example, for diagnostic purposes to label the antibody with a detectable moiety. Numerous detectable labels are available, including radioisotopes, fluorescent labels, enzyme substrate labels and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

Exemplary radioisotopes labels include ³⁵S, ¹⁴C, ¹²⁵I, ³H, and ¹³¹I. The antibody can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the antibody via known techniques, such as those disclosed in Current Protocols in Immunology, for example. Fluorescence can be quantified using a fluorimeter.

There are various well-characterized enzyme-substrate labels known in the art (see, e.g., U.S. Pat. No. 4,275,149 for a review). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73:147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3′,5,5′-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

In another embodiment, the humanized anti-IL1RAP antibody is used unlabeled and detected with a labeled antibody that binds the humanized anti-IL1RAP antibody.

The antibodies described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

The anti-IL1RAP antibody or antigen binding fragment thereof can be used to inhibit the binding of ligand to the IL-36 receptor. Such methods comprise administering an anti-IL1RAP antibody or antigen binding fragment thereof to a cell (e.g., a mammalian cell) or cellular environment, whereby signaling mediated by the IL-36 receptor is inhibited. These methods can be performed in vitro or in vivo. By “cellular environment” is intended the tissue, medium, or extracellular matrix surrounding a cell. The anti-IL1RAP antibody or antigen binding fragment thereof is administered to the cellular environment of a cell in such a manner that the antibody or fragment is capable of binding to IL1RAP molecules outside of and surrounding the cell, therefore, preventing the binding of IL-36 ligand to its receptor.

### Diagnostic Kits

An anti-IL1RAP antibody can be used in a diagnostic kit, i.e., a packaged combination of reagents in predetermined amounts with instructions for performing the diagnostic assay. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Therapeutic Uses

In another embodiment, a humanized anti-IL1RAP antibody disclosed herein is useful in the treatment of various disorders associated with the expression of IL1RAP as described herein. Methods for treating an IL1RAP associated disorder comprise administering a therapeutically effective amount of a humanized anti-IL1RAP antibody to a subject in need thereof.

The humanized anti-IL1RAP antibody or agent is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). The humanized anti-IL1RAP antibody or agent can be administered, for example, as an infusion or as a bolus. Parenteral infusions include intramuscular, intravenous, intraarterial, intraperitoneal, or subcutaneous administration. In addition, the humanized anti-IL1RAP antibody is suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 20 mg/kg (e.g., 0.1-15 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is that disclosed in WO 94/04188.

The term “suppression” is used herein in the same context as “amelioration” and “alleviation” to mean a lessening of one or more characteristics of the disease.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat the disorder associated with IL1RAP expression.

The antibody need not be, but is optionally, formulated with one or more agents currently used to prevent or treat the disorder in question. The effective amount of such other agents depends on the amount of humanized anti-IL1RAP antibody present in the formulation, the type of disorder or treatment, and other factors discussed above. These are generally used in the same dosages and with administration routes as used hereinbefore or about from 1 to 99% of the heretofore employed dosages.

### Pharmaceutical Compositions and Administration Thereof

A composition comprising an IL1RAP binding agent (e.g., an anti-IL1RAP antibody) can be administered to a subject having or at risk of having an immunological disorder, respiratory disorder or a cancer. The invention further provides for the use of a IL1RAP binding agent (e.g., an anti-IL1RAP antibody) in the manufacture of a medicament for prevention or treatment of a cancer, respiratory disorder or immunological disorder. The term “subject” as used herein means any mammalian patient to which an IL1RAP binding agent can be administered, including, e.g., humans and non-human mammals, such as primates, rodents, and dogs. Subjects specifically intended for treatment using the methods described herein include humans. The antibodies or agents can be administered either alone or in combination with other compositions in the prevention or treatment of the immunological disorder, respiratory disorder or cancer. Such compositions which can be administered in combination with the antibodies or agents include methotrexate (MTX) and immunomodulators, e.g. antibodies or small molecules.

Examples of antibodies for use in such pharmaceutical compositions are those that comprise an antibody or antibody fragment having the light chain variable region amino acid sequence of any of SEQ ID NO: 17-66. Examples of antibodies for use in such pharmaceutical compositions are also those that comprise a humanized antibody or antibody fragment having the heavy chain variable region amino acid sequence of any of SEQ ID NO: 67-116.

Further examples of antibodies for use in such pharmaceutical compositions are also those that comprise a humanized antibody or antibody fragment having the light chain variable region amino acid sequence of any of SEQ ID NO:17, 36, 40, 47, 50, 51, and 52. Preferred antibodies for use in such pharmaceutical compositions are also those that comprise a humanized antibody or antibody fragment having the heavy chain variable region amino acid sequence of any of SEQ ID NO:67, 86, 90, 97, 100, 101, and 102.

Further examples of antibodies for use in such pharmaceutical compositions are also those that comprise a humanized antibody or antibody fragment having the light chain variable region and heavy chain variable region of any of SEQ ID NO: 17 and 67, SEQ ID NO: 36 and 86, SEQ ID NO: 40 and 90, SEQ ID NO: 47 and 97, SEQ ID NO: 50 and 100, SEQ ID NO: 51 and 101, and SEQ ID NO: 52 and 102.

Further examples of antibodies for use in such pharmaceutical compositions are also those that comprise Antibody A1, Antibody A2, Antibody A3, Antibody A4, Antibody A5, Antibody A6, or Antibody A7.

Various delivery systems are known and can be used to administer the IL1RAP binding agent. Methods of introduction include but are not limited to intradermal, intramuscular, intraperitoneal, intravenous, subcutaneous, intranasal, epidural, and oral routes. The IL1RAP binding agent can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

In specific embodiments, the IL1RAP binding agent composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the anti-IL1RAP antibody or agent does not absorb are used.

In other embodiments, the anti-IL1RAP antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An IL1RAP binding agent (e.g., an anti-IL1RAP antibody) can be administered as pharmaceutical compositions comprising a therapeutically effective amount of the binding agent and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing a IL1RAP binding agent (e.g., an anti-IL1RAP antibody) in lyophilized form and (b) a second container containing a pharmaceutically acceptable diluent (e.g., sterile water) for injection. The pharmaceutically acceptable diluent can be used for reconstitution or dilution of the lyophilized anti-IL1RAP antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

The amount of the IL1RAP binding agent (e.g., anti-IL1RAP antibody) that is effective in the treatment or prevention of an immunological disorder or cancer can be determined by standard clinical techniques. In addition, in vitro assays may optionally be employed to help identify optimal dosage ranges. The precise dose to be employed in the formulation will also depend on the route of administration, and the stage of immunological disorder or cancer, and should be decided according to the judgment of the practitioner and each patient's circumstances. Effective doses may be extrapolated from dose-response curves derived from in vitro or animal model test systems.

Generally, the dosage of an anti-IL1RAP antibody or IL1RAP binding agent administered to a patient with an immunological disorder or IL1RAP-expressing cancer is typically about 0.1 mg/kg to about 100 mg/kg of the subject's body weight. The dosage administered to a subject is about 0.1 mg/kg to about 50 mg/kg, about 1 mg/kg to about 30 mg/kg, about 1 mg/kg to about 20 mg/kg, about 1 mg/kg to about 15 mg/kg, or about 1 mg/kg to about 10 mg/kg of the subject's body weight.

Exemplary doses include, but are not limited to, from 1 ng/kg to 100 mg/kg. In some embodiments, a dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 16 mg/kg. The dose can be administered, for example, daily, once per week (weekly), twice per week, thrice per week, four times per week, five times per week, six times per week, biweekly or monthly, every two months, or every three months. In specific embodiments, the dose is about 0.5 mg/kg/week, about 1 mg/kg/week, about 2 mg/kg/week, about 3 mg/kg/week, about 4 mg/kg/week, about 5 mg/kg/week, about 6 mg/kg/week, about 7 mg/kg/week, about 8 mg/kg/week, about 9 mg/kg/week, about 10 mg/kg/week, about 11 mg/kg/week, about 12 mg/kg/week, about 13 mg/kg/week, about 14 mg/kg/week, about 15 mg/kg/week or about 16 mg/kg/week. In some embodiments, the dose ranges from about 1 mg/kg/week to about 15 mg/kg/week.

In some embodiments, the pharmaceutical compositions comprising the IL1RAP binding agent can further comprise a therapeutic agent, either conjugated or unconjugated to the binding agent. The anti-IL1RAP antibody or IL1RAP binding agent can be co-administered in combination with one or more therapeutic agents for the treatment or prevention of immunological disorders or cancers.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an anti-IL1RAP antibody or IL1RAP binding agent is administered concurrently with a therapeutic agent. In another specific embodiment, the therapeutic agent is administered prior or subsequent to administration of the anti-IL1RAP antibody or IL1RAP binding agent, by at least an hour and up to several months, for example at least an hour, five hours, 12 hours, a day, a week, a month, or three months, prior or subsequent to administration of the anti-IL1RAP antibody or IL1RAP binding agent.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL1RAP antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

The antibodies of the present invention are further described in the Examples below.

### EXAMPLES

### Example 1: Identification of Anti-Human IL1RAP Antibodies

A) Immunization with Human IL1RAP

Mice were immunized with recombinantly produced human IL1RAP (Genbank Accession NP_002173.1) protein and those which generated a strong titer response taken into traditional hybridoma generation. Fusion products blocking IL33 and IL36 signaling in whole blood and PBMCS assays with a weaker/cell type specific activity against IL1 were subcloned and re-screened. Inhibition of each of the cytokines that signal through IL1RAP is currently explored in clinical studies using either antibodies to the individual cytokines or to the cognate receptors, demonstrating the attractiveness of this pathway family. Variable domains were cloned from the hybridomas using standard PCR primer sets. The variable domains and specific CDRs are described above.

### B) Generation of Chimeric Fab:

Candidate IL1RAP G011 chimeric Fab was generated to benchmark and evaluate the humanized/sequence optimized Fabs in the ELISA binding experiments. Briefly, chimeric Fab was generated by fusing the mouse VK and VH residues to human Ck and CH1 residues respectively. Using the mouse sequence of selected antibody candidates, gene fragments were designed and synthesized for the murine VK and VH. Biotinylated forward primer containing specific sequence to the murine framework 1 region and an overhanging sequence annealed to the end of the Gene III sequence, and reverse primer from the conserved human constant region (CK or CH1 respectively) are used to amplify the V region. PCR was performed using the gene fragments as template and the DNA product was cloned into the M13LE01 vector using standard protocols. E. coli plaques expressing the DNA of interest were selected, and corresponding DNA samples were isolated and sequenced. The E. coli XL1-blue cells grown in exponential phase were induced with 0.5 mM IPTG followed by infection with the correct plaque representing chimeric Fab and were grown over night at 25° C. Either culture supernatants or the bacterial pellet was collected by centrifugation. Pellet was frozen at −80° C. for 15 minutes followed by thawing at RT in lysis buffer (20 mM Tris, 150 mM NaCl, 5 mM EDTA, pH8.0). Pellet were resuspended by vortexing vigorously for 15 minutes. The lysates form the periplasmic extracts containing soluble chimeric Fab were obtained by centrifugation.

### C) ELISA Binding Assay Development:

To identify the positive binders during screenings, ELISA binding assays were developed to identify binders independent of expression of Fabs to ensure the data represented binding activity of Fabs. Briefly, plates were coated with different amounts of anti-Fd (Meridian Life Science, Cat #W90075C) overnight in 96 well format. Assay plates were blocked with 3% milk in PBD for 1 h followed by addition of different amounts of either culture supernatants or periplasmic extracts of chimeric/sequence optimized Fabs. For the primary screen, plates were coated with 800 ng/ml of the anti-Fd antibody, and with 400 ng/ml of the anti-Fd antibody for the confirmatory screening (periplasmic Fab, in triplicate). Biotinylated antigen was used at 30 ng/ml for the 1× primary screening (secreted Fab) and at 10 ng/mL for the 3× confirmatory screenings (periplasmic Fab, in triplicate).

### D) Cloning, Expression and Purification of IgGs

The mouse antibodies were converted to chimeric antibodies consisting of the mouse variable domains on human constant domains (hu IgG1KO/kappa). The hu IgG1KO (knock out) has two replacement mutations (Leu234Ala and Leu235Ala) that eliminate ADCC and CDC activity by reducing effector functions such as FcγR and complement binding. The variable domains of the mouse and chimeric antibodies are identical. Chimeric antibodies are generated to confirm the function of the antibody and to ensure the correct variable domain sequence has been obtained.

To produce antibody constructs, the codon optimized DNA for sequence optimized VH and VK regions were fused to the heavy and light chain constant regions from human IgG1, using the In-Fusion® HD Cloning Kit (Clontech, U.S.A.) to directional clone VK gene into pTT5 huIgK vector and VH gene into pTT5 huIgG1KO vectors. To facilitate In-Fusion® HD Cloning, PCR products were purified and treated with Cloning Enhancer before In-Fusion® HD Cloning. Cloning and transformation were performed according to manufacturer's protocol (Clontech, U.S.A.). The DNA sequence of the subcloned gene V-gene fragments was confirmed by isolating mini-prep DNAs and subjecting isolated DNA to Sanger double stranded sequencing.

Both light and heavy chain expression vector DNA were expanded for transfection into CHO-E cells. Transfected CHO-E cells growing in suspension in serum-free media were cultivated in shake flasks under agitation at 140 rpm, 37° C. and 5% CO₂ and kept at conditions of exponential growth. On the day of transfection, cells were chemically transfected with 1 mg of light chain plasmid and 0.5 mg of heavy chain plasmid. They were then seeded at 1 to 2×10⁶ cells/ml in 1 L of Gibco® FreeStyle™ CHO expression medium (LifeTechnologies, NY, US). Cells were then incubated under orbital shaking for 10 to 12 days with one-time feeding of 150 ml commercial feed solution to allow expression of the proteins. Antibody titers in the cell culture supernatants were determined using an Octet® instrument (Pall ForteBio, CA, US) and protA biosensor tips according to manufacturer's instructions.

Recombinant antibodies were purified from culture supernatant by Protein A affinity chromatography using MabSelect™ (Amersham Biosciences) and stored in 60 mM NaOAc buffer (pH 5.0). Purity and degree of heterogeneity of the samples were assessed by mass spectrometry and analytical ultracentrifugation. All samples were confirmed to have a monomer content of ≥90% and contain <10% impurities prior to functional testing.

### Example 2: Production of Humanized IL1RAP Antibodies

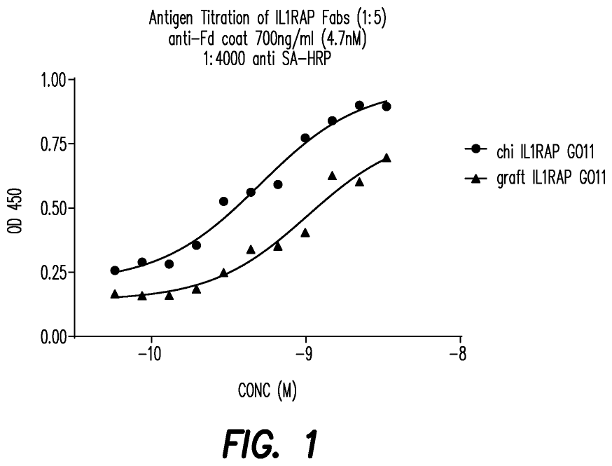

In order to reduce potential immunogenicity following administration in man the mouse anti-human IL1RAP monoclonal antibody GO11 was ‘humanized’ through a design and screening process. Human framework sequences were selected for the mouse leads based on the framework homology, CDR structure, conserved canonical residues, conserved interface packing residues and other parameters. The specific substitution of amino acid residues in these framework positions can improve various aspects of antibody performance including binding affinity and/or stability, over that demonstrated in humanized antibodies formed by “direct swap” of CDRs or HVLs into the human germline framework regions. For framework engineering, we identified the closely matching human germline to the murine GO11 candidate, and the CDRs from mouse lead were grafted on to the human germlines IGKV1-39*01 and IGHV1-69*02. The resulting engrafted variable regions were cloned in M13 based vector and expressed in E. coli cells. The engrafted Fab from E. coli periplasmic extracts was evaluated for binding activity in ELISA experiments and chimeric Fab with mouse variable regions was kept as positive control (FIG. 1).

As seen in FIG. 1, engrafted Fab depicted weaker binding activity than mouse Fab suggesting that the need to identify the critical mouse residues to regain the binding activity.

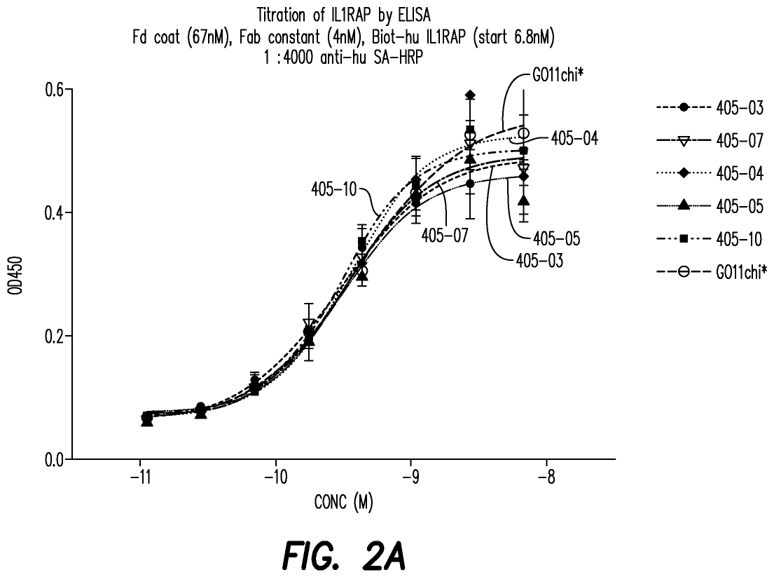

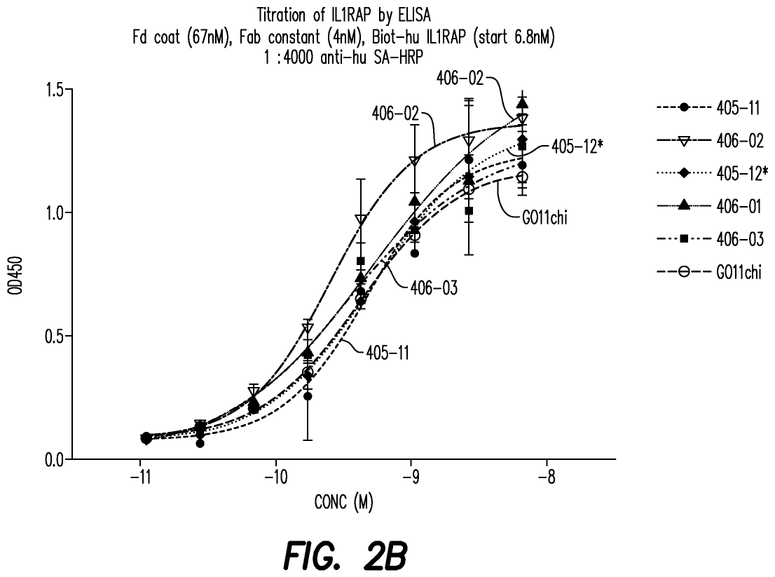

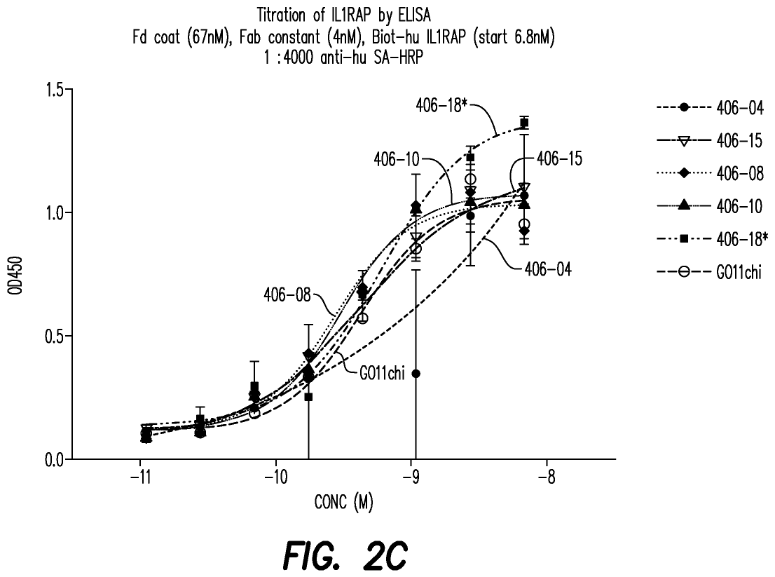

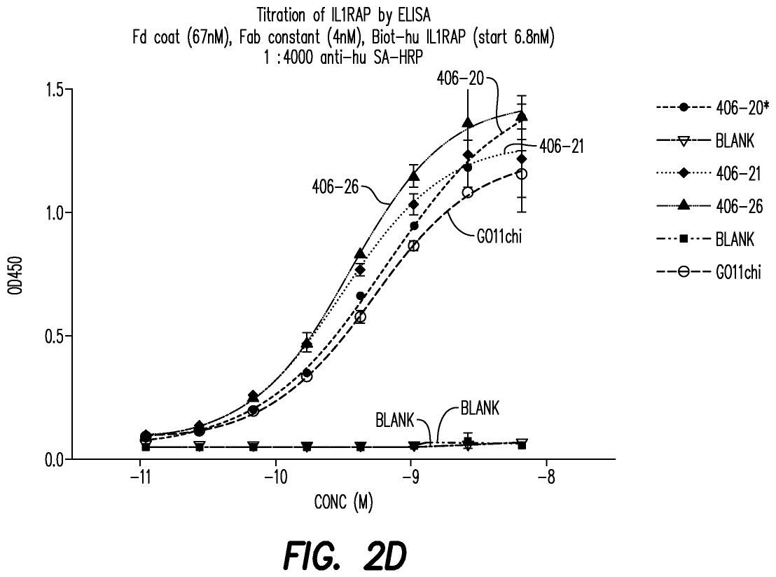

Besides the CDRs, certain mouse framework residues such as canonical and Vernier zone residues have been known to play an important role in positioning the CDR loops. To identify the human framework that retained similar binding of murine lead candidate residues closer to CDRs and Vernier zone and canonical residues were changed from human to mouse residues. A phage library representing various mutations/changes in the framework sequences was constructed, specifically K45Q, I48 V, Y49H and Q100A in VK while for VH, Q3H, K23E, R38K, A40R, M481, R67K, V68A, T69R, 170F, A72V and Y95F were evaluated (FIG. 2A/D). E. coli cells were transformed with the DNA containing different combinations. Approximately 450 VK clone variants and 1350 VH clone variants were selected and screened for binding to huIL1RAP by ELISA. As compared to the chimeric parent anti-IL1RAP Fab.

Molecules that depicted binding similar to positive control were selected were checked for their binding activity again in confirmatory ELISA binding experiments. Plaques representing the selected binders were used to infect the E. coli cells again and cultures were induced with 0.5 mM IPTG. Periplasmic extracts were used to confirm the binding to huIL1RAP antigen. As earlier, we kept chimeric Fab as positive control in our experiments.

As can be seen in FIG. 2A-D, clones 405-10 and 405-12 for VK (SEQ ID NOS: 18 and 19, respectively) and for VH clones 406-18 and 406-20 (SEQ ID NO:68 AND 69, respectively) clones depicted similar binding as chimeric controls. Sequence analysis of these light chain frameworks revealed amino acid mutation Y49H in FW2 was present in all clones those depicted binding similar to positive control chimeric Fab, and thus was considered an important critical mouse residue to be included in optimized light chain framework. For the VH, amino acid mutation A72V in FW3 was present in multiple clones including 406-18 and 406-20 along with other mouse residues and thus was considered important for optimized molecules although not critical. 406-18 had an additional change T69R while 4306-20 had 170F change beside A72V in frameworks 3 but none of these residues appear to be critical for binding. In light of these observations, we decided to use engrafted VH with A72V residue as potential critical residue in the final library and as described later.

### Optimization of CDRs

Germlining has evolved as a novel strategy to change non-critical mouse residues to more human germline residues to improve the percent human content of antibodies derived from hon-human primates and thus to reduce the formation of anti-drug antibodies. We evaluated the role of these mouse residues of IL1RAP in L-CDR1, L-CDR2, and part of L-CDR3, H-CDR1 and H-CDR2 by changing them to human germline residues as point mutants in IgG backbone.

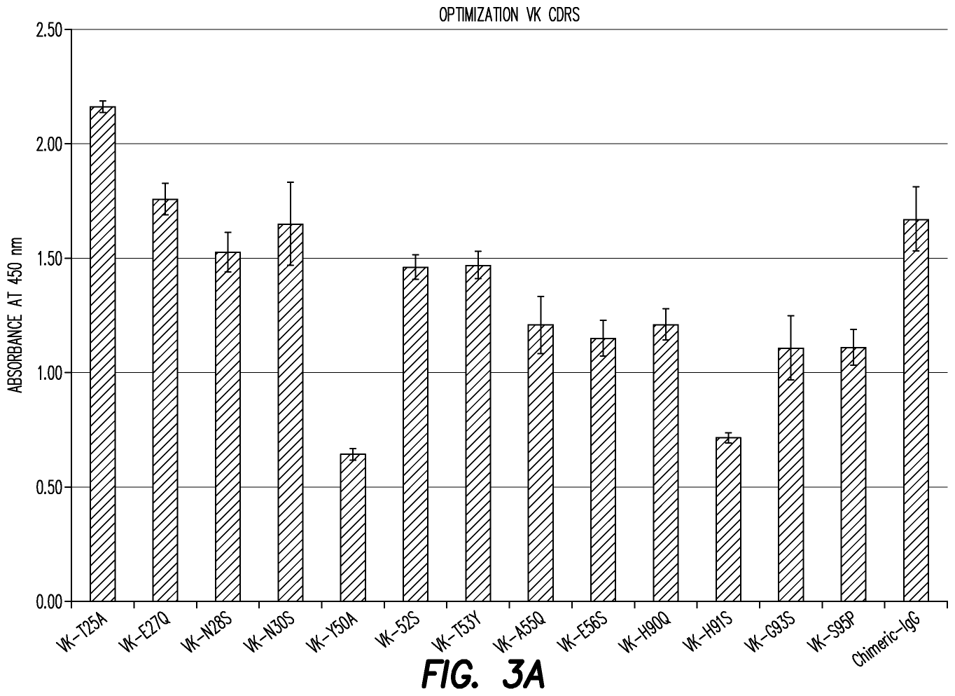

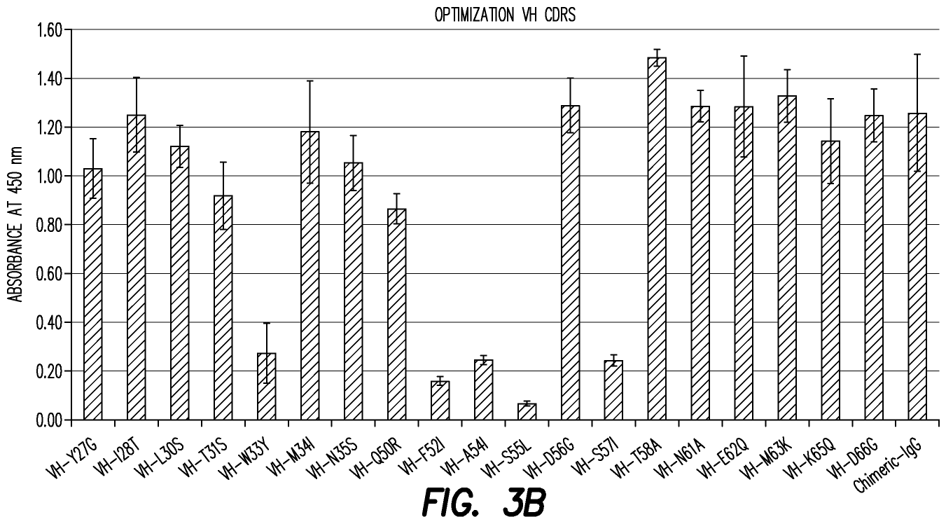

For light chain 13 variants (T25A, E27Q, N28S, N30S, Y50A, K52S, T53Y, A55Q, E56S, H90Q, H91S, G93S and S95P) and 19 heavy chain variants (Y27G, I28T, L30S, T31S, W33Y, M34I, N35S, Q50R, F521, A541, S55L, D56G, S57I, T58A, N61A, E62Q, M63K, K65Q and D66G) were generated separately in the vector pTT5 (See FIG. 3A/B). The light chain mutants were paired with the parental chimeric heavy chain and heavy chain mutants were paired with parental chimeric light chain for expression analysis in CHO-E cells for 7 days. The resulting variants were evaluated for binding in ELISA experiments and compared to the parental chimeric IgG which has mouse variable region but human constant regions. For light chain variants, six point mutants VK-T25A, E27Q, N28S, N30S, K52S and T53Y depicted binding similar to chimeric IgG (+10% control; FIG. 3A) while for heavy chain ten point mutants VH-I28T, L30S, M34I, N35S, D56G, T58A, N61A, E62Q, M63K, K65Q and D66G depicted similar binding as chimeric IgG (+10% control; FIG. 3B).

Fabs that showed better or equal binding and improved expression as compared to the chimeric parent Fab were selected for further characterization.

Representative humanized variable regions for antibody GO11 are shown in the specification section. In this manner, Antibody A1 to Antibody A7 were humanized antibodies derived from mouse antibody 005-GO11 (VL/VH SEQ ID NO:1 AND 2) (cloned into a human IgG1 KO (KO=knock-out)/kappa backbone. Antibodies A1 to A7 are shown in Table 25.

The exemplary anti-IL1RAP Ab of the invention bind to human and cyno IL1RAP (ECD) specifically. The binding affinity of antibody binding to human IL1RAP and cyno IL1RAP was determined with SPR to be 110 and 160 pM, respectively. The anti-IL1RAP antibodies of the invention are not cross-reactive to mouse IL1RAP.

TABLE 27

| Antigen | kₐ (1/Ms) | k_{d} (1/s) | K_{D} (M) | K_{D} (pM) |
|---|---|---|---|---|
| His-IL1RAP human | 1.67 × 10⁵ | 1.84 × 10⁻⁵ | 1.10 × 10⁻¹⁰ | 110 |
| CyIL1RAP | 1.66 × 10⁵ | 2.66 × 10⁻⁵ | 1.60 × 10⁻¹⁰ | 160 |

### Example 3: Bioactivity; Potency of Humanized Anti-Human IL1RAP Antibodies in Functional Human Assays—Blocking of IL-33, IL-36, and IL-1 Signaling

Activation of target cells by IL-1β depends on its interaction with two membrane bound receptors: IL-1 receptor type I (IL-1R1) and IL-1 receptor accessory protein (IL-1RAcP) IL-1R1 is the ligand recognition receptor that binds IL-1β with high affinity. Although IL-1 RAcP does not interact with the IL-1β directly, its recruitment is essential or the formation of a signaling competent complex. IL1RAP is also a functional part of the IL-33 receptor complex, and therefore binding to IL1RAP may inhibit both IL-1 and IL-33 signaling.

In order to test for the capability of potential lead candidate antibodies to block IL-1 and IL-33 signaling IL-1 and IL-33 dependent cell assays were established. The exemplary antibodies of the invention were tested for inhibition of IL-1 β and IL-33 signaling.

### Protocols:

### Generation and Stimulation of Monocyte Derived Macrophages (MDM):

Human monocyte derived macrophages were differentiated and generated as recently descripted in Koss et al. (IL36 is a Critical Upstream Amplifier of Neutrophilic Lung Inflammation in Mice, publication pending). The cells were stimulated with media containing Dulbecco's modified Eagle's medium (DMEM) (1×)+GlutaMAX™-I (GIBCO #31966-021); 10% HI fetal calf serum (FCS; GIBCO #16140-071); 1% NEAA (100× GIBCO #11140-035); 1% P/S (10,000 U/mL Penicillin, 10,000 μg/mL Streptomycin GIBCO #15140-122) and 10 ng/ml recombinant human MCSF (R&D Systems; 216-MCC/CF). 6.2×104 MDMs were seeded in a 96-well plate. After 24 h macrophages were pre-stimulated with the anti-IL1rap AB (0.4, 2, 10, 50, 250 nM) and after 30 min MDMs were stimulated for 24 h with rhIL-36a (33 ng/ML; 6995-IL-010/CF; R&D Systems), rhIL-363 (33 ng/ml; 6834-ILB-025/CF; R&D Systems), rhIL-36γ (33 ng/ml; 2320-IL-025/CF; R&D Systems), rhIL-1α (10 ng/ml; 200-LA-010/CF; R&D Systems), at 37° C. and 5% CO2. The IL12p40 concentrations were measured in the supernatant via single MSD (Meso Scale Discovery, #K151UQK-1) according to the manufacturer's instructions.

### Generation and Stimulation of Monocyte Derived Dendritic Cells (MoDC):

Monocytes were isolated from peripheral blood mononuclear cells from human volunteer donors as described in Koss et al. (IL36 is a Critical Upstream Amplifier of Neutrophilic Lung Inflammation in Mice, publication pending). 1×10⁵ monocytes were seeded in a 96-well plate using the Dendritic Cell Culture Kit (#10985, Stemcel) according to the manufacturer's instructions. After 8 days differentiated MoDC were pre-stimulated for 30 minutes with the anti-IL1rap AB (0.4, 2, 10, 50, 250 nM) and further stimulated with the combination of rhIL-36α (33 ng/ml; 6995-IL-010/CF; R&D Systems), rhIL-36β (33 ng/ml; 6834-ILB-025/CF; R&D Systems) and rhIL-36γ (33 ng/ml; 2320-IL-025/CF; R&D Systems) for 48 h at 37° C. and 5% CO2. IL-8, IL-6 and TNFα concentrations were measured in the supernatant via MSD (Meso Scale Discovery, #K15067L) according to the manufacturer's instructions.

### Whole Blood Assay:

180 μL whole blood from human volunteer donors (diluted 1:2 with RPMI-Medium (RPMI1640 Gibco #61870-010)) were pipetted in a 96-well plate. Anti-IL1rap Abs of the invention (0.07, 0.21, 0.62, 1.8, 5.5, 16.5, 50, 150, 450 nM (MIP1B), 0.1, 1, 10, 100, 1000 nM (IFNγ)) were added to the wells and pre-incubated for 30 min at 37° C. and 5% CO2. rhIL33 (3625-IL-010/CF, R&D Systems, 0.6 nM), rhIL36γ (6835-IL-010/CF, R&D Systems, 10 nM), rhIL-12 (219-IL/CF, R&D Systems, 0.25 nM) were added to the wells for 24 h at 37° C. and 5% CO2. The supernatant was collected and Mip1ß (DY271-05, DuoSet ELISA, R&D Systems) and IFNγ (#555142, ELISA, BD Biosciences) protein concentrations were measures according to manufacturer's instructions.

### Results:

The ability of the anti-IL1RAP Ab to suppress activation of immune cells by IL1 family cytokines was determined. Human monocyte derived macrophages (MDMs) were stimulated as a relevant human innate immune cell in vitro with IL-36α, β, γ alone, IL-1α alone or with a combination of IL-36α, β, γ and IL-1a. IL-12 protein production was measured as a marker of cellular activation downstream of IL1 family cytokine activation.

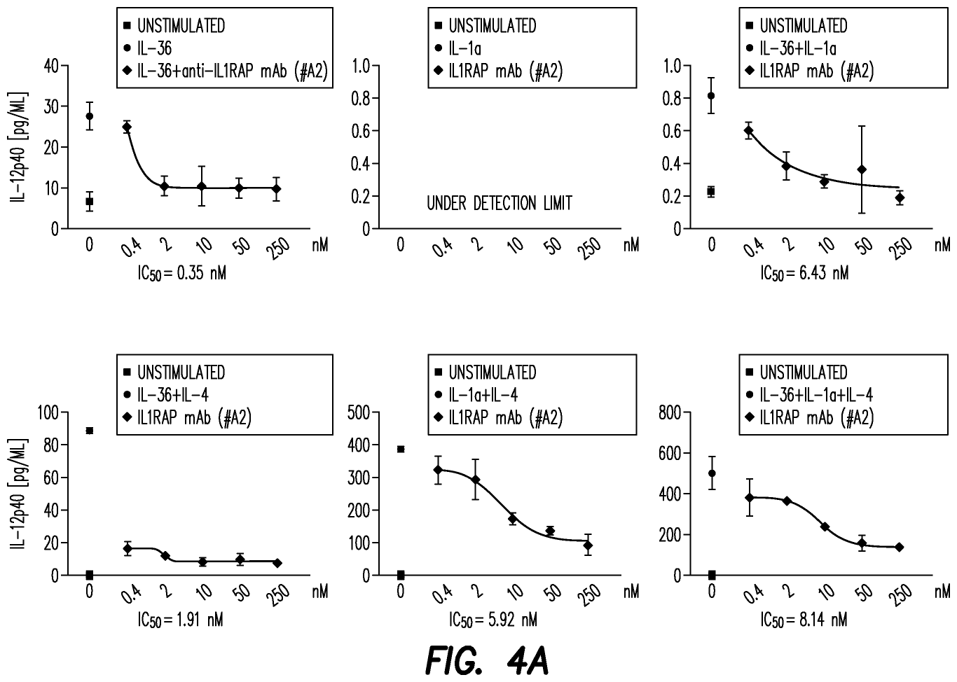

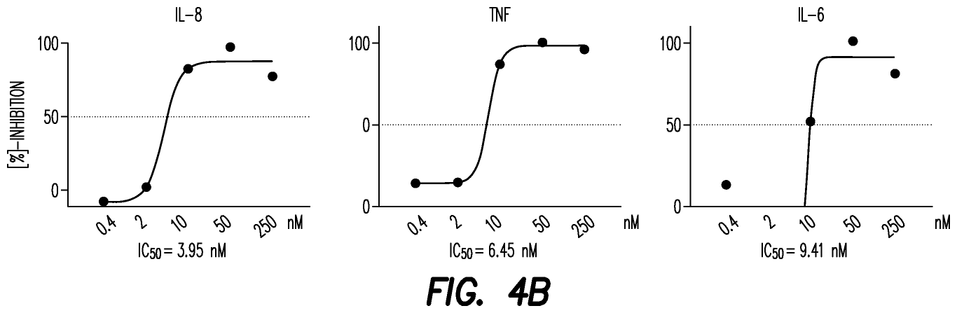

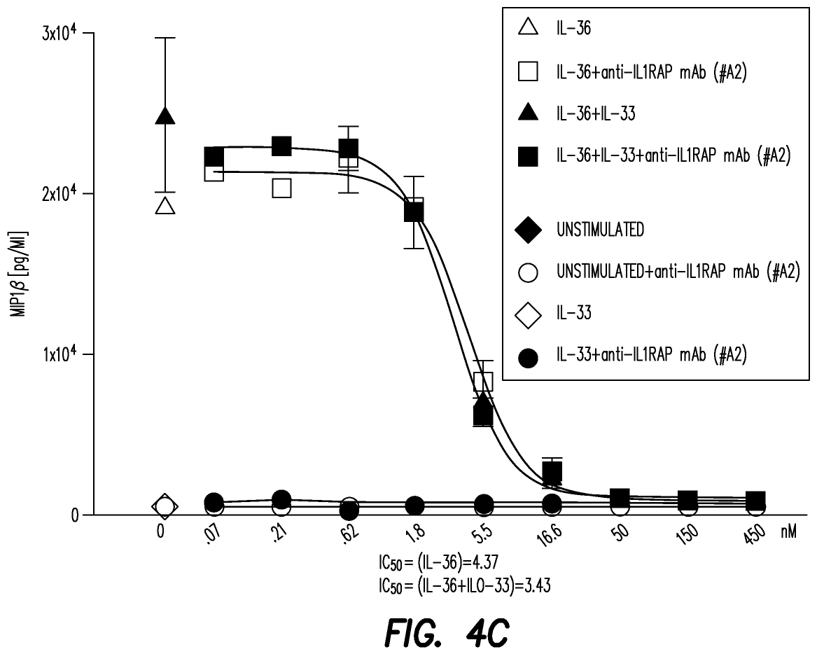

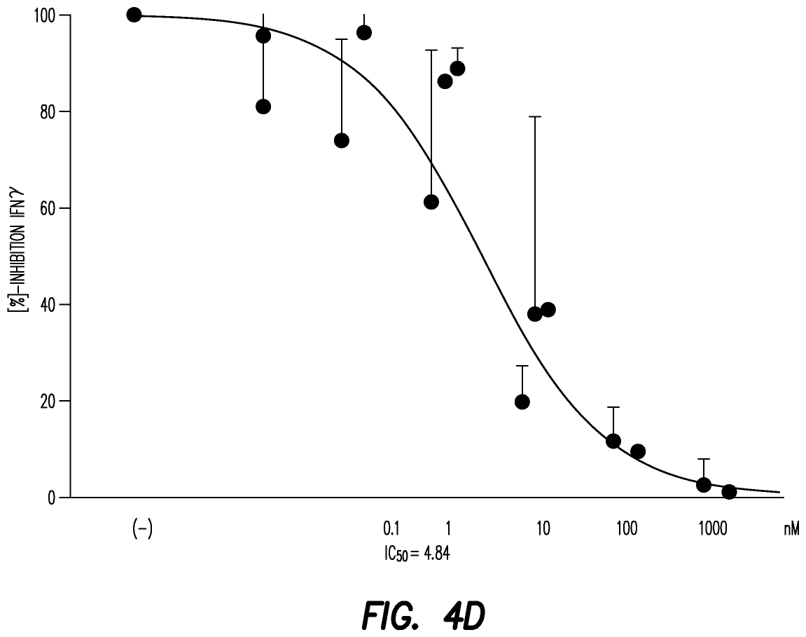

Production of IL-12 in response to stimulation with IL-36α, β, γ or IL-36α, β, γ in combination with IL-1a was markedly inhibited by the anti-IL1RaP Ab (IC50=0.35 nM and IC50=6.48 nM, respectively) (FIG. 4A). FIG. 4A shows the inhibition of IL-12p40 secretion by an anti-IL1RAP Ab of the invention in cytokine stimulated MDMs (depicted are mean values±SD of technical triplicates from one representative of two experiments with MDMs obtained from different donors). Because IL-1a alone was not capable of inducing IL-12 production in MDMs and similar results were obtained after IL-33 stimulation (data not shown), the ability of the anti-IL1RAP Abs to suppress IL-12 protein was measured in response to stimulation with IL-4 followed by exposure to IL-36 α, β, γ, IL-1a or IL-36α, β, γ together with IL-1a. Anti-IL1RAP Ab #A2 markedly suppressed production of IL-12 in response to IL-36α, β, γ (IC50=1.91 nM), IL-1a (IC50=5.92 nM), and IL-36α, β, γ/IL-1a (IC50-8.14 nM) in IL-4 conditioned MDMs (FIG. 4A).

Previous experiments had demonstrated that MoDCs were unresponsive to stimulation with IL-1a or IL-33 (not shown). Therefore, as a next measure of the ability of the IL1RAP antibody we stimulated human monocyte derived dendritic cells (MoDCs), a relevant human adaptive immune cell in vitro, with IL-36α, β, γ, or alone. Protein production of IL-8 (IC50=3.95 nM), TNF (IC50-6.45 nM), and IL-6 (IC50=9.41 nM) was markedly suppressed by the anti-IL1 Rap Ab (FIG. 4B). Consistent with the findings in MDMs and DCs, when we measured production of a canonical myeloid cell derived mediator Mip-1b in whole blood, IL-33 did not induce its production, while IL-36γ potently induced Mip-1b production, which was markedly suppressed by the anti-IL1RAP Ab #A2 (IC50=4.37) (FIG. 4C). Similar data were obtained after combining IL-36γ with IL-33 (IC50=3.43) (FIG. 4C).

IL-33 has been reported to activate production of IFNγ by lymphocytes. Pilot experiments had shown that the highest production of IFNγ in a whole blood assay was observed after combining IL-33 with IL-12 (data not shown). We therefore determined the ability of the anti-IL1RAP Ab to inhibit IL-33/IL-12 mediated production of IFNγ in a whole blood assay, in which production of IFNγ was markedly inhibited (IC50=4.84 nM) (FIG. 4D).

### Example 4: Epitope Binding of Anti-IL1RAP Antibody: The Unique Epitope as Shown by Crystal Structure and HDX MS

To understand where the anti-IL1RAP clones bound to IL1RAP, a structural analysis of the protein was performed using the extracellular domain of the receptor. The “extracellular domain” of IL1RAP forms a cell-membrane bound complex with an IL1 receptor, e.g., IL1 R type I or type II. Human IL1RAP (Accession No. Q9NPH3 UniProtKB/Swiss-Prot) is known to consist of three extracellular domains: Domain I: amino acids 21 to 134; Domain II: amino acids 135-234; and Domain III: amino acids 235 to 367.

### Crystallization of the IL1RAP: Fab Binary Complex:

IL1RAP and the anti-IL1RAP #A2 Fab were mixed in molar ratio of 1.2:1 and concentrated to 20 mg/mL. Crystals were obtained using the hanging drop vapor diffusion method by mixing 1 μl of the complex with 1 μL of a reservoir solution containing 0.1 M ammonium acetate, 0.1 M BIS-TRIS pH 6.0 and 15% w/v polyethylene glycol 10000. Crystals of plate-like shape appeared within one day. Crystals were cryo-protected with the reservoir solution supplemented with 30% v/v glycerol and flash frozen in liquid nitrogen.

### Data Collection and Structure Determination:

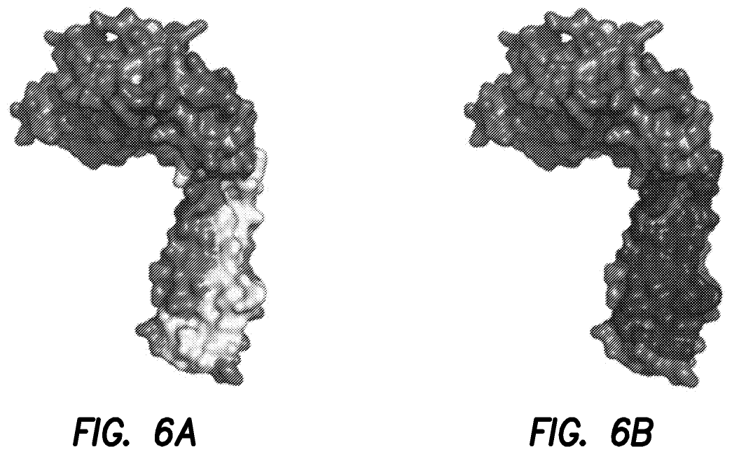

Data were collected at 100 K at the beamline X10SA of the SLS in Villigen, Switzerland. 720 frames with 0.25° per frame were collected on a PILATUS 6M-F detector. The data were processed with autoPROC software (Smart, et al. Global phasing Limited, 2013; Evans, P. (2006) Scaling and assessment of data quality. Acta Crystallogr D Biol Crystallogr 62, 72-82; Kabsch, W. (2010) Xds. Acta Crystallogr D Biol Crystallogr 66, 125-132; Vonrhein, C., Flensburg, C., Keller, P., Sharff, A., Smart, O., Paciorek, W., Womack, T., and Bricogne, G. (2011) Data processing and analysis with the autoPROC toolbox. Acta Crystallogr D Biol Crystallogr 67, 293-302; Tickle, I., Flensburg, C., Keller, P., Paciorek, W., Sharff, A., Vonrhein, C., and Bricogne, G. (2018) STARANISO. Global Phasing Ltd., Cambridge, United Kingdo; Winn, M. D., Ballard, C. C., Cowtan, K. D., Dodson, E. J., Emsley, P., Evans, P. R., Keegan, R. M., Krissinel, E. B., Leslie, A. G., McCoy, A., McNicholas, S. J., Murshudov, G. N., Pannu, N. S., Potterton, E. A., Powell, H. R., Read, R. J., Vagin, A., and Wilson, K. S. (2011) Overview of the CCP4 suite and current developments. Acta Crystallogr D Biol Crystallogr 67, 235-24; and Evans, P. R., and Murshudov, G. N. (2013) How good are my data and what is the resolution? Acta Crystallogr D Biol Crystallogr 69, 1204-1214). A model for molecular replacement was created with SCULPTOR within the PHENIX software suite (Adams, et al. (2010) PHENIX: a comprehensive Python-based system for macromolecular structure solution. Acta Crystallogr D Biol Crystallogr 66, 213-221) using the Fab of pdb:1hzh as template. Molecular replacement was performed with PHASER (McCoy, et al. (2007) Phaser crystallographic software. J Appl Crystallogr 40, 658-674) using the Fab model prepared with SCULPTOR and the Interleukin-1 repector accessory protein from pdb:4dep as templates. The structure was refined with Buster (Bricogne, et al. (2020) BUSTER version 2.11.7. in BUSTER version 2.11.7, 2.11.7 Ed., Global Phasing Ltd., Cambridge, United Kingdom) model building and least square fit analysis was done with COOT (Emsley, et al. (2010) Features and development of Coot. Acta Crystallogr D Biol Crystallogr 66, 486-501). FIGS. 6A-C were prepared with PyMOL (Schrodinger, LLC. (2015) The PyMOL Molecular Graphics System, Version 2.4).

TABLE 28
IL1RAP-#A2

| Wavelength [Å] | 0.99986 | |
|---|---|---|
| Resolution range [Å] | 39-2.974 | (3.08-2.974) |
| Space group | C 1 2 1 | |
| Unit cell [Å] and [°] | 143.103 95.645 102.359 90 106.05 90 | |
| Total reflections | 93331 | (9745) |
| Unique reflections | 27330 | (248) |
| Multiplicity | 3.4 | (3.6) |
| Completeness (%) | 71.15 | (9.03) |
| Mean I/sigma(I) | 4.72 | (0.39) |
| Wilson B-factor | 82.71 | |
| R-merge | 0.09947 | (2.613) |
| R-meas | 0.1182 | (3.078) |
| R-pim | 0.06325 | (1.615) |
| CC½ | 0.997 | (0.259) |
| CC* | 0.999 | (0.641) |
| Reflections used in refinement | 19518 | (248) |
| Reflections used for R-free | 925 | (17) |
| R-work | 0.2600 | (0.3520) |
| R-free | 0.3130 | (0.3569) |
| CC(work) | 0.829 | (0.369) |
| CC(free) | 0.765 | (0.513) |

| | |
|---|---|
| Number of non-hydrogen atoms | 6015 |
| macromolecules | 5995 |
| solvent | 20 |
| Protein residues | 767 |
| RMS(bonds) [Å] | 0.010 |
| RMS(angles) [°] | 1.51 |
| Ramachandran favored (%) | 87.58 |
| Ramachandran allowed (%) | 9.51 |
| Ramachandran outliers (%) | 2.91 |
| Rotamer outliers (%) | 7.52 |
| Clashscore | 0.51 |
| Average B-factor | 88.52 |
| macromolecules | 88.68 |
| solvent | 39.26 |

### Experimental Procedures of HDX MS:

For HDX (Hydrogen/Deuterium Exchange Mass Spectrometry) experiments, IL1RAP antigen was analyzed alone (control) and with anti-IL1RAP #A2 antibody of the invention present at approximately an equimolar ratio (mixed sample). All sample handling was performed by a LEAP HDX PAL system. To identify peptides, the control sample was incubated with H₂O buffer (H₂O 10 mM sodium phosphate pH 7.4). For exchange experiments, the control and mixed samples were incubated with D₂O buffer (D₂O 10 mM sodium phosphate pH 7.4) at 10, 100, and 1000 second time points (in duplicate) by the following procedure: (1) 4 μL of sample was added to 40 μL of H2O/D2O buffer; (2) the mixture was incubated at 20° C. for various time points (0, 10, 100, and 1000 seconds); (3) 40 μL of the incubated sample was transferred to 40 μL of 4° C. quench buffer (4M Urea, 0.4M TCEP-HCl); (4) 60 μL of the quenched sample was injected onto an immobilized protease XIII/pepsin column (1:1 2.1×30 mm, NovaBioAssays) by flowing 200 μL/min of Mobile Phase A (99% H2O, 1% Acetonitrile, and 0.1% Formic acid) for 2 minutes. The digested peptides were desalted on a Vanguard Pre-column (ACQUITY UPLC BEH C18, 130 Å, 1.7 μm, 2.1 mm×5 mm, Waters) for 3 minutes, and then separated by liquid chromatography with an Acquity UPLC BEH C18 Column 1.7 μm, 1 mm×50 mm (Waters) at 4° C. at a flow rate of 160 μl/min. The LC gradient solvent system was composed of mobile phase A (composition above) and mobile phase B (0.1% Formic acid, 5% H2O and 95% Acetonitrile). The percentage of mobile phase B was held at 5% for 5 minutes; increased from 5% to 15% at 5.6 minutes, to 40% at 10.4 minutes, to 90% at 11 minutes; held at 90% to 11.5 minutes; decreased to 5% at 12.4 minutes, and then held at 5% to 14 minutes. After chromatographic separation, the peptides were detected by the Thermo Scientific Orbitrap Fusion mass spectrometer operated in positive electrospray ionization mode. Data was then analyzed by Byonic software (Protein Metrics) to identify peptides and HDExaminer software (Sierra Analytics) to calculate deuterium incorporation.

### Results:

The structure of anti-IL1RAP #A2 Fab in complex with the extracellular domains of IL-1RAcP (residues 21-368, Table 28) was determined. All residues of IL-1RAcP are visible in the electron density except for residues 21-23 and 351-368. The Fab-chains could also be completely traced except for the four C-terminal residues of chain H and the first N-terminal residue of chain L. The crystallographic B-factors are lowest in the D3 domain and the Fab (86 Å2 and 82 Å2) and increase towards the N-terminus of IL-1RAcP (100 Å2 in domain D2 and 160 Å2 in domain D1). This can be explained by the crystal packing of the complex, where the D1 domain has the least number of contacts to neighboring molecules.

The structure of IL-1RAcP resembles the previously determined structures of human IL-1RACP-IL-1RI-IL1β (Thomas et al. (2012) Structure of the activating IL-1 receptor signaling complex. Nature structural & molecular biology 19, 455-457) and IL-1RAcP-IL-1RII IL1β (Wang et al. (2010) Structural insights into the assembly and activation of IL-1 beta with its receptors. Nat Immunol 11, 905-911) with an r.m.s. deviation of all IL-1RAcP backbone Ca-atoms of 2.7 Å and 2.4 Å, respectively. The exemplary anti-IL1RAP #A2 Fab of the invention binds to the D3-domain of IL-1 RAcP, the interface buries ˜900 Å2 of surface area on each binding partner. The epitope residues on the D3 domain coincide largely with the residues involved in the IL1RAP-IL-1RI interface. While the interactions of IL1RAcP with IL-1RI extend to residues in the D2 and D3 domain of IL1RAP, the interface of IL1RAP and anti-IL1RAP antibody of the invention is restricted to the D3-domain.

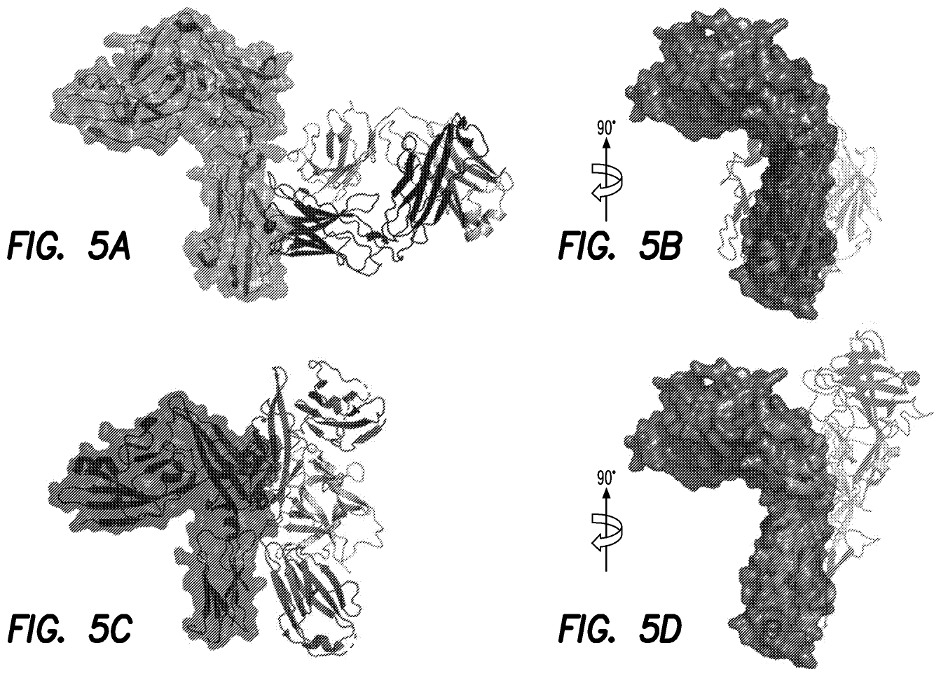

FIG. 5A-D illustrates the structural features of the IL-1RAcP:anti-IL1RAP Ab of the invention and the IL-1RAcP-IL-1RI-IL1β complexes in two different views related by a 90° rotation. In FIG. 5A, the IL-1RAcP is shown in pale blue with semi-transparent surface. The Fab is shown as ribbons. The heavy and light chains are colored in dark and light grey, respectively. FIG. 5B shows the location of the Fab on IL-1RAcP is shown in grey. The Fab is shown as semitransparent ribbon for clarity. FIG. 5C shows the structure of the human IL-1RAcP-IL-1RI-IL1β ternary complex (pdb 4dep) for comparison in the same orientation as FIG. 5A. IL-1RI is colored in red and IL-1β in green. FIG. 5D illustrates the IL-1RAcP-side of the IL-1RAcP-IL-1RI interface (shown in orange). IL-1RI and IL1β are shown as semitransparent ribbons for clarity.

The epitope is formed by amino acid residues 238, 239, 241, 244-247, 249, 251-256, 261, 263, 265, 267, 269, 271, 301, 303, 305-307, 311, 313, and 315 as defined by the program PISA. This is in good agreement to the epitope determined by HDX-MS (residues 226-262 and 269-273) as demonstrated in FIG. 6A-B comparing the HDX and the X-ray epitope on IL1RAP. In FIG. 6A the HDX epitope is shown in yellow comprising residues 226-262 and 269-273. In FIG. 6B the X-ray epitope is shown (as was also shown in FIG. 6B, except in this figure the anti-IL1RAP Fab of the invention is removed for clarity).

The amino acids of the IL1RAP extracellular domain start at amino acid position 21 and extends to amino acid 367 as shown as SEQ ID NO:184. IL1RAP Domain 1 corresponds to amino acids positions 21-134 (SEQ ID NO:185), Domain 2 corresponds to amino acids 135-234 (SEQ ID NO:186) Domain 3 nonresponses to amino acid positions 235-267 (SEQ ID NO:187). Domain 3 is further divided here according to the identified epitope binding regions as defined by X-Ray crystallography (XR-D3A/B corresponding to amino acid positions 235-315 (SEQ ID NO:188), XR D3A corresponding to amino acid positions 235-273 (SEQ ID NO:189), and XR D3B corresponding to amino acids 300-315 (SEQ ID NO: 190)) and HDX mapping (HDX D2/3 A corresponding to amino acid positions 226-262 (SEQ ID NO:191), and HDX D2/3 B corresponding to amino acid positions 226-273 (SEQ ID NO:192)). Underlined and bolded amino acid positions in SEQ ID NOs: 202-207 correspond to identified contact amino acids K238, N239, V241, V244, I245, H246, S247, N249, H251, V252, V253, Y254, E255, K256, E261, L263, P265, T267, Y269, S271, S301, S303, S305, R306, T307, T311, T313, I315 of the epitope that bind the exemplary anti-IL1RAP antibody #A2 by X-ray mapping.

IL1RAP ECD ACCESSION NO: Q9NPH3 (amino acids 21-367)
(SEQ ID NO: 184)
SERCDDWGLDTMRQIQVFEDEPARIKCPLFEHFLKFNYSTAHSAGLTLIWYWTRQDRD
LEEPINFRLPENRISKEKDVLWFRPTLLNDTGNYTCMLRNTTYCSKVAFPLEVVQKDSC
FNSPMKLPVHKLYIEYGIQRITCPNVDGYFPSSVKPTITWYMGCYKIQNFNNVIPEGMN
LSFLIALISNNGNYTCVVTYPENGRTFHLTRTLTVKVVGSPKNAVPPVIHSPNDHVVYEK
EPGEELLIPCTVYFSFLMDSRNEVWWTIDGKKPDDITIDVTINESISHSRTEDETRTQILS
IKKVTSEDLKRSYVCHARSAKGEVAKAAKVKQKVPAPRYTVELACGFGAT
IL1RAP ECD Domain 1 (amino acids 21-134):
(SEQ ID NO: 185)
SERCDDWGLDTMRQIQVFEDEPARIKCPLFEHFLKFNYSTAHSAGLTLIWYWTRQDRD
LEEPINFRLPENRISKEKDVLWFRPTLLNDTGNYTCMLRNTTYCSKVAFPLEVVQK
IL1RAP ECD Domain 2 (amino acids 135-234):
(SEQ ID NO: 186)
DSCFNSPMKLPVHKLYIEYGIQRITCPNVDGYFPSSVKPTITWYMGCYKIQNFNNVIPE
GMNLSFLIALISNNGNYTCVVTYPENGRTFHLTRTLTVKVV
IL1RAP ECD Domain 3 (amino acids 235-367):
(SEQ ID NO: 187)
GSPKNAVPPVIHSPNDHVVYEKEPGEELLIPCTVYFSFLMDSRNEVWWTIDGKKPDDI
TIDVTINESISHSRTEDETRTQILSIKKVTSEDLKRSYVCHARSAKGEVAKAAKVKQKVP
APRYTVELACGFGAT
IL1RAP ECD XR-D3A/B (AMINO ACIDS 235-315):
(SEQ ID NO: 188)
GSPKNAVPPVIHSPNDHVVYEKEPGEELLIPCTVYFSFLMDSRNEVWWTIDGKKPDDI
TIDVTINESISHSRTEDETRIQI
IL1RAP ECD XR D3A (AMINO ACIDS 235-273):
(SEQ ID NO: 189)
GSPKNAVPPVIHSPNDHVVYEKEPGEELLIPCTVYFSFL
IL1RAP ECD XR D3B (AMINO ACIDS 300-315):
(SEQ ID NO: 190)
SISHSRTEDETRIQI
IL1RAP ECD HDX D2/3 A (AMINO ACIDS 226-262):
(SEQ ID NO: 191)
TRTLTVKVVGSPKNAVPPVIHSPNDHVVYEKEPGEELL
IL1RAP ECD HDX D2/3 B (AMINO ACIDS 226-273):
(SEQ ID NO: 192)
TRTLTVKVVGSPKNAVPPVIHSPNDHVVYEKEPGEELLIPCTVYFSFL

The paratope is formed by residues 128 and L30-W33 from CDR-H1, F52, A54, S55, S57 and Y59 from CDR-H2, K74 from a loop following CDR-H2, Y102-G107 and Y109 from CDR-H3. From the light chain, only CDR-L2 (residues E56 and G57) and CDR-L3 (residues G93 and T94) are critical in binding of IL1RAP. Polar interactions comprise ten hydrogen bonds and one salt-bridge. These are summarized in Table 29.

TABLE 29
Polar interactions

| IL-1RAcP | T267 | V253 | H251 | N239 | H246 | N249 | H246 | R306 |
|---|---|---|---|---|---|---|---|---|
| CDR-H1 | T31 | | | | | | | |
| CDR-H2 | | S57 | Y59 | | | | | |
| CDR-H3 | | | | Y104 | Y109 | Y109 | S103 | |
| CDR-L2 | | | | | | | | E56 |

Two antibodies, CAN 03 and CAN04 were reported to bind to domain 3 and 2 respectively and inhibit IL-1 signaling and partial inhibition of IL-33 signaling (see Wang et al., 2010, Nature Immunology, 11:905-912 and WO2020/035577).

CAN04 and CAN03 were synthesized according to VH and VL sequences described in WO2020/035577 (herein incorporated by reference) and used to compare to an exemplary anti-IL1RAP antibody (#A2).

Table 30 summarizes the comparison of CAN04 vs chimeric precursor of exemplary anti-IL1RAP (#A2). The CAN04 Ab didn't show inhibition as broad inhibition as that of #A2 across multiple measured parameters in the Hek-Blue, and NCI-H292 assays, only demonstrating a comparable inhibitory potency against IL36γ in the WB (whole blood) assay.

For the CAN03 Ab, while antibody binding was detected, there was no measurable inhibition in our in vitro screening assays (Data not shown). This was consistent with previous findings reported in WO2020/035577 where only when CAN03 was combined with CAN04 was there measurable IL-1 B inhibition detected, and only at the level of CAN04 alone.

TABLE 30
Comparison of mAb “CAN04” to anti-IL1RAP #A2:

| Parameters vs IL1RAP Ab clone | | CAN04 | GO11 clone | |
|---|---|---|---|---|
| hK_{D} | [nM] | 0.2 | 0.03 | |
| IC90 [nM] | IL-1A | >1000 | 68 | |
| Nf-KB activation | IL1-B | 56 | 22 | |
| (HEK-Blue) | IL-33 | >1000 | 32 | |
| | IL-36G | >1000 | 7 | |
| IC90 [nM] | IL-1A | >1000 | 468 | |
| IL-8 release | IL1-B | >1000 | 2150 | |
| (NCI-H292) | IL-33 | 46 | 74 | |
| | IL-36G | >1000 | 43 | |
| IC90 [nM] MIP-1B | IL-1A | >1000 | >1000 | (650*) |
| (IL-1/IL-36)/IFN-γ | IL1-B | 125 | >1000 | (1221*) |

| | | | |
|---|---|---|---|
| (IL-33) release | IL-33 | >1000 | 123 |
| (WB) | IL-36G | 43 | 68 |

The ‘CAN04’ Ab was reported to bind to domain 2 of IL1 RAP i.e., within amino acids 135 to 234 of IL1 RAP (see Wang et al., 2010, Nature Immunology, 11:905-912), and it was reported that the epitope to which the CAN04 antibody bound may be located within amino acids 135 to 154, 155 to 174, 175 to 194, 195 to 214 or between amino acids 215 to 234 of IL1 RAP. The ‘CAN03’ antibody was reported to bind to domain 3 of IL1 RAP, consisting of the structural region defined by amino acids 235 to 369 of IL1 RAP.

However, when the HDX measurement of the epitopes of #A2, and CAN04 and CAN03 were compared their epitopes were completely different without even an overlapping amino acid. It is possible that this is due the non-linear or conformational nature of the diverse epitopes recognized by the CAN04 and CAN03. This could also explain the differences in the biological activity, particularly of CAN03 which had no inhibitory activity, when compared to the #A2 antibody.

### Example 5: Measurement of Soluble IL1RAP and Cyno PK

Soluble IL1RAP is known to be present in human and cynomolgus monkey blood. To determine how soluble IL1RAP may affect the PK of the anti-IL1RAP antibody in vivo, the levels of the free and total anti-IL1RAP were quantified in cyno serum samples.

### Determination of Free Anti-IL1RAP Antibody:

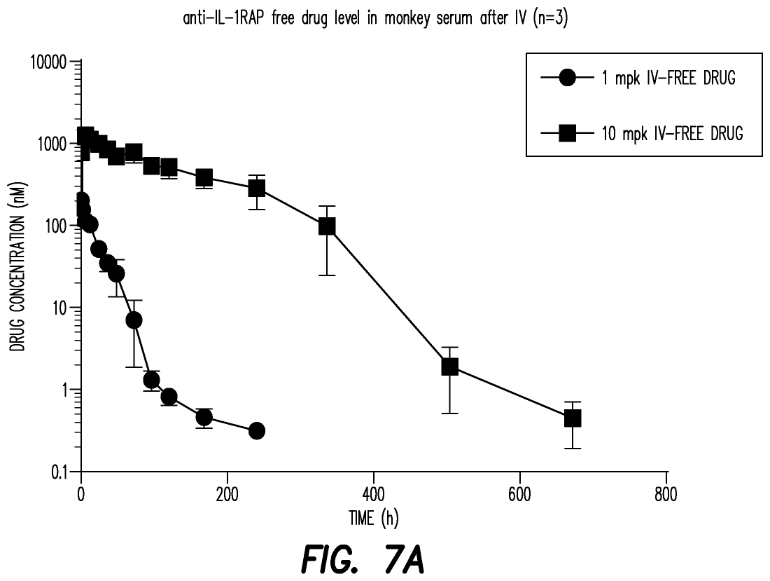

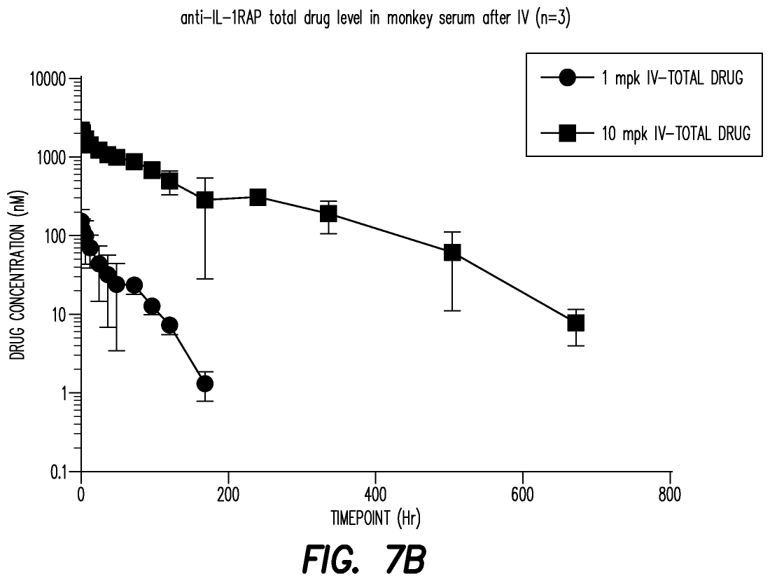

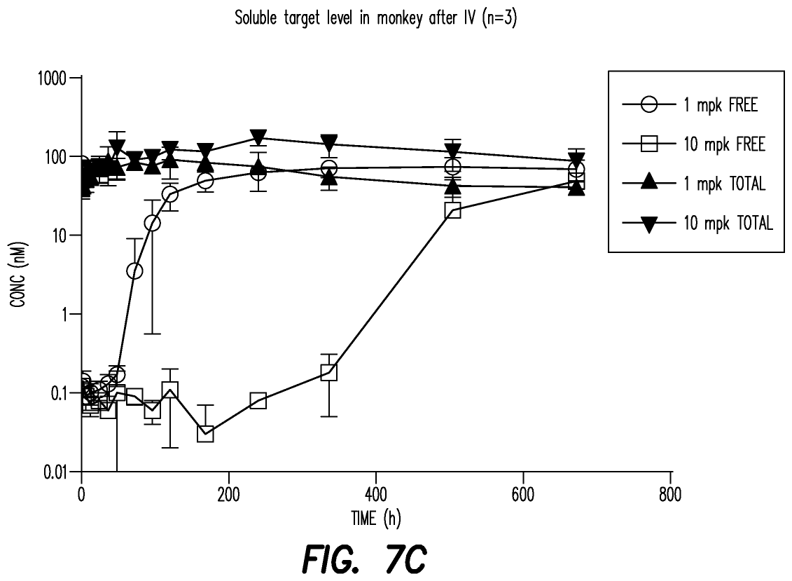

Serum concentrations of free anti-IL1RAP antibodies were measured using an Enzyme Linked Immunosorbent Assay (ELISA) methods. Briefly, microtiter plates (Nunc) were coated with 2 μg/mL of recombinant cynomolgus monkey IL1RAP overnight at 2-8° C. Unbound capture reagent was washed away (1×PBS with 0.05% Tween 20) and the wells were blocked with 5% BSA (Seracare) and incubated for 1 hour at room temperature. Plates were then washed and calibration standards, QCs, and sample serial dilutions were added and incubated for 1 hour at room temperature. Plates were washed and horseradish peroxidase (HRP)-conjugated goat anti-Human IgG, pre-adsorbed against monkey serum proteins (Southern Biotech) was added and incubated for 1 hour at room temperature. Following washing, bound HRP-conjugate was detected with a tetramethyl benzidine (TMB) substrate. The reaction was stopped with 1M H2SO4 and the absorbance was measured using a SpectraMax® microplate reader at 450 and 650 nm dual wavelength. The signal produced was proportional to the amount of anti-Ang1 antibodies present in the sample. Softmax Pro software (v5.4) was used for calibration standard curve fitting using a 4-parameter logistic model and back calculation of all unknown sample concentrations. (See FIG. 7A)

### Determination of Total Anti-IL1RAP Antibody

Serum concentrations of total anti-IL1RAP antibodies were measured using a homogenous Meso Scale Discovery (MSD) electrochemiluminescent assay. Briefly, gold small spot streptavidin plates (MSD part #L45SA) were blocked for 1 hour with 5% BSA (Seracare). Plates were then washed and a capture and detection master mix of biotin and MSD Sulfo-tag conjugated goat anti-Human IgG, pre-adsorbed against monkey serum proteins (Southern Biotech) containing calibration standards, QCs, and sample serial dilutions were added and incubated for 2 hours at room temperature. Following washing, 2×MSD Read Buffer was added and the electrochemiluminescence signal was then measured on the Sector Imager 6000 instrument. Unknown serum concentrations were calculated from standard curves fitted to a four-parameter logistics equation using MSD Discovery Workbench software. (See FIG. 7B)

### Determination of Free and Total Soluble IL1RAP

Free and total soluble IL1RAP serum concentrations were determined using the Gyrolab™ workstation with biotinylated and Alexa Fluor 647-labeled anti-IL1RAP antibodies, as capture and detection reagents, respectively. Total soluble IL1RAP concentrations were measured using monoclonal anti-IL1RAP capture and detection antibodies that recognized distinct epitopes from the therapeutic mAb. Free soluble IL1RAP concentrations were measured using a monoclonal anti-IL1RAP antibody capture antibody and the therapeutic anti-IL1RAP as detection. The reference standard and study samples were diluted in 2% BSA buffer. Samples and reagents were placed in 96 well polypropylene microplates and sealed with microplate foil sealers prior to loading into the instrument. Additions of capture reagent, samples, standards, detection reagent, and wash solutions to the microstructures on the CD were fully automated processes. During the assay, components of the method were loaded robotically onto streptavidin beads inside micro-column structures on a Bioaffy CD beginning with the biotinylated capture antibody, wash buffer, then calibration standards, and samples, wash buffer, and then the AlexaFluor 647-conjugated detection antibody. Following a wash to remove unbound Alexa Fluor, the fluorescence intensity was measured, which was proportional to the quantity of soluble IL1RAP present in the sample. Analyte concentrations were determined from data regression of fluorescence values derived from Alexa647 fluorescence emissions converted to concentration values. Quantitation was based on a four-parameter logistic (1/Y2) regression from recombinant cynomolgus monkey standard curves. (See FIG. 7C)

### Results:

Binding to surface IL1RAP is necessary for blocking activity. While IL1RAP also binds to soluble forms of ILRAP as well as surface IL1RAP, an efficacious dose could be predicted.

Sequences:

| SEQ | | | |
|---|---|---|---|
| ID | | | |
| NO: | NAME OF SEQUENCE | | SEQUENCE |
| 1 | PARENT | FULL LENGTH VL | DIQMTQSPASLSASVGETVTITCRTSENINSYLA |
| | CHIMERIC VL | | WYQQKQGKSPQLLVHYAKTLAEGVPSRFSGS |
| | (mu G011) | | GSGTQFSLKINSLKPEDFGSYYCQHHYGTSLT |
| | | | FGAGTKLELK |
| 2 | PARENT | FULL LENGTH VH | QVHLQQSGPELVRPGTSVKISCEASGYIFLTYW |
| | CHIMERIC | | MNWVKQRPGQGLEWIGQIFPASDSTYYNEMF |
| | VH (mu-G011) | | KDKARFTVDKSSSTAYMQFSSLTSEDTAVYFC |
| | | | ARSGPYSYYAGGYALDYWGQGTSVTVSS |
| 3 | PARENT | VL-CDR1 | RTSENINSYLA |
| | CHIMERIC VL | (KABAT/CCG/CHO | |
| | (mu G011) | THIA) | |
| 4 | | VL-CDR2 | YAKTLAE |
| | | (KABAT/CCG/CHO | |
| | | THIA) | |
| 5 | | VL | QHHYGTSLT |
| | | CDR3(KABAT/CCG/ | |
| | | CHOTHIA/IMGT) | |
| 6 | | VL-CDR1 (IMGT) | ENINSY |
| 7 | | VL-CDR2 (IMGT) | YAK |
| 8 | PARENT | VH-CDR1 | TYWMN |
| | CHIMERIC | (KABAT) | |
| 9 | VH (mu-G011) | VH-CDR2 | QIFPASDSTYYNEMFKD |
| | | (KABAT/CCG) | |
| 10 | | VH-CDR3 | SGPYSYYAGGYALDY |
| | | (KABAT/CCG/CHO | |
| | | THIA) | |
| 11 | | VH-CDR1 (CCG) | GYIFLTYWMN |
| 12 | | VH-CDR1 | GYIFLTY |
| | | (CHOTHIA) | |
| 13 | | VH-CDR2 | FPASDS |
| | | (CHOTHIA) | |
| 14 | | VH-CDR1 (IMGT) | GYIFLTYW |
| 15 | | VH-CDR2 (IMGT) | IFPASDST |
| 16 | | VH-CDR3 (IMGT) | ARSGPYSYYAGGYALDY |
| 17 | HUMANIZED | FULL LENGTH VL- | DIQMTQSPSSLSASVGDRVTITCRTSENINSYL |
| | GO11-(A#1) | A1 | AWYQQKPGKAPKLLIYYAKTLAEGVPSRFSGS |
| | | | GSGTDFTLTISSLQPEDFATYYCQHHYGTSLTF |
| | | | GQGTKLEIK |
| 18 | GO11.405.10 | | DIQMTQSPSSLSASVGDRVTITCRTSENINSYL |
| | \|VK\| (Y49H) | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 19 | GO11.405- | | DIQMTQSPSSLSASVGDRVTITCRTSENINSYL |
| | 12\|VK\| (Y49H) | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 20 | GO11.opt- | | DIQMTQSPSSLSASVGDRVTITCRASQSISSYL |
| | 27\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 21 | GO11.opt- | | DIQMTQSPSSLSASVGDRVTITCRASENINSYL |
| | 28\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 22 | GO11.opt- | | DIQMTQSPSSLSASVGDRVTITCRTSQNINSYL |
| | 29\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 23 | GO11.opt- | | DIQMTQSPSSLSASVGDRVTITCRASQSISSYL |
| | 30\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 24 | GO11.opt- | | DIQMTQSPSSLSASVGDRVTITCRTSESINSYL |
| | 31\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 25 | GO11.opt- | | DIQMTQSPSSLSASVGDRVTITCRASQSINSYL |
| | 32\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 26 | GO11.opt- | | DIQMTQSPSSLSASVGDRVTITCRTSESISSYLA |
| | 33\|VK\| | | WYQQKPGKAPKLLIHYASTLAEGVPSRFSGSG |
| | | | SGTDFTLTISSLQPEDFATYYCQHHYGTSLTFG |
| | | | QGTKLEIK |
| 27 | GO11.opt- | | DIQMTQSPSSLSASVGDRVTITCRASQSISSYL |
| | 34\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 28 | GO11.opt- | | DIQMTQSPSSLSASVGDRVTITCRASQSISSYL |
| | 35\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 29 | GO11.opt- | | DIQMTQSPSSLSASVGDRVTITCRASQSINSYL |
| | 36\|VK\| | | AWYQQKPGKAPKLLIHYASTLAEGVPSRFSGS |
| | | | GSGTDFTLTISSLQPEDFATYYCQHHYGTSLTF |
| | | | GQGTKLEIK |
| 30 | GO11.opt- | | DIQMTQSPSSLSASVGDRVTITCRTSESINSYL |
| | 37\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 31 | GO11.opt- | | DIQMTQSPSSLSASVGDRVTITCRASENISSYL |
| | 38\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 32 | GO11.opt- | | DIQMTQSPSSLSASVGDRVTITCRTSQNISSYL |
| | 39\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 33 | GO11.opt- | | DIQMTQSPSSLSASVGDRVTITCRTSQSINSYL |
| | 40\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 34 | GO11.opt- | | DIQMTQSPSSLSASVGDRVTITCRASQSISSYL |
| | 41\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 35 | GO11.opt- | | DIQMTQSPSSLSASVGDRVTITCRASQSISSYL |
| | 42\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 36 | opt-43\|VK\| | FULL LENGTH VL- | DIQMTQSPSSLSASVGDRVTITCRASQSISSYL |
| | #A2 | A2 | AWYQQKPGKAPKLLIHYAKSLAEGVPSRFSGS |
| | | | GSGTDFTLTISSLQPEDFATYYCQHHYGTSLTF |
| | | | GQGTKLEIK |
| 37 | GO11.opt- | | DIQMTQSPSSLSASVGDRVTITCRASQSISSYL |
| | 44\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 38 | GO11.opt- | | DIQMTQSPSSLSASVGDRVTITCRASQSISSYL |
| | 45\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 39 | GO11.opt- | | DIQMTQSPSSLSASVGDRVTITCRASQSISSYL |
| | 46\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 40 | GO11.opt- | FULL LENGTH VL- | DIQMTQSPSSLSASVGDRVTITCRASQSISSYL |
| | 47\|VK\| #A3 | A3 | AWYQQKPGKAPKLLIHYAKSLAEGVPSRFSGS |
| | | | GSGTDFTLTISSLQPEDFATYYCQHHYGTSLTF |
| | | | GQGTKLEIK |
| 41 | GO11.opt- | | DIQMTQSPSSLSASVGDRVTITCRASQSISSYL |
| | 48\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 42 | GO11.opt- | | DIQMTQSPSSLSASVGDRVTITCRASQSISSYL |
| | 49\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 43 | GO11.opt- | | DIQMTQSPSSLSASVGDRVTITCRASQSISSYL |
| | 50\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 44 | GO11.opt- | | DIQMTQSPSSLSASVGDRVTITCRASQSISSYL |
| | 51\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 45 | GO11.opt- | | DIQMTQSPSSLSASVGDRVTITCRASQSISSYL |
| | 52\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 46 | GO11.opt- | | DIQMTQSPSSLSASVGDRVTITCRASQSISSYL |
| | 53\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 47 | GO11.opt- | FULL LENGTH VL- | DIQMTQSPSSLSASVGDRVTITCRASQSISSYL |
| | 54\|VK\| #A4 | A4 | AWYQQKPGKAPKLLIHYAKSLAEGVPSRFSGS |
| | | | GSGTDFTLTISSLQPEDFATYYCQHHYGTSLTF |
| | | | GQGTKLEIK |
| 48 | GO11.opt- | | DIQMTQSPSSLSASVGDRVTITCRASQSISSYL |
| | 55\|VK\| | | AWYQQKPGKAPKLLIHYAKSLAEGVPSRFSGS |
| | | | GSGTDFTLTISSLQPEDFATYYCQHHYGTSLTF |
| | | | GQGTKLEIK |
| 49 | GO11.opt- | | DIQMTQSPSSLSASVGDRVTITCRASQSISSYL |
| | 56\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 50 | GO11.opt- | FULL LENGTH VL- | DIQMTQSPSSLSASVGDRVTITCRASQSISSYL |
| | 57\|VK\| #A5 | A5 | AWYQQKPGKAPKLLIHYASSLAEGVPSRFSGS |
| | | | GSGTDFTLTISSLQPEDFATYYCQHHYGTSLTF |
| | | | GQGTKLEIK |
| 51 | GO11.opt- | FULL LENGTH VL- | DIQMTQSPSSLSASVGDRVTITCRASQSISSYL |
| | 58\|VK\| #A6 | A6 | AWYQQKPGKAPKLLIHYASSLAEGVPSRFSGS |
| | | | GSGTDFTLTISSLQPEDFATYYCQHHYGTSLTF |
| | | | GQGTKLEIK |
| 52 | GO11.opt- | FULL LENGTH VL- | DIQMTQSPSSLSASVGDRVTITCRASQSISSYL |
| | 59\|VK\| #A7 | A7 | AWYQQKPGKAPKLLIHYASSLAEGVPSRFSGS |
| | | | GSGTDFTLTISSLQPEDFATYYCQHHYGTSLTF |
| | | | GQGTKLEIK |
| 53 | GO11.opt- | | DIQMTQSPSSLSASVGDRVTITCRASQSISSYL |
| | 60\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 54 | GO11.opt- | | DIQMTQSPSSLSASVGDRVTITCRASQSISSYL |
| | 61\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 55 | GO11.opt- | | DIQMTQSPSSLSASVGDRVTITCRASQSISSYL |
| | 62\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 56 | GO11.opt- | | DIQMTQSPSSLSASVGDRVTITCRASQSISSYL |
| | 63\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 57 | GO11.opt- | | DIQMTQSPSSLSASVGDRVTITCRASQSISSYL |
| | 64\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 58 | GO11.opt- | | DIQMTQSPSSLSASVGDRVTITCRASQSISSYL |
| | 65\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 59 | GO11.opt- | | DIQMTQSPSSLSASVGDRVTITCRASQSISSYL |
| | 66\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 60 | GO11.opt- | | DIQMTQSPSSLSASVGDRVTITCRASQSISSYL |
| | 67\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 61 | GO11.opt- | | DIQMTQSPSSLSASVGDRVTITCRASQSISSYL |
| | 68\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 62 | GO11.opt- | | DIQMTQSPSSLSASVGDRVTITCRASQSISSYL |
| | 69\|VK\| | | AWYQQKPGKAPKLLIHYASSLAEGVPSRFSGS |
| | | | GSGTDFTLTISSLQPEDFATYYCQHHYGTSLTF |
| | | | GQGTKLEIK |
| 63 | GO11.opt- | | DIQMTQSPSSLSASVGDRVTITCRTSENISSYL |
| | 70\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 64 | GO11.opt- | | DIQMTQSPSSLSASVGDRVTITCRTSENISSYL |
| | 71\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 65 | GO11.opt- | | DIQMTQSPSSLSASVGDRVTITCRTSENISSYL |
| | 72\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 66 | GO11.opt- | | DIQMTQSPSSLSASVGDRVTITCRTSENISSYL |
| | 73\|VK\| | | AWYQQKPGKAPKLLIHYAKTLAEGVPSRFSGS |
| | | | GSGTDFTLTISSLQPEDFATYYCQHHYGTSLTF |
| | | | GQGTKLEIK |
| 67 | HUMANIZED/ | FULL LENGTH VH- | QVQLVQSGAEVKKPGSSVKVSCKASGYIFLTY |
| | GRAFT \|VH\| | A1 | WMNWVRQAPGQGLEWMGQIFPASDSTYYNE |
| | 005-G011 #A1 | | MFKDRVTITADKSTSTAYMELSSLRSEDTAVYY |
| | | | CARSGPYSYYAGGYALDYWGQGTLVTVSS |
| 68 | GO11.406-v18 | | QVQLVQSGAEVKKPGSSVKVSCKASGYIFLTY |
| | \|VH\| (A72V) | | WMNWVRQAPGQGLEWMGQIFPASDSTYYNE |
| | | | MFKDRVRITVDKSTSTAYMELSSLRSEDTAVYY |
| | | | CARSGPYSYYAGGYALDYWGQGTLVTVSS |
| 69 | GO11.406-v20 | | QVQLVQSGAEVKKPGSSVKVSCKASGYIFLTY |
| | \|VH\| (A72V) | | WMNWVRQAPGQGLEWMGQIFPASDSTYYNE |
| | | | MFKDRVTFTVDKSTSTAYMELSSLRSEDTAVY |
| | | | YCARSGPYSYYAGGYALDYWGQGTLVTVSS |
| 70 | GO11.opt- | | QVQLVQSGAEVKKPGSSVKVSCKASGYIFLTY |
| | 27\|VH\| | | WINWVRQAPGQGLEWMGQIFPASGSAYYAQK |
| | | | FQGRVTITADKSTSTAYMELSSLRSEDTAVYYC |
| | | | ARSGPYSYYAGGYALDYWGQGTLVTVSS |
| 71 | GO11.opt- | | QVQLVQSGAEVKKPGSSVKVSCKASGYIFLTY |
| | 28\|VH\| | | WMNWVRQAPGQGLEWMGQIFPASGSAYYAQ |
| | | | KFQGRVTITVDKSTSTAYMELSSLRSEDTAVYY |
| | | | CARSGPYSYYAGGYALDYWGQGTLVTVSS |
| 72 | GO11.opt- | | QVQLVQSGAEVKKPGSSVKVSCKASGYIFLTY |
| | 29\|VH\| | | WMNWVRQAPGQGLEWMGQIFPASGSAYYNQ |
| | | | KFKGRVTITADKSTSTAYMELSSLRSEDTAVYY |
| | | | CARSGPYSYYAGGYALDYWGQGTLVTVSS |
| 73 | GO11.opt- | | QVQLVQSGAEVKKPGSSVKVSCKASGYIFLTY |
| | 30\|VH\| | | WMNWVRQAPGQGLEWMGQIFPASDSTYYAQ |
| | | | MFQDRVTITADKSTSTAYMELSSLRSEDTAVYY |
| | | | CARSGPYSYYAGGYALDYWGQGTLVTVSS |
| 74 | GO11.opt- | | QVQLVQSGAEVKKPGSSVKVSCKASGYIFLTY |
| | 31\|VH\| | | WINWVRQAPGQGLEWMGQIFPASGSTYYNEK |
| | | | FKGRVTITADKSTSTAYMELSSLRSEDTAVYYC |
| | | | ARSGPYSYYAGGYALDYWGQGTLVTVSS |
| 75 | GO11.opt- | | QVQLVQSGAEVKKPGSSVKVSCKASGYIFLTY |
| | 32\|VH\| | | WINWVRQAPGQGLEWMGQIFPASGSTYYAQK |
| | | | FQDRVTITVDKSTSTAYMELSSLRSEDTAVYYC |
| | | | ARSGPYSYYAGGYALDYWGQGTLVTVSS |
| 76 | GO11.opt-33\|VH\| | | QVQLVQSGAEVKKPGSSVKVSCKASGYIFLTYWINWVR |
| | | | QAPGQGLEWMGQIFPASDSTYYNQKFKGRVTITADKST |
| | | | STAYMELSSLRSEDTAVYYCARSGPYSYYAGGYALDYW |
| | | | GQGTLVTVSS |
| 77 | GO11.opt-34\|VH\| | | QVQLVQSGAEVKKPGSSVKVSCKASGYIFLTYWMNWVR |
| | | | QAPGQGLEWMGQIFPASDSAYYNEKFKGRVTITVDKST |
| | | | STAYMELSSLRSEDTAVYYCARSGPYSYYAGGYALDYW |
| | | | GQGTLVTVSS |
| 78 | GO11.opt-35\|VH\| | | QVQLVQSGAEVKKPGSSVKVSCKASGYIFLTYWINWVR |
| | | | QAPGQGLEWMGQIFPASDSAYYAQKFQDRVTITADKST |
| | | | STAYMELSSLRSEDTAVYYCARSGPYSYYAGGYALDYW |
| | | | GQGTLVTVSS |
| 79 | GO11.opt-36\|VH\| | | QVQLVQSGAEVKKPGSSVKVSCKASGYIFLTYWMNWVR |
| | | | QAPGQGLEWMGQIFPASGSAYYAQKFQGRVTITVDKST |
| | | | STAYMELSSLRSEDTAVYYCARSGPYSYYAGGYALDYW |
| | | | GQGTLVTVSS |
| 80 | GO11.opt-37\|VH\| | | QVQLVQSGAEVKKPGSSVKVSCKASGYIFLTYWMNWVR |
| | | | QAPGQGLEWMGQIFPASGSAYYAQKFQGRVTITADKST |
| | | | STAYMELSSLRSEDTAVYYCARSGPYSYYAGGYALDYW |
| | | | GQGTLVTVSS |
| 81 | GO11.opt-38\|VH\| | | QVQLVQSGAEVKKPGSSVKVSCKASGYIFLTYWMNWVR |
| | | | QAPGQGLEWMGQIFPASGSAYYNEKFKDRVTITVDKST |
| | | | STAYMELSSLRSEDTAVYYCARSGPYSYYAGGYALDYW |
| | | | GQGTLVTVSS |
| 82 | GO11.opt-39\|VH\| | | QVQLVQSGAEVKKPGSSVKVSCKASGYIFLTYWISWVR |
| | | | QAPGQGLEWMGQIFPASDSAYYNQMFKDRVTITVDKST |
| | | | STAYMELSSLRSEDTAVYYCARSGPYSYYAGGYALDYW |
| | | | GQGTLVTVSS |
| 83 | GO11.opt-40\|VH\| | | QVQLVQSGAEVKKPGSSVKVSCKASGYIFLTYWINWVR |
| | | | QAPGQGLEWMGQIFPASGSAYYAQKFQGRVTITADKST |
| | | | STAYMELSSLRSEDTAVYYCARSGPYSYYAGGYALDYW |
| | | | GQGTLVTVSS |
| 84 | GO11.opt-41\|VH\| | | QVQLVQSGAEVKKPGSSVKVSCKASGYIFLTYWINWVR |
| | | | QAPGQGLEWMGQIFPASDSAYYNQMFKDRVTITADKST |
| | | | STAYMELSSLRSEDTAVYYCARSGPYSYYAGGYALDYW |
| | | | GQGTLVTVSS |
| 85 | GO11.opt-42\|VH\| | | QVQLVQSGAEVKKPGSSVKVSCKASGYIFLTYWMNWVR |
| | | | QAPGQGLEWMGQIFPASGSAYYAQKFQGRVTITVDKST |
| | | | STAYMELSSLRSEDTAVYYCARSGPYSYYAGGYALDYW |
| | | | GQGTLVTVSS |
| 86 | GO11.opt-43\|VH\| | FULL LENGTH VH-A2 | QVQLVQSGAEVKKPGSSVKVSCKASGYIFLTYWMNWVR |
| | #A2 | | QAPGQGLEWMGQIFPASGSAYYNQKFKGRVTITADKST |
| | | | STAYMELSSLRSEDTAVYYCARSGPYSYYAGGYALDYW |
| | | | GQGTLVTVSS |
| 87 | GO11.opt-44\|VH\| | | QVQLVQSGAEVKKPGSSVKVSCKASGYIFLTYWMNWVR |
| | | | QAPGQGLEWMGQIFPASDSTYYAQMFQDRVTITADKST |
| | | | STAYMELSSLRSEDTAVYYCARSGPYSYYAGGYALDYW |
| | | | GQGTLVTVSS |
| 88 | GO11.opt-45\|VH\| | | QVQLVQSGAEVKKPGSSVKVSCKASGYIFLTYWINWVR |
| | | | QAPGQGLEWMGQIFPASGSTYYNEKFKGRVTITADKST |
| | | | STAYMELSSLRSEDTAVYYCARSGPYSYYAGGYALDYW |
| | | | GQGTLVTVSS |
| 89 | GO11.opt-46\|VH\| | | QVQLVQSGAEVKKPGSSVKVSCKASGYIFLTYWINWVR |
| | | | QAPGQGLEWMGQIFPASGSTYYAQKFQDRVTITVDKST |
| | | | STAYMELSSLRSEDTAVYYCARSGPYSYYAGGYALDYW |
| | | | GQGTLVTVSS |
| 90 | GO11.opt-47\|VH\| | FULL LENGTH VH-A3 | QVQLVQSGAEVKKPGSSVKVSCKASGYIFLTYWINWVR |
| | #A3 | | QAPGQGLEWMGQIFPASDSTYYNQKFKGRVTITADKST |
| | | | STAYMELSSLRSEDTAVYYCARSGPYSYYAGGYALDYW |
| | | | GQGTLVTVSS |
| 91 | GO11.opt-48\|VH\| | | QVQLVQSGAEVKKPGSSVKVSCKASGYIFLTYWMNWVR |
| | | | QAPGQGLEWMGQIFPASDSAYYNEKFKGRVTITVDKST |
| | | | STAYMELSSLRSEDTAVYYCARSGPYSYYAGGYALDYW |
| | | | GQGTLVTVSS |
| 92 | GO11.opt-49\|VH\| | | QVQLVQSGAEVKKPGSSVKVSCKASGYIFLTYWINWVR |
| | | | QAPGQGLEWMGQIFPASDSAYYAQKFQDRVTITADKST |
| | | | STAYMELSSLRSEDTAVYYCARSGPYSYYAGGYALDYW |
| | | | GQGTLVTVSS |
| 93 | GO11.opt-50\|VH\| | | QVQLVQSGAEVKKPGSSVKVSCKASGYIFLTYWMNWVR |
| | | | QAPGQGLEWMGQIFPASGSAYYAQKFQGRVTITVDKST |
| | | | STAYMELSSLRSEDTAVYYCARSGPYSYYAGGYALDYW |
| | | | GQGTLVTVSS |
| 94 | GO11.opt-51\|VH\| | | QVQLVQSGAEVKKPGSSVKVSCKASGYIFLTYWMNWVR |
| | | | QAPGQGLEWMGQIFPASGSAYYAQKFQGRVTITADKST |
| | | | STAYMELSSLRSEDTAVYYCARSGPYSYYAGGYALDYW |
| | | | GQGTLVTVSS |
| 95 | GO11.opt- | | QVQLVQSGAEVKKPGSSVKVSCKASGYIFLTY |
| | 52\|VH\| | | WMNWVRQAPGQGLEWMGQIFPASGSAYYNE |
| | | | KFKDRVTITVDKSTSTAYMELSSLRSEDTAVYY |
| | | | CARSGPYSYYAGGYALDYWGQGTLVTVSS |
| 96 | GO11.opt- | | QVQLVQSGAEVKKPGSSVKVSCKASGYIFLTY |
| | 53\|VH\| | | WISWVRQAPGQGLEWMGQIFPASDSAYYNQM |
| | | | FKDRVTITVDKSTSTAYMELSSLRSEDTAVYYC |
| | | | ARSGPYSYYAGGYALDYWGQGTLVTVSS |
| 97 | GO11.opt- | FULL LENGTH VH- | QVQLVQSGAEVKKPGSSVKVSCKASGYIFLTY |
| | 54\|VH\| #A4 | A4 | WINWVRQAPGQGLEWMGQIFPASGSAYYAQK |
| | | | FQGRVTITADKSTSTAYMELSSLRSEDTAVYYC |
| | | | ARSGPYSYYAGGYALDYWGQGTLVTVSS |
| 98 | GO11.opt- | | QVQLVQSGAEVKKPGSSVKVSCKASGYIFLTY |
| | 55\|VH\| | | WINWVRQAPGQGLEWMGQIFPASDSAYYNQ |
| | | | MFKDRVTITADKSTSTAYMELSSLRSEDTAVYY |
| | | | CARSGPYSYYAGGYALDYWGQGTLVTVSS |
| 99 | GO11.opt- | | QVQLVQSGAEVKKPGSSVKVSCKASGYIFLTY |
| | 56\|VH\| | | WINWVRQAPGQGLEWMGQIFPASGSAYYAQK |
| | | | FQGRVTITADKSTSTAYMELSSLRSEDTAVYYC |
| | | | ARSGPYSYYAGGYALDYWGQGTLVTVSS |
| 100 | GO11.opt- | FULL LENGTH VH- | QVQLVQSGAEVKKPGSSVKVSCKASGYIFLTY |
| | 57\|VH\| #A5 | A5 | WMNWVRQAPGQGLEWMGQIFPASGSAYYAQ |
| | | | KFQGRVTITVDKSTSTAYMELSSLRSEDTAVYY |
| | | | CARSGPYSYYAGGYALDYWGQGTLVTVSS |
| 101 | GO11.opt- | FULL LENGTH VH- | QVQLVQSGAEVKKPGSSVKVSCKASGYIFLTY |
| | 58\|VH\| #A6 | A6 | WMNWVRQAPGQGLEWMGQIFPASGSAYYNQ |
| | | | KFKGRVTITADKSTSTAYMELSSLRSEDTAVYY |
| | | | CARSGPYSYYAGGYALDYWGQGTLVTVSS |
| 102 | GO11.opt- | FULL LENGTH VH- | QVQLVQSGAEVKKPGSSVKVSCKASGYIFLTY |
| | 59\|VH\|#A7 | A7 | WINWVRQAPGQGLEWMGQIFPASGSTYYNEK |
| | | | FKGRVTITADKSTSTAYMELSSLRSEDTAVYYC |
| | | | ARSGPYSYYAGGYALDYWGQGTLVTVSS |
| 103 | GO11.opt- | | QVQLVQSGAEVKKPGSSVKVSCKASGYIFLTY |
| | 60\|VH\| | | WINWVRQAPGQGLEWMGQIFPASGSTYYAQK |
| | | | FQDRVTITVDKSTSTAYMELSSLRSEDTAVYYC |
| | | | ARSGPYSYYAGGYALDYWGQGTLVTVSS |
| 104 | GO11.opt- | | QVQLVQSGAEVKKPGSSVKVSCKASGYIFLTY |
| | 61\|VH\| | | WINWVRQAPGQGLEWMGQIFPASDSTYYNQK |
| | | | FKGRVTITADKSTSTAYMELSSLRSEDTAVYYC |
| | | | ARSGPYSYYAGGYALDYWGQGTLVTVSS |
| 105 | GO11.opt- | | QVQLVQSGAEVKKPGSSVKVSCKASGYIFLTY |
| | 62\|VH\| | | WMNWVRQAPGQGLEWMGQIFPASDSAYYNE |
| | | | KFKGRVTITVDKSTSTAYMELSSLRSEDTAVYY |
| | | | CARSGPYSYYAGGYALDYWGQGTLVTVSS |
| 106 | GO11.opt- | | QVQLVQSGAEVKKPGSSVKVSCKASGYIFLTY |
| | 63\|VH\| | | WINWVRQAPGQGLEWMGQIFPASDSAYYAQK |
| | | | FQDRVTITADKSTSTAYMELSSLRSEDTAVYYC |
| | | | ARSGPYSYYAGGYALDYWGQGTLVTVSS |
| 107 | GO11.opt- | | QVQLVQSGAEVKKPGSSVKVSCKASGYIFLTY |
| | 64\|VH\| | | WMNWVRQAPGQGLEWMGQIFPASGSAYYAQ |
| | | | KFQGRVTITVDKSTSTAYMELSSLRSEDTAVYY |
| | | | CARSGPYSYYAGGYALDYWGQGTLVTVSS |
| 108 | GO11.opt- | | QVQLVQSGAEVKKPGSSVKVSCKASGYIFLTY |
| | 65\|VH\| | | WMNWVRQAPGQGLEWMGQIFPASGSAYYAQ |
| | | | KFQGRVTITADKSTSTAYMELSSLRSEDTAVYY |
| | | | CARSGPYSYYAGGYALDYWGQGTLVTVSS |
| 109 | GO11.opt- | | QVQLVQSGAEVKKPGSSVKVSCKASGYIFLTY |
| | 66\|VH\| | | WMNWVRQAPGQGLEWMGQIFPASGSAYYNE |
| | | | KFKDRVTITVDKSTSTAYMELSSLRSEDTAVYY |
| | | | CARSGPYSYYAGGYALDYWGQTLVTVSS |
| 110 | GO11.opt- | | QVQLVQSGAEVKKPGSSVKVSCKASGYIFLTY |
| | 67\|VH\| | | WISWVRQAPGQGLEWMGQIFPASDSAYYNQM |
| | | | FKDRVTITVDKSTSTAYMELSSLRSEDTAVYYC |
| | | | ARSGPYSYYAGGYALDYWGQGTLVTVSS |
| 111 | GO11.opt- | | QVQLVQSGAEVKKPGSSVKVSCKASGYIFLTY |
| | 68\|VH\| | | WINWVRQAPGQGLEWMGQIFPASGSAYYAQK |
| | | | FQGRVTITADKSTSTAYMELSSLRSEDTAVYYC |
| | | | ARSGPYSYYAGGYALDYWGQGTLVTVSS |
| 112 | GO11.opt- | | QVQLVQSGAEVKKPGSSVKVSCKASGYIFLTY |
| | 69\|VH\| | | WINWVRQAPGQGLEWMGQIFPASDSAYYNQ |
| | | | MFKDRVTITADKSTSTAYMELSSLRSEDTAVYY |
| | | | CARSGPYSYYAGGYALDYWGQGTLVTVSS |
| 113 | GO11.opt- | | QVQLVQSGAEVKKPGSSVKVSCKASGYIFLTY |
| | 70\|VH\| | | WINWVRQAPGQGLEWMGQIFPASGSTYYNEK |
| | | | FKDRVRITVDKSTSTAYMELSSLRSEDTAVYYC |
| | | | ARSGPYSYYAGGYALDYWGQGTLVTVSS |
| 114 | GO11.opt- | | QVQLVQSGAEVKKPGSSVKVSCKASGYIFLTY |
| | 71\|VH\| | | WINWVRQAPGQGLEWMGQIFPASGSTYYNEM |
| | | | FKDRVRITVDKSTSTAYMELSSLRSEDTAVYYC |
| | | | ARSGPYSYYAGGYALDYWGQGTLVTVSS |
| 115 | GO11.opt- | | QVQLVQSGAEVKKPGSSVKVSCKASGYIFLTY |
| | 72\|VH\| | | WMNWVRQAPGQGLEWMGQIFPASGSTYYNE |
| | | | KFKDRVRITVDKSTSTAYMELSSLRSEDTAVYY |
| | | | CARSGPYSYYAGGYALDYWGQGTLVTVSS |
| 116 | GO11.opt- | | QVQLVQSGAEVKKPGSSVKVSCKASGYIFLTY |
| | 73\|VH\| | | WMNWVRQAPGQGLEWMGQIFPASGSTYYNE |
| | | | MFKDRVRITVDKSTSTAYMELSSLRSEDTAVYY |
| | | | CARSGPYSYYAGGYALDYWGQGTLVTVSS |
| 117 | OPT 27, 30, | VL-CDR1 | RASQSISSYLA |
| | 34, 35, 41-69) | (KABAT/CCG/CHO | |
| | (OPT 43 | THIA) | |
| | (#A2), 47 | | |
| | (#A3), 54 | | |
| | (#A4), 57 | | |
| | (#A5), 58 | | |
| | (#A6), 59 | | |
| | (#A7) | | |
| 118 | OPT 28 | VL-CDR1 | RASENINSYLA |
| | | (KABAT/CCG/CHO | |
| | | THIA) | |
| 119 | OPT 29 | VL-CDR1 | RTSQNINSYLA |
| | | (KABAT/CCG/CHO | |
| | | THIA) | |
| 120 | OPT 31, 37 | VL-CDR1 | RTSESINSYLA |
| | | (KABAT/CCG/CHO | |
| | | THIA) | |
| 121 | OPT 32 | VL-CDR1 | RASQSINSYLA |
| | | (KABAT/CCG/CHO | |
| | | THIA) | |
| 122 | OPT 33, 36 | VL-CDR1 | RTSESISSYLA, |
| | | (KABAT/CCG/CHO | |
| | | THIA) | |
| 123 | OPT 38 | VL-CDR1 | RASENISSYLA |
| | | (KABAT/CCG/CHO | |
| | | THIA) | |
| 124 | OPT 39 | VL-CDR1 | RTSQNISSYLA |
| | | (KABAT/CCG/CHO | |
| | | THIA) | |
| 125 | OPT 40 | VL-CDR1 | RTSQSINSYLA |
| | | (KABAT/CCG/CHO | |
| | | THIA) | |
| 126 | OPT 70-73 | VL-CDR1 | RTSENISSYLA |
| | | (KABAT/CCG/CHO | |
| | | THIA) | |
| 127 | OPT 27, 30, | VL-CDR1 (IMGT) | QSISSY |
| | 34, 35, 41-69, | | |
| | (OPT 43 | | |
| | (#A2), 47 | | |
| | (#A3), 54 | | |
| | (#A4), 57 | | |
| | (#A5), 58 | | |
| | (#A6), 59 | | |
| | (#A7) | | |
| 128 | OPT 29 | VL-CDR1 (IMGT) | QNINSY |
| 129 | OPT 31, 37 | VL-CDR1 (IMGT) | ESINSY |
| 130 | OPT 32, 36, 40 | VL-CDR1 (IMGT) | QSINSY |
| 131 | OPT 33, | VL-CDR1 (IMGT) | ESISSY |
| 132 | OPT 38, 70-73 | VL-CDR1 (IMGT) | ENISSY |
| 133 | OPT 39 | VL-CDR1 (IMGT) | QNISSY |
| 134 | CONSENSUS | VL-CDR1 | RX1SX2X3IX4SYLA (X1 = A, T; X2 = Q, E; X3 = S, |
| | | CONSENSUS | N; X4 = S, N) |
| | | (KABAT/CCG/CHO | |
| | | THIA) | |
| 135 | CONSENSUS | VL-CDR1 | X1X2IX3SY (X1 = Q, E; X2 = S, N; X3 = S, N) |
| | | CONSENSUS | |
| | | (IMGT) | |
| 136 | OPT-27 | VL-CDR2 | YAKSLAE |
| | | (KABAT/CCG/CHO | |
| | | THIA) | |
| 137 | OPT-30 | VL-CDR2 | YASSLAE |
| | | (KABAT/CCG/CHO | |
| | | THIA) | |
| 138 | OPT-33 | VL-CDR2 | YASTLAE |
| | | (KABAT/CCG/CHO | |
| | | THIA) | |
| 139 | OPT-30 | VL-CDR2 (IMGT) | YAS |
| 140 | CONSENSUS | VL-CDR2 | YAX1X2LAE (X1 = S, K; X2 = S,T) |
| | | CONSENSUS | |
| | | (KABAT/CCG/CHO | |
| | | THIA) | |
| 141 | CONSENSUS | VL-CDR2 | YAX1 (X1 = S, K) |
| | | CONSENSUS | |
| | | (IMGT) | |
| 142 | OPT 27, 31, | VH-CDR1 (KABAT) | TYWIN |
| | 32, 33, 35, 40, | | |
| | 41, 45, | | |
| | 46, 47, 49, 59, | | |
| | 60, 61, 63 | | |
| 143 | OPT 39, 53, 67 | VH-CDR1 (KABAT) | TYWIS |
| 144 | OPT 27, 31, | VH-CDR1 (CCG) | GYIFLTYWIN |
| | 32, 33, 35, 40, | | |
| | 41, 45, | | |
| | 46, 47, 49, 59, | | |
| | 60, 61, 63 | | |
| 145 | OPT 39 | VH-CDR1 (CCG) | GYIFLTYWIS |
| 146 | CONSENSUS | VH-CDR1 | TYWX1X2 (X1 = M, I; X2 = N, S) |
| | | CONSENSUS | |
| | | (KABAT) | |
| 147 | CONSENSUS | VH-CDR1 | GYIFLTYWX1NX2 (X1 = M, I; X2 = N, S) |
| | | CONSENSUS | |
| | | (CCG) | |
| 148 | OPT 27, 28, | VH-CDR2 | QIFPASGSAYYAQKFQG |
| | 36, 37, 40, 50, | (KABAT, CCG) | |
| | 51, 54 (#A4), | | |
| | 56, 57 (#A5), | | |
| | 64, 65, 68 | | |
| 149 | OPT 29, 43 | VH-CDR2 | QIFPASGSAYYNQKFKG |
| | (#A2), OPT-58 | (KABAT, CCG) | |
| | (#A6) | | |
| 150 | OPT 30 | VH-CDR2 | QIFPASDSTYYAQMFQD |
| | | (KABAT, CCG) | |
| 151 | OPT 31, 45, 59 | VH-CDR2 | QIFPASGSTYYNEKFKG |
| | (#A7) | (KABAT, CCG) | |
| 152 | OPT 32, 46 | VH-CDR2 | QIFPASGSTYYAQKFQD |
| | | (KABAT, CCG) | |
| 153 | OPT 33, 47 | VH-CDR2 | QIFPASDSTYYNQKFKG |
| | (#A3), 61 | (KABAT, CCG) | |
| 154 | OPT 34, 48, | VH-CDR2 | QIFPASDSAYYNEKFKG |
| | 62 | (KABAT, CCG) | |
| 155 | OPT 35, 49, | VH-CDR2 | QIFPASDSAYYAQKFQDNEKFKG |
| | 63 | (KABAT, CCG) | |
| 156 | OPT 38, 52,66 | VH-CDR2 | QIFPASGSAYYNEKFKD |
| | | (KABAT, CCG) | |
| 157 | OPT 39, 41, | VH-CDR2 | QIFPASDSAYYNQMFKD |
| | 53, 55, 67, 69 | (KABAT, CCG) | |
| 158 | OPT 44 | VH-CDR2 | QIFPASDSTYYAQMFQD |
| | | (KABAT, CCG) | |
| 159 | OPT 70, 72 | VH-CDR2 | QIFPASGSTYYNEKFKD |
| | | (KABAT, CCG) | |
| 160 | OPT 71, 73 | VH-CDR2 | QIFPASGSTYYNEMFKD |
| | | (KABAT, CCG) | |
| 161 | OPT 27, 28, | VH-CDR2 | FPASGS |
| | 29, 31, 32, 36, | (CHOTHIA) | |
| | 37, 38, 40, 42, | | |
| | 43 (#A2), 45, | | |
| | 46, 50, 51, 52, | | |
| | 54 (#A4), 56, | | |
| | 57 (#54), 58 | | |
| | (#A60, 59 | | |
| | (#A7), 60, 64, | | |
| | 65, 66, 68, 70, | | |
| | 71, 72, 73 | | |
| 162 | OPT 27, | VH-CDR2 (IMGT) | IFPASGSA |
| | 28, 29, 36, 37, | | |
| | 38, 40, 42, 43 | | |
| | (#A1), 50, 51, | | |
| | 52, 54 (#A4), | | |
| | 56, 57 (#A5), | | |
| | 58 (#A6), 64, | | |
| | 65, 66, 68 | | |
| 163 | OPT 31, 32, | VH-CDR2 (IMGT) | IFPASGDST |
| | 45, 46, 59 | | |
| | (#A7), 60, 70, | | |
| | 71, 72, 73 | | |
| 164 | OPT 34, 35, | VH-CDR2 (IMGT) | IFPASDSA |
| | 39, 41, 48, 49, | | |
| | 53, 55, 62, 63, | | |
| | 67, 69 | | |
| 165 | CONSENSUS | VH-CDR2 | QIFPASX1SX2YYX3X4X5FX6X7 (X1 = D, G; X2 = |
| | | CONSENSUS | A, T; X3 = N, A; X4 = Q, E; X5 = M, K; X6 = Q, |
| | | (KABAT, CCG) | K; X7 = D, G) |
| 166 | CONSENSUS | VH-CDR2 | FPASX1S (X1 = D, G) |
| | | CONSENSUS | |
| | | (CHOTHIA) | |
| 167 | CONSENSUS | VH-CDR2 | IFPASX1SX2 (X1 = D, G; X2 = A, T) |
| | | CONSENSUS | |
| | | (IMGT) | |
| 168 | HEAVY | | ASTKGPSVFPLAPSSKSTSGGTAALGCLVKDY |
| | CHAIN | | FPEPVTVSWNSGALTSGVHTFPAVLQSSGLYS |
| | CONSTANT | | LSSVVTVPSSSLGTQTYICNVNHKPSNTKVDKR |
| | REGION | | VEPKSCDKTHTCPPCPAPEAAGGPSVFLFPPK |
| | | | PKDTLMISRTPEVTCVVVDVSHEDPEVKFNWY |
| | | | VDGVEVHNAKTKPREEQYNSTYRVVSVLTVLH |
| | | | QDWLNGKEYKCKVSNKALPAPIEKTISKAKGQ |
| | | | PREPQVYTLPPSREEMTKNQVSLTCLVKGFYP |
| | | | SDIAVEWESNGQPENNYKTTPPVLDSDGSFFL |
| | | | YSKLTVDKSRWQQGNVFSCSVMHEALHNHYT |
| | | | QKSLSLSPG |
| 169 | Light Chain | | RTVAAPSVFIFPPSDEQLKSGTASVVCLLNNFY |
| | Constant | | PREAKVQWKVDNALQSGNSQESVTEQDSKDS |
| | region | | TYSLSSTLTLSKADYEKHKVYACEVTHQGLSSP |
| | | | VTKSFNRGEC |
| 170 | anti-IL 1RAP | FULL LENGTH | DIQMTQSPASLSASVGETVTITCRTSENINSYLA |
| | PARENT | LIGHT CHAIN | WYQQKQGKSPQLLVHYAKTLAEGVPSRFSGS |
| | CLONE VL | PARENT | GSGTQFSLKINSLKPEDFGSYYCQHHYGTSLT |
| | | | FGAGTKLELKRTVAAPSVFIFPPSDEQLKSGTA |
| | | | SVVCLLNNFYPREAKVQWKVDNALQSGNSQE |
| | | | SVTEQDSKDSTYSLSSTLTLSKADYEKHKVYAC |
| | | | EVTHQGLSSPVTKSFNRGEC |
| 171 | IL1RAP-opt- | FULL LENGTH | DIQMTQSPSSLSASVGDRVTITCRASQSISSYL |
| | 43 VL | LIGHT CHAIN | AWYQQKPGKAPKLLIHYAKSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 172 | IL1RAP-opt- | FULL LENGTH | DIQMTQSPSSLSASVGDRVTITCRASQSISSYL |
| | 47 VL | LIGHT CHAIN | AWYQQKPGKAPKLLIHYAKSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 173 | IL1RAP-opt- | FULL LENGTH | DIQMTQSPSSLSASVGDRVTITCRASQSISSYL |
| | 54 VL | LIGHT CHAIN | AWYQQKPGKAPKLLIHYAKSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 174 | IL1RAP-opt- | FULL LENGTH | DIQMTQSPSSLSASVGDRVTITCRASQSISSYL |
| | 57 VL | LIGHT CHAIN | AWYQQKPGKAPKLLIHYASSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 175 | IL1RAP-opt- | FULL LENGTH | DIQMTQSPSSLSASVGDRVTITCRASQSISSYL |
| | 58 VL | LIGHT CHAIN | AWYQQKPGKAPKLLIHYASSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 176 | IL1RAP-opt- | FULL LENGTH | DIQMTQSPSSLSASVGDRVTITCRASQSISSYL |
| | 59 VL | LIGHT CHAIN | AWYQQKPGKAPKLLIHYASSLAEGVPSRFSGS |
| | | | GSGTDFTLTISSLQPEDFATYYCQHHYGTSLTF |
| | | | GQGTKLEIKRTVAAPSVFIFPPSDEQLKSGTAS |
| | | | VVCLLNNFYPREAKVQWKVDNALQSGNSQES |
| | | | VTEQDSKDSTYSLSSTLTLSKADYEKHKVYACE |
| | | | VTHQGLSSPVTKSFNRGEC |
| 177 | IL1RAP_HCVR- | FULL LENGTH | QVHLQQSGPELVRPGTSVKISCEASGYIFLTYW |
| | Fc | HEAVY CHAIN | MNWVKQRPGQGLEWIGQIFPASDSTYYNEMF |
| | | | KDKARFTVDKSSSTAYMQFSSLTSEDTAVYFC |
| | | | ARSGPYSYYAGGYALDYWGQGTSVTVSSAST |
| | | | KGPSVFPLAPSSKSTSGGTAALGCLVKDYFPE |
| | | | PVTVSWNSGALTSGVHTFPAVLQSSGLYSLSS |
| | | | VVTVPSSSLGTQTYICNVNHKPSNTKVDKRVE |
| | | | PKSCDKTHTCPPCPAPEAAGGPSVFLFPPKPK |
| | | | DTLMISRTPEVTCVVVDVSHEDPEVKFNWYVD |
| | | | GVEVHNAKTKPREEQYNSTYRVVSVLTVLHQD |
| | | | WLNGKEYKCKVSNKALPAPIEKTISKAKGQPRE |
| | | | PQVYTLPPSREEMTKNQVSLTCLVKGFYPSDIA |
| | | | VEWESNGQPENNYKTTPPVLDSDGSFFLYSKL |
| | | | TVDKSRWQQGNVFSCSVMHEALHNHYTQKSL |
| | | | SLSPG |
| 178 | IL1RAP_HCVR- | FULL LENGTH | QVQLVQSGAEVKKPGSSVKVSCKASGYIFLTY |
| | Fc | HEAVY CHAIN | WMNWVRQAPGQGLEWMGQIFPASGSAYYNQ |
| | (IL1RAP-opt- | | KFKGRVTITADKSTSTAYMELSSLRSEDTAVYY |
| | 43) | | CARSGPYSYYAGGYALDYWGQGTLVTVSSAS |
| | | | TKGPSVFPLAPSSKSTSGGTAALGCLVKDYFP |
| | | | EPVTVSWNSGALTSGVHTFPAVLQSSGLYSLS |
| | | | SVVTVPSSSLGTQTYICNVNHKPSNTKVDKRV |
| | | | EPKSCDKTHTCPPCPAPEAAGGPSVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPAPIEKTISKAKGQPR |
| | | | EPQVYTLPPSREEMTKNQVSLTCLVKGFYPSDI |
| | | | AVEWESNGQPENNYKTTPPVLDSDGSFFLYSK |
| | | | LTVDKSRWQQGNVFSCSVMHEALHNHYTQKS |
| | | | LSLSPG |
| 179 | IL1RAP_HCVR- | FULL LENGTH | QVQLVQSGAEVKKPGSSVKVSCKASGYIFLTY |
| | Fc | HEAVY CHAIN | WINWVRQAPGQGLEWMGQIFPASDSTYYNQK |
| | (IL1RAP-opt- | | FKGRVTITADKSTSTAYMELSSLRSEDTAVYYC |
| | 47) | | ARSGPYSYYAGGYALDYWGQGTLVTVSSAST |
| | | | KGPSVFPLAPSSKSTSGGTAALGCLVKDYFPE |
| | | | PVTVSWNSGALTSGVHTFPAVLQSSGLYSLSS |
| | | | VVTVPSSSLGTQTYICNVNHKPSNTKVDKRVE |
| | | | PKSCDKTHTCPPCPAPEAAGGPSVFLFPPKPK |
| | | | DTLMISRTPEVTCVVVDVSHEDPEVKFNWYVD |
| | | | GVEVHNAKTKPREEQYNSTYRVVSVLTVLHQD |
| | | | WLNGKEYKCKVSNKALPAPIEKTISKAKGQPRE |
| | | | PQVYTLPPSREEMTKNQVSLTCLVKGFYPSDIA |
| | | | VEWESNGQPENNYKTTPPVLDSDGSFFLYSKL |
| | | | TVDKSRWQQGNVFSCSVMHEALHNHYTQKSL |
| | | | SLSPG |
| 180 | IL1RAP_HCVR- | FULL LENGTH | QVQLVQSGAEVKKPGSSVKVSCKASGYIFLTY |
| | Fc | HEAVY CHAIN | WINWVRQAPGQGLEWMGQIFPASGSAYYAQK |
| | (IL1RAP-opt- | | FQGRVTITADKSTSTAYMELSSLRSEDTAVYYC |
| | 54) | | ARSGPYSYYAGGYALDYWGQGTLVTVSSAST |
| | | | KGPSVFPLAPSSKSTSGGTAALGCLVKDYFPE |
| | | | PVTVSWNSGALTSGVHTFPAVLQSSGLYSLSS |
| | | | VVTVPSSSLGTQTYICNVNHKPSNTKVDKRVE |
| | | | PKSCDKTHTCPPCPAPEAAGGPSVFLFPPKPK |
| | | | DTLMISRTPEVTCVVVDVSHEDPEVKFNWYVD |
| | | | GVEVHNAKTKPREEQYNSTYRVVSVLTVLHQD |
| | | | WLNGKEYKCKVSNKALPAPIEKTISKAKGQPRE |
| | | | PQVYTLPPSREEMTKNQVSLTCLVKGFYPSDIA |
| | | | VEWESNGQPENNYKTTPPVLDSDGSFFLYSKL |
| | | | TVDKSRWQQGNVFSCSVMHEALHNHYTQKSL |
| | | | SLSPG |
| 181 | IL1RAP_HCVR- | FULL LENGTH | QVQLVQSGAEVKKPGSSVKVSCKASGYIFLTY |
| | Fc | HEAVY CHAIN | WMNWVRQAPGQGLEWMGQIFPASGSAYYAQ |
| | (IL1RAP-opt- | | KFQGRVTITVDKSTSTAYMELSSLRSEDTAVYY |
| | 57) | | CARSGPYSYYAGGYALDYWGQGTLVTVSSAS |
| | | | TKGPSVFPLAPSSKSTSGGTAALGCLVKDYFP |
| | | | EPVTVSWNSGALTSGVHTFPAVLQSSGLYSLS |
| | | | SVVTVPSSSLGTQTYICNVNHKPSNTKVDKRV |
| | | | EPKSCDKTHTCPPCPAPEAAGGPSVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPAPIEKTISKAKGQPR |
| | | | EPQVYTLPPSREEMTKNQVSLTCLVKGFYPSDI |
| | | | AVEWESNGQPENNYKTTPPVLDSDGSFFLYSK |
| | | | LTVDKSRWQQGNVFSCSVMHEALHNHYTQKS |
| | | | LSLSPG |
| 182 | IL1RAP_HCVR- | FULL LENGTH | QVQLVQSGAEVKKPGSSVKVSCKASGYIFLTY |
| | Fc | HEAVY CHAIN | WMNWVRQAPGQGLEWMGQIFPASGSAYYNQ |
| | (IL1RAP-opt- | | KFKGRVTITADKSTSTAYMELSSLRSEDTAVYY |
| | 58) | | CARSGPYSYYAGGYALDYWGQGTLVTVSSAS |
| | | | TKGPSVFPLAPSSKSTSGGTAALGCLVKDYFP |
| | | | EPVTVSWNSGALTSGVHTFPAVLQSSGLYSLS |
| | | | SVVTVPSSSLGTQTYICNVNHKPSNTKVDKRV |
| | | | EPKSCDKTHTCPPCPAPEAAGGPSVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPAPIEKTISKAKGQPR |
| | | | EPQVYTLPPSREEMTKNQVSLTCLVKGFYPSDI |
| | | | AVEWESNGQPENNYKTTPPVLDSDGSFFLYSK |
| | | | LTVDKSRWQQGNVFSCSVMHEALHNHYTQKS |
| | | | LSLSPG |
| 183 | IL1RAP_HCVR- | FULL LENGTH | QVQLVQSGAEVKKPGSSVKVSCKASGYIFLTY |
| | Fc | HEAVY CHAIN | WINWVRQAPGQGLEWMGQIFPASGSTYYNEK |
| | (IL1RAP-opt- | | FKGRVTITADKSTSTAYMELSSLRSEDTAVYYC |
| | 59) | | ARSGPYSYYAGGYALDYWGQGTLVTVSSAST |
| | | | KGPSVFPLAPSSKSTSGGTAALGCLVKDYFPE |
| | | | PVTVSWNSGALTSGVHTFPAVLQSSGLYSLSS |
| | | | VVTVPSSSLGTQTYICNVNHKPSNTKVDKRVE |
| | | | PKSCDKTHTCPPCPAPEAAGGPSVFLFPPKPK |
| | | | DTLMISRTPEVTCVVVDVSHEDPEVKFNWYVD |
| | | | GVEVHNAKTKPREEQYNSTYRVVSVLTVLHQD |
| | | | WLNGKEYKCKVSNKALPAPIEKTISKAKGQPRE |
| | | | PQVYTLPPSREEMTKNQVSLTCLVKGFYPSDIA |
| | | | VEWESNGQPENNYKTTPPVLDSDGSFFLYSKL |
| | | | TVDKSRWQQGNVFSCSVMHEALHNHYTQKSL |
| | | | SLSPG |
| 184 | IL1RAP | AA 21-367 | SERCDDWGLDTMRQIQVFEDEPARIKCPLFEH |
| | EXTRACELLU | | FLKFNYSTAHSAGLTLIWYWTRQDRDLEEPINF |
| | LAR DOMAIN | | RLPENRISKEKDVLWFRPTLLNDTGNYTCMLR |
| | UNIPROT | | NTTYCSKVAFPLEVVQKDSCFNSPMKLPVHKL |
| | Q9NPH3 (AA- | | YIEYGIQRITCPNVDGYFPSSVKPTITWYMGCY |
| | 21-367) | | KIQNFNNVIPEGMNLSFLIALISNNGNYTCVVTY |
| | | | PENGRTFHLTRTLTVKVVGSPKNAVPPVIHSPN |
| | | | DHVVYEKEPGEELLIPCTVYFSFLMDSRNEVW |
| | | | WTIDGKKPDDITIDVTINESISHSRTEDETRTQIL |
| | | | SIKKVTSEDLKRSYVCHARSAKGEVAKAAKVK |
| | | | QKVPAPRYTVELACGFGAT |
| 185 | IL1RAP ECD | AA 21-134 | SERCDDWGLDTMRQIQVFEDEPARIKCPLFEH |
| | Domain 1 | | FLKFNYSTAHSAGLTLIWYWTRQDRDLEEPINF |
| | | | RLPENRISKEKDVLWFRPTLLNDTGNYTCMLR |
| | | | NTTYCSKVAFPLEVVQK |
| 186 | IL1RAP ECD | AA 135-234 | DSCFNSPMKLPVHKLYIEYGIQRITCPNVDGYF |
| | Domain 2 | | PSSVKPTITWYMGCYKIQNFNNVIPEGMNLSFL |
| | | | IALISNNGNYTCVVTYPENGRTFHLTRTLTVKVV |
| 187 | IL1RAP ECD | AA 235-367 | GSPKNAVPPVIHSPNDHVVYEKEPGEELLIPCT |
| | Domain 3 | | VYFSFLMDSRNEVWWTIDGKKPDDITIDVTINE |
| | | | SISHSRTEDETRTQILSIKKVTSEDLKRSYVCHA |
| | | | RSAKGEVAKAAKVKQKVPAPRYTVELACGFGA |
| | | | T |
| 188 | IL1RAP ECD | AA 235-315 | GSPKNAVPPVIHSPNDHVVYEKEPGEELLIPCT |
| | XR-D3A/B | | VYFSFLMDSRNEVWWTIDGKKPDDITIDVTINE |
| | | | SISHSRTEDETRTQI |
| 189 | IL1RAP ECD | AA 235-273 | GSPKNAVPPVIHSPNDHVVYEKEPGEELLIPCT |
| | XR D3A | | VYFSFL |
| 190 | IL1RAP ECD | AA 300-315 | SISHSRTEDETRTQI |
| | XR D3B | | |
| 191 | IL1RAP ECD | AA 226-262 | TRTLTVKVVGSPKNAVPPVIHSPNDHVVYEKEP |
| | HDX D2/3 A | | GEEL |
| 192 | IL1RAP ECD | AA 226-273 | TRTLTVKVVGSPKNAVPPVIHSPNDHVVYEKEP |
| | HDX D2/3 B | | GEELLIPCTVYFSFL |

1. An anti-IL1RAP antibody or antigen-binding fragment thereof, which binds to human IL1RAP at a K_{D} equal to or <0.1 nM.

2. An anti-IL1RAP antibody or antigen-binding fragment thereof according to claim 1, wherein the said antibody or antigen-binding fragment is a monoclonal antibody or antigen-binding fragment thereof.

3. An anti-IL1RAP antibody or antigen-binding fragment thereof according to claim 1, wherein the said antibody or antigen-binding fragment is a humanized antibody or antigen-binding fragment thereof.

4. An anti-IL1RAP antibody or antigen-binding fragment thereof according to claim 3, which binds to human and cynomolgus IL1RAP at a K_{D} equal to or <200 pM.

5. An anti-IL1RAP antibody or antigen-binding fragment thereof according to claim 1, wherein the antibody or antigen-binding fragment thereof comprises:
a) a light chain variable region comprising the amino acid sequence of SEQ ID NO: 3, 6, 117, 118, 119, 121, 122, 123, 124, 125, 126, 127, 128, 129, 130, 131, 132, 133, 134, or 135 (L-CDR1); the amino acid sequence of SEQ ID NO: 4, 7, 136, 137, 138, 139, 140, or 141 (L-CDR2); the amino acid sequence of SEQ ID NO: 5 (L-CDR3); and
b) a heavy chain variable region comprising the amino acid sequence of SEQ ID NO: 8, 11, 12, 14, 142, 143, 144, 145, 146, or 147 (H-CDR1); the amino acid sequence of SEQ ID NO: 9, 13, 15, 148, 149, 150, 151, 152, 153, 154, 155, 156, 157, 158, 159, 160, 161, 162, 163, 164, 165, 166, or 167 (H-CDR2); the amino acid sequence of SEQ ID NO: 10, or 16 (H-CDR3).

6. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
f) a heavy chain variable region comprising the amino acid sequence of SEQ ID NO: 112 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

7. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO: 7 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

8. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 146, wherein amino acids X1=M or I and X2=N or S (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
c) a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and
d) a heavy chain variable region comprising the amino acid sequence of SEQ ID NO: 147, wherein amino acids X1=M, I and X2=N or S) (H-CDR1); the amino acid sequence of SEQ ID NO: 165, wherein amino acids X1=D or G; X2=A or T; X3=N or A; X4=Q or E; X5=M or K; X6=Q or K; and X7=D or G (H-CDR2); the amino acid sequence of SEQ ID NO: 10 (H-CDR3), or
e) a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and
f) a heavy chain variable region comprising the amino acid sequence of SEQ ID NO: 112 (H-CDR1); the amino acid sequence of SEQ ID NO: 166, wherein amino acids X1=D or G (H-CDR2); the amino acid sequence of SEQ ID NO: 10 (H-CDR3), or
g) a light chain variable region comprising the amino acid sequence of SEQ ID NO: 127 (L-CDR1); the amino acid sequence of SEQ ID NO:139 (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 167, wherein amino acids X1=D or G and X2=T or A (H-CDR2); the amino acid sequence of SEQ ID NO: 16 (H-CDR3).

9. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO:9 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO:9 (H-CDR2); the amino acid sequence of SEQ ID NO: 10 (H-CDR3), or.
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO:13 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO:15 (H-CDR2); the amino acid sequence of SEQ ID NO: 16 (H-CDR3).

10. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

11. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 153 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 13 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 15 (H-CDR2); the amino acid sequence of SEQ ID NO: 16 (H-CDR3).

12. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

13. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 148 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

14. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 11 (H-CDR1); the amino acid sequence of SEQ ID NO: 149 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 162 (H-CDR2); the amino acid sequence of SEQ ID NO: 16 (H-CDR3).

15. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 151 (H-CDR2); the amino acid sequence of SEQ ID NO: 10 (H-CDR3), or
c) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
d) a heavy chain variable region comprising the amino acid sequence of SEQ ID NO: 144 (H-CDR1); the amino acid sequence of SEQ ID NO: 151 (H-CDR2); the amino acid sequence of SEQ ID NO: 10 (H-CDR3), or
e) a light chain variable region comprising the amino acid sequence of SEQ ID NO: 134, wherein amino acids X1=A or T; X2=Q or E; X3=S or N; and X4=N or S (L-CDR1); the amino acid sequence of SEQ ID NO: 140 wherein amino acids X1=K or S; and X2=S or T (L-CDR2); the amino acid sequence of SEQ ID NO: 5 (L-CDR3); and
f) a heavy chain variable region comprising the amino acid sequence of SEQ ID NO: 12 (H-CDR1); the amino acid sequence of SEQ ID NO: 161 (H-CDR2); the amino acid sequence of SEQ ID NO: 10 (H-CDR3), or
g) a light chain variable region comprising the amino acid sequence of SEQ ID NO: 135, wherein amino acids X1=Q or E; X2=S or N; and X3=N or S (L-CDR1); the amino acid sequence of SEQ ID NO: 141 wherein amino acids X1=K or S (L-CDR2); the amino acid sequence of SEQ ID NO: 5 (L-CDR3); and
h) a heavy chain variable region comprising the amino acid sequence of SEQ ID NO: 14 (H-CDR1); the amino acid sequence of SEQ ID NO: 163 (H-CDR2); the amino acid sequence of SEQ ID NO: 16 (H-CDR3).

16. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 3 (L-CDR1); the amino acid sequence of SEQ ID NO: 4 (L-CDR2); the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO:9 (H-CDR2); the amino acid sequence of SEQ ID NO: 10 (H-CDR3).

17. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3).

18. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3).

19. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 136; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3).

20. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3).

21. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3).

22. An anti-IL1RAP antibody or antigen-binding fragment thereof according to claim 5, wherein the antibody or antigen-binding fragment thereof comprises:
a) a light chain variable region comprising the amino acid sequence of SEQ ID NO: 117 (L-CDR1); the amino acid sequence of SEQ ID NO: 137; the amino acid sequence of SEQ ID NO: 5 (L-CDR3); and
b) a heavy chain variable region comprising the amino acid sequence of SEQ ID NO: 142 (H-CDR1); the amino acid sequence of SEQ ID NO: 151 (H-CDR2); the amino acid sequence of SEQ ID NO: 10 (H-CDR3).

23. An anti-IL1RAP antibody or antigen-binding fragment thereof according to claim 1, wherein the antibody or antigen-binding fragment thereof comprises a light chain variable region comprising the amino acid sequence of any one of SEQ ID NO: 17, 36, 40, 47, 50, 51, or 52; and a heavy chain variable region comprising the amino acid sequence of any one of SEQ ID NO: 67, 86, 97, 100, 101, or 102.

24. An anti-IL1RAP antibody or antigen-binding fragment thereof according to claim 23, wherein the antibody or antigen-binding fragment thereof comprises
a light chain variable region comprising the amino acid sequence of SEQ ID NO: 17; and a heavy chain variable region comprising the amino acid sequence of SEQ ID NO: 67; or
a light chain variable region comprising the amino acid sequence of SEQ ID NO: 36; and a heavy chain variable region comprising the amino acid sequence of SEQ ID NO: 86; or
a light chain variable region comprising the amino acid sequence of SEQ ID NO: 40; and a heavy chain variable region comprising the amino acid sequence of SEQ ID NO: 90; or
a light chain variable region comprising the amino acid sequence of SEQ ID NO: 47; and a heavy chain variable region comprising the amino acid sequence of SEQ ID NO: 97; or
a light chain variable region comprising the amino acid sequence of SEQ ID NO: 50; and a heavy chain variable region comprising the amino acid sequence of SEQ ID NO: 100; or
a light chain variable region comprising the amino acid sequence of SEQ ID NO: 51; and a heavy chain variable region comprising the amino acid sequence of SEQ ID NO: 101; or
a light chain variable region comprising the amino acid sequence of SEQ ID NO: 52; and a heavy chain variable region comprising the amino acid sequence of SEQ ID NO: 102.

25. An anti-IL1RAP antibody according to claim 1, wherein the antibody comprises a light chain comprising the amino acid sequence of any one of SEQ ID NO: 170, 171, 172, 173, 174, 175, or 176; and a heavy chain comprising the amino acid sequence of any one of SEQ ID NO: 177, 178, 179, 180, 181, 182, or 183.

26. An anti-IL1RAP antibody according to claim 25, wherein the antibody comprises a light chain comprising the amino acid sequence of SEQ ID NO: 170; and a heavy chain comprising the amino acid sequence of SEQ ID NO: 177.

27. An anti-IL1RAP antibody according to claim 25, wherein the antibody comprises a light chain comprising the amino acid sequence of SEQ ID NO: 171; and a heavy chain comprising the amino acid sequence of SEQ ID NO: 178.

28. An anti-IL1RAP antibody according to claim 25, wherein the antibody comprises a light chain comprising the amino acid sequence of SEQ ID NO: 172; and a heavy chain comprising the amino acid sequence of SEQ ID NO: 179.

29. An anti-IL1RAP antibody according to claim 25, wherein the antibody comprises a light chain comprising the amino acid sequence of SEQ ID NO: 173; and a heavy chain comprising the amino acid sequence of SEQ ID NO: 180.

30. An anti-IL1RAP antibody according to claim 25, wherein the antibody comprises a light chain comprising the amino acid sequence of SEQ ID NO: 174; and a heavy chain comprising the amino acid sequence of SEQ ID NO: 181.

31. An anti-IL1RAP antibody according to claim 25, wherein the antibody comprises a light chain comprising the amino acid sequence of SEQ ID NO: 175; and a heavy chain comprising the amino acid sequence of SEQ ID NO: 182.

32. An anti-IL1RAP antibody according to claim 25, wherein the antibody comprises a light chain comprising the amino acid sequence of SEQ ID NO: 176; and a heavy chain comprising the amino acid sequence of SEQ ID NO: 183.

33. An anti-IL-36R antibody or antigen-binding fragment thereof according to claim 1, wherein the antibody or antigen-binding fragment thereof comprises:
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 118 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 119 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 149 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 150 (H-CDR2); the amino acid sequence of SEQ ID NO: 10 (H-CDR3); or
a light chain variable region comprising the amino acid sequence of SEQ ID NO: 120 (L-CDR1); the amino acid sequence of SEQ ID NO: 136 (L-CDR2); the amino acid sequence of SEQ ID NO: 5L-CDR3); and a heavy chain variable region comprising the amino acid sequence of SEQ ID NO: 142 (H-CDR1); the amino acid sequence of SEQ ID NO: 152 (H-CDR2); the amino acid sequence of SEQ ID NO: 10 (H-CDR3); or
a light chain variable region comprising the amino acid sequence of SEQ ID NO: 121 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 152 (H-CDR2); the amino acid sequence of SEQ ID NO: 10 (H-CDR3); or
a light chain variable region comprising the amino acid sequence of SEQ ID NO: 122 (L-CDR1); the amino acid sequence of SEQ ID NO: 138 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 154 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 155 (H-CDR2); the amino acid sequence of SEQ ID NO: 10 (H-CDR3); or
a light chain variable region comprising the amino acid sequence of SEQ ID NO: 121 (L-CDR1); the amino acid sequence of SEQ ID NO: 138 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 120 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 122 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 156 (H-CDR2); the amino acid sequence of SEQ ID NO: 10 (H-CDR3); or
a light chain variable region comprising the amino acid sequence of SEQ ID NO: 123 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 143 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 124 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 158 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 151 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 152 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 154 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 155 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 156 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 143 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 136 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 152 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 153 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 154 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 155 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 156 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 143 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 148 (H-CDR2); the amino acid sequence of SEQ ID NO: 10 (H-CDR3); or
a light chain variable region comprising the amino acid sequence of SEQ ID NO: 117 (L-CDR1); the amino acid sequence of SEQ ID NO: 137 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 157 (H-CDR2); the amino acid sequence of SEQ ID NO: 10 (H-CDR3); or
a light chain variable region comprising the amino acid sequence of SEQ ID NO: 125 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 159 (H-CDR2); the amino acid sequence of SEQ ID NO: 10 (H-CDR3); or
a light chain variable region comprising the amino acid sequence of SEQ ID NO: 125 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 142 (H-CDR1); the amino acid sequence of SEQ ID NO: 160 (H-CDR2); the amino acid sequence of SEQ ID NO: 10 (H-CDR3); or
a light chain variable region comprising the amino acid sequence of SEQ ID NO: 125 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 159 (H-CDR2); the amino acid sequence of SEQ ID NO: 10 (H-CDR3); or
a light chain variable region comprising the amino acid sequence of SEQ ID NO: 125 (L-CDR1); the amino acid sequence of SEQ ID NO: 4 (L-CDR2); the amino acid sequence of SEQ ID NO: 5 (L-CDR3); and a heavy chain variable region comprising the amino acid sequence of SEQ ID NO: 8 (H-CDR1); the amino acid sequence of SEQ ID NO: 160 (H-CDR2); the amino acid sequence of SEQ ID NO: 10 (H-CDR3).

34. An anti-IL1RAP antibody or antigen-binding fragment thereof according to claim 33, wherein the antibody or antigen-binding fragment thereof comprises
a light chain variable region comprising the amino acid sequence of SEQ ID NO: 20; and a heavy chain variable region comprising the amino acid sequence of SEQ ID NO: 70; or
a light chain variable region comprising the amino acid sequence of SEQ ID NO: 21; and a heavy chain variable region comprising the amino acid sequence of SEQ ID NO: 71; or
a light chain variable region comprising the amino acid sequence of SEQ ID NO: 22; and a heavy chain variable region comprising the amino acid sequence of SEQ ID NO: 72; or
a light chain variable region comprising the amino acid sequence of SEQ ID NO: 23; and a heavy chain variable region comprising the amino acid sequence of SEQ ID NO: 73; or
a light chain variable region comprising the amino acid sequence of SEQ ID NO: 24; and a heavy chain variable region comprising the amino acid sequence of SEQ ID NO: 74; or
a light chain variable region comprising the amino acid sequence of SEQ ID NO: 25; and a heavy chain variable region comprising the amino acid sequence of SEQ ID NO: 75; or
a light chain variable region comprising the amino acid sequence of SEQ ID NO: 26; and a heavy chain variable region comprising the amino acid sequence of SEQ ID NO: 76; or
a light chain variable region comprising the amino acid sequence of SEQ ID NO: 27; and a heavy chain variable region comprising the amino acid sequence of SEQ ID NO: 77; or
a light chain variable region comprising the amino acid sequence of SEQ ID NO: 28; and a heavy chain variable region comprising the amino acid sequence of SEQ ID NO: 78; or
a light chain variable region comprising the amino acid sequence of SEQ ID NO: 29; and a heavy chain variable region comprising the amino acid sequence of SEQ ID NO: 79; or
a light chain variable region comprising the amino acid sequence of SEQ ID NO: 30; and a heavy chain variable region comprising the amino acid sequence of SEQ ID NO: 80; or
a light chain variable region comprising the amino acid sequence of SEQ ID NO: 31; and a heavy chain variable region comprising the amino acid sequence of SEQ ID NO: 81; or
a light chain variable region comprising the amino acid sequence of SEQ ID NO: 32; and a heavy chain variable region comprising the amino acid sequence of SEQ ID NO: 82; or
a light chain variable region comprising the amino acid sequence of SEQ ID NO: 33; and a heavy chain variable region comprising the amino acid sequence of SEQ ID NO: 83; or
a light chain variable region comprising the amino acid sequence of SEQ ID NO: 34; and a heavy chain variable region comprising the amino acid sequence of SEQ ID NO: 84; or
a light chain variable region comprising the amino acid sequence of SEQ ID NO: 35; and a heavy chain variable region comprising the amino acid sequence of SEQ ID NO: 85; or
a light chain variable region comprising the amino acid sequence of SEQ ID NO: 37; and a heavy chain variable region comprising the amino acid sequence of SEQ ID NO: 87; or
a light chain variable region comprising the amino acid sequence of SEQ ID NO: 38; and a heavy chain variable region comprising the amino acid sequence of SEQ ID NO: 88; or
a light chain variable region comprising the amino acid sequence of SEQ ID NO: 39; and a heavy chain variable region comprising the amino acid sequence of SEQ ID NO: 89; or
a light chain variable region comprising the amino acid sequence of SEQ ID NO: 41; and a heavy chain variable region comprising the amino acid sequence of SEQ ID NO: 91; or
a light chain variable region comprising the amino acid sequence of SEQ ID NO: 42; and a heavy chain variable region comprising the amino acid sequence of SEQ ID NO: 92; or
a light chain variable region comprising the amino acid sequence of SEQ ID NO: 43; and a heavy chain variable region comprising the amino acid sequence of SEQ ID NO: 93; or
a light chain variable region comprising the amino acid sequence of SEQ ID NO: 44; and a heavy chain variable region comprising the amino acid sequence of SEQ ID NO: 94; or
a light chain variable region comprising the amino acid sequence of SEQ ID NO: 45; and a heavy chain variable region comprising the amino acid sequence of SEQ ID NO: 95; or
a light chain variable region comprising the amino acid sequence of SEQ ID NO: 46; and a heavy chain variable region comprising the amino acid sequence of SEQ ID NO: 96; or
a light chain variable region comprising the amino acid sequence of SEQ ID NO: 48; and a heavy chain variable region comprising the amino acid sequence of SEQ ID NO: 98; or
a light chain variable region comprising the amino acid sequence of SEQ ID NO: 49; and a heavy chain variable region comprising the amino acid sequence of SEQ ID NO: 99; or
a light chain variable region comprising the amino acid sequence of SEQ ID NO: 53; and a heavy chain variable region comprising the amino acid sequence of SEQ ID NO: 103; or
a light chain variable region comprising the amino acid sequence of SEQ ID NO: 54; and a heavy chain variable region comprising the amino acid sequence of SEQ ID NO: 104; or
a light chain variable region comprising the amino acid sequence of SEQ ID NO: 55; and a heavy chain variable region comprising the amino acid sequence of SEQ ID NO: 105; or
a light chain variable region comprising the amino acid sequence of SEQ ID NO: 56; and a heavy chain variable region comprising the amino acid sequence of SEQ ID NO: 106; or
a light chain variable region comprising the amino acid sequence of SEQ ID NO: 57; and a heavy chain variable region comprising the amino acid sequence of SEQ ID NO: 107; or
a light chain variable region comprising the amino acid sequence of SEQ ID NO: 58; and a heavy chain variable region comprising the amino acid sequence of SEQ ID NO: 108; or
a light chain variable region comprising the amino acid sequence of SEQ ID NO: 59; and a heavy chain variable region comprising the amino acid sequence of SEQ ID NO: 109; or
a light chain variable region comprising the amino acid sequence of SEQ ID NO: 60; and a heavy chain variable region comprising the amino acid sequence of SEQ ID NO: 110; or
a light chain variable region comprising the amino acid sequence of SEQ ID NO: 61; and a heavy chain variable region comprising the amino acid sequence of SEQ ID NO: 111; or
a light chain variable region comprising the amino acid sequence of SEQ ID NO: 62; and a heavy chain variable region comprising the amino acid sequence of SEQ ID NO: 112; or
a light chain variable region comprising the amino acid sequence of SEQ ID NO: 63; and a heavy chain variable region comprising the amino acid sequence of SEQ ID NO: 113; or
a light chain variable region comprising the amino acid sequence of SEQ ID NO: 64; and a heavy chain variable region comprising the amino acid sequence of SEQ ID NO: 114; or
a light chain variable region comprising the amino acid sequence of SEQ ID NO: 65; and a heavy chain variable region comprising the amino acid sequence of SEQ ID NO: 115; or
a light chain variable region comprising the amino acid sequence of SEQ ID NO: 66; and a heavy chain variable region comprising the amino acid sequence of SEQ ID NO: 116.

35. (canceled)

36. A pharmaceutical composition comprising an antibody or antigen-binding fragment according to claim 1, and a pharmaceutically acceptable carrier.

37. A method of treating a disease comprising administering the antibody or antigen-binding fragment according to claim 1, or a pharmaceutical composition thereof, to a patient in need thereof, wherein the disease is selected from an inflammatory disease, an autoimmune disease, a respiratory disease, a metabolic disorder, an epithelial mediated inflammatory disorder, fibrosis and cancer.

38. An anti-IL1RAP antibody or antigen-binding fragment according to claim 37 for use in treating a disease, wherein the disease is selected from an inflammatory disease, an autoimmune disease, a respiratory disease, a metabolic disorder, an epithelial mediated inflammatory disorder, fibrosis and cancer.

39. (canceled)

40. A method according to claim 37, wherein the disease is selected from psoriasis, psoriatic arthritis, multiple sclerosis, rheumatoid arthritis, COPD, chronic asthma and ankylosing spondylitis.

41. An isolated polynucleotide according to claim 1, encoding a sequence as defined by one or more of SEQ ID NOs. 1 to 167, or 170-183.

42. A vector comprising a polynucleotide according to claim 41, preferably an expression vector, more preferred a vector comprising the polynucleotide according to the invention in functional association with an expression control sequence.

43. A host cell comprising a polynucleotide according to claim 42, and a vector.

44. Method for the production of an anti-IL1RAP antibody or antigen-binding fragment according to claim 5, comprising the steps (a) cultivating the host cell under conditions allowing the expression of the anti-IL1RAP antibody or antigen-binding fragment and (b) recovering the anti-IL1RAP antibody or antigen-binding fragment.

45. Diagnostic kit or diagnostic method comprising an anti-IL1RAP antibody or antigen-binding fragment according to claim 5, or the use thereof.

46. Diagnostic kit or diagnostic method according to claim 45, for the diagnosis of an inflammatory disease, an autoimmune disease, a respiratory disease, a metabolic disorder, an epithelial mediated inflammatory disorder, fibrosis, cancer, psoriasis, psoriatic arthritis, multiple sclerosis, rheumatoid arthritis, COPD, chronic asthma, or ankylosing spondylitis.

47. An isolated antibody or antigen-binding fragment wherein when bound to IL1RAP, the antibody binds to the following residues K238, N239, V241, V244, I245, H246, S247, N249, H251, V252, V253, Y254, E255, K256, E261, L263, P265, T267, Y269, and S271, and wherein the antibody blocks binding of any of the antibodies of claim 5.

48. The isolated monoclonal antibody of claim 47 wherein the antibody blocks the binding of anti-IL1RAP to IL1RAP by at least 80%.


SEQUENCE LISTING (194 sequences)

SEQ ID NO: 1 - protein, 107 aa, Artificial Sequence
  source: /note=“Description of Artificial Sequence Synthetic polypeptide”
DIQMTQSPASLSASVGETVTITCRTSENINSYLAWYQQKQGKSPQLLVHYAKTLAEGVPSRFSGSGSGTQFSLKINSLKPEDFGSYYCQHHYGTSLTFGAGTKLELK

SEQ ID NO: 2 - protein, 124 aa, Artificial Sequence
  source: /note=“Description of Artificial Sequence Synthetic polypeptide”
QVHLQQSGPELVRPGTSVKISCEASGYIFLTYWMNWVKQRPGQGLEWIGQIFPASDSTYYNEMFKDKARFTVDKSSSTAYMQFSSLTSEDTAVYFCARSGPYSYYAGGYALDYWGQGTSVTVSS

SEQ ID NO: 3 - protein, 11 aa, Artificial Sequence
  source: /note=“Description of Artificial Sequence Synthetic peptide”
RTSENINSYLA

SEQ ID NO: 4 - protein, 7 aa, Artificial Sequence
  source: /note=“Description of Artificial Sequence Synthetic peptide”
YAKTLAE

SEQ ID NO: 5 - protein, 9 aa, Artificial Sequence
  source: /note=“Description of Artificial Sequence Synthetic peptide”
QHHYGTSLT

SEQ ID NO: 6 - protein, 6 aa, Artificial Sequence
  source: /note=“Description of Artificial Sequence Synthetic peptide”
ENINSY

SEQ ID NO: 7 - protein, 3 aa, Artificial Sequence
  source: /note=“Description of Artificial Sequence Synthetic peptide”
YAK

SEQ ID NO: 8 - protein, 5 aa, Artificial Sequence
  source: /note=“Description of Artificial Sequence Synthetic peptide”
TYWMN

SEQ ID NO: 9 - protein, 17 aa, Artificial Sequence
  source: /note=“Description of Artificial Sequence Synthetic peptide”
QIFPASDSTYYNEMFKD

SEQ ID NO: 10 - protein, 15 aa, Artificial Sequence
  source: /note=“Description of Artificial Sequence Synthetic peptide”
SGPYSYYAGGYALDY

SEQ ID NO: 11 - protein, 10 aa, Artificial Sequence
  source: /note=“Description of Artificial Sequence Synthetic peptide”
GYIFLTYWMN

SEQ ID NO: 12 - protein, 7 aa, Artificial Sequence
  source: /note=“Description of Artificial Sequence Synthetic peptide”
GYIFLTY

SEQ ID NO: 13 - protein, 6 aa, Artificial Sequence
  source: /note=“Description of Artificial Sequence Synthetic peptide”
FPASDS

SEQ ID NO: 14 - protein, 8 aa, Artificial Sequence
  source: /note=“Description of Artificial Sequence Synthetic peptide”
GYIFLTYW

SEQ ID NO: 15 - protein, 8 aa, Artificial Sequence
  source: /note=“Description of Artificial Sequence Synthetic peptide”
IFPASDST

SEQ ID NO: 16 - protein, 17 aa, Artificial Sequence
  source: /note=“Description of Artificial Sequence Synthetic peptide”
ARSGPYSYYAGGYALDY

SEQ ID NO: 17 - protein, 107 aa, Artificial Sequence
  source: /note=“Description of Artificial Sequence Synthetic polypeptide”
DIQMTQSPSSLSASVGDRVTITCRTSENINSYLAWYQQKPGKAPKLLIYYAKTLAEGVPSRFSGSGSGTDFTLTISSLQPEDFATYYCQHHYGTSLTFGQGTKLEIK

SEQ ID NO: 18 - protein, 107 aa, Artificial Sequence
  source: /note=“Description of Artificial Sequence Synthetic polypeptide”
DIQMTQSPSSLSASVGDRVTITCRTSENINSYLAWYQQKPGKAPKLLIHYAKTLAEGVPSRFSGSGSGTDFTLTISSLQPEDFATYYCQHHYGTSLTFGQGTKLEIK

SEQ ID NO: 19 - protein, 107 aa, Artificial Sequence; identical to SEQ ID NO: 18
  source: /note=“Description of Artificial Sequence Synthetic polypeptide”

SEQ ID NO: 20 - protein, 107 aa, Artificial Sequence
  source: /note=“Description of Artificial Sequence Synthetic polypeptide”
DIQMTQSPSSLSASVGDRVTITCRASQSISSYLAWYQQKPGKAPKLLIHYAKSLAEGVPSRFSGSGSGTDFTLTISSLQPEDFATYYCQHHYGTSLTFGQGTKLEIK

SEQ ID NO: 21 - protein, 107 aa, Artificial Sequence
  source: /note=“Description of Artificial Sequence Synthetic polypeptide”
DIQMTQSPSSLSASVGDRVTITCRASENINSYLAWYQQKPGKAPKLLIHYAKTLAEGVPSRFSGSGSGTDFTLTISSLQPEDFATYYCQHHYGTSLTFGQGTKLEIK

SEQ ID NO: 22 - protein, 107 aa, Artificial Sequence
  source: /note=“Description of Artificial Sequence Synthetic polypeptide”
DIQMTQSPSSLSASVGDRVTITCRTSQNINSYLAWYQQKPGKAPKLLIHYAKTLAEGVPSRFSGSGSGTDFTLTISSLQPEDFATYYCQHHYGTSLTFGQGTKLEIK

SEQ ID NO: 23 - protein, 107 aa, Artificial Sequence
  source: /note=“Description of Artificial Sequence Synthetic polypeptide”
DIQMTQSPSSLSASVGDRVTITCRASQSISSYLAWYQQKPGKAPKLLIHYASSLAEGVPSRFSGSGSGTDFTLTISSLQPEDFATYYCQHHYGTSLTFGQGTKLEIK

SEQ ID NO: 24 - protein, 107 aa, Artificial Sequence
  source: /note=“Description of Artificial Sequence Synthetic polypeptide”
DIQMTQSPSSLSASVGDRVTITCRTSESINSYLAWYQQKPGKAPKLLIHYAKSLAEGVPSRFSGSGSGTDFTLTISSLQPEDFATYYCQHHYGTSLTFGQGTKLEIK

SEQ ID NO: 25 - protein, 107 aa, Artificial Sequence
  source: /note=“Description of Artificial Sequence Synthetic polypeptide”
DIQMTQSPSSLSASVGDRVTITCRASQSINSYLAWYQQKPGKAPKLLIHYAKTLAEGVPSRFSGSGSGTDFTLTISSLQPEDFATYYCQHHYGTSLTFGQGTKLEIK

SEQ ID NO: 26 - protein, 107 aa, Artificial Sequence
  source: /note=“Description of Artificial Sequence Synthetic polypeptide”
DIQMTQSPSSLSASVGDRVTITCRTSESISSYLAWYQQKPGKAPKLLIHYASTLAEGVPSRFSGSGSGTDFTLTISSLQPEDFATYYCQHHYGTSLTFGQGTKLEIK

SEQ ID NO: 27 - protein, 107 aa, Artificial Sequence
  source: /note=“Description of Artificial Sequence Synthetic polypeptide”
DIQMTQSPSSLSASVGDRVTITCRASQSISSYLAWYQQKPGKAPKLLIHYAKTLAEGVPSRFSGSGSGTDFTLTISSLQPEDFATYYCQHHYGTSLTFGQGTKLEIK

SEQ ID NO: 28 - protein, 107 aa, Artificial Sequence; identical to SEQ ID NO: 20
  source: /note=“Description of Artificial Sequence Synthetic polypeptide”

SEQ ID NO: 29 - protein, 107 aa, Artificial Sequence
  source: /note=“Description of Artificial Sequence Synthetic polypeptide”
DIQMTQSPSSLSASVGDRVTITCRASQSINSYLAWYQQKPGKAPKLLIHYASTLAEGVPSRFSGSGSGTDFTLTISSLQPEDFATYYCQHHYGTSLTFGQGTKLEIK

SEQ ID NO: 30 - protein, 107 aa, Artificial Sequence; identical to SEQ ID NO: 24
  source: /note=“Description of Artificial Sequence Synthetic polypeptide”

SEQ ID NO: 31 - protein, 107 aa, Artificial Sequence
  source: /note=“Description of Artificial Sequence Synthetic polypeptide”
DIQMTQSPSSLSASVGDRVTITCRASENISSYLAWYQQKPGKAPKLLIHYAKSLAEGVPSRFSGSGSGTDFTLTISSLQPEDFATYYCQHHYGTSLTFGQGTKLEIK

SEQ ID NO: 32 - protein, 107 aa, Artificial Sequence
  source: /note=“Description of Artificial Sequence Synthetic polypeptide”
DIQMTQSPSSLSASVGDRVTITCRTSQNISSYLAWYQQKPGKAPKLLIHYAKSLAEGVPSRFSGSGSGTDFTLTISSLQPEDFATYYCQHHYGTSLTFGQGTKLEIK

SEQ ID NO: 33 - protein, 107 aa, Artificial Sequence
  source: /note=“Description of Artificial Sequence Synthetic polypeptide”
DIQMTQSPSSLSASVGDRVTITCRTSQSINSYLAWYQQKPGKAPKLLIHYASSLAEGVPSRFSGSGSGTDFTLTISSLQPEDFATYYCQHHYGTSLTFGQGTKLEIK

SEQ ID NO: 34 - protein, 107 aa, Artificial Sequence; identical to SEQ ID NO: 27
  source: /note=“Description of Artificial Sequence Synthetic polypeptide”

SEQ ID NO: 35 - protein, 107 aa, Artificial Sequence; identical to SEQ ID NO: 20
  source: /note=“Description of Artificial Sequence Synthetic polypeptide”

SEQ ID NO: 36 - protein, 107 aa, Artificial Sequence; identical to SEQ ID NO: 20
  source: /note=“Description of Artificial Sequence Synthetic polypeptide”

SEQ ID NO: 37 - protein, 107 aa, Artificial Sequence; identical to SEQ ID NO: 20
  source: /note=“Description of Artificial Sequence Synthetic polypeptide”

SEQ ID NO: 38 - protein, 107 aa, Artificial Sequence; identical to SEQ ID NO: 20
  source: /note=“Description of Artificial Sequence Synthetic polypeptide”

SEQ ID NO: 39 - protein, 107 aa, Artificial Sequence; identical to SEQ ID NO: 20
  source: /note=“Description of Artificial Sequence Synthetic polypeptide”

SEQ ID NO: 40 - protein, 107 aa, Artificial Sequence; identical to SEQ ID NO: 20
  source: /note=“Description of Artificial Sequence Synthetic polypeptide”

SEQ ID NO: 41 - protein, 107 aa, Artificial Sequence; identical to SEQ ID NO: 20
  source: /note=“Description of Artificial Sequence Synthetic polypeptide”

SEQ ID NO: 42 - protein, 107 aa, Artificial Sequence; identical to SEQ ID NO: 20
  source: /note=“Description of Artificial Sequence Synthetic polypeptide”

SEQ ID NO: 43 - protein, 107 aa, Artificial Sequence; identical to SEQ ID NO: 20
  source: /note=“Description of Artificial Sequence Synthetic polypeptide”

SEQ ID NO: 44 - protein, 107 aa, Artificial Sequence; identical to SEQ ID NO: 20
  source: /note=“Description of Artificial Sequence Synthetic polypeptide”

SEQ ID NO: 45 - protein, 107 aa, Artificial Sequence; identical to SEQ ID NO: 20
  source: /note=“Description of Artificial Sequence Synthetic polypeptide”

SEQ ID NO: 46 - protein, 107 aa, Artificial Sequence; identical to SEQ ID NO: 20
  source: /note=“Description of Artificial Sequence Synthetic polypeptide”

SEQ ID NO: 47 - protein, 107 aa, Artificial Sequence; identical to SEQ ID NO: 20
  source: /note=“Description of Artificial Sequence Synthetic polypeptide”

SEQ ID NO: 48 - protein, 107 aa, Artificial Sequence; identical to SEQ ID NO: 20
  source: /note=“Description of Artificial Sequence Synthetic polypeptide”

SEQ ID NO: 49 - protein, 107 aa, Artificial Sequence; identical to SEQ ID NO: 23
  source: /note=“Description of Artificial Sequence Synthetic polypeptide”

SEQ ID NO: 50 - protein, 107 aa, Artificial Sequence; identical to SEQ ID NO: 23
  source: /note=“Description of Artificial Sequence Synthetic polypeptide”

SEQ ID NO: 51 - protein, 107 aa, Artificial Sequence; identical to SEQ ID NO: 23
  source: /note=“Description of Artificial Sequence Synthetic polypeptide”

SEQ ID NO: 52 - protein, 107 aa, Artificial Sequence; identical to SEQ ID NO: 23
  source: /note=“Description of Artificial Sequence Synthetic polypeptide”

SEQ ID NO: 53 - protein, 107 aa, Artificial Sequence; identical to SEQ ID NO: 23
  source: /note=“Description of Artificial Sequence Synthetic polypeptide”

SEQ ID NO: 54 - protein, 107 aa, Artificial Sequence; identical to SEQ ID NO: 23
  source: /note=“Description of Artificial Sequence Synthetic polypeptide”

SEQ ID NO: 55 - protein, 107 aa, Artificial Sequence; identical to SEQ ID NO: 23
  source: /note=“Description of Artificial Sequence Synthetic polypeptide”

SEQ ID NO: 56 - protein, 107 aa, Artificial Sequence; identical to SEQ ID NO: 23
  source: /note=“Description of Artificial Sequence Synthetic polypeptide”

SEQ ID NO: 57 - protein, 107 aa, Artificial Sequence; identical to SEQ ID NO: 23
  source: /note=“Description of Artificial Sequence Synthetic polypeptide”

SEQ ID NO: 58 - protein, 107 aa, Artificial Sequence; identical to SEQ ID NO: 23
  source: /note=“Description of Artificial Sequence Synthetic polypeptide”

SEQ ID NO: 59 - protein, 107 aa, Artificial Sequence; identical to SEQ ID NO: 23
  source: /note=“Description of Artificial Sequence Synthetic polypeptide”

SEQ ID NO: 60 - protein, 107 aa, Artificial Sequence; identical to SEQ ID NO: 23
  source: /note=“Description of Artificial Sequence Synthetic polypeptide”

SEQ ID NO: 61 - protein, 107 aa, Artificial Sequence; identical to SEQ ID NO: 23
  source: /note=“Description of Artificial Sequence Synthetic polypeptide”

SEQ ID NO: 62 - protein, 107 aa, Artificial Sequence; identical to SEQ ID NO: 23
  source: /note=“Description of Artificial Sequence Synthetic polypeptide”

SEQ ID NO: 63 - protein, 107 aa, Artificial Sequence
  source: /note=“Description of Artificial Sequence Synthetic polypeptide”
DIQMTQSPSSLSASVGDRVTITCRTSENISSYLAWYQQKPGKAPKLLIHYAKTLAEGVPSRFSGSGSGTDFTLTISSLQPEDFATYYCQHHYGTSLTFGQGTKLEIK

SEQ ID NO: 64 - protein, 107 aa, Artificial Sequence; identical to SEQ ID NO: 63
  source: /note=“Description of Artificial Sequence Synthetic polypeptide”

SEQ ID NO: 65 - protein, 107 aa, Artificial Sequence; identical to SEQ ID NO: 63
  source: /note=“Description of Artificial Sequence Synthetic polypeptide”

SEQ ID NO: 66 - protein, 107 aa, Artificial Sequence; identical to SEQ ID NO: 63
  source: /note=“Description of Artificial Sequence Synthetic polypeptide”

SEQ ID NO: 67 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASDSTYYNEMFKDRVTITADKSTSTAYMELSSLRSEDTAVYYCARSGPYSYYAGGYALDYWGQGTLVTVSS

SEQ ID NO: 68 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASDSTYYNEMFKDRVRITVDKSTSTAYMELSSLRSEDTAVYYCARSGPYSYYAGGYALDYWGQGTLVTVSS

SEQ ID NO: 69 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASDSTYYNEMFKDRVTFTVDKSTSTAYMELSSLRSEDTAVYYCARSGPYSYYAGGYALDYWGQGTLVTVSS

SEQ ID NO: 70 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSAYYAQKFQGRVTITADKSTSTAYMELSSLRSEDTAVYYCARSGPYSYYAGGYALDYWGQGTLVTVSS

SEQ ID NO: 71 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYAQKFQGRVTITVDKSTSTAYMELSSLRSEDTAVYYCARSGPYSYYAGGYALDYWGQGTLVTVSS

SEQ ID NO: 72 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYNQKFKGRVTITADKSTSTAYMELSSLRSEDTAVYYCARSGPYSYYAGGYALDYWGQGTLVTVSS

SEQ ID NO: 73 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASDSTYYAQMFQDRVTITADKSTSTAYMELSSLRSEDTAVYYCARSGPYSYYAGGYALDYWGQGTLVTVSS

SEQ ID NO: 74 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSTYYNEKFKGRVTITADKSTSTAYMELSSLRSEDTAVYYCARSGPYSYYAGGYALDYWGQGTLVTVSS

SEQ ID NO: 75 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSTYYAQKFQDRVTITVDKSTSTAYMELSSLRSEDTAVYYCARSGPYSYYAGGYALDYWGQGTLVTVSS

SEQ ID NO: 76 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASDSTYYNQKFKGRVTITADKSTSTAYMELSSLRSEDTAVYYCARSGPYSYYAGGYALDYWGQGTLVTVSS

SEQ ID NO: 77 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASDSAYYNEKFKGRVTITVDKSTSTAYMELSSLRSEDTAVYYCARSGPYSYYAGGYALDYWGQGTLVTVSS

SEQ ID NO: 78 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASDSAYYAQKFQDRVTITADKSTSTAYMELSSLRSEDTAVYYCARSGPYSYYAGGYALDYWGQGTLVTVSS

SEQ ID NO: 79 - protein, 124 aa, Artificial Sequence; identical to SEQ ID NO: 71
  source: /note=“Description of Artificial Sequence Synthetic polypeptide”

SEQ ID NO: 80 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYAQKFQGRVTITADKSTSTAYMELSSLRSEDTAVYYCARSGPYSYYAGGYALDYWGQGTLVTVSS

SEQ ID NO: 81 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYNEKFKDRVTITVDKSTSTAYMELSSLRSEDTAVYYCARSGPYSYYAGGYALDYWGQGTLVTVSS

SEQ ID NO: 82 - protein, 124 aa, Artificial Sequence
  source: /note=“Description of Artificial Sequence Synthetic polypeptide”
QVQLVQSGAEVKKPGSSVKVSCKASGYIFLTYWISWVRQAPGQGLEWMGQIFPASDSAYYNQMFKDRVTITVDKSTSTAYMELSSLRSEDTAVYYCARSGPYSYYAGGYALDYWGQGTLVTVSS

SEQ ID NO: 83 - protein, 124 aa, Artificial Sequence; identical to SEQ ID NO: 70
  source: /note=“Description of Artificial Sequence Synthetic polypeptide”

SEQ ID NO: 84 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASDSAYYNQMFKDRVTITADKSTSTAYMELSSLRSEDTAVYYCARSGPYSYYAGGYALDYWGQGTLVTVSS

SEQ ID NO: 85 - protein, 124 aa, Artificial Sequence; identical to SEQ ID NO: 71
  source: /note=“Description of Artificial Sequence Synthetic polypeptide”

SEQ ID NO: 86 - protein, 124 aa, Artificial Sequence; identical to SEQ ID NO: 72
  source: /note=“Description of Artificial Sequence Synthetic polypeptide”

SEQ ID NO: 87 - protein, 124 aa, Artificial Sequence; identical to SEQ ID NO: 73
  source: /note=“Description of Artificial Sequence Synthetic polypeptide”

SEQ ID NO: 88 - protein, 124 aa, Artificial Sequence; identical to SEQ ID NO: 74
  source: /note=“Description of Artificial Sequence Synthetic polypeptide”

SEQ ID NO: 89 - protein, 124 aa, Artificial Sequence; identical to SEQ ID NO: 75
  source: /note=“Description of Artificial Sequence Synthetic polypeptide”

SEQ ID NO: 90 - protein, 124 aa, Artificial Sequence; identical to SEQ ID NO: 76
  source: /note=“Description of Artificial Sequence Synthetic polypeptide”

SEQ ID NO: 91 - protein, 124 aa, Artificial Sequence; identical to SEQ ID NO: 77
  source: /note=“Description of Artificial Sequence Synthetic polypeptide”

SEQ ID NO: 92 - protein, 124 aa, Artificial Sequence; identical to SEQ ID NO: 78
  source: /note=“Description of Artificial Sequence Synthetic polypeptide”

SEQ ID NO: 93 - protein, 124 aa, Artificial Sequence; identical to SEQ ID NO: 71
  source: /note=“Description of Artificial Sequence Synthetic polypeptide”

SEQ ID NO: 94 - protein, 124 aa, Artificial Sequence; identical to SEQ ID NO: 80
  source: /note=“Description of Artificial Sequence Synthetic polypeptide”

SEQ ID NO: 95 - protein, 124 aa, Artificial Sequence; identical to SEQ ID NO: 81
  source: /note=“Description of Artificial Sequence Synthetic polypeptide”

SEQ ID NO: 96 - protein, 124 aa, Artificial Sequence; identical to SEQ ID NO: 82
  source: /note=“Description of Artificial Sequence Synthetic polypeptide”

SEQ ID NO: 97 - protein, 124 aa, Artificial Sequence; identical to SEQ ID NO: 70
  source: /note=“Description of Artificial Sequence Synthetic polypeptide”

SEQ ID NO: 98 - protein, 124 aa, Artificial Sequence; identical to SEQ ID NO: 84
  source: /note=“Description of Artificial Sequence Synthetic polypeptide”

SEQ ID NO: 99 - protein, 124 aa, Artificial Sequence; identical to SEQ ID NO: 70
  source: /note=“Description of Artificial Sequence Synthetic polypeptide”

SEQ ID NO: 100 - protein, 124 aa, Artificial Sequence; identical to SEQ ID NO: 71
  source: /note=“Description of Artificial Sequence Synthetic polypeptide”

SEQ ID NO: 101 - protein, 124 aa, Artificial Sequence; identical to SEQ ID NO: 72
  source: /note=“Description of Artificial Sequence Synthetic polypeptide”

SEQ ID NO: 102 - protein, 124 aa, Artificial Sequence; identical to SEQ ID NO: 74
  source: /note=“Description of Artificial Sequence Synthetic polypeptide”

SEQ ID NO: 103 - protein, 124 aa, Artificial Sequence; identical to SEQ ID NO: 75
  source: /note=“Description of Artificial Sequence Synthetic polypeptide”

SEQ ID NO: 104 - protein, 124 aa, Artificial Sequence; identical to SEQ ID NO: 76
  source: /note=“Description of Artificial Sequence Synthetic polypeptide”

SEQ ID NO: 105 - protein, 124 aa, Artificial Sequence; identical to SEQ ID NO: 77
  source: /note=“Description of Artificial Sequence Synthetic polypeptide”

SEQ ID NO: 106 - protein, 124 aa, Artificial Sequence; identical to SEQ ID NO: 78
  source: /note=“Description of Artificial Sequence Synthetic polypeptide”

SEQ ID NO: 107 - protein, 124 aa, Artificial Sequence; identical to SEQ ID NO: 71
  source: /note=“Description of Artificial Sequence Synthetic polypeptide”

SEQ ID NO: 108 - protein, 124 aa, Artificial Sequence; identical to SEQ ID NO: 80
  source: /note=“Description of Artificial Sequence Synthetic polypeptide”

SEQ ID NO: 109 - protein, 123 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYNEKFKDRVTITVDKSTSTAYMELSSLRSEDTAVYYCARSGPYSYYAGGYALDYWGQTLVTVSS

SEQ ID NO: 110 - protein, 124 aa, Artificial Sequence; identical to SEQ ID NO: 82
  source: /note=“Description of Artificial Sequence Synthetic polypeptide”

SEQ ID NO: 111 - protein, 124 aa, Artificial Sequence; identical to SEQ ID NO: 70
  source: /note=“Description of Artificial Sequence Synthetic polypeptide”

SEQ ID NO: 112 - protein, 124 aa, Artificial Sequence; identical to SEQ ID NO: 84
  source: /note=“Description of Artificial Sequence Synthetic polypeptide”

SEQ ID NO: 113 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSTYYNEKFKDRVRITVDKSTSTAYMELSSLRSEDTAVYYCARSGPYSYYAGGYALDYWGQGTLVTVSS

SEQ ID NO: 114 - protein, 124 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSTYYNEMFKDRVRITVDKSTSTAYMELSSLRSEDTAVYYCARSGPYSYYAGGYALDYWGQGTLVTVSS

SEQ ID NO: 115 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSTYYNEKFKDRVRITVDKSTSTAYMELSSLRSEDTAVYYCARSGPYSYYAGGYALDYWGQGTLVTVSS

SEQ ID NO: 116 - protein, 124 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSTYYNEMFKDRVRITVDKSTSTAYMELSSLRSEDTAVYYCARSGPYSYYAGGYALDYWGQGTLVTVSS

SEQ ID NO: 117 - protein, 11 aa, Artificial Sequence
  source: /note=“Description of Artificial Sequence Synthetic peptide”
RASQSISSYLA

SEQ ID NO: 118 - protein, 11 aa, Artificial Sequence
  source: /note=“Description of Artificial Sequence Synthetic peptide”
RASENINSYLA

SEQ ID NO: 119 - protein, 11 aa, Artificial Sequence
  source: /note=“Description of Artificial Sequence Synthetic peptide”
RTSQNINSYLA

SEQ ID NO: 120 - protein, 11 aa, Artificial Sequence
  source: /note=“Description of Artificial Sequence Synthetic peptide”
RTSESINSYLA

SEQ ID NO: 121 - protein, 11 aa, Artificial Sequence
  source: /note=“Description of Artificial Sequence Synthetic peptide”
RASQSINSYLA

SEQ ID NO: 122 - protein, 11 aa, Artificial Sequence
  source: /note=“Description of Artificial Sequence Synthetic peptide”
RTSESISSYLA

SEQ ID NO: 123 - protein, 11 aa, Artificial Sequence
  source: /note=“Description of Artificial Sequence Synthetic peptide”
RASENISSYLA

SEQ ID NO: 124 - protein, 11 aa, Artificial Sequence
  source: /note=“Description of Artificial Sequence Synthetic peptide”
RTSQNISSYLA

SEQ ID NO: 125 - protein, 11 aa, Artificial Sequence
  source: /note=“Description of Artificial Sequence Synthetic peptide”
RTSQSINSYLA

SEQ ID NO: 126 - protein, 11 aa, Artificial Sequence
  source: /note=“Description of Artificial Sequence Synthetic peptide”
RTSENISSYLA

SEQ ID NO: 127 - protein, 6 aa, Artificial Sequence
  source: /note=“Description of Artificial Sequence Synthetic peptide”
QSISSY

SEQ ID NO: 128 - protein, 6 aa, Artificial Sequence
  source: /note=“Description of Artificial Sequence Synthetic peptide”
QNINSY

SEQ ID NO: 129 - protein, 6 aa, Artificial Sequence
  source: /note=“Description of Artificial Sequence Synthetic peptide”
ESINSY

SEQ ID NO: 130 - protein, 6 aa, Artificial Sequence
  source: /note=“Description of Artificial Sequence Synthetic peptide”
QSINSY

SEQ ID NO: 131 - protein, 6 aa, Artificial Sequence
  source: /note=“Description of Artificial Sequence Synthetic peptide”
ESISSY

SEQ ID NO: 132 - protein, 6 aa, Artificial Sequence
  source: /note=“Description of Artificial Sequence Synthetic peptide”
ENISSY

SEQ ID NO: 133 - protein, 6 aa, Artificial Sequence
  source: /note=“Description of Artificial Sequence Synthetic peptide”
QNISSY

SEQ ID NO: 134 - protein, 11 aa, Artificial Sequence
  source: /note=“Description of Artificial Sequence Synthetic peptide”
  VARIANT (2)..(2): /replace=“T”
  VARIANT (4)..(4): /replace=“E”
  VARIANT (5)..(5): /replace=“N”
  VARIANT (7)..(7): /replace=“S”
  SITE (1)..(11): /note=“Variant residues given in the sequence have no preference with respect to those in the annotations for variant positions”
RASQSINSYLA

SEQ ID NO: 135 - protein, 6 aa, Artificial Sequence
  source: /note=“Description of Artificial Sequence Synthetic peptide”
  VARIANT (1)..(1): /replace=“E”
  VARIANT (2)..(2): /replace=“N”
  VARIANT (4)..(4): /replace=“N”
  SITE (1)..(6): /note=“Variant residues given in the sequence have no preference with respect to those in the annotations for variant positions”
QSISSY

SEQ ID NO: 136 - protein, 7 aa, Artificial Sequence
  source: /note=“Description of Artificial Sequence Synthetic peptide”
YAKSLAE

SEQ ID NO: 137 - protein, 7 aa, Artificial Sequence
  source: /note=“Description of Artificial Sequence Synthetic peptide”
YASSLAE

SEQ ID NO: 138 - protein, 7 aa, Artificial Sequence
  source: /note=“Description of Artificial Sequence Synthetic peptide”
YASTLAE

SEQ ID NO: 139 - protein, 3 aa, Artificial Sequence
  source: /note=“Description of Artificial Sequence Synthetic peptide”
YAS

SEQ ID NO: 140 - protein, 7 aa, Artificial Sequence
  source: /note=“Description of Artificial Sequence Synthetic peptide”
  VARIANT (3)..(3): /replace=“K”
  VARIANT (4)..(4): /replace=“T”
  SITE (1)..(7): /note=“Variant residues given in the sequence have no preference with respect to those in the annotations for variant positions”
YASSLAE

SEQ ID NO: 141 - protein, 3 aa, Artificial Sequence
  source: /note=“Description of Artificial Sequence Synthetic peptide”
  VARIANT (3)..(3): /replace=“S”
  SITE (1)..(3): /note=“Variant residues given in the sequence have no preference with respect to those in the annotations for variant positions”
YAK

SEQ ID NO: 142 - protein, 5 aa, Artificial Sequence
  source: /note=“Description of Artificial Sequence Synthetic peptide”
TYWIN

SEQ ID NO: 143 - protein, 5 aa, Artificial Sequence
  source: /note=“Description of Artificial Sequence Synthetic peptide”
TYWIS

SEQ ID NO: 144 - protein, 10 aa, Artificial Sequence
  source: /note=“Description of Artificial Sequence Synthetic peptide”
GYIFLTYWIN

SEQ ID NO: 145 - protein, 10 aa, Artificial Sequence
  source: /note=“Description of Artificial Sequence Synthetic peptide”
GYIFLTYWIS

SEQ ID NO: 146 - protein, 5 aa, Artificial Sequence
  source: /note=“Description of Artificial Sequence Synthetic peptide”
  VARIANT (4)..(4): /replace=“M”
  VARIANT (5)..(5): /replace=“N”
  SITE (1)..(5): /note=“Variant residues given in the sequence have no preference with respect to those in the annotations for variant positions”
TYWIS

SEQ ID NO: 147 - protein, 11 aa, Artificial Sequence
  source: /note=“Description of Artificial Sequence Synthetic peptide”
  VARIANT (9)..(9): /replace=“I”
  VARIANT (11)..(11): /replace=“S”
  SITE (1)..(11): /note=“Variant residues given in the sequence have no preference with respect to those in the annotations for variant positions”
GYIFLTYWMNN

SEQ ID NO: 148 - protein, 17 aa, Artificial Sequence
  source: /note=“Description of Artificial Sequence Synthetic peptide”
QIFPASGSAYYAQKFQG

SEQ ID NO: 149 - protein, 17 aa, Artificial Sequence
  source: /note=“Description of Artificial Sequence Synthetic peptide”
QIFPASGSAYYNQKFKG

SEQ ID NO: 150 - protein, 17 aa, Artificial Sequence
  source: /note=“Description of Artificial Sequence Synthetic peptide”
QIFPASDSTYYAQMFQD

SEQ ID NO: 151 - protein, 17 aa, Artificial Sequence
  source: /note=“Description of Artificial Sequence Synthetic peptide”
QIFPASGSTYYNEKFKG

SEQ ID NO: 152 - protein, 17 aa, Artificial Sequence
  source: /note=“Description of Artificial Sequence Synthetic peptide”
QIFPASGSTYYAQKFQD

SEQ ID NO: 153 - protein, 17 aa, Artificial Sequence
  source: /note=“Description of Artificial Sequence Synthetic peptide”
QIFPASDSTYYNQKFKG

SEQ ID NO: 154 - protein, 17 aa, Artificial Sequence
  source: /note=“Description of Artificial Sequence Synthetic peptide”
QIFPASDSAYYNEKFKG

SEQ ID NO: 155 - protein, 17 aa, Artificial Sequence
  source: /note=“Description of Artificial Sequence Synthetic peptide”
QIFPASDSAYYAQKFQD

SEQ ID NO: 156 - protein, 17 aa, Artificial Sequence
  source: /note=“Description of Artificial Sequence Synthetic peptide”
QIFPASGSAYYNEKFKD

SEQ ID NO: 157 - protein, 17 aa, Artificial Sequence
  source: /note=“Description of Artificial Sequence Synthetic peptide”
QIFPASDSAYYNQMFKD

SEQ ID NO: 158 - protein, 17 aa, Artificial Sequence
  source: /note=“Description of Artificial Sequence Synthetic peptide”
QIFPASDSTYYAQMFQD

SEQ ID NO: 159 - protein, 17 aa, Artificial Sequence
  source: /note=“Description of Artificial Sequence Synthetic peptide”
QIFPASGSTYYNEKFKD

SEQ ID NO: 160 - protein, 17 aa, Artificial Sequence
  source: /note=“Description of Artificial Sequence Synthetic peptide”
QIFPASGSTYYNEMFKD

SEQ ID NO: 161 - protein, 6 aa, Artificial Sequence
  source: /note=“Description of Artificial Sequence Synthetic peptide”
FPASGS

SEQ ID NO: 162 - protein, 8 aa, Artificial Sequence
  source: /note=“Description of Artificial Sequence Synthetic peptide”
IFPASGSA

SEQ ID NO: 163 - protein, 8 aa, Artificial Sequence
  source: /note=“Description of Artificial Sequence Synthetic peptide”
IFPASGST

SEQ ID NO: 164 - protein, 8 aa, Artificial Sequence
  source: /note=“Description of Artificial Sequence Synthetic peptide”
IFPASDSA

SEQ ID NO: 165 - protein, 17 aa, Artificial Sequence
  source: /note=“Description of Artificial Sequence Synthetic peptide”
  VARIANT (7)..(7): /replace=“D”
  VARIANT (9)..(9): /replace=“T”
  VARIANT (12)..(12): /replace=“N”
  VARIANT (13)..(13): /replace=“E”
  VARIANT (14)..(14): /replace=“M”
  VARIANT (16)..(16): /replace=“Q”
  VARIANT (17)..(17): /replace=“D”
  SITE (1)..(17): /note=“Variant residues given in the sequence have no preference with respect to those in the annotations for variant positions”
QIFPASGSAYYAQKFKG

SEQ ID NO: 166 - protein, 6 aa, Artificial Sequence
  source: /note=“Description of Artificial Sequence Synthetic peptide”
  VARIANT (5)..(5): /replace=“D”
  SITE (1)..(6): /note=“Variant residues given in the sequence have no preference with respect to those in the annotations for variant positions”
FPASGS

SEQ ID NO: 167 - protein, 8 aa, Artificial Sequence
  source: /note=“Description of Artificial Sequence Synthetic peptide”
  VARIANT (6)..(6): /replace=“G”
  VARIANT (8)..(8): /replace=“A”
  SITE (1)..(8): /note=“Variant residues given in the sequence have no preference with respect to those in the annotations for variant positions”
IFPASDST

SEQ ID NO: 168 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 107 aa, Artificial Sequence [sequence omitted]

SEQ ID NO: 170 - protein, 214 aa, Artificial Sequence
  source: /note=“Description of Artificial Sequence Synthetic polypeptide”
DIQMTQSPASLSASVGETVTITCRTSENINSYLAWYQQKQGKSPQLLVHYAKTLAEGVPSRFSGSGSGTQFSLKINSLKPEDFGSYYCQHHYGTSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 214 aa, Artificial Sequence
  source: /note=“Description of Artificial Sequence Synthetic polypeptide”
DIQMTQSPSSLSASVGDRVTITCRASQSISSYLAWYQQKPGKAPKLLIHYAKSLAEGVPSRFSGSGSGTDFTLTISSLQPEDFATYYCQHHYGTSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 214 aa, Artificial Sequence; identical to SEQ ID NO: 171
  source: /note=“Description of Artificial Sequence Synthetic polypeptide”

SEQ ID NO: 173 - protein, 214 aa, Artificial Sequence; identical to SEQ ID NO: 171
  source: /note=“Description of Artificial Sequence Synthetic polypeptide”

SEQ ID NO: 174 - protein, 214 aa, Artificial Sequence
  source: /note=“Description of Artificial Sequence Synthetic polypeptide”
DIQMTQSPSSLSASVGDRVTITCRASQSISSYLAWYQQKPGKAPKLLIHYASSLAEGVPSRFSGSGSGTDFTLTISSLQPEDFATYYCQHHYGTSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 214 aa, Artificial Sequence; identical to SEQ ID NO: 174
  source: /note=“Description of Artificial Sequence Synthetic polypeptide”

SEQ ID NO: 176 - protein, 214 aa, Artificial Sequence; identical to SEQ ID NO: 174
  source: /note=“Description of Artificial Sequence Synthetic polypeptide”

SEQ ID NO: 177 - protein, 453 aa, Artificial Sequence
  source: /note=“Description of Artificial Sequence Synthetic polypeptide”
QVHLQQSGPELVRPGTSVKISCEASGYIFLTYWMNWVKQRPGQGLEWIGQIFPASDSTYYNEMFKDKARFTVDKSSSTAYMQFSSLTSEDTAVYFCARSGPYSYYAGGYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 453 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYNQKFKGRVTITADKSTSTAYMELSSLRSEDTAVYYCARSGPYSYYAGGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 453 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASDSTYYNQKFKGRVTITADKSTSTAYMELSSLRSEDTAVYYCARSGPYSYYAGGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 453 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSAYYAQKFQGRVTITADKSTSTAYMELSSLRSEDTAVYYCARSGPYSYYAGGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 453 aa, Artificial Sequence
  source: /note=“Description of Artificial Sequence Synthetic polypeptide”
QVQLVQSGAEVKKPGSSVKVSCKASGYIFLTYWMNWVRQAPGQGLEWMGQIFPASGSAYYAQKFQGRVTITVDKSTSTAYMELSSLRSEDTAVYYCARSGPYSYYAGGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 453 aa, Artificial Sequence; identical to SEQ ID NO: 178
  source: /note=“Description of Artificial Sequence Synthetic polypeptide”

SEQ ID NO: 183 - protein, 453 aa, Artificial Sequence
  source: /note=“Description of Artificial Sequence Synthetic polypeptide”
QVQLVQSGAEVKKPGSSVKVSCKASGYIFLTYWINWVRQAPGQGLEWMGQIFPASGSTYYNEKFKGRVTITADKSTSTAYMELSSLRSEDTAVYYCARSGPYSYYAGGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 347 aa, Homo sapiens
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGAT

SEQ ID NO: 185 - protein, 114 aa, Homo sapiens
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQK

SEQ ID NO: 186 - protein, 100 aa, Homo sapiens
DSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVV

SEQ ID NO: 187 - protein, 133 aa, Homo sapiens
GSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGAT

SEQ ID NO: 188 - protein, 81 aa, Homo sapiens
GSPKNAVPPVIHSPNDHVVYEKEPGEELLIPCTVYFSFLMDSRNEVWWTIDGKKPDDITIDVTINESISHSRTEDETRTQI

SEQ ID NO: 189 - protein, 39 aa, Homo sapiens
GSPKNAVPPVIHSPNDHVVYEKEPGEELLIPCTVYFSFL

SEQ ID NO: 190 - protein, 15 aa, Homo sapiens
SISHSRTEDETRTQI

SEQ ID NO: 191 - protein, 37 aa, Homo sapiens
TRTLTVKVVGSPKNAVPPVIHSPNDHVVYEKEPGEEL

SEQ ID NO: 192 - protein, 48 aa, Homo sapiens
TRTLTVKVVGSPKNAVPPVIHSPNDHVVYEKEPGEELLIPCTVYFSFL

SEQ ID NO: 193 - protein, 10 aa, Artificial Sequence
  source: /note=“Description of Artificial Sequence Synthetic peptide”
  VARIANT (9)..(9): /replace=“M”
  SITE (1)..(10): /note=“Variant residues given in the sequence have no preference with respect to those in the annotations for variant positions”
GYIFLTYWIN

SEQ ID NO: 194 - protein, 10 aa, Artificial Sequence
  source: /note=“Description of Artificial Sequence Synthetic peptide”
  VARIANT (9)..(9): /replace=“I”
  VARIANT (10)..(10): /replace=“S”
  SITE (1)..(10): /note=“Variant residues given in the sequence have no preference with respect to those in the annotations for variant positions”
GYIFLTYWMN